(19) United States
(12) Patent Application Publication
(10) Pub. No.: US 2024/0032417 A1
(43) Pub. Date: Jan. 25, 2024

(54) ORGANIC ELECTROLUMINESCENT MATERIAL AND DEVICE THEREOF

(71) Applicant: BEIJING SUMMER SPROUT TECHNOLOGY CO., LTD., Beijing (CN)

(72) Inventors: Feng LI, Beijing (CN); Yang WANG, Beijing (CN); Jianfei YAO, Beijing (CN); Gang YANG, Beijing (CN); Chi Yuen Raymond KWONG, Hong Kong (CN); Chuanjun XIA, Beijing (CN)

(21) Appl. No.: 18/303,351

(22) Filed: Apr. 19, 2023

(30) Foreign Application Priority Data
Apr. 22, 2022 (CN) 202210410969.0

(51) Int. Cl. H10K 85/60; C07D 405/04; H10K 85/30; C09K 11/02; C09K 11/06

(52) U.S. Cl. CPC H10K 85/654; C07D 405/04; H10K 85/615; H10K 85/6574; H10K 85/6572; H10K 85/342; C09K 11/02; C09K 11/06; H10K 50/12

(57) ABSTRACT

Provided are an organic electroluminescent material and a device comprising the same. The organic electroluminescent material is a compound having a structure of Formula 1. These new compounds can be applied in organic electroluminescent devices, for example, as host materials, transport materials (e.g., electron transport materials), etc., in organic electroluminescent devices, and can provide better device performance and especially improve the device lifetime. Further provided are an organic electroluminescent device including the compound and a compound composition including the compound.

### CROSS-REFERENCE TO RELATED APPLICATION(S)

This application claims priority to Chinese Patent Application No. 202210410969.0 filed on Apr. 22, 2022, the disclosure of which is incorporated herein by reference in its entirety.

### TECHNICAL FIELD

The present disclosure relates to compounds for organic electronic devices such as organic electroluminescent devices. In particular, the present disclosure relates to a compound having a structure of Formula 1, an organic electroluminescent device comprising the compound and a compound composition comprising the compound.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which includes an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may include multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may include one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

CN113993863A discloses an organic compound having the following formula and an organic light-emitting device including the same:

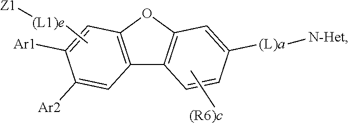

wherein N-Het is a monocyclic or polycyclic C2 to C60 heterocyclic group substituted or unsubstituted and comprising one or more Nheterocyclic group; Z1 is substituted or unsubstituted C6 to C60 aryl group, or is represented by Formula A:

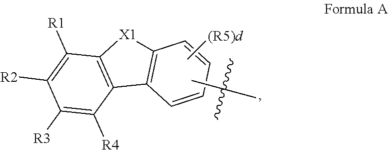

wherein X1 is O, S, CR₁₁R₁₂ or NR₁₃. This application further discloses the following compounds:

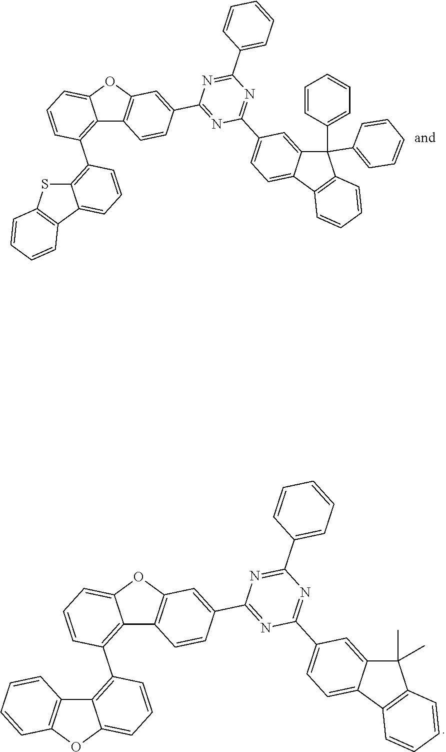 [SMILES: c1ccc(-c2nc(-c3ccc4c(c3)C(c3ccccc3)(c3ccccc3)c3ccccc3-4)nc(-c3ccc4c(c3)oc3cccc(-c5cccc6c5sc5ccccc56)c34)n2)cc1 ; CC1(C)c2ccccc2-c2ccc(-c3nc(-c4ccccc4)nc(-c4ccc5c(c4)oc4cccc(-c6cccc7oc8ccccc8c67)c45)n3)cc21]

This application does not disclose and teach the compound having a structure of Formula 1 of the present application and its use in organic electroluminescent devices.

WO2020009381A1 discloses an organic compound having the following formula and an organic light-emitting device including the same:

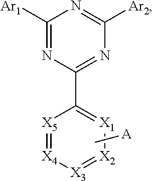

wherein X₁ to X₅ represent, at each occurrence identically or differently, N or CR, and one of X₁ to X₅ is N; A is a substituent represented by Formula 2:

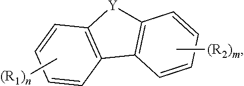

wherein Y is selected from O, S or CR₃R₄. This application further discloses the following compounds:

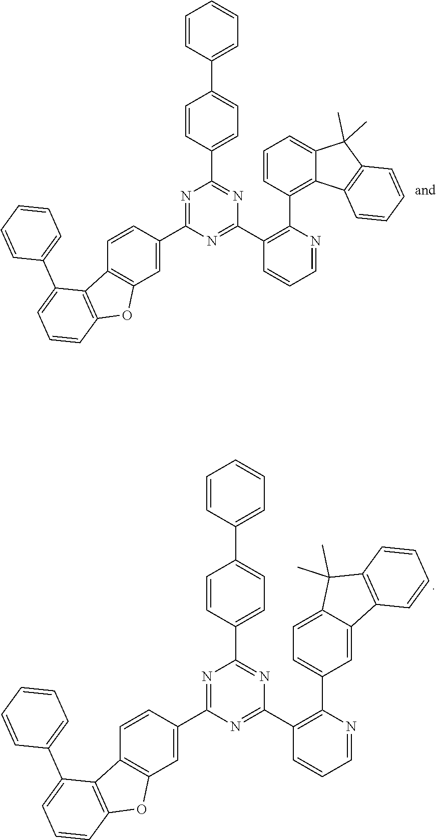 [SMILES: CC1(C)c2ccccc2-c2c(-c3ncccc3-c3nc(-c4ccc(-c5ccccc5)cc4)nc(-c4ccc5c(c4)oc4cccc(-c6ccccc6)c45)n3)cccc21 ; CC1(C)c2ccccc2-c2cc(-c3ncccc3-c3nc(-c4ccc(-c5ccccc5)cc4)nc(-c4ccc5c(c4)oc4cccc(-c6ccccc6)c45)n3)ccc21]

This application discloses a compound in which a group having a structure of Formula 2 and triazine are joined with a bridging group including a pyridyl group but does not disclose and teach the compound having a structure of Formula 1 of the present application and its use in organic electroluminescent devices.

CN113260615A discloses an organic compound having the following formula and an organic light-emitting device including the same:

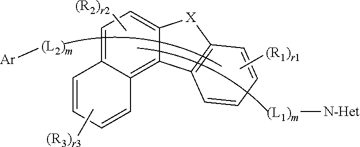

wherein X is O, S or NR₂₁; Ar is a substituted or unsubstituted aryl group; a substituted or unsubstituted heteroaryl group; or a substituted or unsubstituted amine group; N-Het is a monocyclic or multicyclic heteroaryl group substituted or unsubstituted and including one or more N. This application further discloses the following compounds:

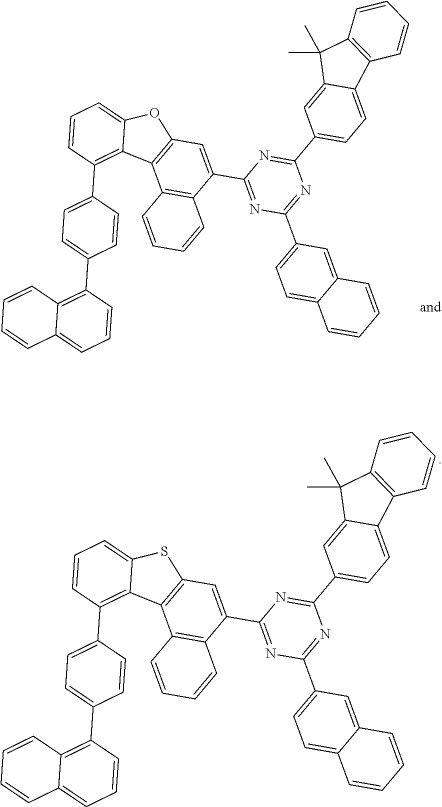 [SMILES: CC1(C)c2ccccc2-c2ccc(-c3nc(-c4ccc5ccccc5c4)nc(-c4cc5oc6cccc(-c7ccc(-c8cccc9ccccc89)cc7)c6c5c5ccccc45)n3)cc21 ; CC1(C)c2ccccc2-c2ccc(-c3nc(-c4ccc5ccccc5c4)nc(-c4cc5sc6cccc(-c7ccc(-c8cccc9ccccc89)cc7)c6c5c5ccccc45)n3)cc21]

This application only discloses compounds having a skeleton structure of benzodibenzofuran (benzodibenzothiophene or benzocarbazole) and does not disclose and teach compounds having a skeleton structure of dibenzofuran (dibenzothiophene or carbazole), in particular, the compound having a structure of Formula 1 of the present application and its use in organic electroluminescent devices.

WO2019132632A discloses an organic compound having the following formula and an organic light-emitting device including the same:

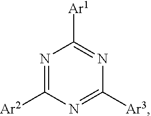

wherein Ar¹ is substituted or unsubstituted C6 to C60 aryl; Ar² and Ar³ are each selected from any of the following structures:

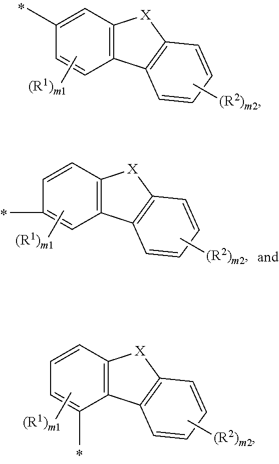

wherein X is O or S. This application further discloses the following compounds:

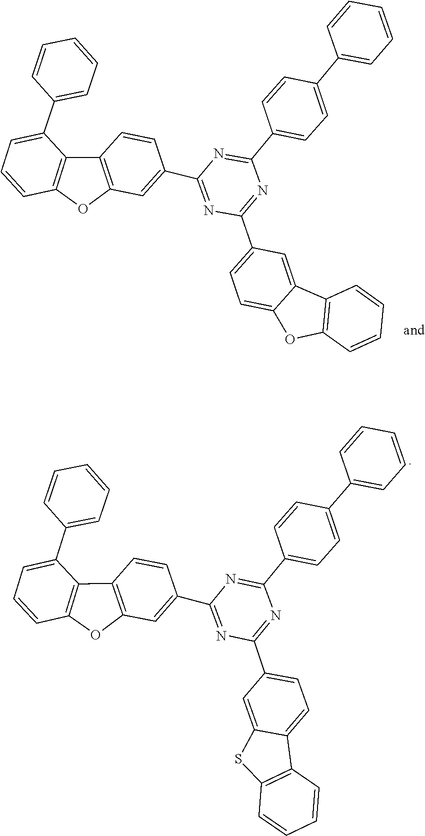 [SMILES: c1ccc(-c2ccc(-c3nc(-c4ccc5c(c4)oc4cccc(-c6ccccc6)c45)nc(-c4ccc5oc6ccccc6c5c4)n3)cc2)cc1 ; c1ccc(-c2ccc(-c3nc(-c4ccc5c(c4)oc4cccc(-c6ccccc6)c45)nc(-c4ccc5c(c4)sc4ccccc45)n3)cc2)cc1]

This application only discloses compounds including two dibenzofuran (or dibenzothiophene) and does not disclose and teach compounds including a fluorene group of the present application, in particular, the compound having a structure of Formula 1 of the present application and its use in organic electroluminescent devices.

CN111247650A discloses an organic light-emitting device, wherein the organic layer includes an organic compound having the following structure general formula:

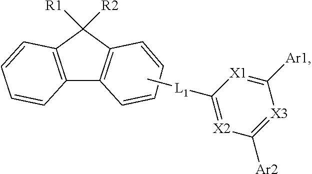

wherein at least one of X1 to X3 is N, and each remaining one is CH. This application further discloses the following compounds:

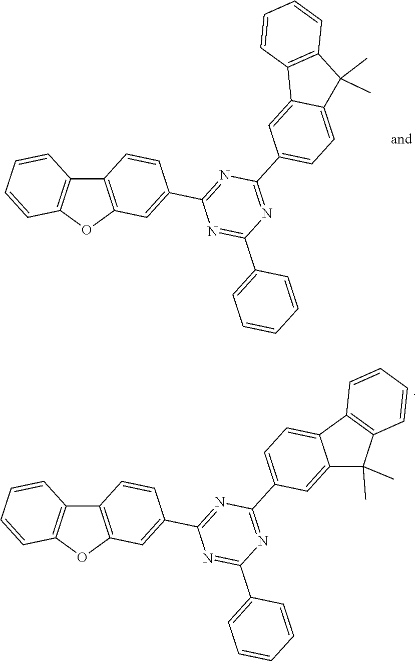 [SMILES: CC1(C)c2ccccc2-c2cc(-c3nc(-c4ccccc4)nc(-c4ccc5c(c4)oc4ccccc45)n3)ccc21 ; CC1(C)c2ccccc2-c2ccc(-c3nc(-c4ccccc4)nc(-c4ccc5c(c4)oc4ccccc45)n3)cc21]

This application does not disclose and teach the compound having a structure of Formula 1 of the present application and especially does not disclose and teach compounds having a substituent in a specific position on dibenzofuran, in particular, the compound having a structure of Formula 1 of the present application and its use in organic electroluminescent devices.

### SUMMARY

The present disclosure aims to provide a series of compounds having a structure of Formula 1 to solve at least part of the preceding problems. These new compounds can be applied in organic electroluminescent devices, for example, as host materials, transport materials (e.g., electron transport materials), etc., in organic electroluminescent devices, and can provide better device performance and especially improve the device lifetime.

According to an embodiment of the present disclosure, a compound having a structure of Formula 1 is disclosed:

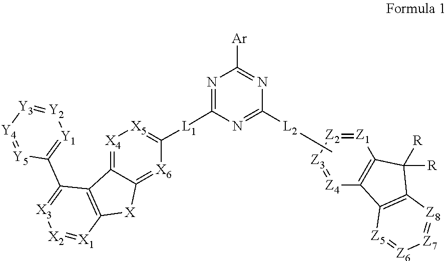

wherein X is selected from O, S or Se;
X₁ to X₆ are, at each occurrence identically or differently, selected from CRₓ or N;
Y₁ to Y₅ are, at each occurrence identically or differently, selected from CR_{y} or N;
Z₁ to Z₈ are, at each occurrence identically or differently, selected from C, CR_{z} or N, and one of Z₁ to Z₄ is selected from C and joined to L₂;
Ar is, at each occurrence identically or differently, selected from substituted or unsubstituted aryl having 6 to 30 carbon atoms, substituted or unsubstituted heteroaryl having 3 to 30 carbon atoms or combinations thereof,
L₁ is, at each occurrence identically or differently, selected from a single bond, substituted or unsubstituted arylene having 6 to 30 carbon atoms, substituted or unsubstituted heteroarylene having 3 to 12 carbon atoms or combinations thereof,
L₂ is, at each occurrence identically or differently, selected from a single bond or substituted or unsubstituted arylene having 6 to 30 carbon atoms;
R, Rₓ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a sulfinyl group, a sulfonyl group, a phosphino group, and combinations thereof,
adjacent substituents R can be optionally joined to form a ring;
adjacent substituents R_{z} can be optionally joined to form a ring;
adjacent substituents R_{y} can be optionally joined to form a carbocyclic ring or a heterocycle including one or more of N, Si, P, Ge and B atoms.

According to another embodiment of the present disclosure, an organic electroluminescent device is disclosed, wherein the organic electroluminescent device includes an anode, a cathode, and an organic layer disposed between the anode and the cathode, and at least one layer of the organic layer comprises the compound in the preceding embodiment.

According to yet another embodiment of the present disclosure, a compound composition is further disclosed, wherein the compound composition comprises the compound in the preceding embodiment.

The present disclosure provides a series of compounds having a structure of Formula 1. These new compounds can be applied in organic electroluminescent devices, can provide better device performance and especially improve the device lifetime, and can greatly the overall performance of the devices.

### BRIEF DESCRIPTION OF DRAWINGS

FIG. 1 is a schematic diagram of an organic light-emitting apparatus that may contain a compound and a compound composition disclosed herein.

FIG. 2 is a schematic diagram of another organic light-emitting apparatus that may contain a compound and a compound composition disclosed herein.

### DETAILED DESCRIPTION

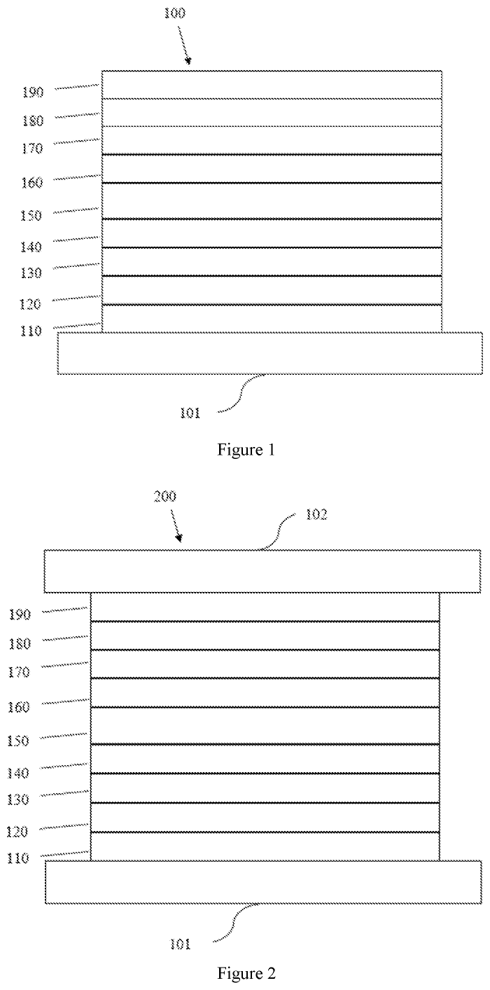

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small ΔE_{S-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcyclohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcyclohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butylmethylgermanylmethyl, triethylgermanylmethyl, triethylgermanylethyl, triisopropylgermanylmethyl, triisopropylgermanylethyl, trimethylsilylmethyl, trimethylsilylethyl, and trimethylsilylisopropyl, triisopropylsilylmethyl, triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butanedienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenyl, 4″-t-butyl-p-terphenyl-4-yl, o-cumenyl, m-cumenyl, p-cumenyl, 2,3-xylyl, 3,4-xylyl, 2,5-xylyl, mesityl, and m-quaterphenyl. Additionally, the aryl group may be optionally substituted.

Heterocyclic groups or heterocycle—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, alkylgermanyl, arylgermanyl, amino, acyl, carbonyl, a carboxylic acid group, an ester group, sulfinyl, sulfonyl, and phosphino may be substituted with one or more moieties selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl having 6 to 20 carbon atoms, unsubstituted alkylgermanyl having 3 to 20 carbon atoms, unsubstituted arylgermanyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen can also be replaced by their other stable isotopes. The replacement by other stable isotopes in the compounds may be preferred due to its enhancements of device efficiency and stability.

In the compounds mentioned in the present disclosure, multiple substitutions refer to a range that includes a di-substitution, up to the maximum available substitution. When substitution in the compounds mentioned in the present disclosure represents multiple substitution (including di-, tri-, and tetra-substitutions, etc.), that means the substituent may exist at a plurality of available substitution positions on its linking structure, the substituents present at a plurality of available substitution positions may be the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fused cyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

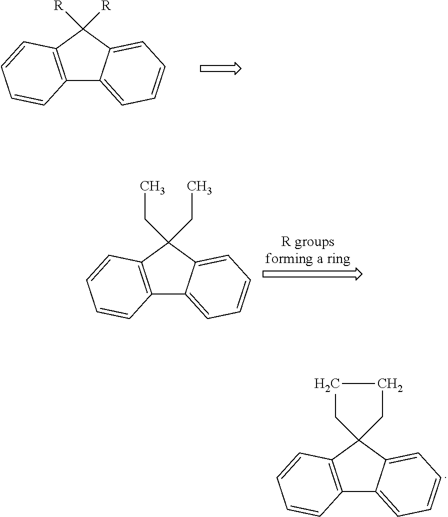

The expression that adjacent substituents can be optionally joined to form a ring is also intended to mean that two substituents bonded to carbon atoms which are directly bonded to each other are joined to each other via a chemical bond to form a ring, which can be exemplified by the following formula:

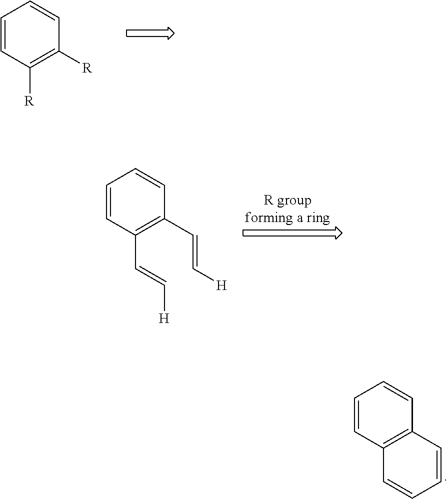

The expression that adjacent substituents can be optionally joined to form a ring is also intended to mean that two substituents bonded to a further distant carbon atom are joined to each other via a chemical bond to form a ring, which can be exemplified by the following formula:

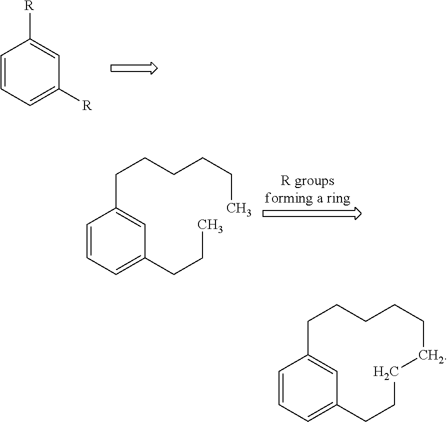

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

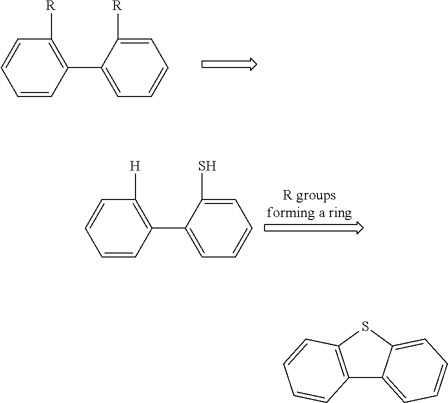

According to an embodiment of the present disclosure, a compound having a structure of Formula 1 is disclosed:

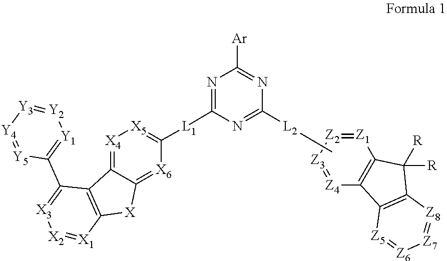

wherein X is selected from O, S or Se;
X₁ to X₆ are, at each occurrence identically or differently, selected from CRₓ or N;
Y₁ to Y₅ are, at each occurrence identically or differently, selected from CR_{y} or N;
Z₁ to Z₈ are, at each occurrence identically or differently, selected from C, CR_{z} or N, and one of Z₁ to Z₄ is selected from C and joined to L₂;
Ar is, at each occurrence identically or differently, selected from substituted or unsubstituted aryl having 6 to 30 carbon atoms, substituted or unsubstituted heteroaryl having 3 to 30 carbon atoms or combinations thereof,
L₁ is, at each occurrence identically or differently, selected from a single bond, substituted or unsubstituted arylene having 6 to 30 carbon atoms, substituted or unsubstituted heteroarylene having 3 to 12 carbon atoms or combinations thereof,
L₂ is, at each occurrence identically or differently, selected from a single bond or substituted or unsubstituted arylene having 6 to 30 carbon atoms;
R, Rₓ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof,
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a sulfinyl group, a sulfonyl group, a phosphino group, and combinations thereof,
adjacent substituents R can be optionally joined to form a ring;
adjacent substituents R_{z} can be optionally joined to form a ring;
adjacent substituents R_{y} can be optionally joined to form a carbocyclic ring or a heterocycle including one or more of N, Si, P, Ge and B atoms.

Herein, the expression that “adjacent substituents R can be optionally joined to form a ring” is intended to mean that adjacent two substituents R may be joined to form a ring. Obviously, it is also possible that none of these substituents are joined to form a ring.

Herein, the expression that “adjacent substituents R_{z} can be optionally joined to form a ring” is intended to mean that any one or more of groups of adjacent substituents, such as any two substituents R_{z}, may be joined to form a ring. Obviously, it is also possible that none of these substituents are joined to form a ring.

Herein, the expression that “adjacent substituents R_{y} can be optionally joined to form a carbocyclic ring or a heterocycle including one or more of N, Si, P, Ge and B atoms” is intended to mean that any one or more of groups of adjacent substituents, such as any two substituents R_{y}, may be joined to form a ring, wherein the ring may be a carbocyclic ring (which may be an aromatic or non-aromatic carbocyclic ring) or a heterocyclic ring (which may be an aromatic or non-aromatic heterocyclic ring) including at least one or more of N, Si, P, Ge and B atoms. Obviously, it is also possible that none of these substituents are joined to form a ring.

According to an embodiment of the present disclosure, adjacent substituents R_{y} can be optionally joined to form a carbocyclic ring.

According to an embodiment of the present disclosure, adjacent substituents R_{y} can be optionally joined to form an aromatic carbocyclic ring.

According to an embodiment of the present disclosure, adjacent substituents R_{y} can be optionally joined to form an aromatic ring.

According to an embodiment of the present disclosure, X is selected from O or S.

According to an embodiment of the present disclosure, X is O.

According to an embodiment of the present disclosure, X₁ to X₆ are, at each occurrence identically or differently, selected from CRₓ.

According to an embodiment of the present disclosure, X₁ to X₆ are, at each occurrence identically or differently, selected from CRₓ or N, and at least one of X₁ to X₆ is selected from N, for example, one or two of X₁ to X₆ are selected from N.

According to an embodiment of the present disclosure, Z₁ to Z₈ are, at each occurrence identically or differently, selected from C or CR_{z}, and one of Z₁ to Z₄ is selected from C and joined to L₂.

According to an embodiment of the present disclosure, Z₃ or Z₄ is selected from C and joined to L₂.

According to an embodiment of the present disclosure, Z₁ to Z₈ are, at each occurrence identically or differently, selected from C, CR_{z} or N, and one of Z₁ to Z₄ is selected from C and joined to L₂, wherein at least one of Z₁ to Z₈ is selected from N, for example, one or two of Z₁ to Z₈ are selected from N.

According to an embodiment of the present disclosure, L₁ is, at each occurrence identically or differently, selected from a single bond, substituted or unsubstituted arylene having 6 to 20 carbon atoms, substituted or unsubstituted heteroarylene having 3 to 12 carbon atoms or combinations thereof.

According to an embodiment of the present disclosure, L₁ is, at each occurrence identically or differently, selected from a single bond, substituted or unsubstituted arylene having 6 to 12 carbon atoms, substituted or unsubstituted heteroarylene having 3 to 12 carbon atoms or combinations thereof.

According to an embodiment of the present disclosure, L₁ is, at each occurrence identically or differently, selected from a single bond, substituted or unsubstituted phenylene, substituted or unsubstituted biphenylene, substituted or unsubstituted naphthylene, substituted or unsubstituted pyridylene or combinations thereof.

According to an embodiment of the present disclosure, L₁ is, at each occurrence identically or differently, selected from a single bond or substituted or unsubstituted phenylene.

According to an embodiment of the present disclosure, L₂ is, at each occurrence identically or differently, selected from a single bond or substituted or unsubstituted arylene having 6 to 20 carbon atoms.

According to an embodiment of the present disclosure, L₂ is, at each occurrence identically or differently, selected from a single bond or substituted or unsubstituted arylene having 6 to 12 carbon atoms.

According to an embodiment of the present disclosure, L₂ is, at each occurrence identically or differently, selected from a single bond, substituted or unsubstituted phenylene, substituted or unsubstituted biphenylene, substituted or unsubstituted naphthylene or combinations thereof.

According to an embodiment of the present disclosure, L₂ is, at each occurrence identically or differently, selected from a single bond or substituted or unsubstituted phenylene.

According to an embodiment of the present disclosure, Rₓ, R_{y} and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, cyano, and combinations thereof.

According to an embodiment of the present disclosure, Rₓ, R_{y} and R_{z} are, at each occurrence identically or differently, selected from the group consisting of: hydrogen, deuterium, halogen, substituted or unsubstituted alkyl having 1 to 20 carbon atoms, substituted or unsubstituted aryl having 6 to 20 carbon atoms, substituted or unsubstituted heteroaryl having 3 to 20 carbon atoms, cyano, and combinations thereof.

According to an embodiment of the present disclosure, Rₓ, R_{y} and R_{z} are, at each occurrence identically or differently, selected from the group consisting of: hydrogen, deuterium, halogen, substituted or unsubstituted aryl having 6 to 20 carbon atoms, substituted or unsubstituted heteroaryl having 3 to 20 carbon atoms, cyano, and combinations thereof.

According to an embodiment of the present disclosure, Rₓ, R_{y} and R_{z} are, at each occurrence identically or differently, selected from the group consisting of: hydrogen, deuterium, substituted or unsubstituted phenyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted carbazolyl, substituted or unsubstituted dibenzofuryl, substituted or unsubstituted dibenzothienyl, cyano, and combinations thereof.

According to an embodiment of the present disclosure, R is, at each occurrence identically or differently, selected from the group consisting of: hydrogen, deuterium, substituted or unsubstituted alkyl having 1 to 20 carbon atoms, substituted or unsubstituted cycloalkyl having 3 to 20 ring carbon atoms, substituted or unsubstituted aryl having 6 to 20 carbon atoms, substituted or unsubstituted heteroaryl having 3 to 20 carbon atoms, and combinations thereof.

According to an embodiment of the present disclosure, R is, at each occurrence identically or differently, selected from the group consisting of: hydrogen, deuterium, substituted or unsubstituted alkyl having 1 to 20 carbon atoms, substituted or unsubstituted aryl having 6 to 20 carbon atoms, and combinations thereof.

According to an embodiment of the present disclosure, R is, at each occurrence identically or differently, selected from the group consisting of: hydrogen, deuterium, substituted or unsubstituted alkyl having 1 to 6 carbon atoms, substituted or unsubstituted aryl having 6 to 12 carbon atoms, and combinations thereof.

According to an embodiment of the present disclosure, R is, at each occurrence identically or differently, selected from the group consisting of: hydrogen, deuterium, substituted or unsubstituted methyl, substituted or unsubstituted ethyl, substituted or unsubstituted propyl, substituted or unsubstituted butyl, substituted or unsubstituted pentyl, substituted or unsubstituted hexyl, substituted or unsubstituted cyclopentyl, substituted or unsubstituted adamantyl, substituted or unsubstituted cyclohexyl, substituted or unsubstituted phenyl, substituted or unsubstituted pyridyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted carbazolyl, substituted or unsubstituted dibenzofuryl, substituted or unsubstituted dibenzothienyl, and combinations thereof.

According to an embodiment of the present disclosure, Ar is, at each occurrence identically or differently, selected from substituted or unsubstituted aryl having 6 to 20 carbon atoms, substituted or unsubstituted heteroaryl having 3 to 20 carbon atoms or combinations thereof.

According to an embodiment of the present disclosure, Ar is, at each occurrence identically or differently, selected from substituted or unsubstituted aryl having 6 to 12 carbon atoms, substituted or unsubstituted heteroaryl having 3 to 12 carbon atoms or combinations thereof.

According to an embodiment of the present disclosure, Ar is, at each occurrence identically or differently, selected from the group consisting of: substituted or unsubstituted phenyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted carbazolyl, substituted or unsubstituted dibenzofuryl, substituted or unsubstituted dibenzothienyl, and combinations thereof.

According to an embodiment of the present disclosure, the compound is selected from the group consisting of Compound A-1 to Compound A-566, wherein for the specific structures of Compound A-1 to Compound A-566, reference is made to claim 9.

According to an embodiment of the present disclosure, hydrogens in Compound A-1 to Compound A-566 can be partially or fully substituted with deuterium.

According to an embodiment of the present disclosure, an organic electroluminescent device is disclosed, wherein the organic electroluminescent device includes an anode, a cathode, and an organic layer disposed between the anode and the cathode, and at least one layer of the organic layer comprises the compound in any one of the preceding embodiments.

According to an embodiment of the present disclosure, the organic layer comprising the compound is an electron transport layer, and the compound is an electron transporting compound.

According to an embodiment of the present disclosure, the organic layer comprising the compound is an emissive layer, the compound is a host compound, and the emissive layer at least includes a first metal complex.

According to an embodiment of the present disclosure, the first metal complex has a general formula of M(Lₐ)ₘ(L_{b})ₙ(L_{c})_{q};
the metal M is selected from a metal with a relative atomic mass greater than 40;
ligands Lₐ, L_{b} and L_{c} are a first ligand, a second ligand and a third ligand coordinated to the metal M, respectively, and ligands Lₐ, L_{b} and L_{c} may be the same or different;
ligands Lₐ, L_{b} and L_{c} can be optionally joined to form a multidentate ligand; for example, any two of Lₐ, L_{b} and L_{c} may be joined to form a tetradentate ligand; in another example, Lₐ, L_{b} and L_{c} may be joined to each other to form a hexadentate ligand; in another example, none of Lₐ, L_{b} and L_{c} are joined so that no multidentate ligand is formed;
m is 1, 2 or 3, n is 0, 1 or 2, q is 0, 1 or 2, and the sum of m, n and q is equal to an oxidation state of the metal M; when m is greater than or equal to 2, a plurality of Lₐ may be the same or different; when n is 2, two L_{b} may be the same or different; when q is 2, two L_{c} may be the same or different;
the ligand Lₐ has a structure represented by Formula 2:

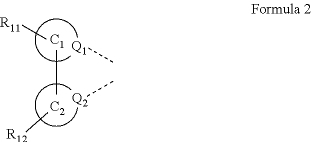

wherein the ring C₁ and the ring C₂ are, at each occurrence identically or differently, selected from an aromatic ring having 5 to 30 ring atoms, a heteroaromatic ring having 5 to 30 ring atoms or combinations thereof,
Q₁ and Q₂ are, at each occurrence identically or differently, selected from C or N;
R₁₁ and R₁₂ represent, at each occurrence identically or differently, mono-substitution, multiple substitutions or non-substitution;
R₁₁ and R₁₂ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof,
adjacent substituents R₁₁, R₁₂ can be optionally joined to form a ring;
ligands L_{b} and L_{c} are, at each occurrence identically or differently, selected from a monoanionic bidentate ligand.

According to an embodiment of the present disclosure, ligands L_{b} and L_{c} are, at each occurrence identically or differently, selected from the group consisting of the following structures:

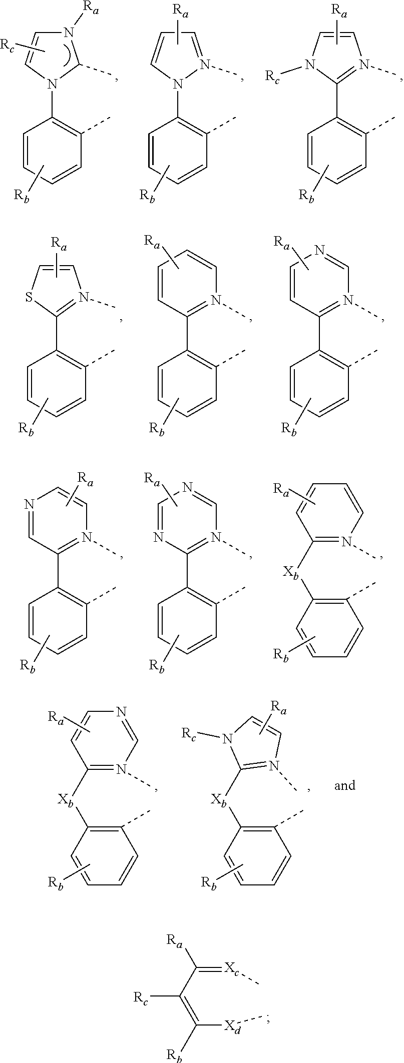

wherein
Rₐ and R_{b} represent, at each occurrence identically or differently, mono-substitution, multiple substitutions or non-substitution;
X_{b} is, at each occurrence identically or differently, selected from the group consisting of: O, S, Se, NR_{N1}, and CR_{C1}R_{C2};
X_{c} and X_{d} are, at each occurrence identically or differently, selected from the group consisting of: O, S, Se, and NR_{N2},
Rₐ, R_{b}, R_{c}, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof,
adjacent substituents Rₐ, R_{b}, R_{c}, R_{N1}, R_{N2}, R_{C1} and R_{C2} can be optionally joined to form a ring.

In this embodiment, the expression that “adjacent substituents Rₐ, R_{b}, R_{c}, R_{N1}, R_{N2}, R_{C1} and R_{C2} can be optionally joined to form a ring” is intended to mean that any one or more of groups of adjacent substituents, such as two substituents Rₐ, two substituents R_{b}, substituents Rₐ and R_{b}, substituents Rₐ and R_{c}, substituents R_{b} and R_{c}, substituents Rₐ and R_{N1}, substituents R_{b} and R_{N1}, substituents Rₐ and R_{C1}, substituents Rₐ and R_{C2}, substituents R_{b} and R_{C1}, substituents R_{b} and R_{C2}, substituents R_{C1} and R_{C2}, substituents Rₐ and R_{N2}, and substituents R_{b} and R_{N2}, may be joined to form a ring. Obviously, it is also possible that none of these substituents are joined to form a ring.

According to an embodiment of the present disclosure, the first metal complex has a general structure of Ir(Lₐ)ₘ(L_{b})₃₋ₘ and a structure represented by Formula 5:

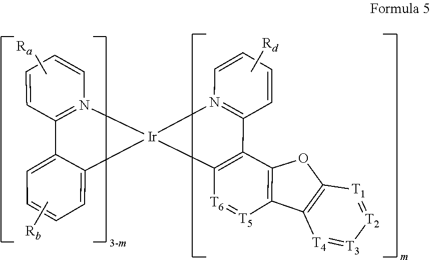

wherein
m is 0, 1, 2 or 3; when m is 2 or 3, a plurality of Lₐ are the same or different; when m is 0 or 1, a plurality of L_{b} are the same or different;
T₁ to T₆ are, at each occurrence identically or differently, selected from CR_{T} or N;
Rₐ, R_{b} and R_{d} represent, at each occurrence identically or differently, mono-substitution, multiple substitutions or non-substitution;
Rₐ, R_{b}, R_{d} and R_{T} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof,
adjacent substituents Rₐ, R_{b} can be optionally joined to form a ring;
adjacent substituents R_{d}, R_{T} can be optionally joined to form a ring.

In this embodiment, the expression that “adjacent substituents Rₐ, R_{b} can be optionally joined to form a ring” is intended to mean that any one or more of groups of adjacent substituents, such as two substituents Rₐ, two substituents R_{b}, and substituents Rₐ and R_{b}, may be joined to form a ring. Obviously, it is also possible that none of these substituents are joined to form a ring.

In this embodiment, the expression that “adjacent substituents R_{d}, R_{T} can be optionally joined to form a ring” is intended to mean that any one or more of groups of adjacent substituents, such as two substituents R_{T}, and two substituents R_{d}, may be joined to form a ring. Obviously, it is also possible that none of these substituents are joined to form a ring.

According to an embodiment of the present disclosure, at least one of T₁ to T₆ is selected from CR_{T}, and R_{T} is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms or substituted or unsubstituted heteroaryl having 3 to 30 carbon atoms.

According to an embodiment of the present disclosure, at least one of T₁ to T₆ is selected from CR_{T}, and R_{T} is selected from fluorine or cyano.

According to an embodiment of the present disclosure, at least two of T₁ to T₆ are selected from CR_{T}, one R_{T} is selected from fluorine or cyano, and the other R_{T} is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms or substituted or unsubstituted heteroaryl having 3 to 30 carbon atoms.

According to an embodiment of the present disclosure, T₁ to T₆ are, at each occurrence identically or differently, selected from CR_{T} or N, and at least one of T₁ to T₆ is selected from N, for example, one or two of T₁ to T₆ are selected from N.

According to an embodiment of the present disclosure, the first metal complex is selected from the group consisting of, but not limited to, GD1 to GD76, wherein the specific structures of GD1 to GD76 are described below:

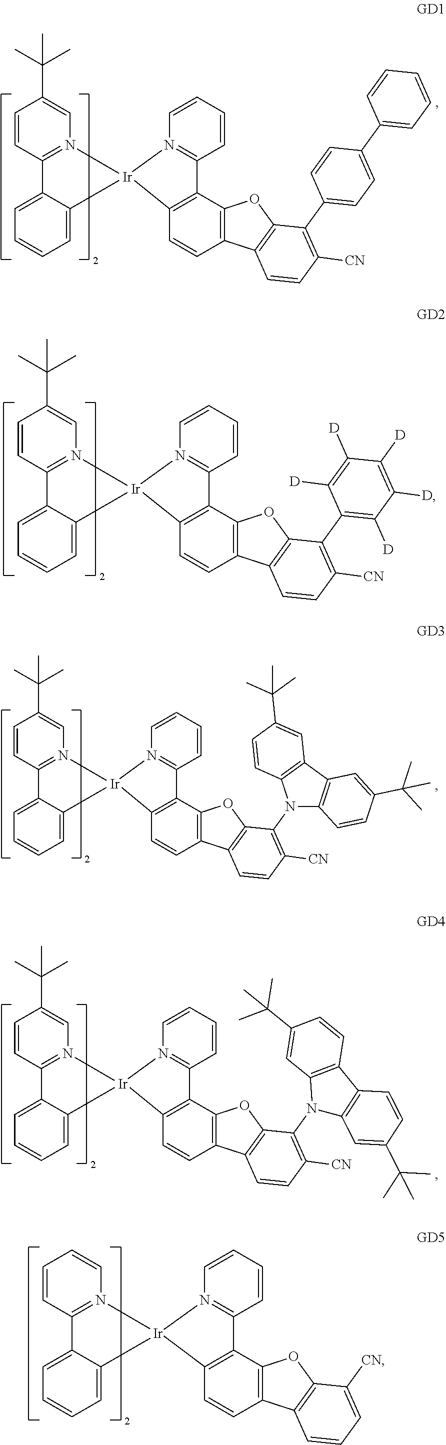

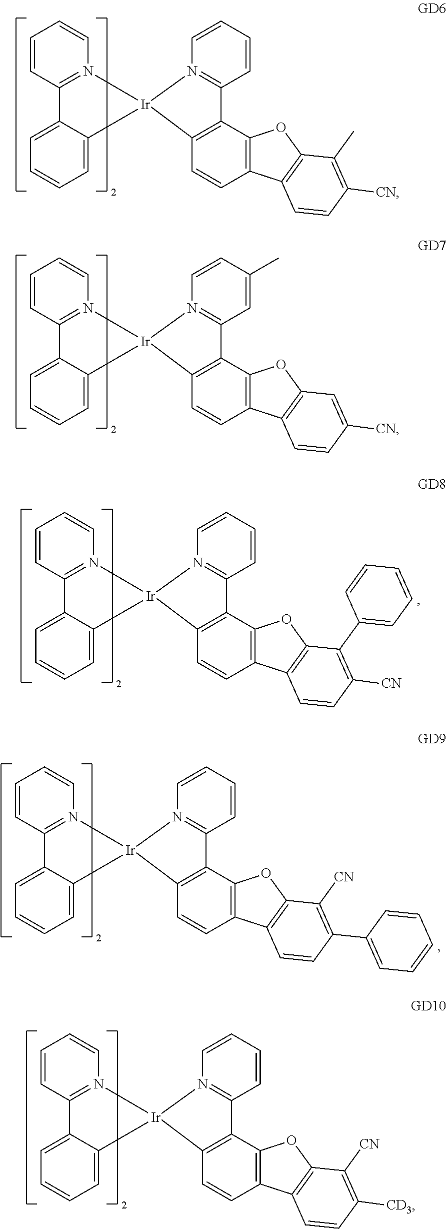

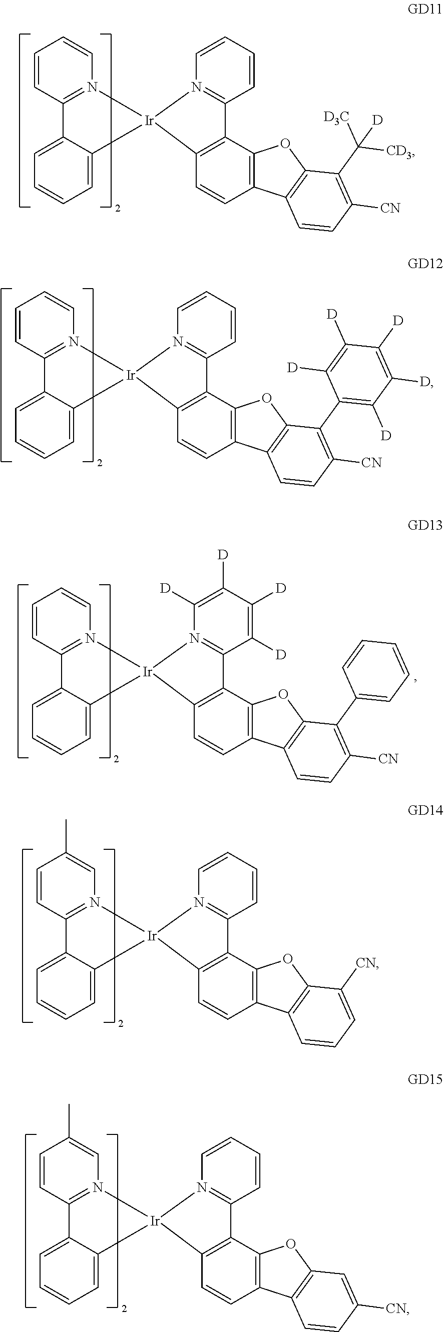

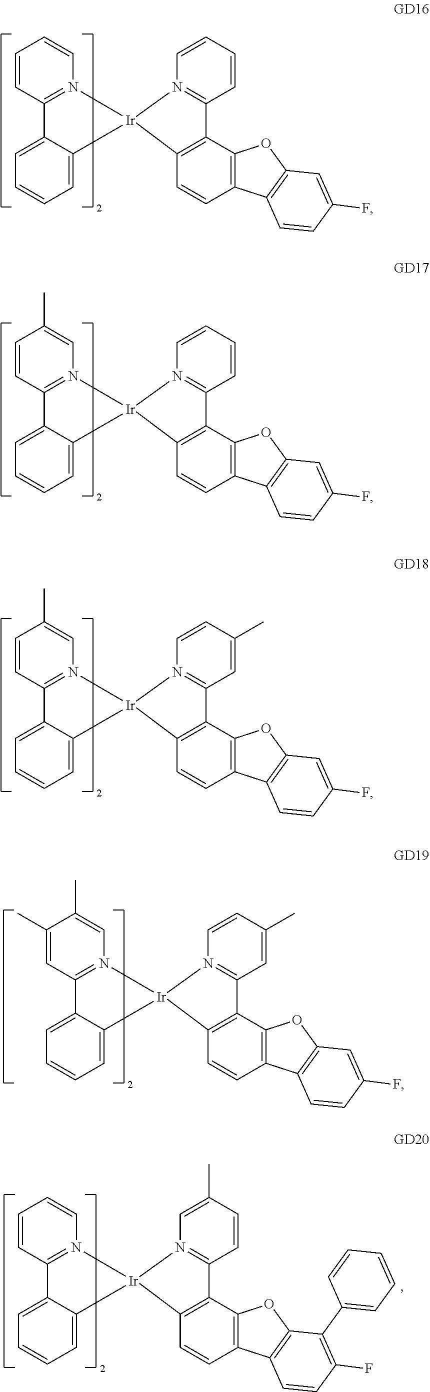

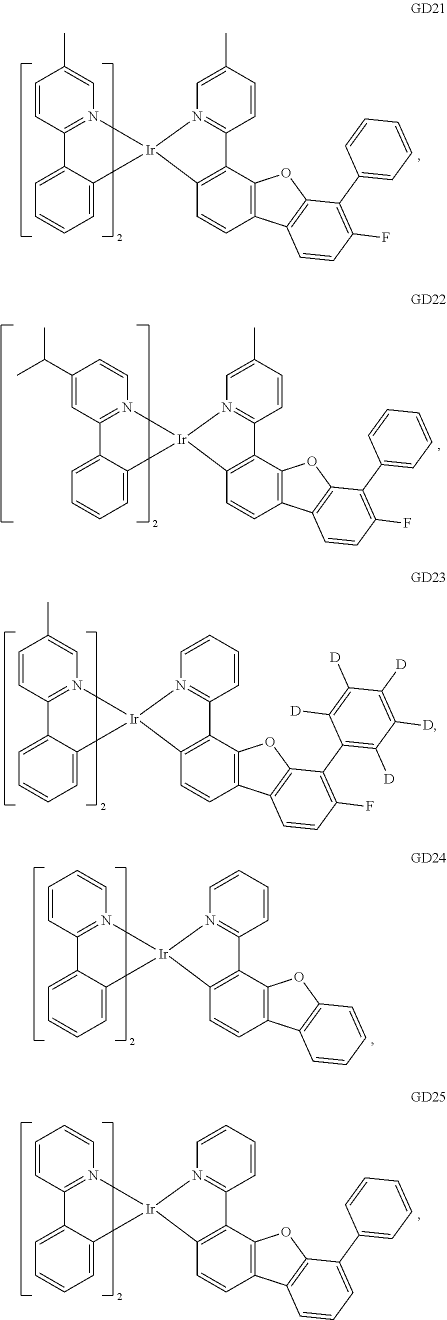

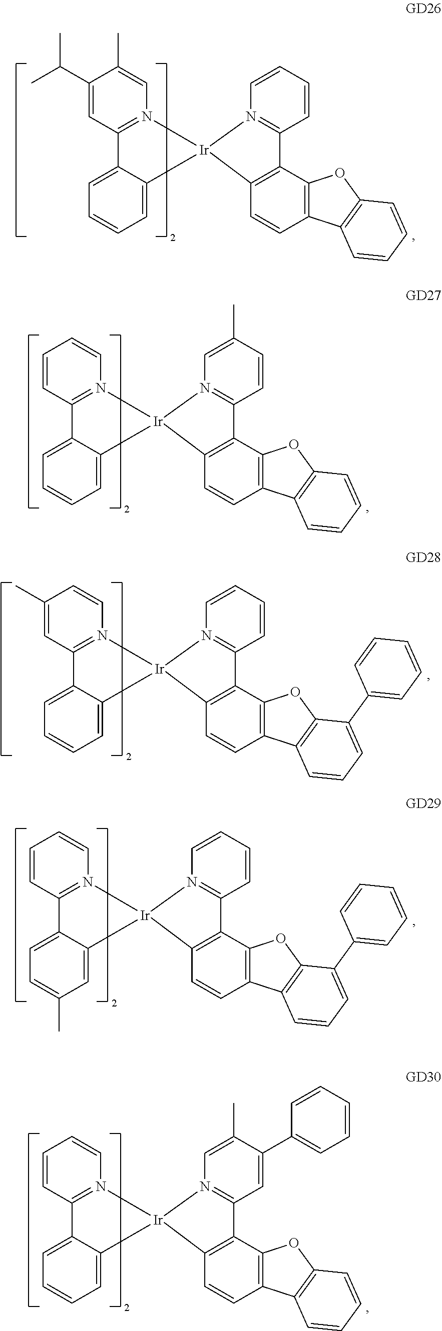

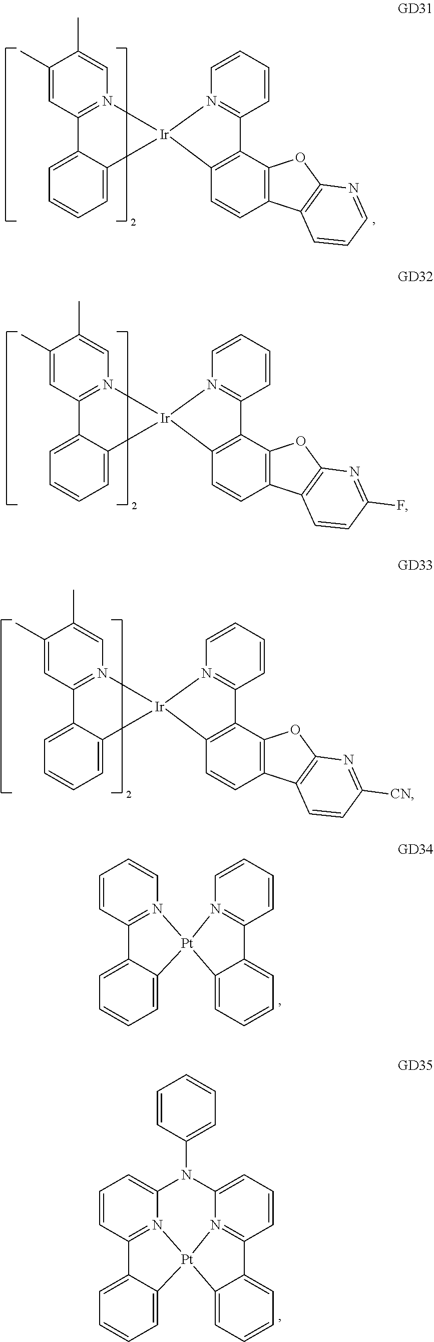

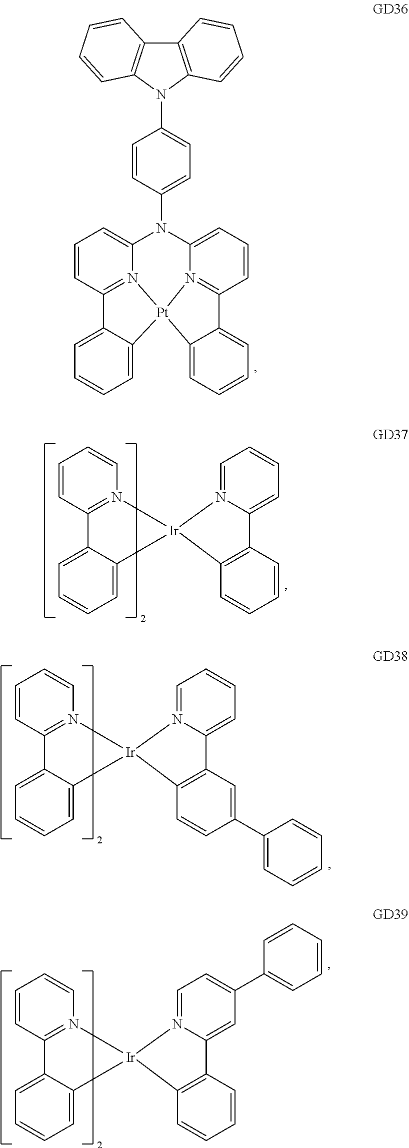

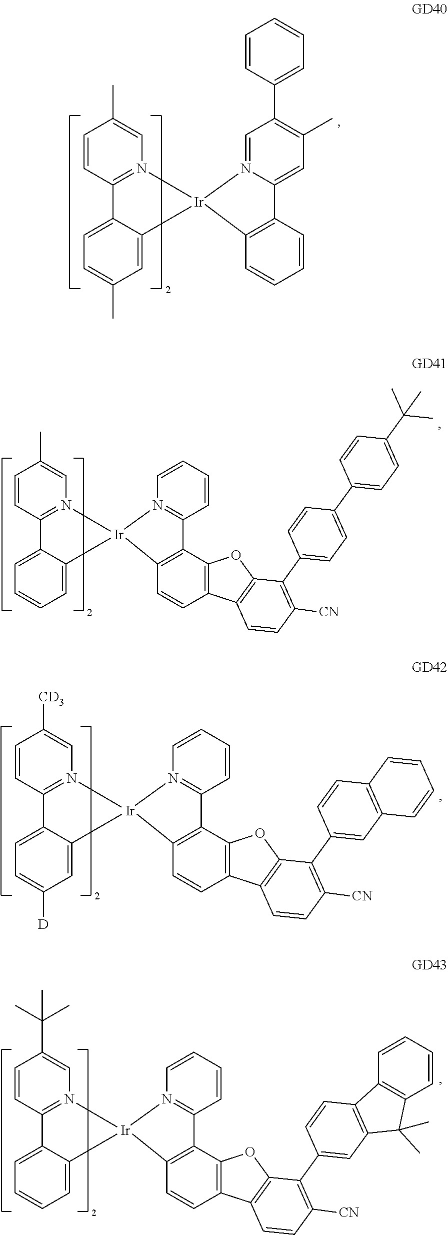

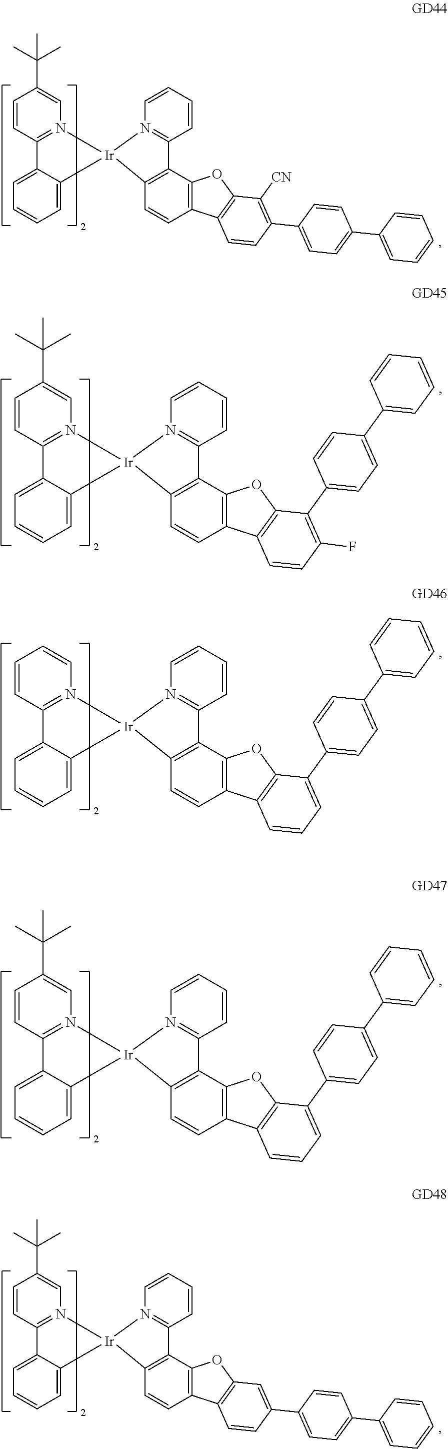

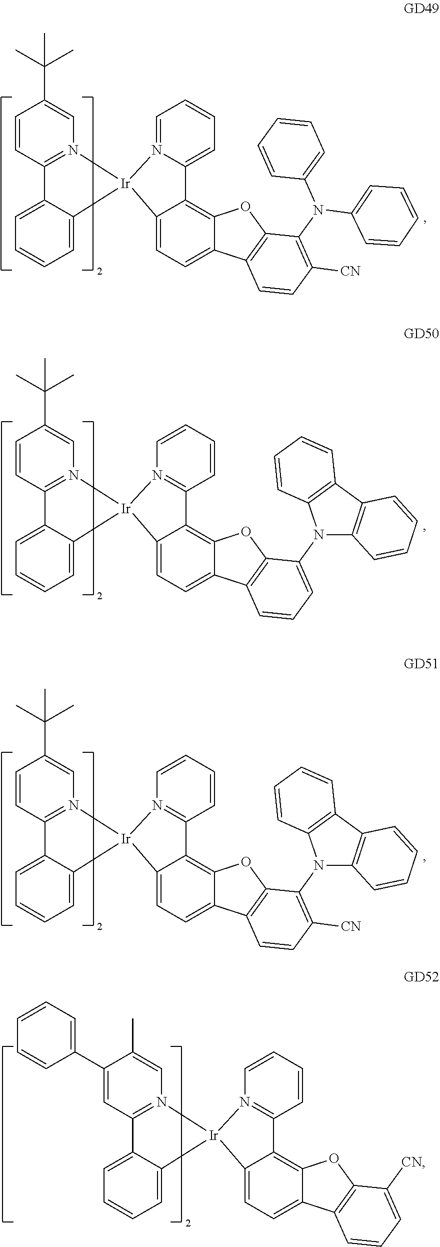

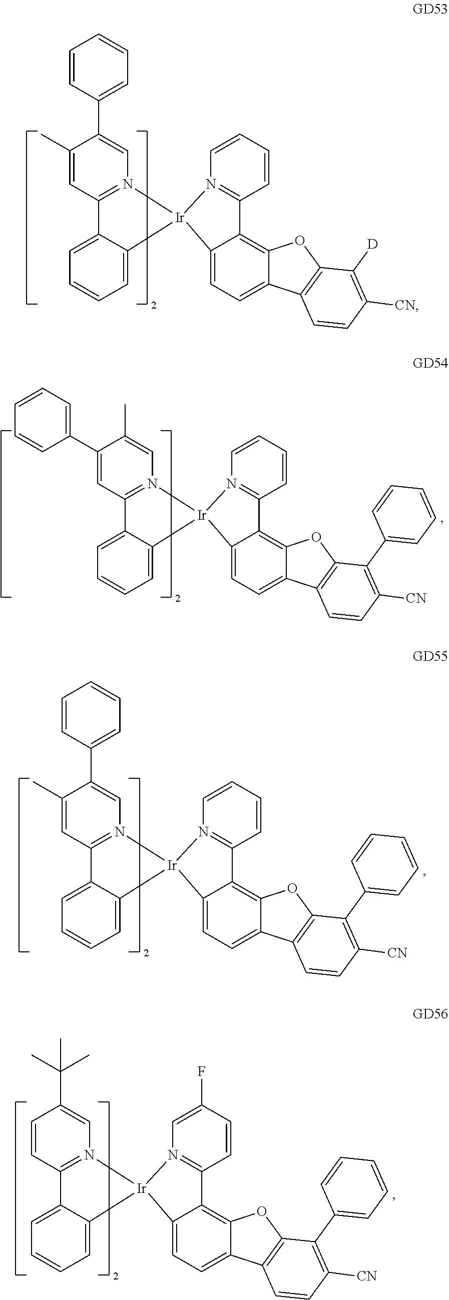

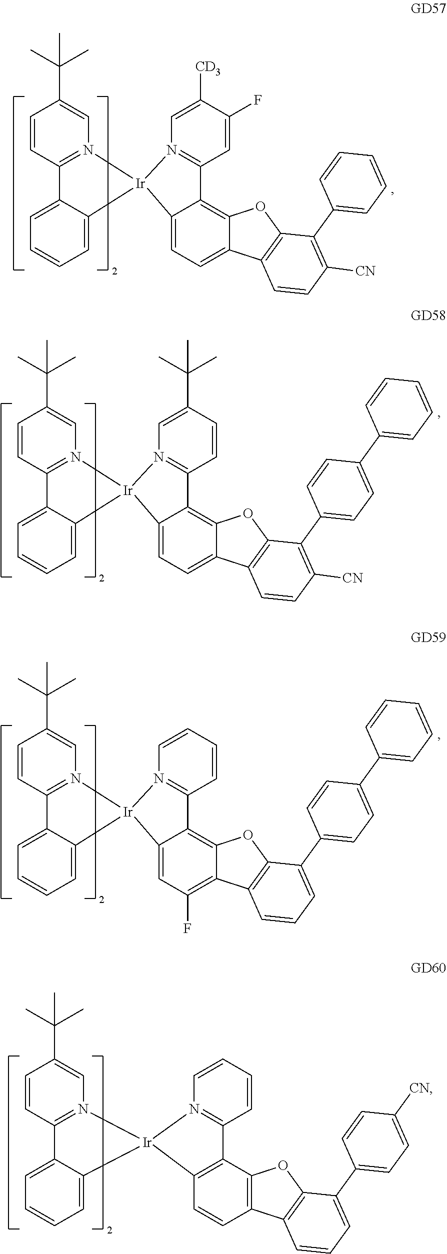

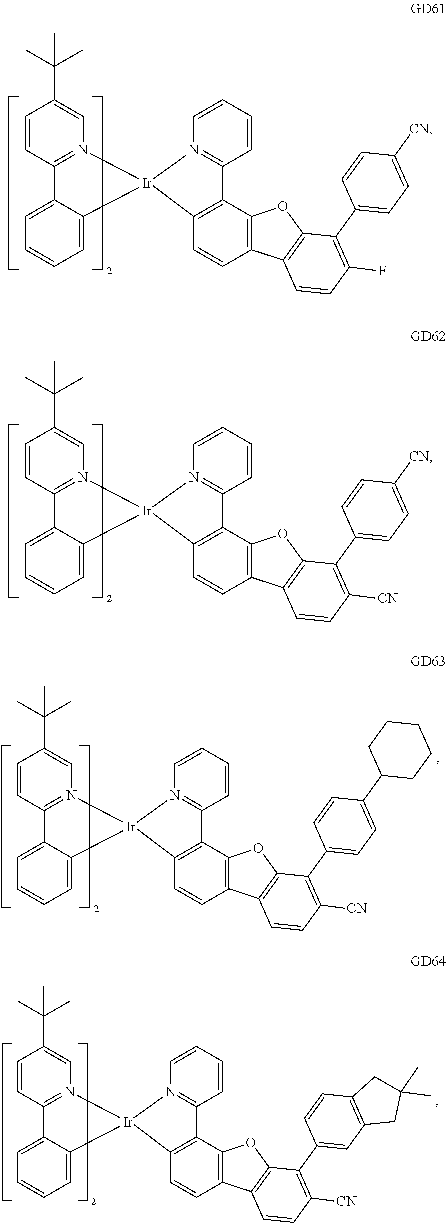

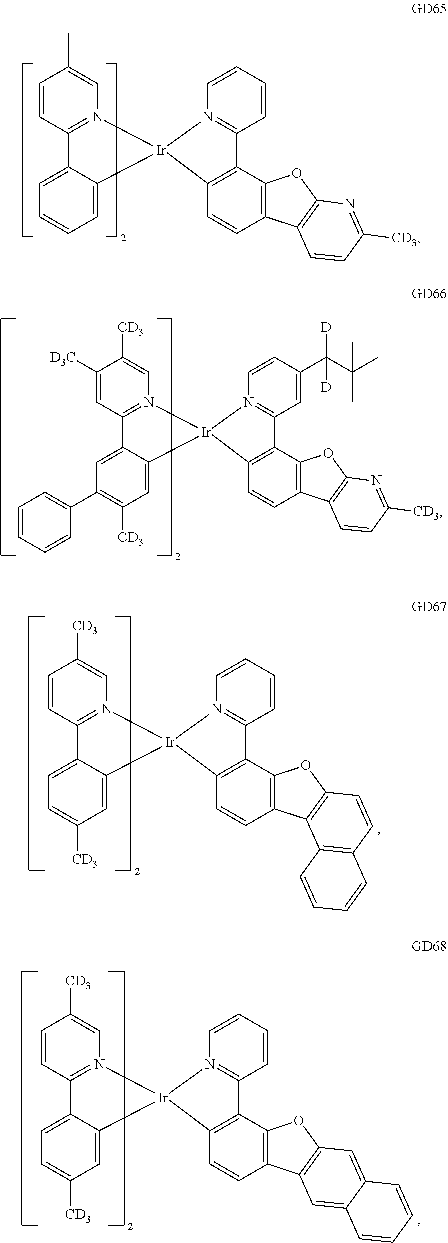

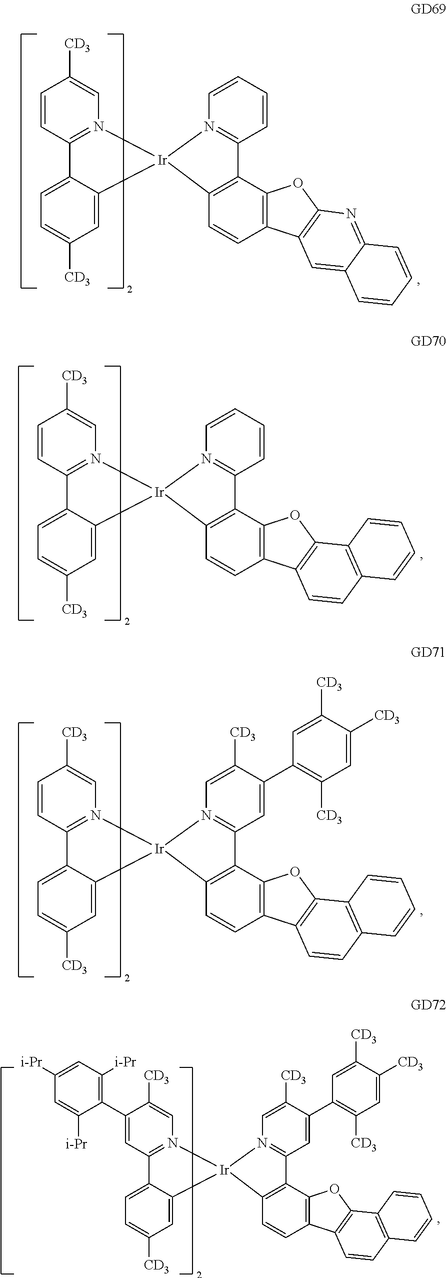

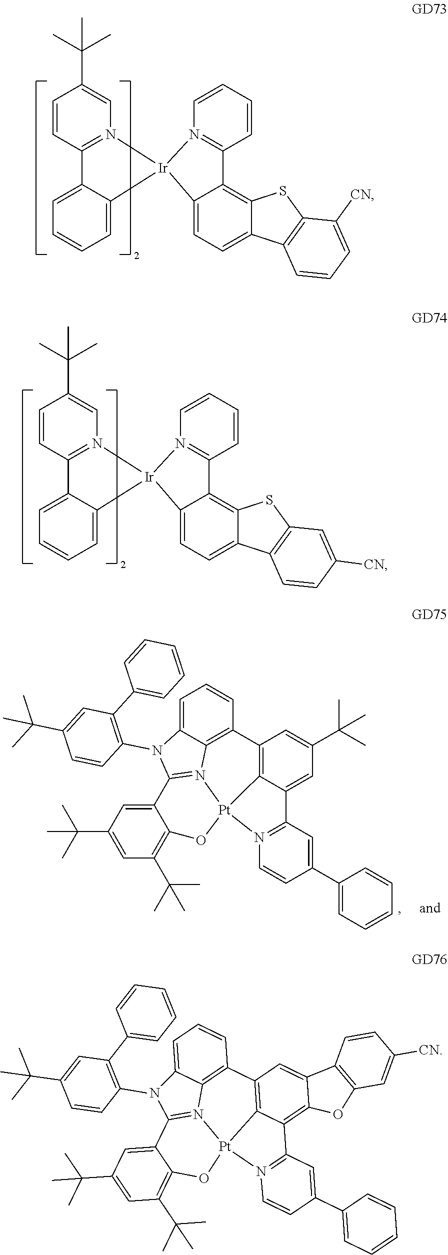

According to an embodiment of the present disclosure, the emissive layer in the organic electroluminescent device further includes a second host compound, wherein the second host compound includes at least one chemical group selected from the group consisting of: benzene, pyridine, pyrimidine, triazine, carbazole, azacarbazole, indolocarbazole, dibenzothiophene, azadibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene, and combinations thereof.

According to an embodiment of the present disclosure, the emissive layer in the organic electroluminescent device further includes a second host compound, wherein the second host compound includes at least one chemical group selected from the group consisting of: benzene, carbazole, indolocarbazole, fluorene, silafluorene, and combinations thereof.

According to an embodiment of the present disclosure, the second host compound in the organic electroluminescent device has a structure represented by Formula 3:

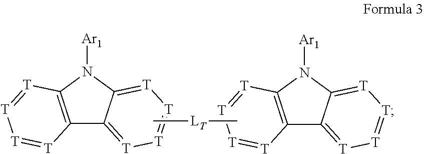

wherein
L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or combinations thereof,
T is, at each occurrence identically or differently, selected from C, CRₜ or N;
Rₜ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Ar₁ is, at each occurrence identically or differently, selected from substituted or unsubstituted aryl having 6 to 30 carbon atoms, substituted or unsubstituted heteroaryl having 3 to 30 carbon atoms or combinations thereof,
adjacent substituents Rₜ can be optionally joined to form a ring.

Herein, the expression that “adjacent substituents Rₜ can be optionally joined to form a ring” is intended to mean that any one or more of groups of adjacent substituents, such as any two substituents Rₜ, may be joined to form a ring. Obviously, it is also possible that none of these substituents are joined to form a ring.

According to an embodiment of the present disclosure, the second host compound in the organic electroluminescent device has a structure represented by Formula 4:

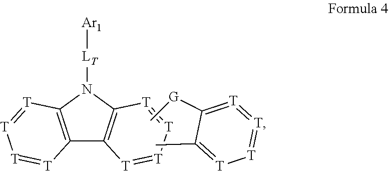

wherein
G is, at each occurrence identically or differently, selected from C(R_{g})₂, NR_{g}, O or S;
T is, at each occurrence identically or differently, selected from C, CRₜ or N;
L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or combinations thereof;
Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof,
Ar₁ is, at each occurrence identically or differently, selected from substituted or unsubstituted aryl having 6 to 30 carbon atoms, substituted or unsubstituted heteroaryl having 3 to 30 carbon atoms or combinations thereof,
adjacent substituents Rₜ, R_{g} can be optionally joined to form a ring.

Herein, the expression that “adjacent substituents Rₜ, R_{g} can be optionally joined to form a ring” is intended to mean that any one or more of groups of adjacent substituents, such as two substituents Rₜ, two substituents R_{g}, and substituents Rₜ and R_{g}, may be joined to form a ring. Obviously, it is also possible that none of these substituents are joined to form a ring.

According to an embodiment of the present disclosure, in Formula 3 and Formula 4, T is, at each occurrence identically or differently, selected from C or CRₜ.

According to an embodiment of the present disclosure, in Formula 3, T is, at each occurrence identically or differently, selected from C, CRₜ or N, and at least one of T is selected from N, for example, one or two T are selected from N.

According to an embodiment of the present disclosure, in Formula 4, T is, at each occurrence identically or differently, selected from C, CRₜ or N, and at least one of T is selected from N, for example, one or two T are selected from N.

According to an embodiment of the present disclosure, the second host compound in the organic electroluminescent device has a structure represented by one of Formulas 3-a to 3-j:

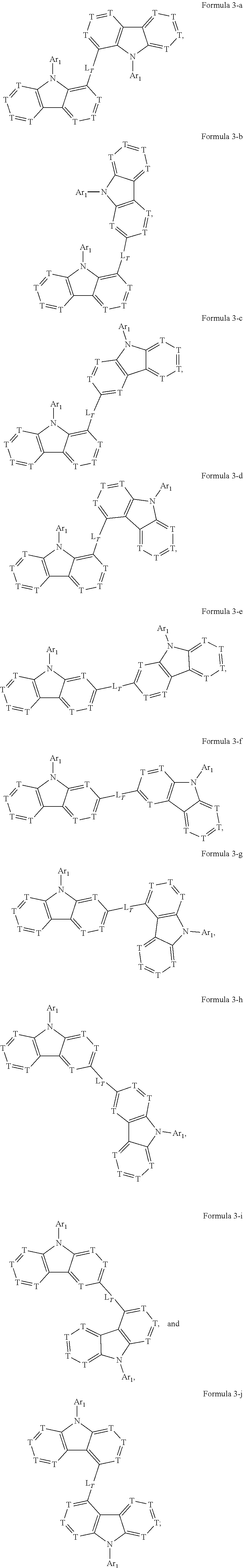

wherein
L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or combinations thereof,
T is, at each occurrence identically or differently, selected from CRₜ or N;
Rₜ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof,
Ar₁ is, at each occurrence identically or differently, selected from substituted or unsubstituted aryl having 6 to 30 carbon atoms, substituted or unsubstituted heteroaryl having 3 to 30 carbon atoms or combinations thereof,
adjacent substituents Rₜ can be optionally joined to form a ring.

According to an embodiment of the present disclosure, in Formulas 3-a to 3-j, T is, at each occurrence identically or differently, selected from CRₜ.

According to an embodiment of the present disclosure, in Formulas 3-a to 3-j, T is, at each occurrence identically or differently, selected from CRₜ or N, and at least one of T is selected from N, for example, one or two T are selected from N.

According to an embodiment of the present disclosure, the second host compound in the organic electroluminescent device has a structure represented by one of Formulas 4-a to 4-f:

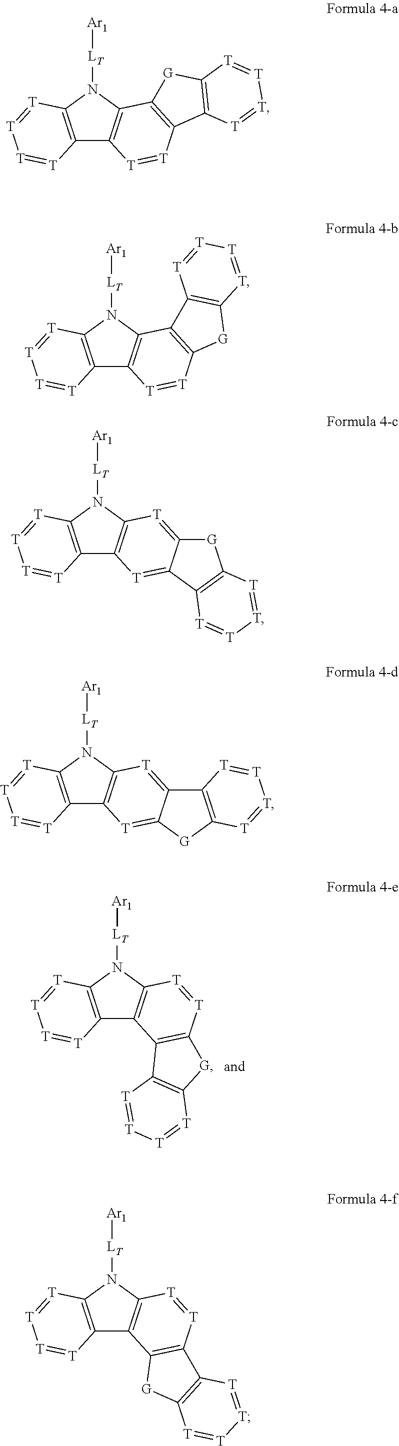

wherein
G is, at each occurrence identically or differently, selected from C(R_{g})₂, NR_{g}, O or S;
T is, at each occurrence identically or differently, selected from CRₜ or N;
L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or combinations thereof,
Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, sulfanyl group, a sulfinyl group, a sulfonyl group, a phosphino group, and combinations thereof,
Ar₁ is, at each occurrence identically or differently, selected from substituted or unsubstituted aryl having 6 to 30 carbon atoms, substituted or unsubstituted heteroaryl having 3 to 30 carbon atoms or combinations thereof, adjacent substituents Rₜ, R_{g} can be optionally joined to form a ring.

According to an embodiment of the present disclosure, in Formulas 4-a to 4-f, T is, at each occurrence identically or differently, selected from CRₜ.

According to an embodiment of the present disclosure, in Formulas 4-a to 4-f, T is, at each occurrence identically or differently, selected from CRₜ or N, and at least one of T is selected from N, for example, one or two T are selected from N.

According to an embodiment of the present disclosure, the second host compound is selected from the group consisting of, but not limited to, the following compounds:

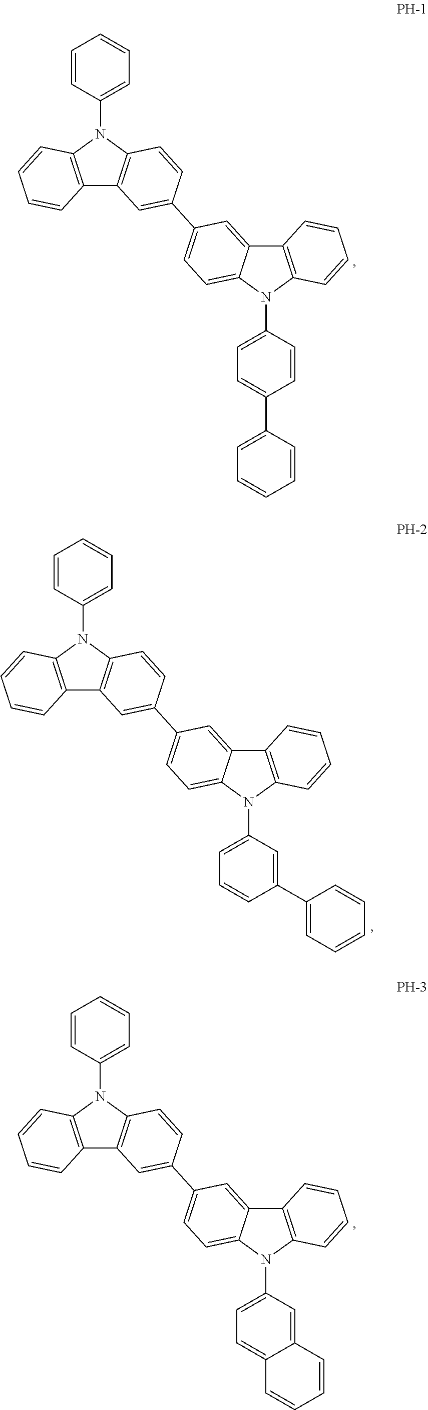 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4ccc5ccccc5c4)ccc32)cc1]

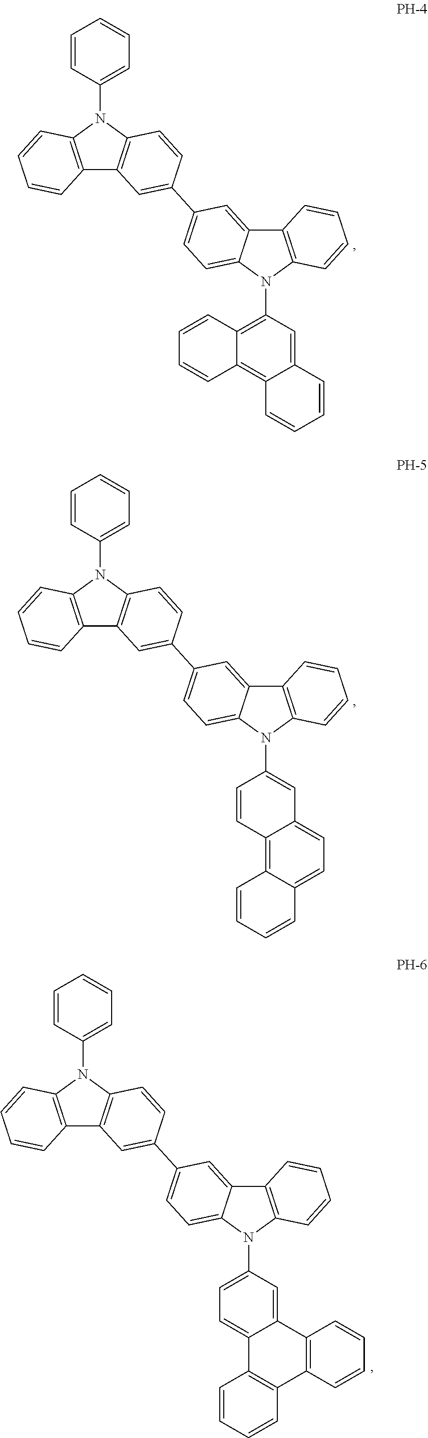 [SMILES: c1ccc(-n2c3ccccc3c3cc(-c4ccc5c(c4)c4ccccc4n5-c4cc5ccccc5c5ccccc45)ccc32)cc1 ; c1ccc(-n2c3ccccc3c3cc(-c4ccc5c(c4)c4ccccc4n5-c4ccc5c(ccc6ccccc65)c4)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

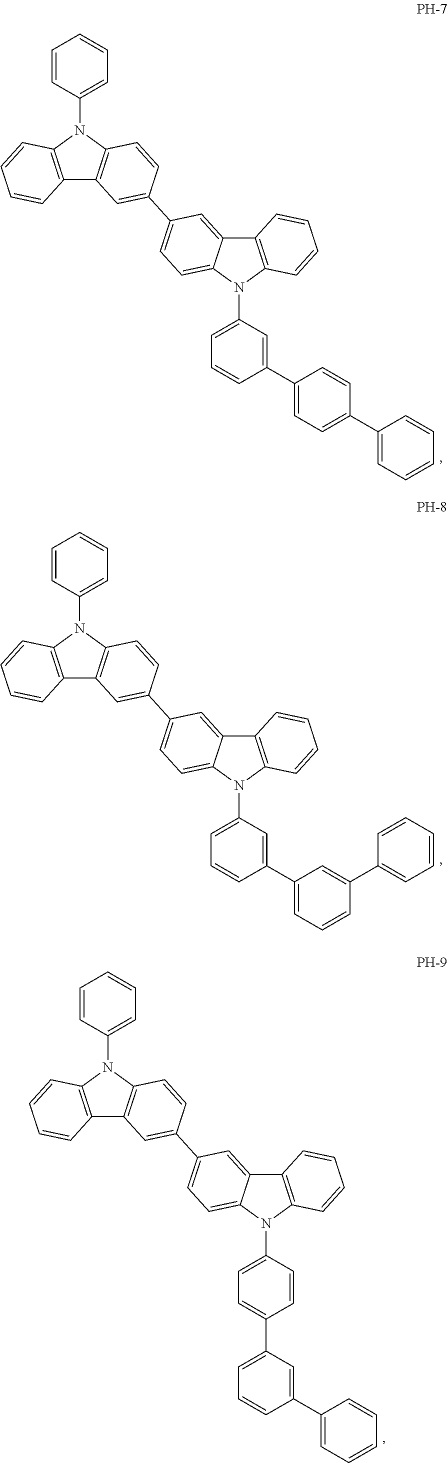 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

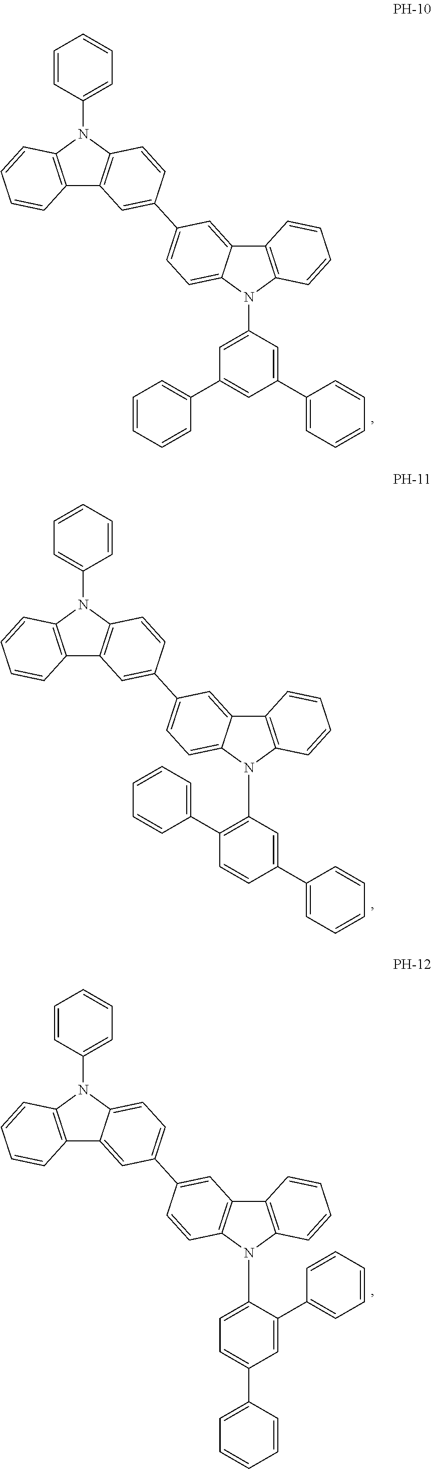 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-c3ccccc3)c(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)c(-c3ccccc3)c2)cc1]

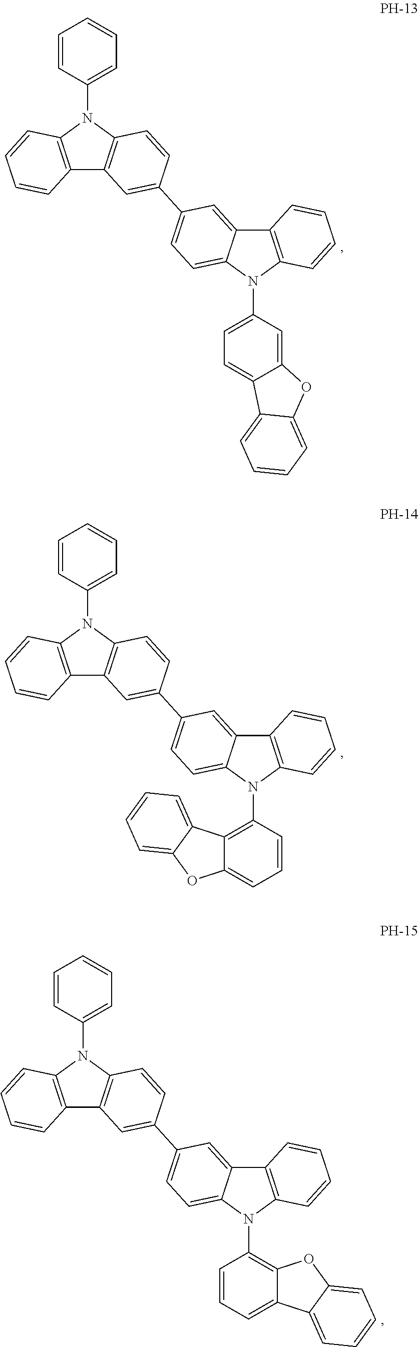 [SMILES: c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4cccc5c4oc4ccccc45)ccc32)cc1]

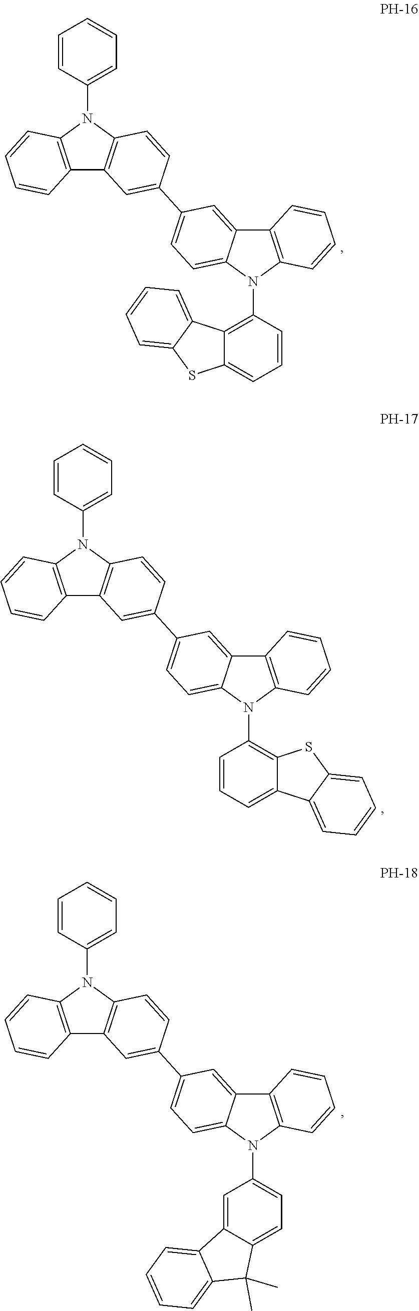 [SMILES: c1ccc(-n2c3ccccc3c3cc(-c4ccc5c(c4)c4ccccc4n5-c4cccc5sc6ccccc6c45)ccc32)cc1 ; c1ccc(-n2c3ccccc3c3cc(-c4ccc5c(c4)c4ccccc4n5-c4cccc5c4sc4ccccc45)ccc32)cc1 ; CC1(C)c2ccccc2-c2cc(-n3c4ccccc4c4cc(-c5ccc6c(c5)c5ccccc5n6-c5ccccc5)ccc43)ccc21]

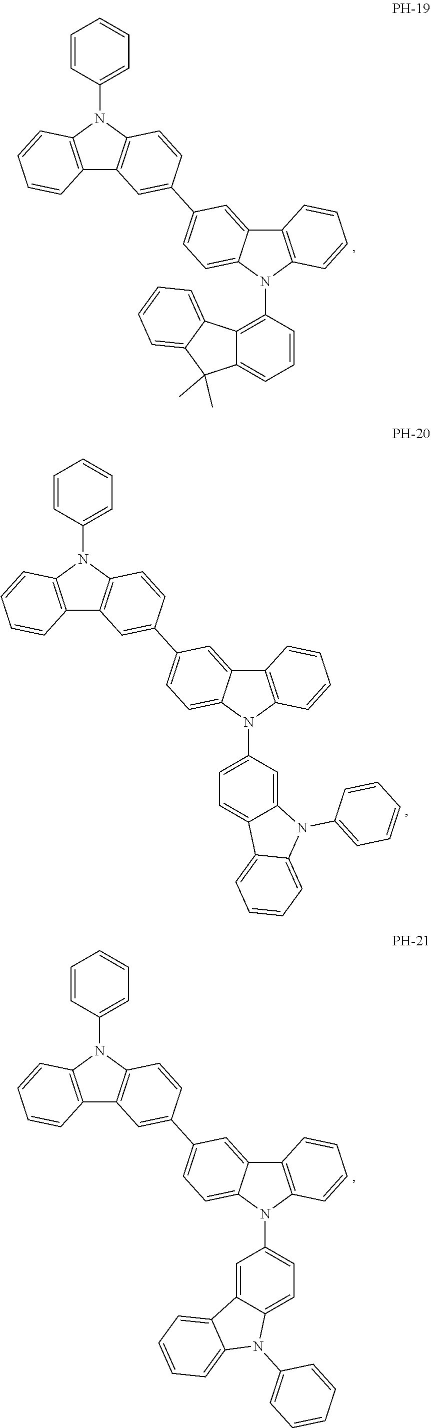 [SMILES: CC1(C)c2ccccc2-c2c(-n3c4ccccc4c4cc(-c5ccc6c(c5)c5ccccc5n6-c5ccccc5)ccc43)cccc21 ; c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-n2c3ccccc3c3cc(-c4ccc5c(c4)c4ccccc4n5-c4ccc5c(c4)c4ccccc4n5-c4ccccc4)ccc32)cc1]

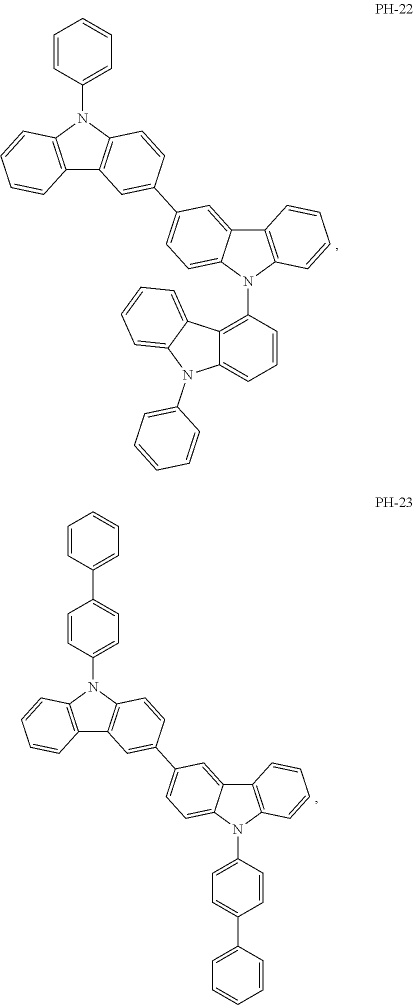 [SMILES: c1ccc(-n2c3ccccc3c3cc(-c4ccc5c(c4)c4ccccc4n5-c4cccc5c4c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

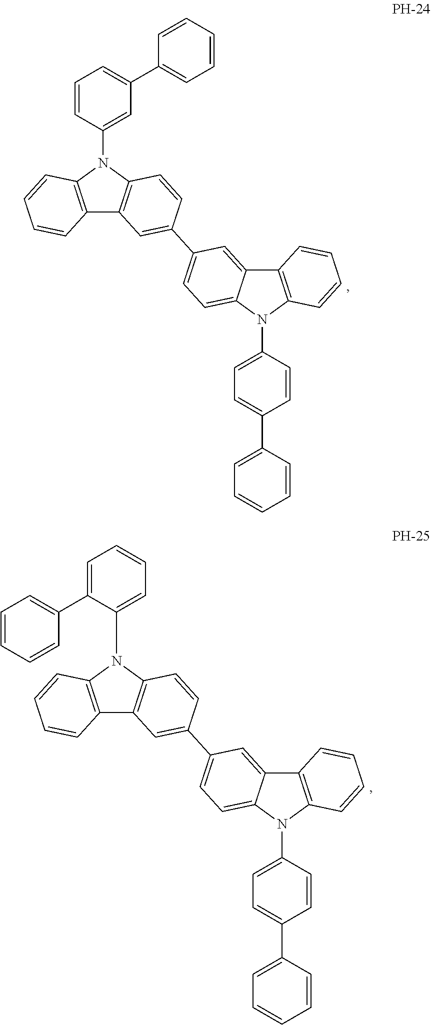 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1]

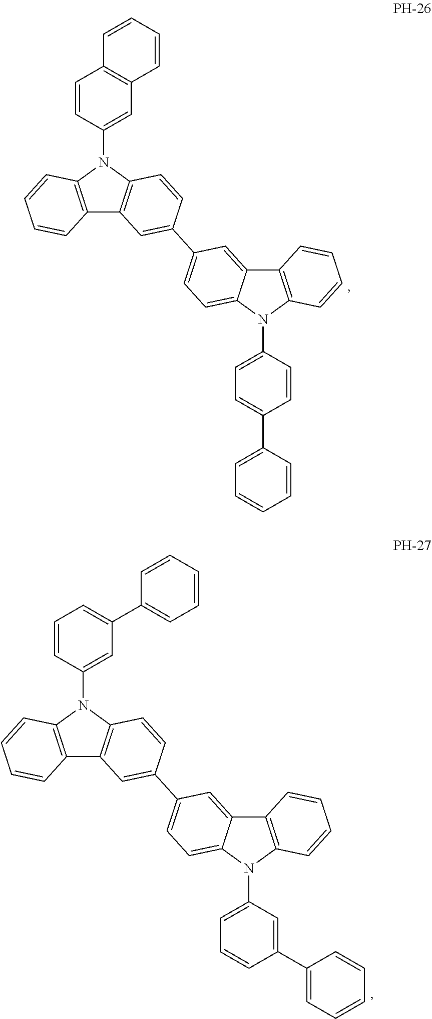 [SMILES: c1ccc(-c2ccc(-n3c4ccccc4c4cc(-c5ccc6c(c5)c5ccccc5n6-c5ccc6ccccc6c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

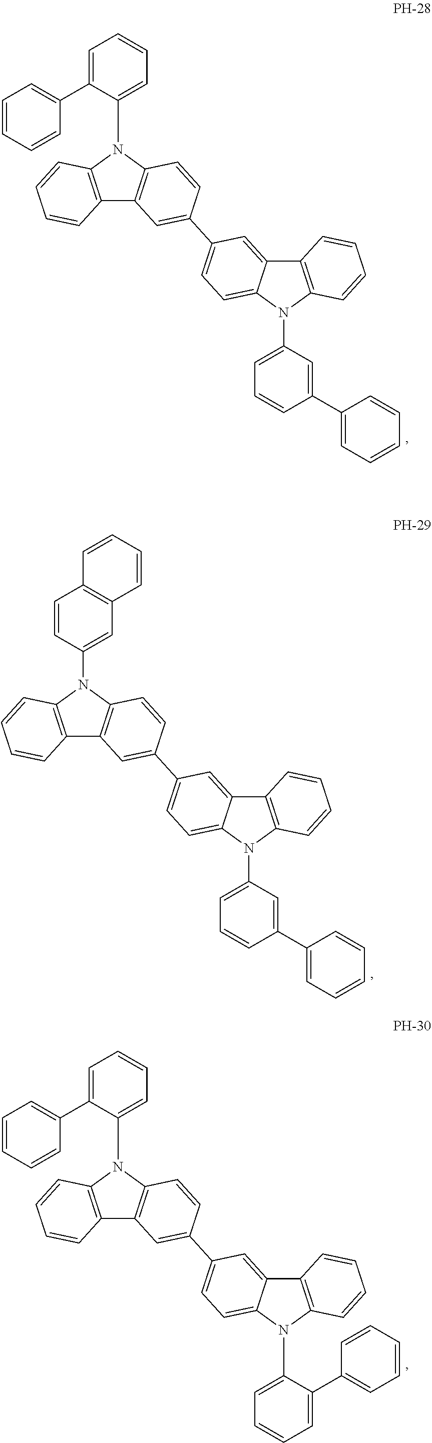 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2ccccc2-n2c3ccccc3c3cc(-c4ccc5c(c4)c4ccccc4n5-c4ccccc4-c4ccccc4)ccc32)cc1]

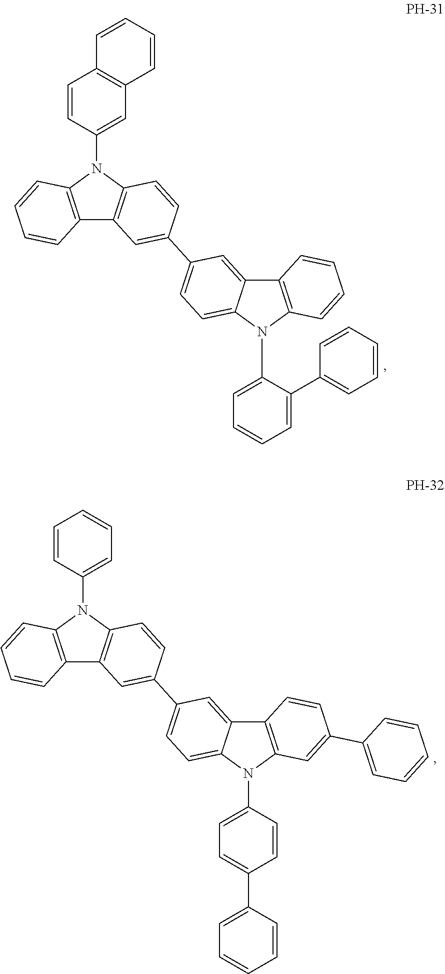 [SMILES: c1ccc(-c2ccccc2-n2c3ccccc3c3cc(-c4ccc5c(c4)c4ccccc4n5-c4ccc5ccccc5c4)ccc32)cc1 ; c1ccc(-c2ccc(-n3c4ccc(-c5ccc6c(c5)c5ccccc5n6-c5ccccc5)cc4c4ccc(-c5ccccc5)cc43)cc2)cc1]

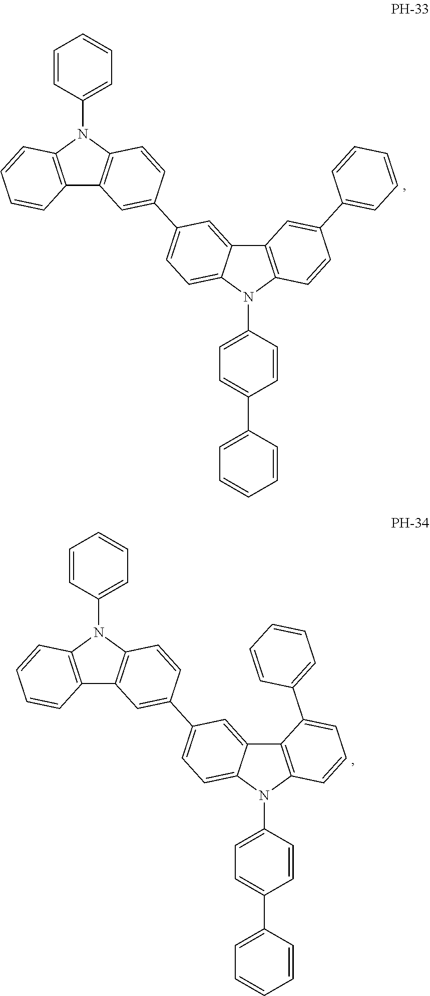 [SMILES: c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2ccc(-n3c4ccc(-c5ccc6c(c5)c5ccccc5n6-c5ccccc5)cc4c4c(-c5ccccc5)cccc43)cc2)cc1]

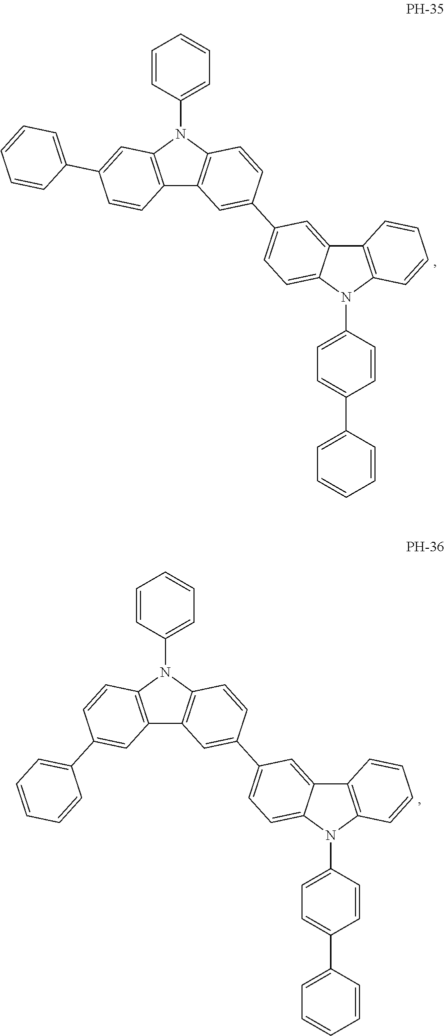 [SMILES: c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

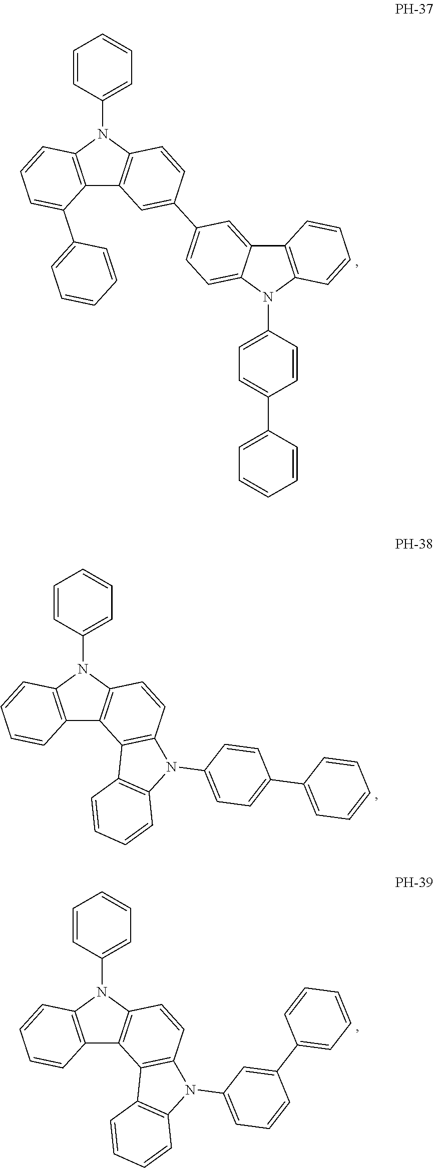 [SMILES: c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1]

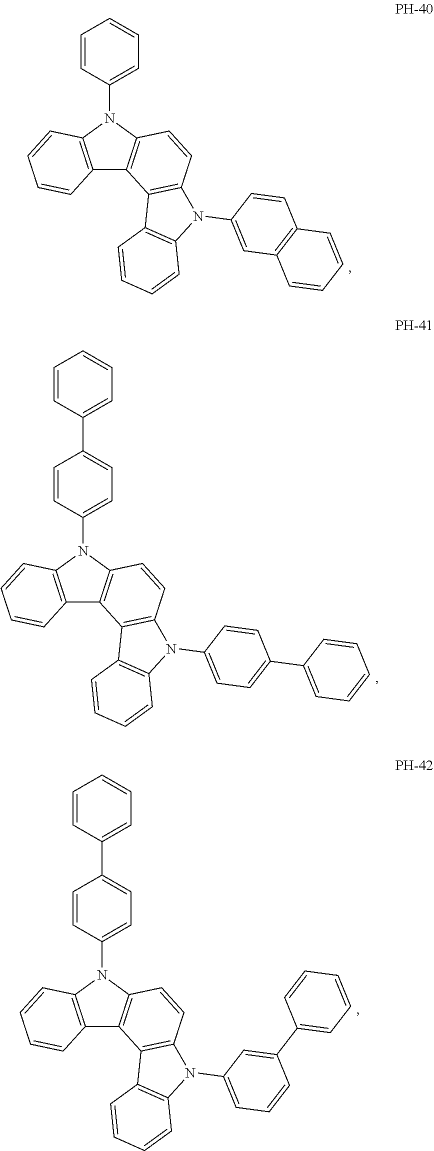 [SMILES: c1ccc(-n2c3ccccc3c3c4c5ccccc5n(-c5ccc6ccccc6c5)c4ccc32)cc1 ; c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1]

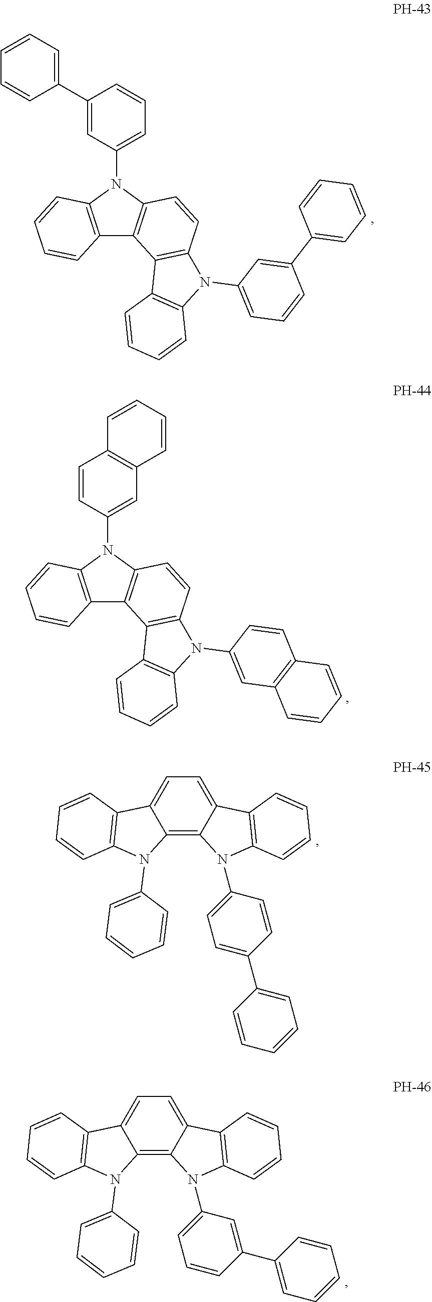

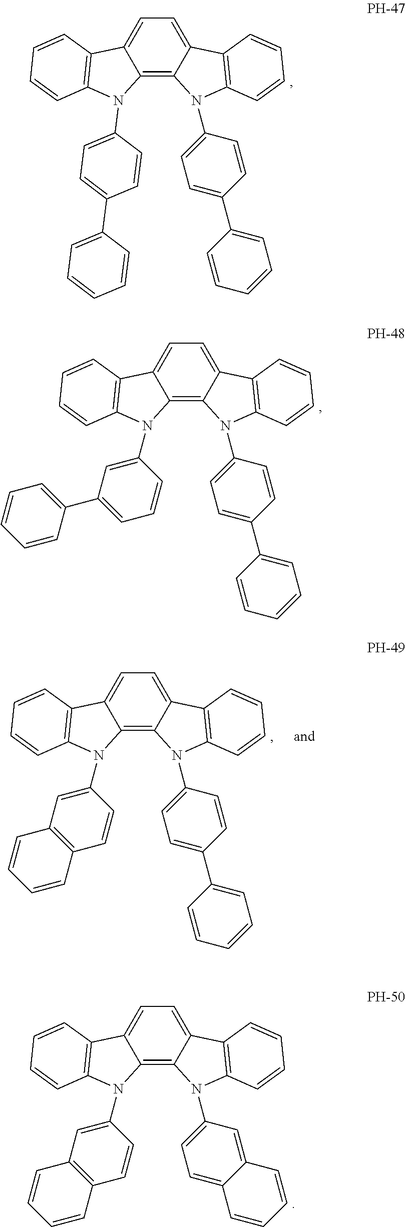 [SMILES: c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6cccc(-c7ccccc7)c6)c5c43)cc2)cc1 ; c1ccc(-c2ccc(-n3c4ccccc4c4ccc5c6ccccc6n(-c6ccc7ccccc7c6)c5c43)cc2)cc1 ; c1ccc2cc(-n3c4ccccc4c4ccc5c6ccccc6n(-c6ccc7ccccc7c6)c5c43)ccc2c1]

According to an embodiment of the present disclosure, the organic electroluminescent device emits green light.

According to an embodiment of the present disclosure, the organic electroluminescent device emits white light.

According to an embodiment of the present disclosure, the first metal complex is doped in the compound and the second host compound, and the weight of the first metal complex accounts for 1% to 30% of the total weight of the emissive layer.

According to an embodiment of the present disclosure, the first metal complex is doped in the compound and the second host compound, and the weight of the first metal complex accounts for 3% to 13% of the total weight of the emissive layer.

According to an embodiment of the present disclosure, a compound composition is disclosed, wherein the compound composition comprises the compound in any one of the preceding embodiments.

According to an embodiment of the present disclosure, an electronic device is disclosed, wherein the electronic device includes the organic electroluminescent device in any one of the preceding embodiments.

Combination with Other Materials

The materials described in the present disclosure for a particular layer in an organic light-emitting device can be used in combination with various other materials present in the device. The combinations of these materials are described in more detail in U.S. Pat. Pub. No. 20160359122 at paragraphs 0132-0161, which is incorporated by reference herein in its entirety.

The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light-emitting device may be used in combination with a variety of other materials present in the device. For example, dopants disclosed herein may be used in combination with a wide variety of hosts, transport layers, blocking layers, injection layers, electrodes and other layers that may be present. The combination of these materials is described in detail in paragraphs 0080-0101 of U.S. patent. Pub. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FATAR, life testing system produced by SUZHOU FATAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this patent.

### Material Synthesis Example

The method for preparing the compound of the present disclosure is not limited herein. Typically, the following compounds are used as examples without limitation, and the synthesis routes and preparation methods thereof are described below.

### Synthesis Example 1: Synthesis of Compound A-6

### Step 1: Synthesis of Intermediate B

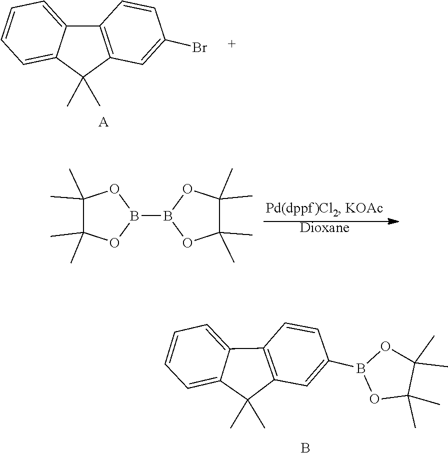

A (15.0 g, 54.9 mmol), bis(pinacolato)diboron (20.9 g, 82.5 mmol), Pd(dppf)Cl₂ (0.81 g, 1.1 mmol), KOAc (10.8 g, 110 mmol), and 200 mL of 1,4-dioxane were successively added to a three-necked round-bottom flask. Under the protection of N₂, the mixture was heated to reflux overnight. After the completion of the reaction was verified by a TLC plate, heating was stopped, and the reaction was allowed to cool to room temperature. The reaction system was filtered through Celite, and the filtrate was concentrated under reduced pressure. The crude product was subjected to silica gel column chromatography (PE/DCM=10:1 to 2:1) to give Intermediate B (15.0 g, 46.8 mmol) as a white solid with a yield of 85.2%.

### Step 2: Synthesis of Intermediate D

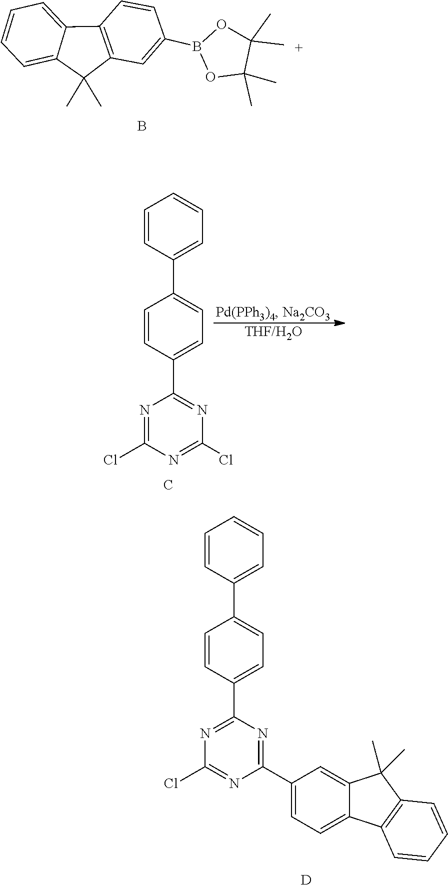

B (8.97 g, 28.0 mmol), C (12.7 g, 42.0 mmol), Pd(PPh₃)₄ (1.62 g, 1.4 mmol), Na₂CO₃ (5.9 g, 56.0 mmol), 160 mL of THF, and 40 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was subjected to liquid separation. The aqueous phase was extracted with DCM. The organic phase was combined, dried with anhydrous Na₂SO₄, filtered, and concentrated under reduced pressure. The crude product was subjected to silica gel column chromatography (PE/DCM=5:1 to 2:1) to give Intermediate D (5.0 g, 10.87 mmol) as a white solid with a yield of 38.8%.

### Step 3: Synthesis of Compound A-6

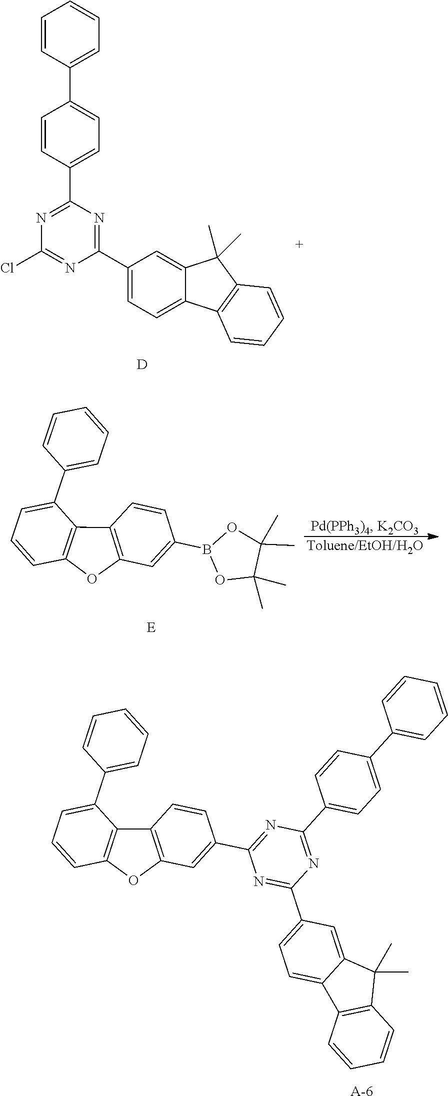

D (5.0 g, 10.87 mmol), E (4.23 g, 11.41 mmol), Pd(PPh₃)₄ (0.25 g, 0.22 mmol), K₂CO₃ (3.0 g, 21.74 mmol), 60 mL of toluene, 15 mL of EtOH, and 15 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was filtered by suction under reduced pressure. The resulting solid was washed with water and ethanol in sequence. The crude product was subjected to silica gel column chromatography (PE/DCM=10:1 to 2:1) to give a white solid (6.0 g, 8.98 mmol) with a yield of 82.6%. The product was confirmed as the target product A-6 with a molecular weight of 667.3.

### Synthesis Example 2: Synthesis of Compound A-7

### Step 1: Synthesis of Intermediate G

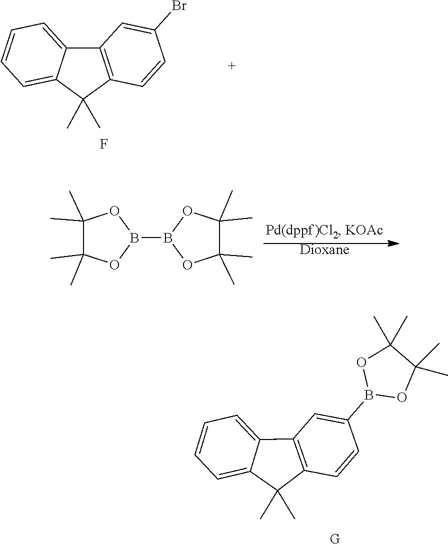

F (5.0 g, 18.38 mmol), bis(pinacolato)diboron (7.0 g, 27.57 mmol), Pd(dppf)Cl₂ (0.27 g, 0.37 mmol), KOAc (5.4 g, 55.14 mmol), and 100 mL of 1,4-dioxane were successively added to a three-necked round-bottom flask. Under the protection of N₂, the mixture was heated to reflux overnight. After the completion of the reaction was verified by a TLC plate, heating was stopped, and the reaction was allowed to cool to room temperature. The reaction system was filtered through Celite, and the filtrate was concentrated under reduced pressure. The crude product was subjected to silica gel column chromatography (PE/DCM=5:1 to 2:1) to give Intermediate G (2.8 g, 8.74 mmol) as a white solid with a yield of 47.6%.

### Step 2: Synthesis of Intermediate H

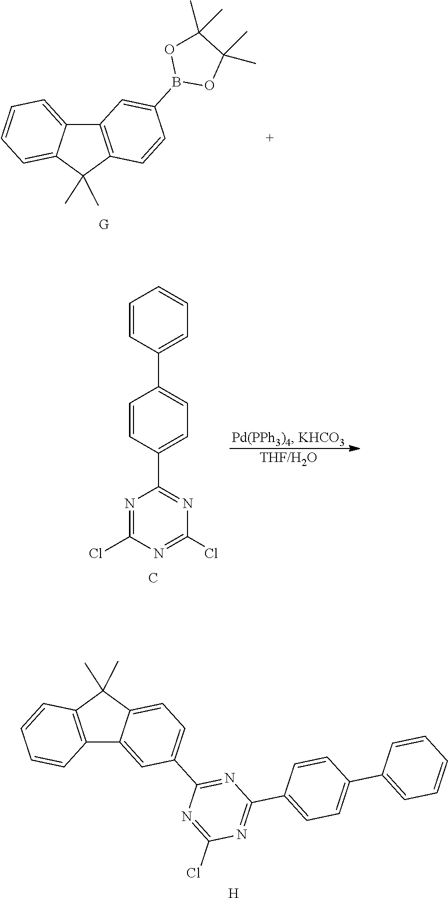

G (4.5 g, 14.0 mmol), C (4.23 g, 14.0 mmol), Pd(PPh₃)₄ (0.33 g, 0.28 mmol), KHCO₃ (2.8 g, 28.1 mmol), 80 mL of THF, and 20 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was subjected to liquid separation. The aqueous phase was extracted with DCM. The organic phase was combined, dried with anhydrous Na₂SO₄, filtered, and concentrated under reduced pressure. The crude product was subjected to silica gel column chromatography (PE/DCM=4:1) to give Intermediate H (2.6 g, 5.66 mmol) as a white solid with a yield of 40.4%.

### Step 3: Synthesis of Compound A-7

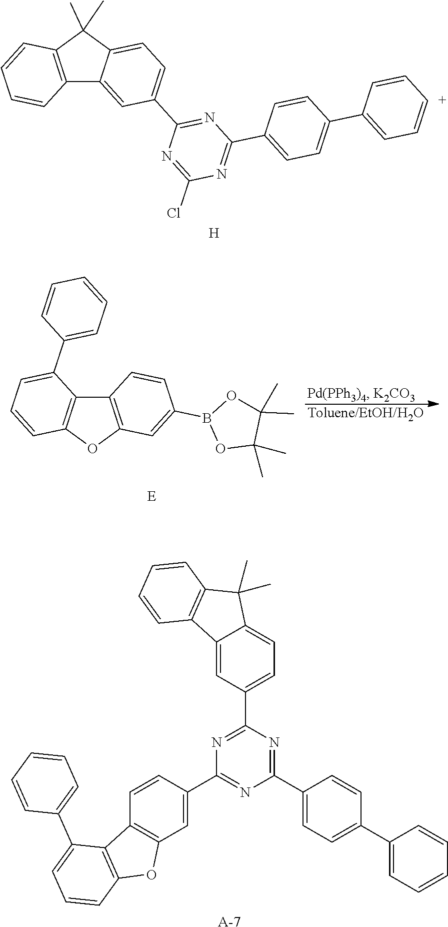

H (2.6 g, 5.66 mmol), E (2.1 g, 5.66 mmol), Pd(PPh₃)₄ (0.13 g, 0.11 mmol), K₂CO₃ (1.56 g, 11.3 mmol), 80 mL of toluene, 10 mL of EtOH, and 10 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was filtered by suction under reduced pressure. The resulting solid was washed with water and ethanol in sequence. The solid was recrystallized from toluene/acetonitrile to give a white solid (3.4 g, 5.1 mmol) with a yield of 90.1%. The product was confirmed as the target product A-7 with a molecular weight of 667.3.

### Synthesis Example 3: Synthesis of Compound A-8

### Step 1: Synthesis of Intermediate J

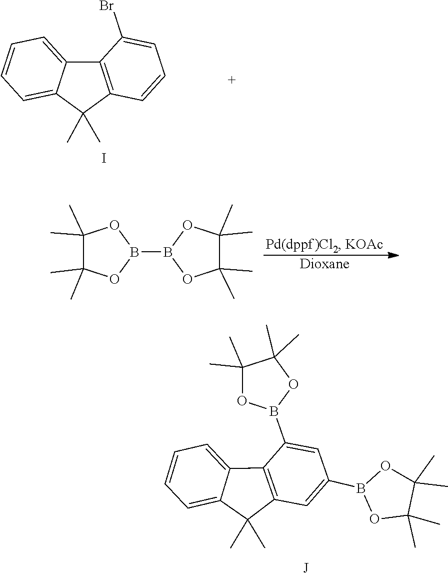

I (27.3 g, 100.0 mmol), bis(pinacolato)diboron (50.8 g, 200.0 mmol), Pd(dppf)Cl₂ (1.5 g, 2.0 mmol), KOAc (19.6 g, 200.0 mmol), and 200 mL of 1,4-dioxane were successively added to a three-necked round-bottom flask. Under the protection of N₂, the mixture was heated to reflux overnight. After the completion of the reaction was verified by a TLC plate, heating was stopped, and the reaction was allowed to cool to room temperature. The reaction system was filtered through Celite, and the filtrate was concentrated under reduced pressure. The crude product was subjected to silica gel column chromatography (PE/DCM=5:1 to 2:1) to give Intermediate J (2.8 g, 87.4 mmol) as a white solid with a yield of 87.4%.

### Step 2: Synthesis of Intermediate K

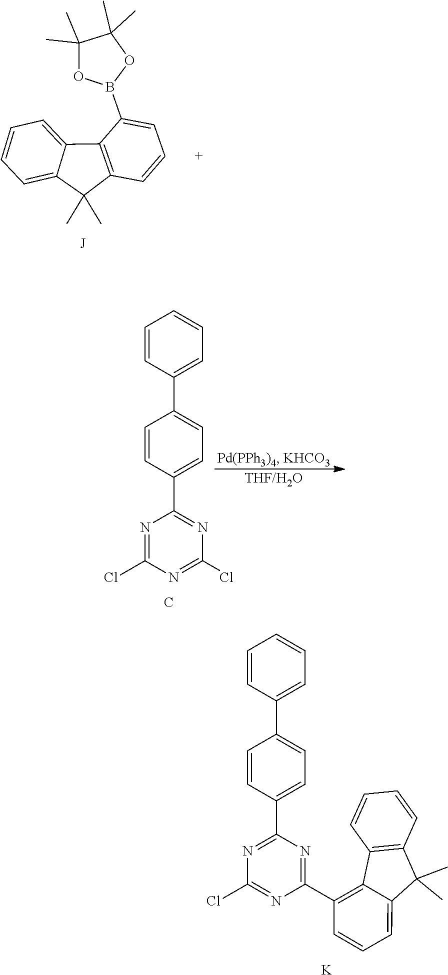

J (9.0 g, 28.1 mmol), C (8.5 g, 28.1 mmol), Pd(PPh₃)₄ (0.97 g, 0.84 mmol), KHCO₃ (5.6 g, 56.2 mmol), 160 mL of THF, and 40 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was subjected to liquid separation. The aqueous phase was extracted with DCM. The organic phase was combined, dried with anhydrous Na₂SO₄, filtered, and concentrated under reduced pressure. The crude product was subjected to silica gel column chromatography (PE/DCM=4:1) to give Intermediate K (5.2 g, 11.3 mmol) as a white solid with a yield of 40.2%.

### Step 3: Synthesis of Compound A-8

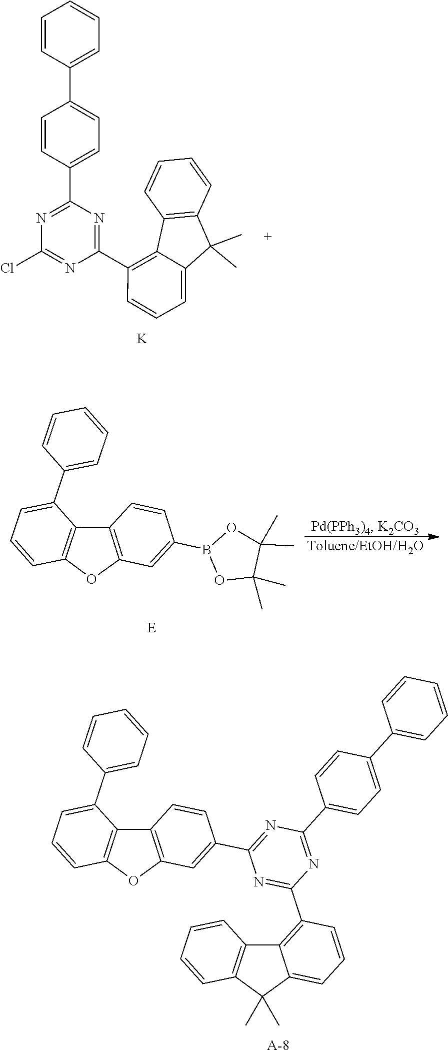

K (2.6 g, 5.66 mmol), E (2.1 g, 5.66 mmol), Pd(PPh₃)₄ (0.13 g, 0.11 mmol), K₂CO₃ (1.56 g, 11.3 mmol), 80 mL of toluene, 10 mL of EtOH, and 10 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was filtered by suction under reduced pressure. The resulting solid was washed with water and ethanol in sequence. The solid was recrystallized from toluene/acetonitrile to give a white solid (2.9 g, 4.2 mmol) with a yield of 74.2%. The product was confirmed as the target product A-8 with a molecular weight of 667.3.

### Synthesis Example 4: Synthesis of Compound A-12

### Step 1: Synthesis of Intermediate M

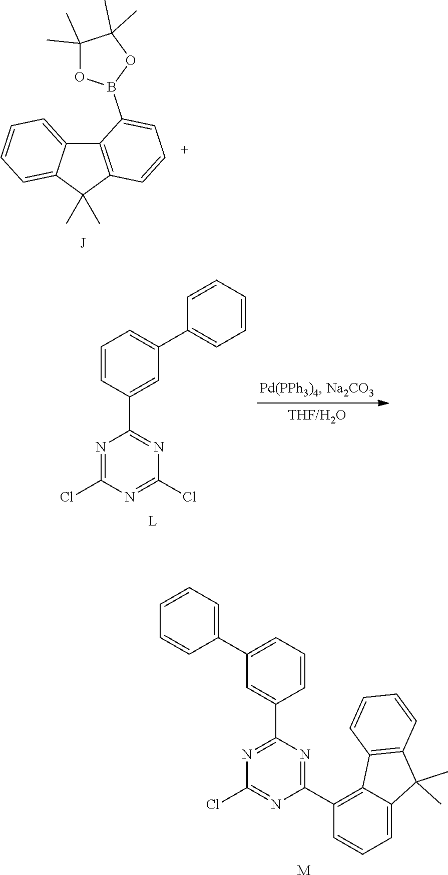

J (10.0 g, 31.3 mmol), L (11.9 g, 39.4 mmol), Pd(PPh₃)₄ (1.8 g, 1.6 mmol), Na₂CO₃ (6.6 g, 62.6 mmol), 160 mL of THF, and 40 mL of H₂O were successively added to a three-necked round-bottom flask. Under the protection of N₂, the mixture was heated to reflux. After 10 h, the completion of the reaction was verified by a TLC plate, heating was stopped, and the reaction was allowed to cool to room temperature. The mixture was subjected to liquid separation. The aqueous phase was extracted with DCM. The organic phase was combined, dried with anhydrous Na₂SO₄, filtered, and concentrated under reduced pressure. The crude product was subjected to silica gel column chromatography (PE/DCM=4:1) to give Intermediate M (6.7 g, 14.6 mmol) as a white solid with a yield of 46.5%.

### Step 2: Synthesis of Compound A-12

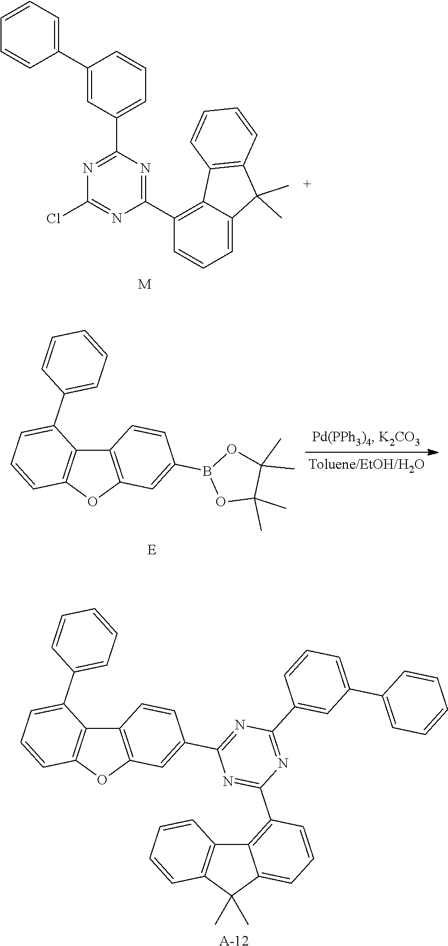

M (4.8 g, 10.4 mmol), E (4.1 g, 11.0 mmol), Pd(PPh₃)₄ (0.60 g, 0.52 mmol), K₂CO₃ (4.3 g, 31.3 mmol), 120 mL of toluene, 30 mL of EtOH, and 30 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was subjected to liquid separation. The aqueous phase was extracted with DCM. The organic phase was combined, dried with anhydrous Na₂SO₄, filtered, and concentrated under reduced pressure. The solid was recrystallized from toluene/ethanol to give a white solid (5.3 g, 7.9 mmol) with a yield of 76.3%. The product was confirmed as the target product A-12 with a molecular weight of 667.3.

### Synthesis Example 5: Synthesis of Compound A-138

### Step 1: Synthesis of Intermediate O

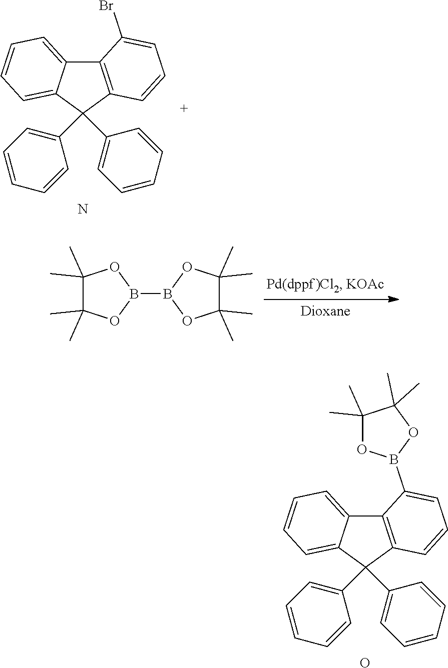

N (9.9 g, 25.0 mmol), bis(pinacolato)diboron (9.5 g, 37.5 mmol), Pd(dppf)Cl₂ (0.55 g, 0.75 mmol), KOAc (4.9 g, 50.0 mmol), and 80 mL of 1,4-dioxane were successively added to a three-necked round-bottom flask. Under the protection of N₂, the mixture was heated to reflux overnight. After the completion of the reaction was verified by a TLC plate, heating was stopped, and the reaction was allowed to cool to room temperature. The reaction system was filtered through Celite, and the filtrate was concentrated under reduced pressure. The crude product was subjected to silica gel column chromatography (PE/DCM=5:1 to 4:1) to give Intermediate O (8.1 g, 18.2 mmol) as a white solid with a yield of 72.9%.

### Step 2: Synthesis of Intermediate P

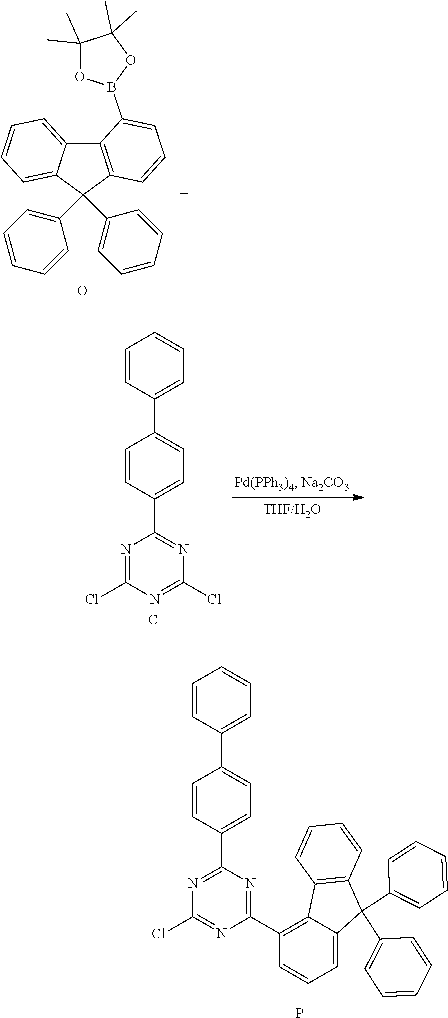

O (8.0 g, 18.1 mmol), C (8.7 g, 29.0 mmol), Pd(PPh₃)₄ (1.0 g, 0.87 mmol), Na₂CO₃ (3.8 g, 36.2 mmol), 96 mL of THF, and 24 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was subjected to liquid separation. The aqueous phase was extracted with DCM. The organic phase was combined, dried with anhydrous Na₂SO₄, filtered, and concentrated under reduced pressure. The crude product was subjected to silica gel column chromatography (PE/DCM=3:1) to give Intermediate P (4.0 g, 6.8 mmol) as a white solid with a yield of 37.8%.

### Step 3: Synthesis of Compound A-138

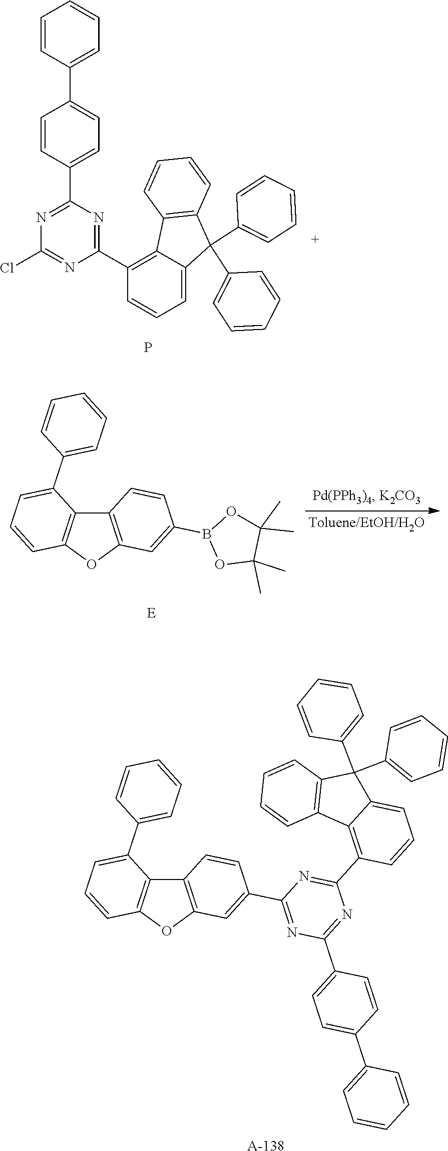

P (3.7 g, 7.4 mmol), E (2.4 g, 7.4 mmol), Pd(PPh₃)₄ (0.37 g, 0.32 mmol), K₂CO₃ (2.2 g, 16.0 mmol), 60 mL of toluene, 15 mL of EtOH, and 15 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was filtered by suction under reduced pressure. The resulting solid was washed with water and ethanol in sequence. The solid was recrystallized from toluene/ethanol to give a white solid (4.3 g, 5.4 mmol) with a yield of 73.0%. The product was confirmed as the target product A-138 with a molecular weight of 791.3.

### Synthesis Example 6: Synthesis of Compound A-230

### Step 1: Synthesis of Intermediate R

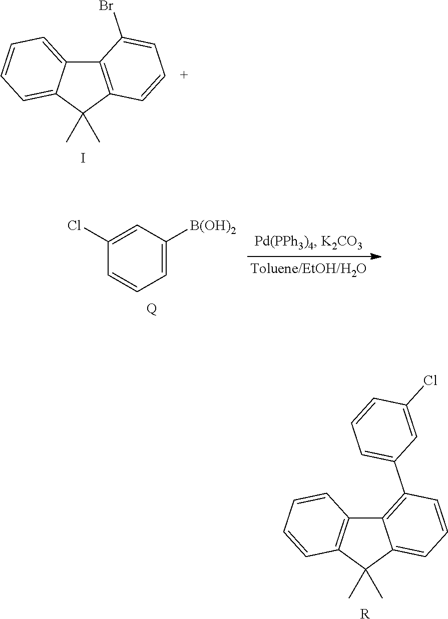

I (10.0 g, 36.61 mmol), Q (6.3 g, 40.27 mmol), Pd(PPh₃)₄ (0.85 g, 0.73 mmol), K₂CO₃ (10.1 g, 73.22 mmol), 100 mL of toluene, 25 mL of EtOH, and 25 mL of H₂O were successively added to a three-necked round-bottom flask. Under the protection of N₂, the mixture was heated to reflux overnight. Heating was stopped, and the reaction was allowed to cool to room temperature. The mixture was subjected to liquid separation. DCM was added to the aqueous phase to extract the aqueous phase multiple times. The organic phase was combined, dried with anhydrous Na₂SO₄, filtered, and concentrated under reduced pressure. The crude product was subjected to silica gel column chromatography (PE) to give Intermediate R (10.8 g, 35.43 mmol) as a white solid with a yield of 96.8%.

### Step 2: Synthesis of Intermediate S

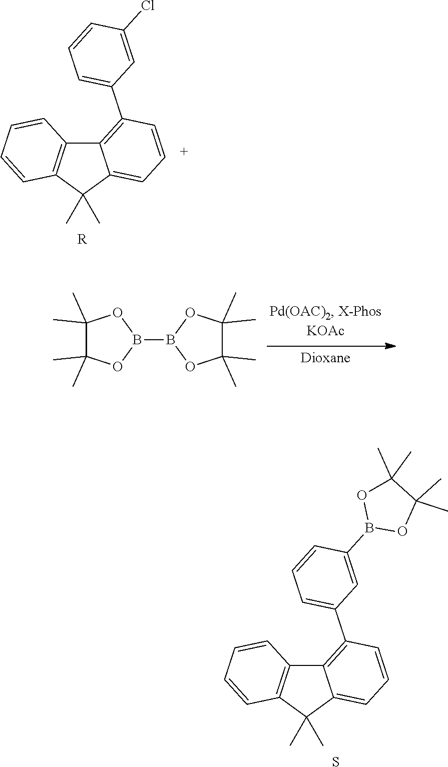

R (10.8 g, 35.43 mmol), bis(pinacolato)diboron (14.0 g, 55.11 mmol), Pd(OAc)₂ (0.17 g, 0.73 mmol), 2-dicyclohexylphosphino-2,4,6-triisopropylbiphenyl (X-Phos, 0.70 g, 1.47 mmol), KOAc (7.21 g, 73.48 mmol), and 100 mL of 1,4-dioxane were successively added to a three-necked round-bottom flask. Under the protection of N₂, the mixture was heated to reflux overnight. After the completion of the reaction was verified by a TLC plate, heating was stopped, and the reaction was allowed to cool to room temperature. The reaction system was filtered through Celite, and the filtrate was concentrated under reduced pressure. The crude product was subjected to silica gel column chromatography (PE/DCM=5:1 to 2:1) to give Intermediate S (13.1 g, 33.05 mmol) as a white solid with a yield of 90.0%.

### Step 3: Synthesis of Intermediate U

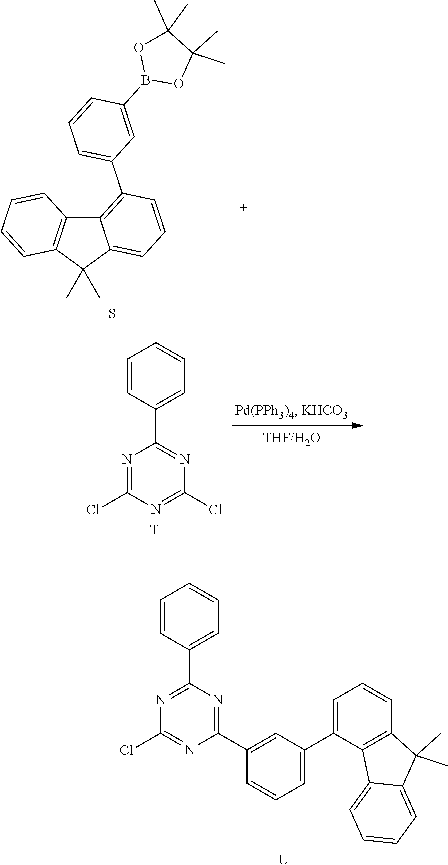

S (9.2 g, 23.2 mmol), T (7.87 g, 34.8 mmol), Pd(PPh₃)₄ (1.07 g, 0.93 mmol), KHCO₃ (5.81 g, 58.0 mmol), 160 mL of THF, and 40 mL of H₂O were successively added to a three-necked round-bottom flask. Under the protection of N₂, the mixture was heated to reflux. After 4 h, the completion of the reaction was verified by a TLC plate, heating was stopped, and the reaction was allowed to cool to room temperature. The mixture was subjected to liquid separation. The aqueous phase was extracted with DCM. The organic phase was combined, dried with anhydrous Na₂SO₄, filtered, and concentrated under reduced pressure. The crude product was subjected to silica gel column chromatography (PE/DCM=6:1 to 4:1) to give Intermediate U (6.3 g, 13.7 mmol) as a white solid with a yield of 59.0%.

### Step 4: Synthesis of Compound A-230

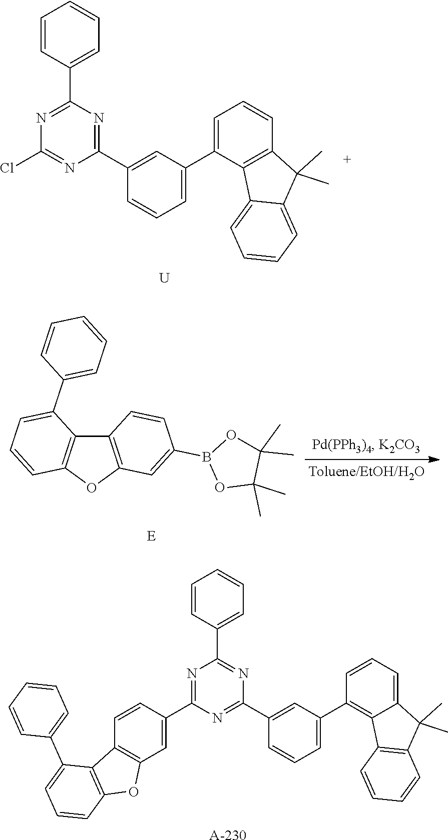

U (4.6 g, 10.0 mmol), E (3.7 g, 10.0 mmol), Pd(PPh₃)₄ (0.35 g, 0.30 mmol), K₂CO₃ (2.76 g, 20.0 mmol), 40 mL of toluene, 10 mL of EtOH, and 10 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was filtered by suction under reduced pressure. The resulting solid was washed with water and ethanol in sequence. The solid was recrystallized from toluene/acetonitrile to give a white solid (4.7 g, 7.04 mmol) with a yield of 70.4%. The product was confirmed as the target product A-230 with a molecular weight of 667.3.

### Synthesis Example 7: Synthesis of Compound A-410

### Step 1: Synthesis of Intermediate W

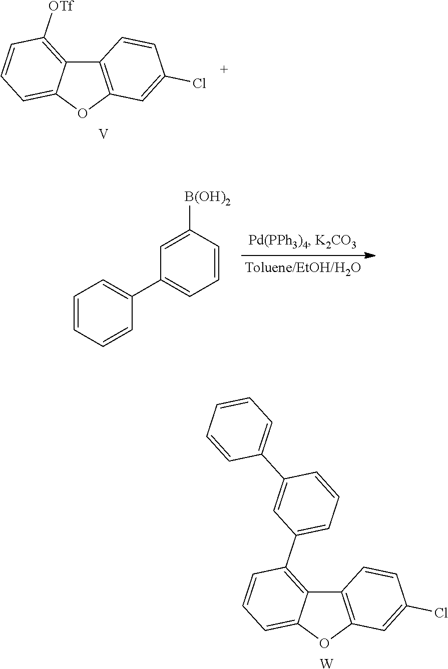

V (6.0 g, 17.1 mmol), 3-biphenylboronic acid (3.70 g, 18.81 mmol), Pd(PPh₃)₄ (0.59 g, 0.51 mmol), K₂CO₃ (4.72 g, 34.2 mmol), 56 mL of toluene, 14 mL of EtOH, and 14 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The organic phase was taken. DCM was added to the aqueous phase to extract the aqueous phase multiple times. The organic phase was combined, dried with anhydrous Na₂SO₄, filtered, and concentrated under reduced pressure. The crude product was purified through column chromatography (PE/DCM=50:1) to give Intermediate W (5.6 g, 15.8 mmol) as a colorless oil with a yield of 92.3%.

### Step 2: Synthesis of Intermediate X

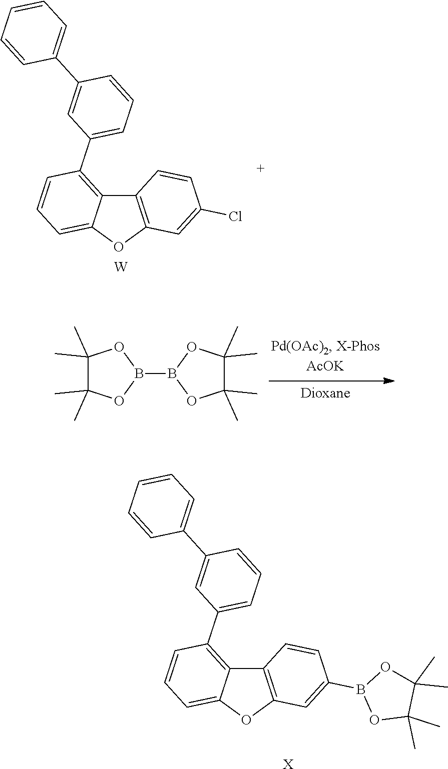

W (6.0 g, 17.47 mmol), bis(pinacolato)diboron (6.65 g, 26.2 mmol), Pd(OAc)₂ (0.08 g, 0.35 mmol), 2-dicyclohexylphosphino-2,4,6-triisopropylbiphenyl (X-Phos, 0.33 g, 0.67 mmol), KOAc (3.43 g, 34.94 mmol), and 87 mL of 1,4-dioxane were successively added to a three-necked round-bottom flask. Under the protection of N₂, the mixture was heated to reflux overnight. After the completion of the reaction was verified by a TLC plate, heating was stopped, and the reaction was allowed to cool to room temperature. The reaction system was filtered through Celite, and the filtrate was concentrated under reduced pressure. The crude product was purified through column chromatography (PE/DCM=4:1 to 2:1) to give Intermediate X (4.71 g, 10.55 mmol) as a white solid with a yield of 60.4%.

### Step 3: Synthesis of Intermediate Y

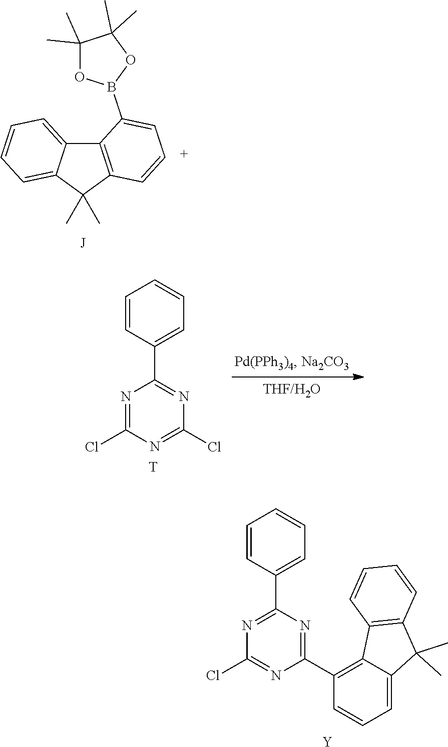

J (10.0 g, 31.2 mmol), T (8.5 g, 37.5 mmol), Pd(PPh₃)₄ (1.1 g, 0.98 mmol), Na₂CO₃ (6.6 g, 62.4 mmol), 240 mL of THF, and 60 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was subjected to liquid separation. The aqueous phase was extracted with DCM. The organic phase was combined, dried with anhydrous Na₂SO₄, filtered, and concentrated under reduced pressure. The crude product was subjected to silica gel column chromatography (PE/DCM=8:1 to 4:1) to give Intermediate Y (6.9 g, 18.0 mmol) as a light yellow solid with a yield of 57.7%.

### Step 4: Synthesis of Compound A-410

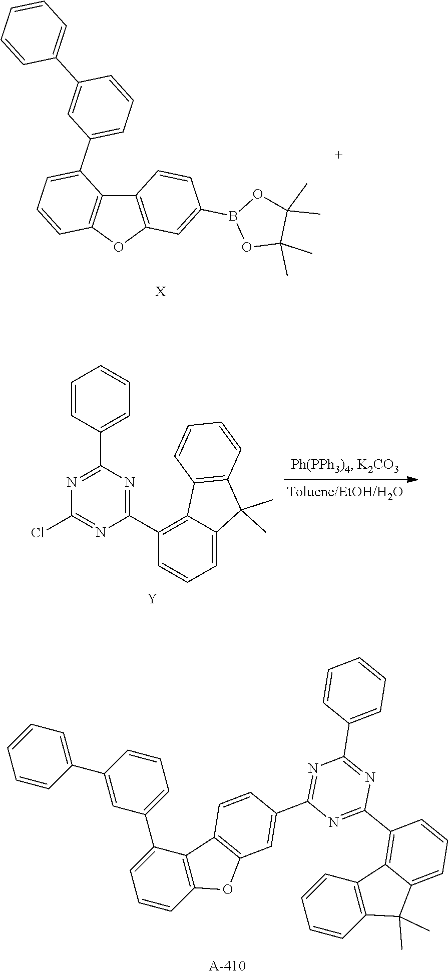

X (4.5 g, 10.0 mmol), Y (3.8 g, 10.0 mmol), Pd(PPh₃)₄ (0.35 g, 0.30 mmol), K₂CO₃ (2.76 g, 20.0 mmol), 40 mL of toluene, 10 mL of EtOH, and 10 mL of H₂O were successively added to a three-necked round-bottom flask. Under the protection of N₂, the mixture was heated to reflux overnight. After the completion of the reaction was verified by a TLC plate, heating was stopped, and the reaction was allowed to cool to room temperature. The mixture was filtered by suction under reduced pressure. The resulting solid was washed with water and ethanol in sequence. The solid was recrystallized from toluene/acetonitrile to give a white solid (5.9 g, 8.8 mmol) with a yield of 88.0%. The product was confirmed as the target product A-410 with a molecular weight of 667.3.

Those skilled in the art will appreciate that the above preparation methods are merely exemplary. Those skilled in the art can obtain other compound structures of the present disclosure through the modifications of the preparation methods.

### Device Example

### Device Example 1

First, a glass substrate having an indium tin oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second at a vacuum degree of about 10⁻⁸ torr. Compound HI was used as a hole injection layer (HIL). Compound HT was used as a hole transport layer (HTL). Compound PH-23 was used as an electron blocking layer (EBL). Compound GD23 was doped in Compound PH-23 and Compound A-8 of the present disclosure, and the resulting mixture was co-deposited for use as an emissive layer (EML). Compound H2 was used as a hole blocking layer (HBL). On the HBL, Compound ET and 8-hydroxyquinolinolato-lithium (Liq) were co-deposited for use as an electron transport layer (ETL). Finally, 8-hydroxyquinolinolato-lithium (Liq) with a thickness of 1 nm was deposited as an electron injection layer, and Al with a thickness of 120 nm was deposited as a cathode. The device was transferred back to the glovebox and encapsulated with a glass lid such that the device was completed.

### Device Example 2

Device Example 2 was prepared by the same method as Device Example 1 except that in the EML, Compound A-8 was replaced with Compound A-12.

### Device Example 3

Device Example 3 was prepared by the same method as Device Example 1 except that in the EML, Compound A-8 was replaced with Compound A-230.

### Device Example 4

Device Example 4 was prepared by the same method as Device Example 1 except that in the EML, Compound A-8 was replaced with Compound A-410.

### Device Example 5

Device Example 5 was prepared by the same method as Device Example 1 except that in the EML, Compound A-8 was replaced with Compound A-7.

### Device Example 6

Device Example 6 was prepared by the same method as Device Example 1 except that in the EML, Compound A-8 was replaced with Compound A-6.

### Device Comparative Example 1

Device Comparative Example 1 was prepared by the same method as Device Example 1 except that in the EML, Compound A-8 was replaced with Compound C-1.

### Device Comparative Example 2

Device Comparative Example 2 was prepared by the same method as Device Example 1 except that in the EML, Compound A-8 was replaced with Compound C-2.

### Device Comparative Example 3

Device Comparative Example 3 was prepared by the same method as Device Example 1 except that in the EML, Compound A-8 was replaced with Compound C-3.

### Device Comparative Example 4

Device Comparative Example 4 was prepared by the same method as Device Example 1 except that in the EML, Compound A-8 was replaced with Compound C-4.

Detailed structures and thicknesses of layers of the devices are shown in the following table. A layer using more than one material is obtained by doping different compounds at their weight ratios as recorded.

TABLE 1
Structures of devices of Device Examples 1 to 6 and Device Comparative Examples 1 to 4

| Device ID | HIL | HTL | EBL | EML | HBL | ETL |
|---|---|---|---|---|---|---|
| Example 1 | Compound | Compound | Compound | Compound PH-23: | Compound | Compound |
| | HI | HT | PH-23 | Compound A-8: | H2 | ET:Liq |
| | (100 Å) | (350 Å) | (50 Å) | Compound GD23 | (50 Å) | (40:60) |
| | | | | (69:23:8) (400 Å) | | (350 Å) |
| Example 2 | Compound | Compound | Compound | Compound PH-23: | Compound | Compound |
| | HI | HT | PH-23 | Compound A-12: | H2 | ET:Liq |
| | (100 Å) | (350 Å) | (50 Å) | Compound GD23 | (50 Å) | (40:60) |
| | | | | (69:23:8) (400 Å) | | (350 Å) |
| Example 3 | Compound | Compound | Compound | Compound PH-23: | Compound | Compound |
| | HI | HT | PH-23 | Compound A-230: | H2 | ET:Liq |
| | (100 Å) | (350 Å) | (50 Å) | Compound GD23 | (50 Å) | (40:60) |
| | | | | (69:23:8) (400 Å) | | (350 Å) |
| Example 4 | Compound | Compound | Compound | Compound PH-23: | Compound | Compound |
| | HI | HT | PH-23 | Compound A-410: | H2 | ET:Liq |
| | (100 Å) | (350 Å) | (50 Å) | Compound GD23 | (50 Å) | (40:60) |
| | | | | (69:23:8) (400 Å) | | (350 Å) |
| Comparative | Compound | Compound | Compound | Compound PH-23: | Compound | Compound |
| Example 1 | HI | HT | PH-23 | Compound C-1: | H2 | ET:Liq |
| | (100 Å) | (350 Å) | (50 Å) | Compound GD23 | (50 Å) | (40:60) |
| | | | | (69:23:8) (400 Å) | | (350 Å) |
| Example 5 | Compound | Compound | Compound | Compound PH-23: | Compound | Compound |
| | HI | HT | PH-23 | Compound A-7: | H2 | ET:Liq |
| | (100 Å) | (350 Å) | (50 Å) | Compound GD23 | (50 Å) | (40:60) |
| | | | | (69:23:8) (400 Å) | | (350 Å) |
| Comparative | Compound | Compound | Compound | Compound PH-23: | Compound | Compound |
| Example 2 | HI | HT | PH-23 | Compound C-2: | H2 | ET:Liq |
| | (100 Å) | (350 Å) | (50 Å) | Compound GD23 | (50 Å) | (40:60) |
| | | | | (69:23:8) (400 Å) | | (350 Å) |
| Comparative | Compound | Compound | Compound | Compound PH-23: | Compound | Compound |
| Example 3 | HI | HT | PH-23 | Compound C-3: | H2 | ET:Liq |
| | (100 Å) | (350 Å) | (50 Å) | Compound GD23 | (50 Å) | (40:60) |
| | | | | (69:23:8) (400 Å) | | (350 Å) |
| Example 6 | Compound | Compound | Compound | Compound PH-23: | Compound | Compound |
| | HI | HT | PH-23 | Compound A-6: | H2 | ET:Liq |
| | (100 Å) | (350 Å) | (50 Å) | Compound GD23 | (50 Å) | (40:60) |
| | | | | (69:23:8) (400 Å) | | (350 Å) |
| Comparative | Compound | Compound | Compound | Compound PH-23: | Compound | Compound |
| Example 4 | HI | HT | PH-23 | Compound C-4: | H2 | ET:Liq |
| | (100 Å) | (350 Å) | (50 Å) | Compound GD23 | (50 Å) | (40:60) |
| | | | | (69:23:8) (400 Å) | | (350 Å) |

The materials used in the devices have the following structures:

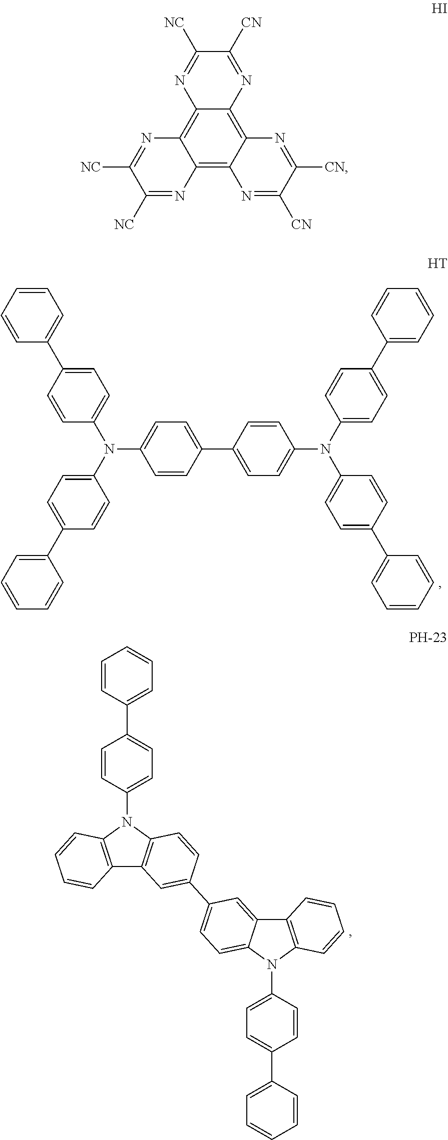 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

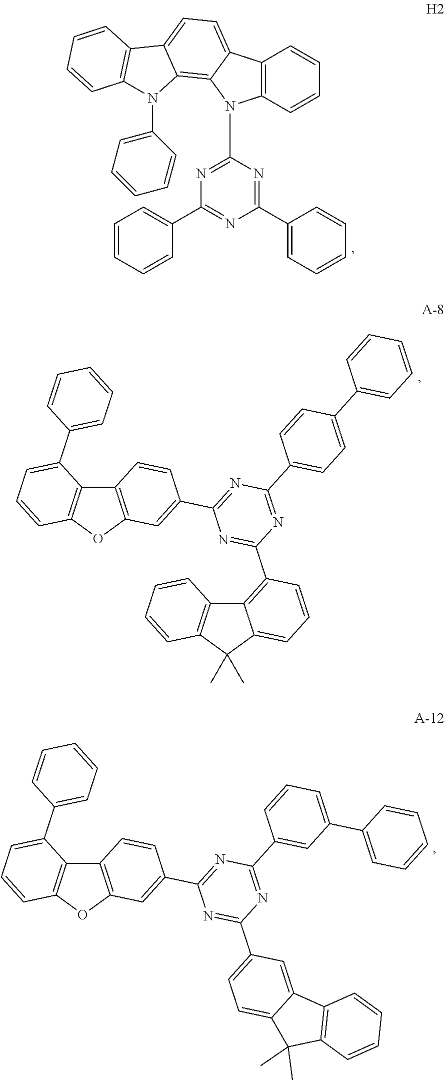 [SMILES: c1ccc(-c2nc(-c3ccccc3)nc(-n3c4ccccc4c4ccc5c6ccccc6n(-c6ccccc6)c5c43)n2)cc1 ; CC1(C)c2ccccc2-c2c(-c3nc(-c4ccc(-c5ccccc5)cc4)nc(-c4ccc5c(c4)oc4cccc(-c6ccccc6)c45)n3)cccc21 ; CC1(C)c2ccccc2-c2cc(-c3nc(-c4cccc(-c5ccccc5)c4)nc(-c4ccc5c(c4)oc4cccc(-c6ccccc6)c45)n3)ccc21]

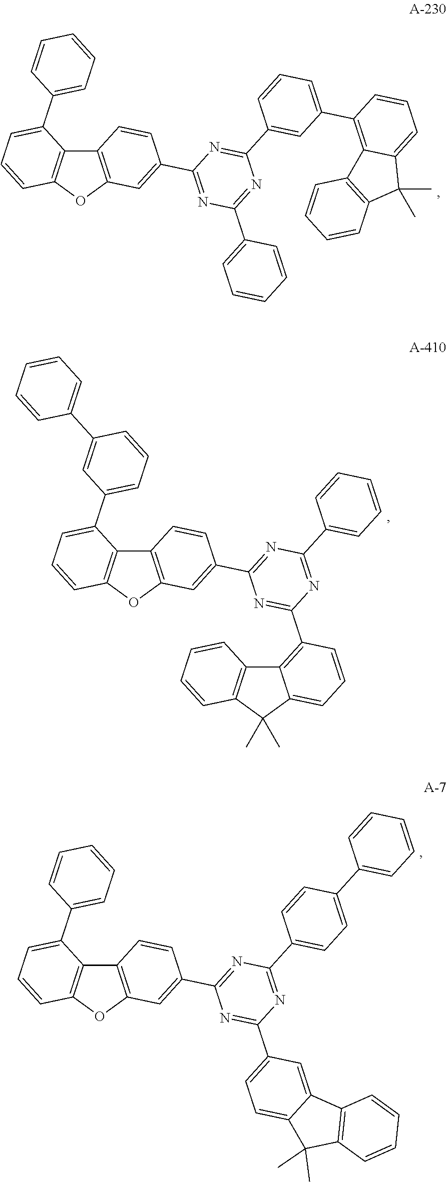 [SMILES: CC1(C)c2ccccc2-c2c(-c3cccc(-c4nc(-c5ccccc5)nc(-c5ccc6c(c5)oc5cccc(-c7ccccc7)c56)n4)c3)cccc21 ; CC1(C)c2ccccc2-c2c(-c3nc(-c4ccccc4)nc(-c4ccc5c(c4)oc4cccc(-c6cccc(-c7ccccc7)c6)c45)n3)cccc21 ; CC1(C)c2ccccc2-c2cc(-c3nc(-c4ccc(-c5ccccc5)cc4)nc(-c4ccc5c(c4)oc4cccc(-c6ccccc6)c45)n3)ccc21]

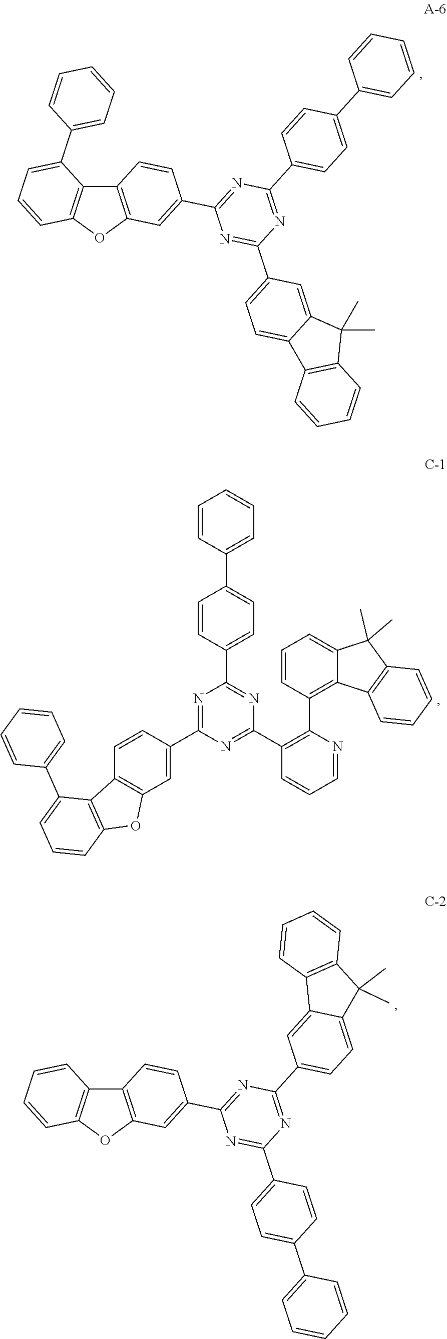 [SMILES: CC1(C)c2ccccc2-c2ccc(-c3nc(-c4ccc(-c5ccccc5)cc4)nc(-c4ccc5c(c4)oc4cccc(-c6ccccc6)c45)n3)cc21 ; CC1(C)c2ccccc2-c2c(-c3ncccc3-c3nc(-c4ccc(-c5ccccc5)cc4)nc(-c4ccc5c(c4)oc4cccc(-c6ccccc6)c45)n3)cccc21 ; CC1(C)c2ccccc2-c2cc(-c3nc(-c4ccc(-c5ccccc5)cc4)nc(-c4ccc5c(c4)oc4ccccc45)n3)ccc21]

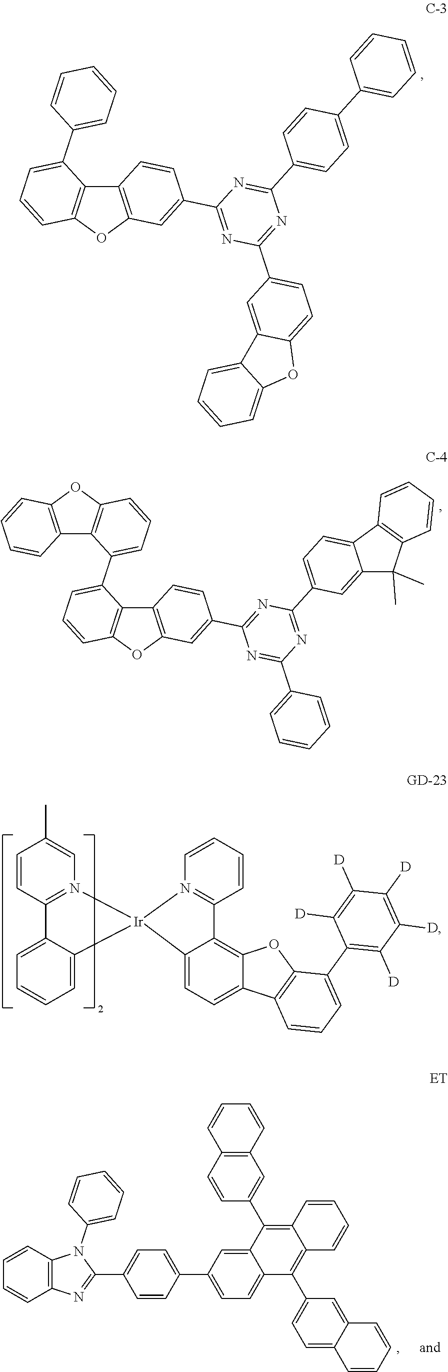

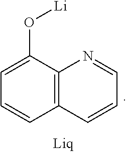

Table 2 shows the CIE data, external quantum efficiency (EQE), and current efficiency (CE) measured at a constant current of 15 mA/cm² and the device lifetime (LT95) measured at a constant current of 80 mA/cm².

TABLE 2
Device data of Examples 1 to 6 and Comparative Examples 1 to 4

| | | CIE | EQE | CE | LT97 |
|---|---|---|---|---|---|
| Device ID | EML | (x, y) | (%) | (cd/A) | (h) |
| Example 1 | PH-23:A-8:GD23 | (0.357, | 21.8 | 83 | 41.9 |
| | (69:23:8) | 0.619) | | | |
| Example 2 | PH-23:A-12:GD23 | (0.355, | 21.8 | 83 | 33.7 |
| | (69:23:8) | 0.620) | | | |
| Example 3 | PH-23:A-230:GD23 | (0.352, | 21.9 | 84 | 20.9 |
| | (69:23:8) | 0.623) | | | |
| Example 4 | PH-23:A-410:GD23 | (0.353, | 22.0 | 84 | 39.5 |
| | (69:23:8) | 0.622) | | | |
| Comparative | PH-23:C-1:GD23 | (0.359, | 21.3 | 81 | 0.4 |
| Example 1 | (69:23:8) | 0.617) | | | |
| Example 5 | PH-23:A-7:GD23 | (0.357, | 21.8 | 83 | 39.0 |
| | (69:23:8) | 0.618) | | | |
| Comparative | PH-23:C-2:GD23 | (0.356, | 21.6 | 83 | 24.8 |
| Example 2 | (69:23:8) | 0.619) | | | |
| Comparative | PH-23:C-3:GD23 | (0.356, | 21.8 | 83 | 4.0 |
| Example 3 | (69:23:8) | 0.619) | | | |
| Example 6 | PH-23:A-6:GD23 | (0.357, | 21.6 | 83 | 22.5 |
| | (69:23:8) | 0.619) | | | |
| Comparative | PH-23:C-4:GD23 | (0.356, | 21.6 | 83 | 9.0 |
| Example 4 | (69:23:8) | 0.619) | | | |

### DISCUSSION

In Example 1, Example 3, and Comparative Example 1, the phosphorescent dopant GD23 was doped in Compound A-8 and Compound A-230 of the present disclosure and Compound C-1 that was not provided in the present disclosure, respectively. Compound A-8, Compound A-230, and Compound C-1 differed mainly in that fluorene was joined to triazine through different groups. Fluorene was directly bonded to triazine in Compound A-8, fluorene was joined to triazine through phenylene in Compound A-230, and fluorine was joined to triazine through pyridylene in Compound C-1. Compared with Comparative Example 1, the EQE and CE of both Example 1 and Example 3 were improved, and in particular, the device lifetime was greatly improved by 103.7 times and 51.2 times, respectively. Meanwhile, compared with Comparative Example 1, the EQE and CE of the devices of Example 2 and Example 4 using compounds A-12 and A-410 of the present disclosure were improved, and the device lifetime was greatly improved by 83.2 times and 97.7 times, respectively. It indicates that when applied to organic electroluminescent devices, the compound of the present disclosure having the structure of Formula 1, in which fluorene and triazine are joined through a direct bond or an arylene group, achieve higher efficiency and longer device lifetime compared than the compound including an heteroarylene group as the bridging group between fluorene and triazine.

In Example 5 and Comparative Example 2, the phosphorescent dopant GD23 was doped in Compound A-7 of the present disclosure and Compound C-2 that was not provided in the present disclosure, respectively. Compound A-7 and Compound C-2 differed only in that dibenzofuryl had an aryl substituent at the position 1. Compared with Comparative Example 2, the EQE and CE of Example 5 were comparable to those of Comparative Example 2, but the device lifetime of Example 5 was improved by 57.2%. It indicates that when applied to organic electroluminescent devices, the compound of the present disclosure having the structure of Formula 1, which has a specific substituent at the specific position of the dibenzo five-membered ring, achieve longer device lifetime compared than the compound without a substituent on the dibenzo five-membered ring.

In Example 5 and Comparative Example 3, the phosphorescent dopant GD23 was doped in Compound A-7 of the present disclosure and Compound C-3 that was not provided in the present disclosure, respectively. Compound A-7 and Compound C-3 differed only in that dimethylfluorenyl was replaced with dibenzofuryl. The EQE and CE of Example 5 were comparable to those of Comparative Example 3, but the device lifetime of Example 5 was greatly improved by 8.7 times. It indicates that when applied to organic electroluminescent devices, the compound of the present disclosure having the structure of Formula 1, which has a dibenzo five-membered ring-triazine-fluorene backbone, achieve longer device lifetime compared than the compound having a dibenzofuran-triazine-dibenzofuran backbone structure.

In Example 6 and Comparative Example 4, the phosphorescent dopant GD23 was doped in Compound A-6 of the present disclosure and Compound C-4 that was not provided in the present disclosure, respectively. Compound A-6 and Compound C-4 differed mainly in that an oxygen-containing heterocyclic ring was formed in phenyl at the position 1 of dibenzofuran in Compound C-4. The EQE and CE of Example 6 were comparable to those of Comparative Example 4, but the device lifetime of Example 6 was greatly improved by 1.5 times. It indicates that when applied to organic electroluminescent devices, the compound of the present disclosure having the structure of Formula 1, which has a specific substituent at the specific position of the dibenzo five-membered ring, achieve longer device lifetime compared than the compound having an oxygen-containing heterocyclic substituent at the position 1 of the dibenzo five-membered ring.

In summary, the compound of the present disclosure, when applied to the organic electroluminescent devices, can improve the electron-hole transport balance of the material. Compared with the compound that is not provided in the present disclosure, the compound of the present disclosure can improve the efficiency (EQE and CE) of the device to which the compound of the present disclosure is applied greatly or to some extent, enable the device lifetime to greatly increase unexpectedly, and greatly improve the overall performance of the device. The compound of the present disclosure is of great help to the industry.

It is to be understood that various embodiments described herein are merely examples and not intended to limit the scope of the present disclosure. Therefore, it is apparent to the persons skilled in the art that the present disclosure as claimed may include variations of specific embodiments and preferred embodiments described herein. Many of materials and structures described herein may be substituted with other materials and structures without departing from the spirit of the present disclosure. It is to be understood that various theories as to why the present disclosure works are not intended to be limitative.

What is claimed is:

1. A compound, having a structure represented by Formula 1: 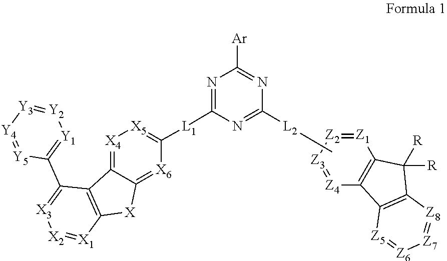
wherein X is selected from O, S or Se;
X₁ to X₆ are, at each occurrence identically or differently, selected from CRₓ or N;
Y₁ to Y₅ are, at each occurrence identically or differently, selected from CR_{y} or N;
Z₁ to Z₈ are, at each occurrence identically or differently, selected from C, CR_{z} or N, and one of Z₁ to Z₄ is selected from C and joined to L₂;
Ar is, at each occurrence identically or differently, selected from substituted or unsubstituted aryl having 6 to 30 carbon atoms, substituted or unsubstituted heteroaryl having 3 to 30 carbon atoms or combinations thereof,
L₁ is, at each occurrence identically or differently, selected from a single bond, substituted or unsubstituted arylene having 6 to 30 carbon atoms, substituted or unsubstituted heteroarylene having 3 to 12 carbon atoms or combinations thereof,
L₂ is, at each occurrence identically or differently, selected from a single bond or substituted or unsubstituted arylene having 6 to 30 carbon atoms;
R, Rₓ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof,
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a sulfinyl group, a sulfonyl group, a phosphino group, and combinations thereof,
adjacent substituents R can be optionally joined to form a ring;
adjacent substituents R_{z} can be optionally joined to form a ring; and
adjacent substituents R_{y} can be optionally joined to form a carbocyclic ring or a heterocycle comprising one or more of N, Si, P, Ge and B atoms.

2. The compound according to claim 1, wherein X is selected from O or S; preferably, X is O.

3. The compound according to claim 1, wherein X₁ to X₆ are, at each occurrence identically or differently, selected from CRₓ; and/or Y₁ to Y₅ are, at each occurrence identically or differently, selected from CR_{y}; and/or Z₁ to Z₈ are, at each occurrence identically or differently, selected from C or CR_{z}, and one of Z₁ to Z₄ is selected from C and joined to L₂.

4. The compound according to claim 1, wherein L₁ is, at each occurrence identically or differently, selected from a single bond, substituted or unsubstituted arylene having 6 to 20 carbon atoms, substituted or unsubstituted heteroarylene having 3 to 12 carbon atoms or combinations thereof,
preferably, L₁ is, at each occurrence identically or differently, selected from a single bond, substituted or unsubstituted phenylene, substituted or unsubstituted biphenylene, substituted or unsubstituted naphthylene, substituted or unsubstituted pyridylene or combinations thereof.

5. The compound according to claim 1, wherein L₂ is, at each occurrence identically or differently, selected from a single bond or substituted or unsubstituted arylene having 6 to 20 carbon atoms;
preferably, L₂ is, at each occurrence identically or differently, selected from a single bond, substituted or unsubstituted phenylene, substituted or unsubstituted biphenylene, substituted or unsubstituted naphthylene or combinations thereof.

6. The compound according to claim 1, wherein Rₓ, R_{y} and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, cyano, and combinations thereof,
preferably, Rₓ, R_{y} and R_{z} are, at each occurrence identically or differently, selected from the group consisting of: hydrogen, deuterium, substituted or unsubstituted phenyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted carbazolyl, substituted or unsubstituted dibenzofuryl, substituted or unsubstituted dibenzothienyl, cyano, and combinations thereof.

7. The compound according to claim 1, wherein R is, at each occurrence identically or differently, selected from the group consisting of: hydrogen, deuterium, substituted or unsubstituted alkyl having 1 to 20 carbon atoms, substituted or unsubstituted cycloalkyl having 3 to 20 ring carbon atoms, substituted or unsubstituted aryl having 6 to 20 carbon atoms, substituted or unsubstituted heteroaryl having 3 to 20 carbon atoms, and combinations thereof,
preferably, R is, at each occurrence identically or differently, selected from the group consisting of: hydrogen, deuterium, substituted or unsubstituted methyl, substituted or unsubstituted ethyl, substituted or unsubstituted propyl, substituted or unsubstituted butyl, substituted or unsubstituted pentyl, substituted or unsubstituted hexyl, substituted or unsubstituted cyclopentyl, substituted or unsubstituted adamantyl, substituted or unsubstituted cyclohexyl, substituted or unsubstituted phenyl, substituted or unsubstituted pyridyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted carbazolyl, substituted or unsubstituted dibenzofuryl, substituted or unsubstituted dibenzothienyl, and combinations thereof.

8. The compound according to claim 1, wherein Ar is, at each occurrence identically or differently, selected from substituted or unsubstituted aryl having 6 to 20 carbon atoms, substituted or unsubstituted heteroaryl having 3 to 20 carbon atoms or combinations thereof,
preferably, Ar is, at each occurrence identically or differently, selected from the group consisting of: substituted or unsubstituted phenyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted carbazolyl, substituted or unsubstituted dibenzofuryl, substituted or unsubstituted dibenzothienyl, and combinations thereof.

9. The compound according to claim 1, wherein the compound is selected from the group consisting of the following compounds: 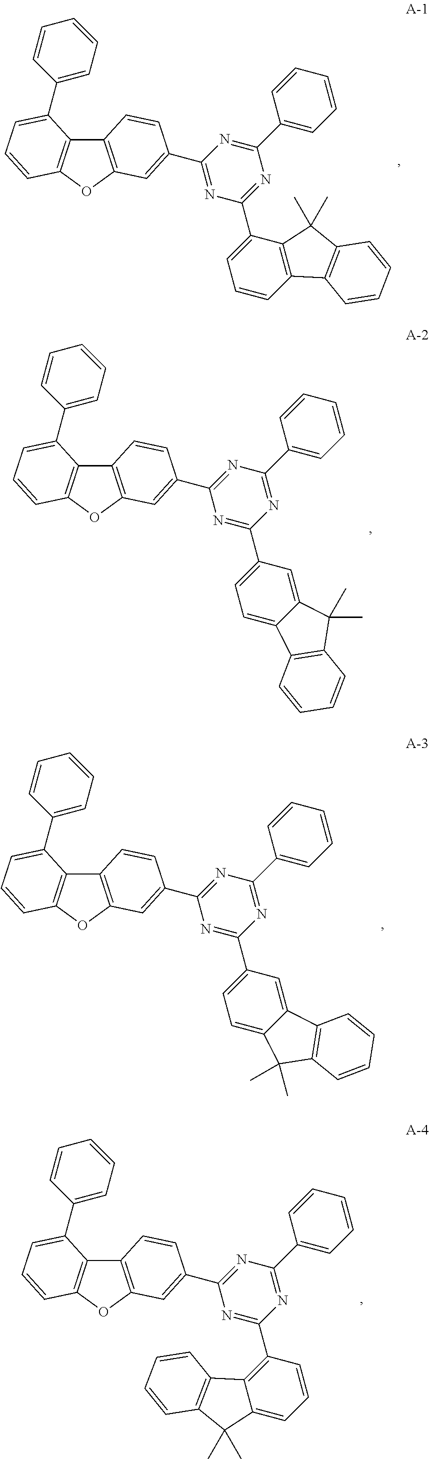 [SMILES: CC1(C)c2ccccc2-c2cccc(-c3nc(-c4ccccc4)nc(-c4ccc5c(c4)oc4cccc(-c6ccccc6)c45)n3)c21 ; CC1(C)c2ccccc2-c2ccc(-c3nc(-c4ccccc4)nc(-c4ccc5c(c4)oc4cccc(-c6ccccc6)c45)n3)cc21 ; CC1(C)c2ccccc2-c2cc(-c3nc(-c4ccccc4)nc(-c4ccc5c(c4)oc4cccc(-c6ccccc6)c45)n3)ccc21 ; CC1(C)c2ccccc2-c2c(-c3nc(-c4ccccc4)nc(-c4ccc5c(c4)oc4cccc(-c6ccccc6)c45)n3)cccc21] 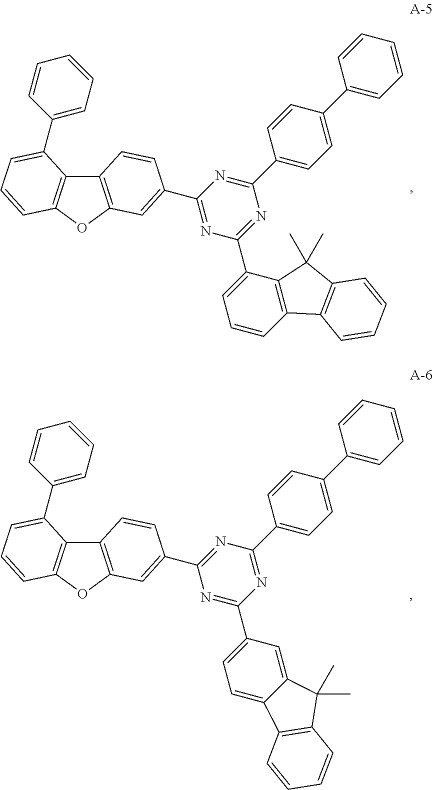 [SMILES: CC1(C)c2ccccc2-c2cccc(-c3nc(-c4ccc(-c5ccccc5)cc4)nc(-c4ccc5c(c4)oc4cccc(-c6ccccc6)c45)n3)c21 ; CC1(C)c2ccccc2-c2ccc(-c3nc(-c4ccc(-c5ccccc5)cc4)nc(-c4ccc5c(c4)oc4cccc(-c6ccccc6)c45)n3)cc21] 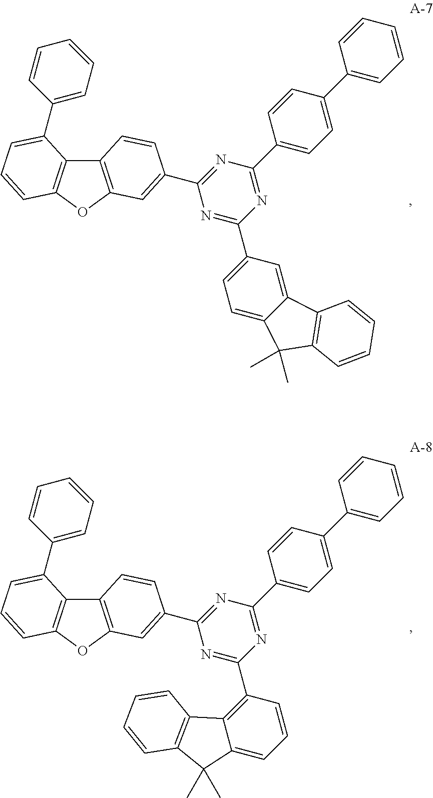 [SMILES: CC1(C)c2ccccc2-c2cc(-c3nc(-c4ccc(-c5ccccc5)cc4)nc(-c4ccc5c(c4)oc4cccc(-c6ccccc6)c45)n3)ccc21 ; CC1(C)c2ccccc2-c2c(-c3nc(-c4ccc(-c5ccccc5)cc4)nc(-c4ccc5c(c4)oc4cccc(-c6ccccc6)c45)n3)cccc21] 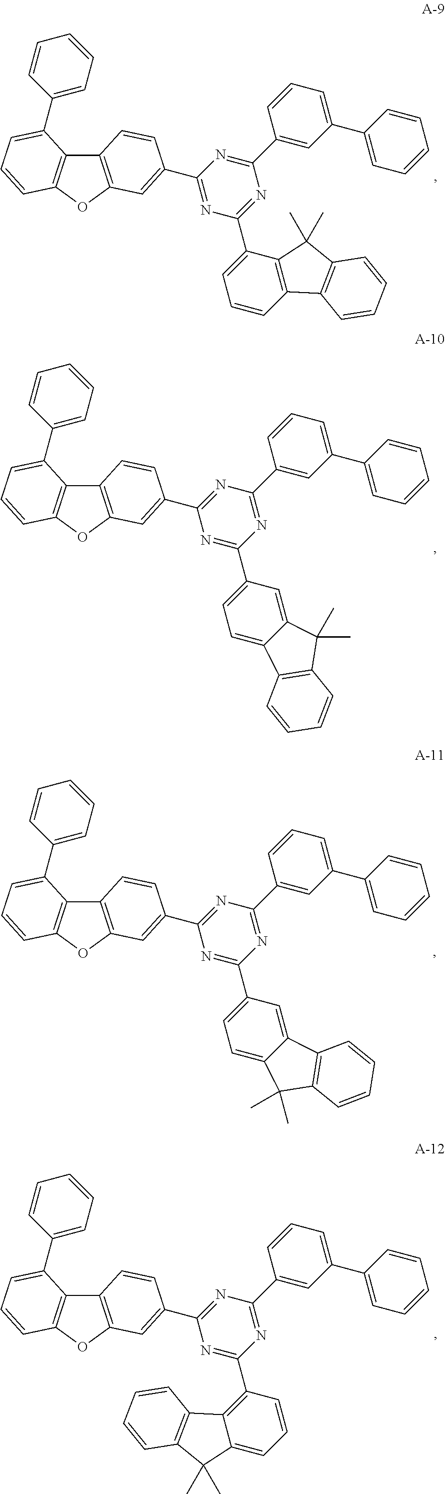 [SMILES: CC1(C)c2ccccc2-c2cccc(-c3nc(-c4cccc(-c5ccccc5)c4)nc(-c4ccc5c(c4)oc4cccc(-c6ccccc6)c45)n3)c21 ; CC1(C)c2ccccc2-c2ccc(-c3nc(-c4cccc(-c5ccccc5)c4)nc(-c4ccc5c(c4)oc4cccc(-c6ccccc6)c45)n3)cc21 ; CC1(C)c2ccccc2-c2cc(-c3nc(-c4cccc(-c5ccccc5)c4)nc(-c4ccc5c(c4)oc4cccc(-c6ccccc6)c45)n3)ccc21 ; CC1(C)c2ccccc2-c2c(-c3nc(-c4cccc(-c5ccccc5)c4)nc(-c4ccc5c(c4)oc4cccc(-c6ccccc6)c45)n3)cccc21] 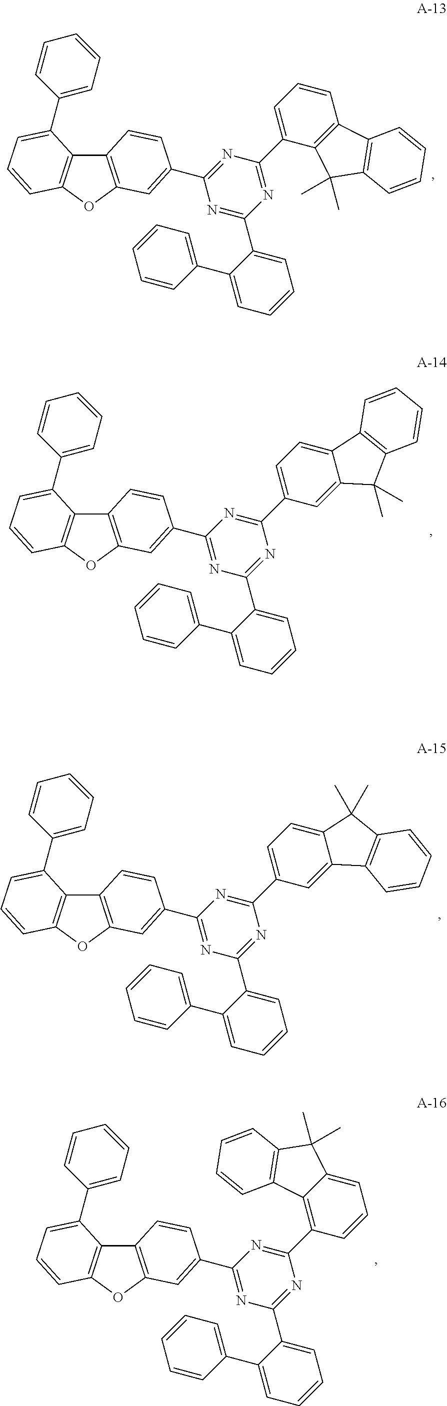 [SMILES: CC1(C)c2ccccc2-c2cccc(-c3nc(-c4ccc5c(c4)oc4cccc(-c6ccccc6)c45)nc(-c4ccccc4-c4ccccc4)n3)c21 ; CC1(C)c2ccccc2-c2ccc(-c3nc(-c4ccc5c(c4)oc4cccc(-c6ccccc6)c45)nc(-c4ccccc4-c4ccccc4)n3)cc21 ; CC1(C)c2ccccc2-c2cc(-c3nc(-c4ccc5c(c4)oc4cccc(-c6ccccc6)c45)nc(-c4ccccc4-c4ccccc4)n3)ccc21 ; CC1(C)c2ccccc2-c2c(-c3nc(-c4ccc5c(c4)oc4cccc(-c6ccccc6)c45)nc(-c4ccccc4-c4ccccc4)n3)cccc21] 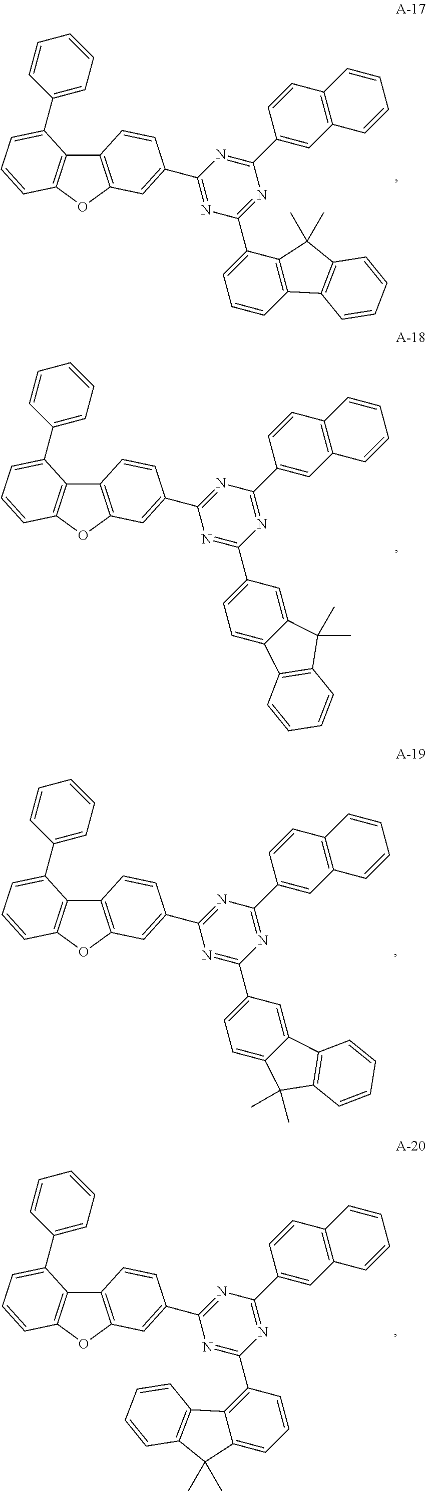 [SMILES: CC1(C)c2ccccc2-c2cccc(-c3nc(-c4ccc5ccccc5c4)nc(-c4ccc5c(c4)oc4cccc(-c6ccccc6)c45)n3)c21 ; CC1(C)c2ccccc2-c2ccc(-c3nc(-c4ccc5ccccc5c4)nc(-c4ccc5c(c4)oc4cccc(-c6ccccc6)c45)n3)cc21 ; CC1(C)c2ccccc2-c2cc(-c3nc(-c4ccc5ccccc5c4)nc(-c4ccc5c(c4)oc4cccc(-c6ccccc6)c45)n3)ccc21 ; CC1(C)c2ccccc2-c2c(-c3nc(-c4ccc5ccccc5c4)nc(-c4ccc5c(c4)oc4cccc(-c6ccccc6)c45)n3)cccc21] 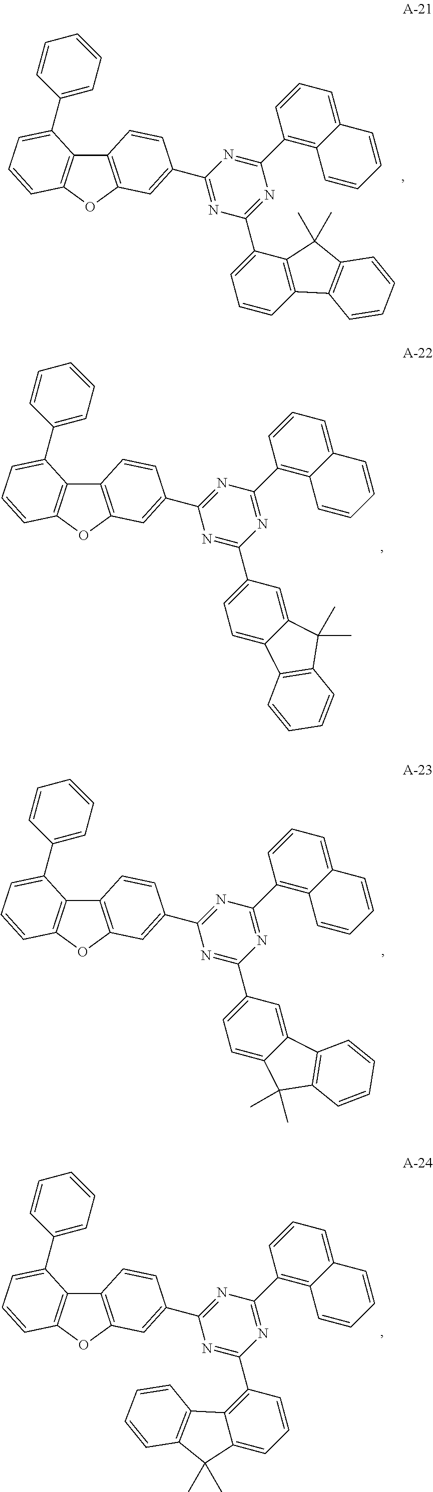 [SMILES: CC1(C)c2ccccc2-c2cccc(-c3nc(-c4ccc5c(c4)oc4cccc(-c6ccccc6)c45)nc(-c4cccc5ccccc45)n3)c21 ; CC1(C)c2ccccc2-c2ccc(-c3nc(-c4ccc5c(c4)oc4cccc(-c6ccccc6)c45)nc(-c4cccc5ccccc45)n3)cc21 ; CC1(C)c2ccccc2-c2cc(-c3nc(-c4ccc5c(c4)oc4cccc(-c6ccccc6)c45)nc(-c4cccc5ccccc45)n3)ccc21 ; CC1(C)c2ccccc2-c2c(-c3nc(-c4ccc5c(c4)oc4cccc(-c6ccccc6)c45)nc(-c4cccc5ccccc45)n3)cccc21] 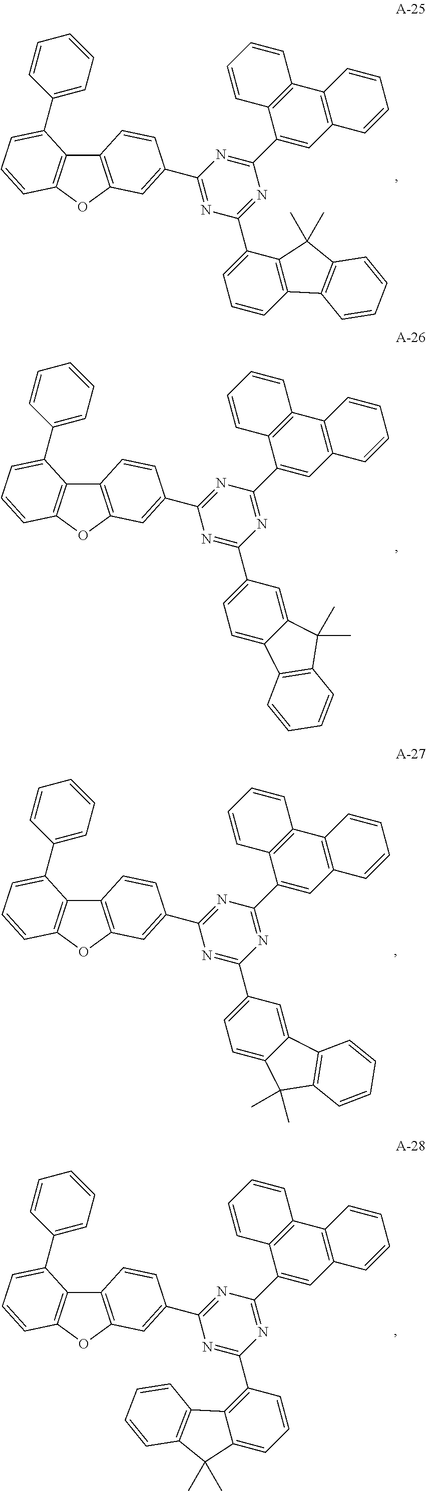 [SMILES: CC1(C)c2ccccc2-c2cccc(-c3nc(-c4ccc5c(c4)oc4cccc(-c6ccccc6)c45)nc(-c4cc5ccccc5c5ccccc45)n3)c21 ; CC1(C)c2ccccc2-c2ccc(-c3nc(-c4ccc5c(c4)oc4cccc(-c6ccccc6)c45)nc(-c4cc5ccccc5c5ccccc45)n3)cc21 ; CC1(C)c2ccccc2-c2cc(-c3nc(-c4ccc5c(c4)oc4cccc(-c6ccccc6)c45)nc(-c4cc5ccccc5c5ccccc45)n3)ccc21 ; CC1(C)c2ccccc2-c2c(-c3nc(-c4ccc5c(c4)oc4cccc(-c6ccccc6)c45)nc(-c4cc5ccccc5c5ccccc45)n3)cccc21] 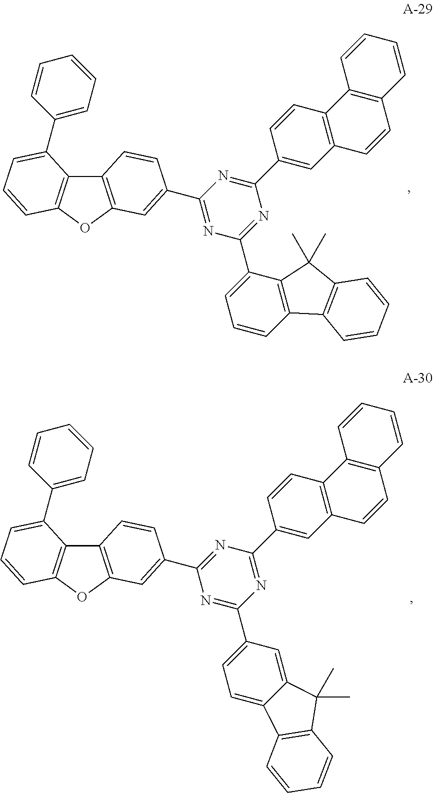 [SMILES: CC1(C)c2ccccc2-c2cccc(-c3nc(-c4ccc5c(ccc6ccccc65)c4)nc(-c4ccc5c(c4)oc4cccc(-c6ccccc6)c45)n3)c21 ; CC1(C)c2ccccc2-c2ccc(-c3nc(-c4ccc5c(ccc6ccccc65)c4)nc(-c4ccc5c(c4)oc4cccc(-c6ccccc6)c45)n3)cc21] 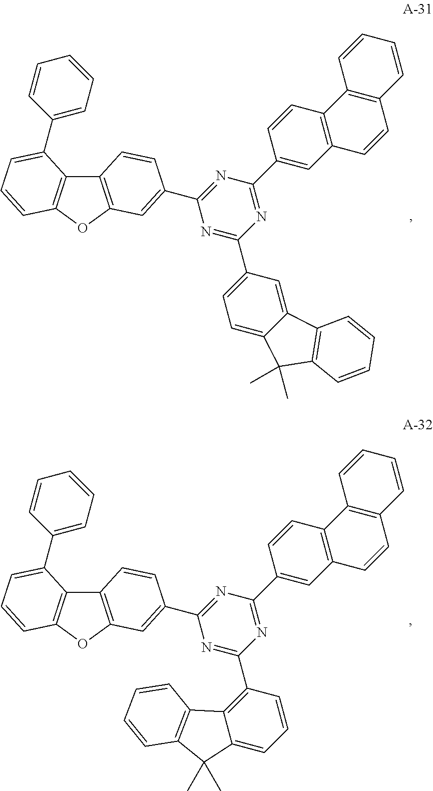 [SMILES: CC1(C)c2ccccc2-c2cc(-c3nc(-c4ccc5c(ccc6ccccc65)c4)nc(-c4ccc5c(c4)oc4cccc(-c6ccccc6)c45)n3)ccc21 ; CC1(C)c2ccccc2-c2c(-c3nc(-c4ccc5c(ccc6ccccc65)c4)nc(-c4ccc5c(c4)oc4cccc(-c6ccccc6)c45)n3)cccc21] 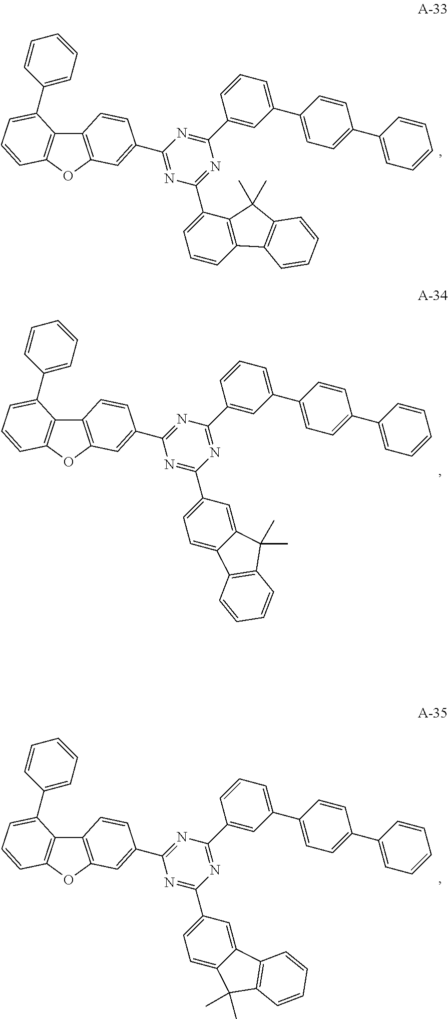 [SMILES: CC1(C)c2ccccc2-c2cccc(-c3nc(-c4cccc(-c5ccc(-c6ccccc6)cc5)c4)nc(-c4ccc5c(c4)oc4cccc(-c6ccccc6)c45)n3)c21 ; CC1(C)c2ccccc2-c2ccc(-c3nc(-c4cccc(-c5ccc(-c6ccccc6)cc5)c4)nc(-c4ccc5c(c4)oc4cccc(-c6ccccc6)c45)n3)cc21 ; CC1(C)c2ccccc2-c2cc(-c3nc(-c4cccc(-c5ccc(-c6ccccc6)cc5)c4)nc(-c4ccc5c(c4)oc4cccc(-c6ccccc6)c45)n3)ccc21] 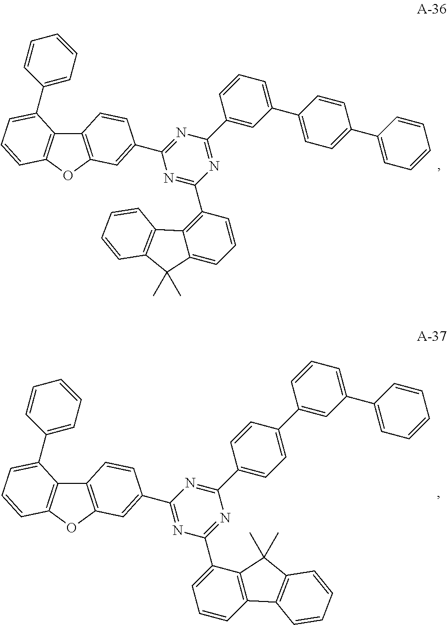 [SMILES: CC1(C)c2ccccc2-c2c(-c3nc(-c4cccc(-c5ccc(-c6ccccc6)cc5)c4)nc(-c4ccc5c(c4)oc4cccc(-c6ccccc6)c45)n3)cccc21 ; CC1(C)c2ccccc2-c2cccc(-c3nc(-c4ccc(-c5cccc(-c6ccccc6)c5)cc4)nc(-c4ccc5c(c4)oc4cccc(-c6ccccc6)c45)n3)c21] 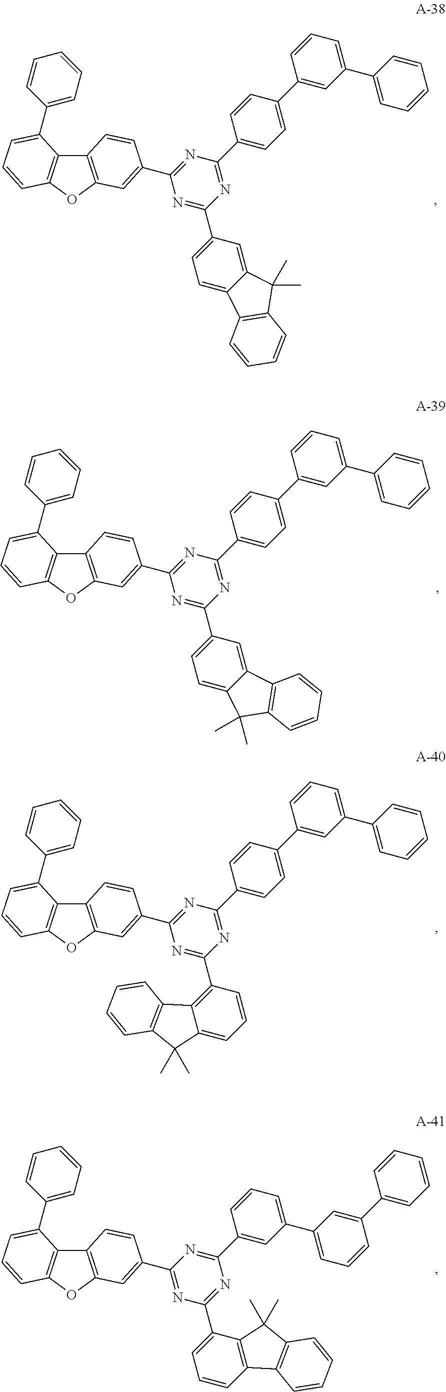 [SMILES: CC1(C)c2ccccc2-c2ccc(-c3nc(-c4ccc(-c5cccc(-c6ccccc6)c5)cc4)nc(-c4ccc5c(c4)oc4cccc(-c6ccccc6)c45)n3)cc21 ; CC1(C)c2ccccc2-c2cc(-c3nc(-c4ccc(-c5cccc(-c6ccccc6)c5)cc4)nc(-c4ccc5c(c4)oc4cccc(-c6ccccc6)c45)n3)ccc21 ; CC1(C)c2ccccc2-c2c(-c3nc(-c4ccc(-c5cccc(-c6ccccc6)c5)cc4)nc(-c4ccc5c(c4)oc4cccc(-c6ccccc6)c45)n3)cccc21 ; CC1(C)c2ccccc2-c2cccc(-c3nc(-c4cccc(-c5cccc(-c6ccccc6)c5)c4)nc(-c4ccc5c(c4)oc4cccc(-c6ccccc6)c45)n3)c21] 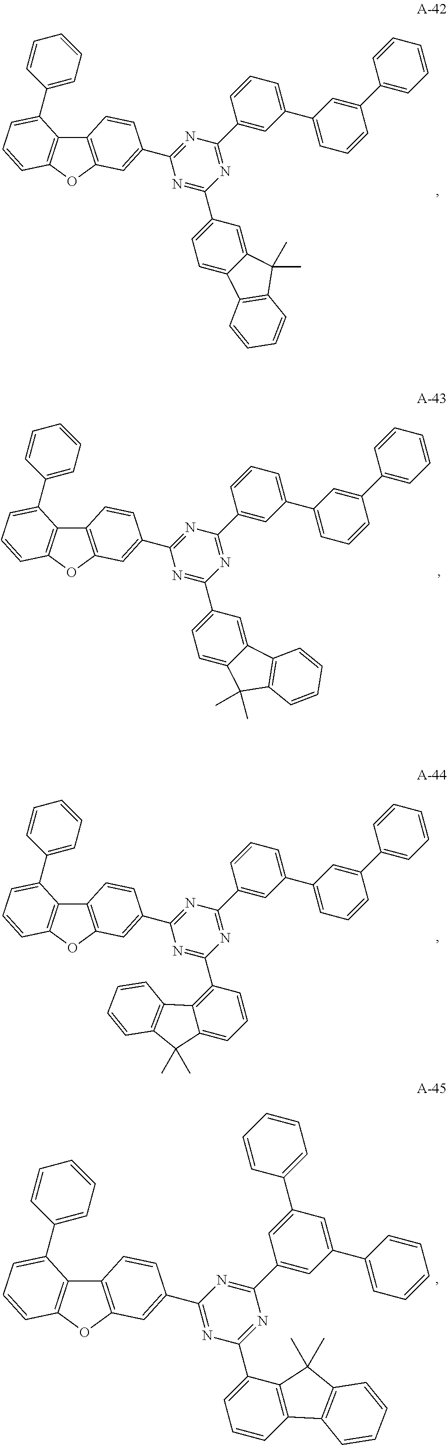 [SMILES: CC1(C)c2ccccc2-c2ccc(-c3nc(-c4cccc(-c5cccc(-c6ccccc6)c5)c4)nc(-c4ccc5c(c4)oc4cccc(-c6ccccc6)c45)n3)cc21 ; CC1(C)c2ccccc2-c2cc(-c3nc(-c4cccc(-c5cccc(-c6ccccc6)c5)c4)nc(-c4ccc5c(c4)oc4cccc(-c6ccccc6)c45)n3)ccc21 ; CC1(C)c2ccccc2-c2c(-c3nc(-c4cccc(-c5cccc(-c6ccccc6)c5)c4)nc(-c4ccc5c(c4)oc4cccc(-c6ccccc6)c45)n3)cccc21 ; CC1(C)c2ccccc2-c2cccc(-c3nc(-c4cc(-c5ccccc5)cc(-c5ccccc5)c4)nc(-c4ccc5c(c4)oc4cccc(-c6ccccc6)c45)n3)c21] 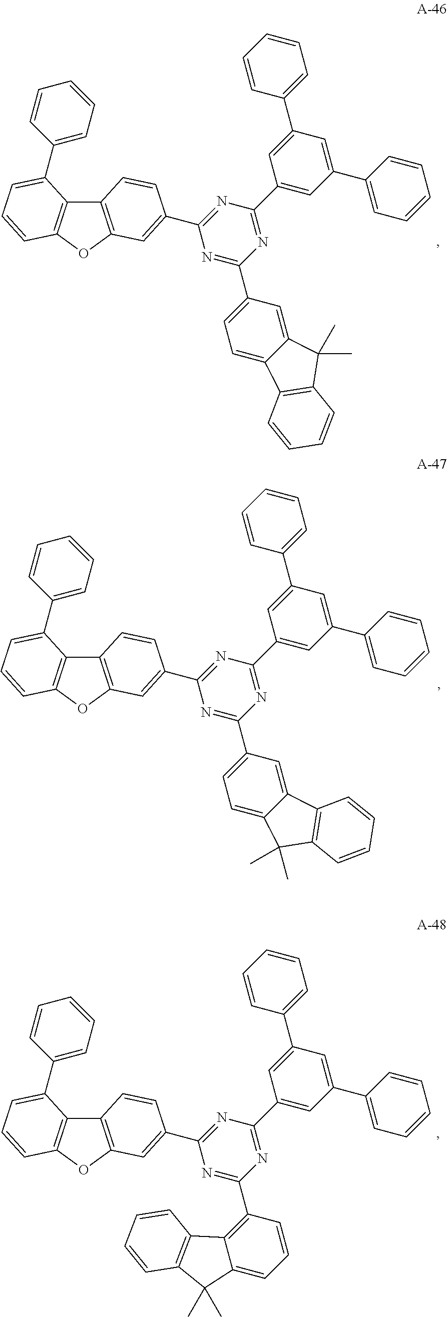 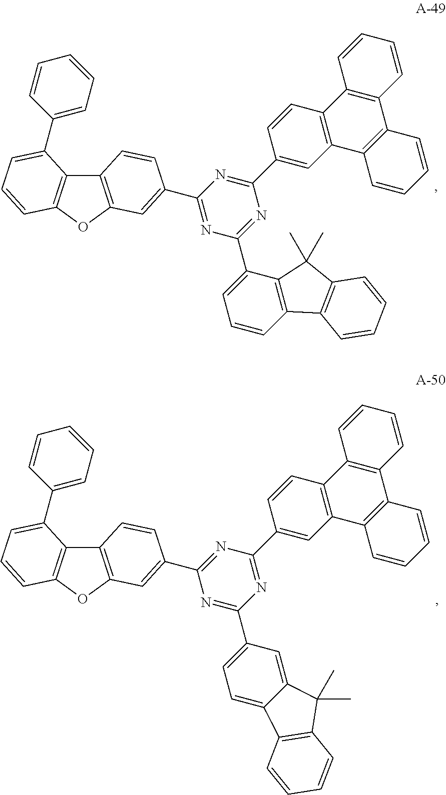 [SMILES: CC1(C)c2ccccc2-c2cccc(-c3nc(-c4ccc5c(c4)oc4cccc(-c6ccccc6)c45)nc(-c4ccc5c6ccccc6c6ccccc6c5c4)n3)c21 ; CC1(C)c2ccccc2-c2ccc(-c3nc(-c4ccc5c(c4)oc4cccc(-c6ccccc6)c45)nc(-c4ccc5c6ccccc6c6ccccc6c5c4)n3)cc21] 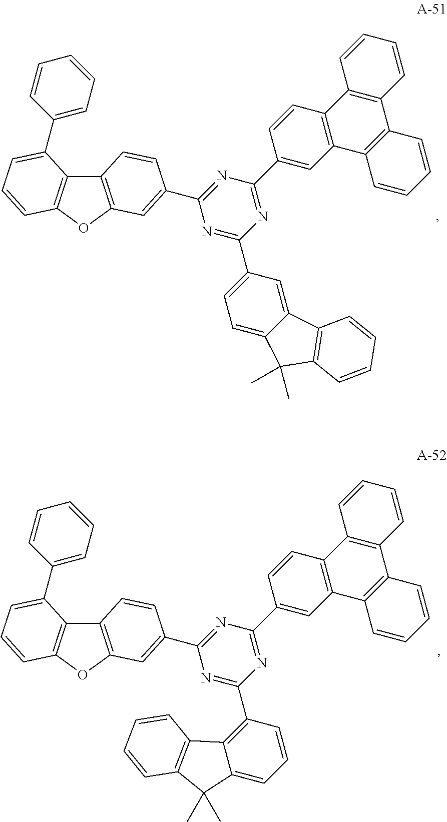 [SMILES: CC1(C)c2ccccc2-c2cc(-c3nc(-c4ccc5c(c4)oc4cccc(-c6ccccc6)c45)nc(-c4ccc5c6ccccc6c6ccccc6c5c4)n3)ccc21 ; CC1(C)c2ccccc2-c2c(-c3nc(-c4ccc5c(c4)oc4cccc(-c6ccccc6)c45)nc(-c4ccc5c6ccccc6c6ccccc6c5c4)n3)cccc21] 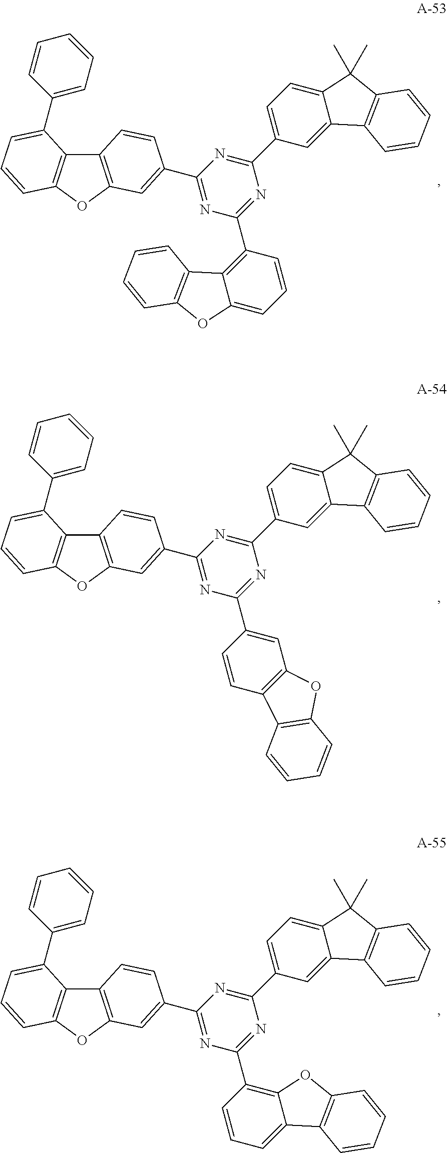 [SMILES: CC1(C)c2ccccc2-c2cc(-c3nc(-c4ccc5c(c4)oc4cccc(-c6ccccc6)c45)nc(-c4cccc5oc6ccccc6c45)n3)ccc21 ; CC1(C)c2ccccc2-c2cc(-c3nc(-c4ccc5c(c4)oc4ccccc45)nc(-c4ccc5c(c4)oc4cccc(-c6ccccc6)c45)n3)ccc21 ; CC1(C)c2ccccc2-c2cc(-c3nc(-c4ccc5c(c4)oc4cccc(-c6ccccc6)c45)nc(-c4cccc5c4oc4ccccc45)n3)ccc21] 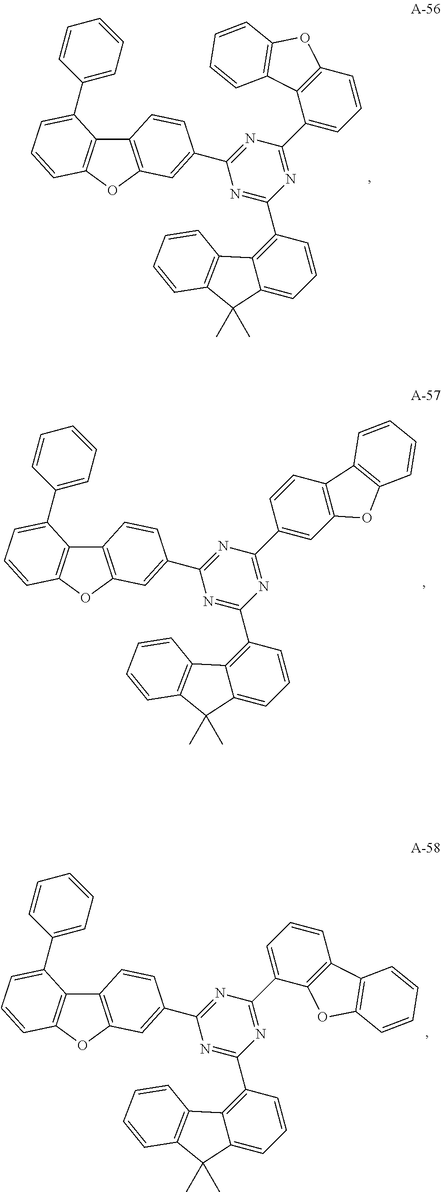 [SMILES: CC1(C)c2ccccc2-c2c(-c3nc(-c4ccc5c(c4)oc4cccc(-c6ccccc6)c45)nc(-c4cccc5oc6ccccc6c45)n3)cccc21 ; CC1(C)c2ccccc2-c2c(-c3nc(-c4ccc5c(c4)oc4ccccc45)nc(-c4ccc5c(c4)oc4cccc(-c6ccccc6)c45)n3)cccc21 ; CC1(C)c2ccccc2-c2c(-c3nc(-c4ccc5c(c4)oc4cccc(-c6ccccc6)c45)nc(-c4cccc5c4oc4ccccc45)n3)cccc21] 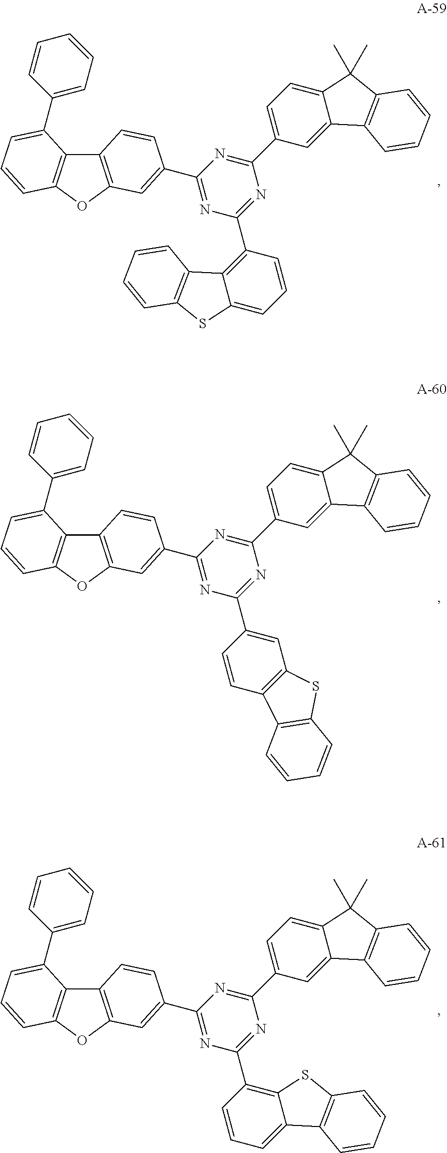 [SMILES: CC1(C)c2ccccc2-c2cc(-c3nc(-c4ccc5c(c4)oc4cccc(-c6ccccc6)c45)nc(-c4cccc5sc6ccccc6c45)n3)ccc21 ; CC1(C)c2ccccc2-c2cc(-c3nc(-c4ccc5c(c4)oc4cccc(-c6ccccc6)c45)nc(-c4ccc5c(c4)sc4ccccc45)n3)ccc21 ; CC1(C)c2ccccc2-c2cc(-c3nc(-c4ccc5c(c4)oc4cccc(-c6ccccc6)c45)nc(-c4cccc5c4sc4ccccc45)n3)ccc21] 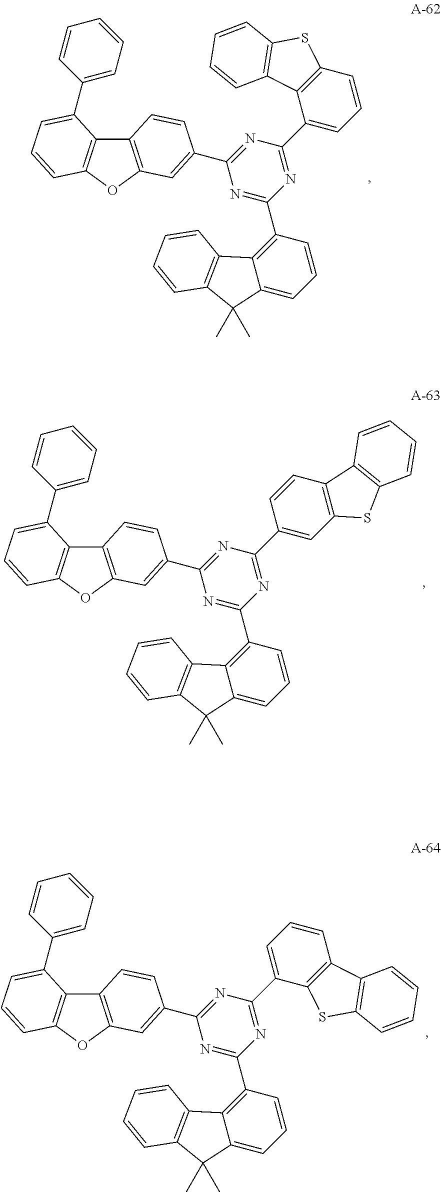 [SMILES: CC1(C)c2ccccc2-c2c(-c3nc(-c4ccc5c(c4)oc4cccc(-c6ccccc6)c45)nc(-c4cccc5sc6ccccc6c45)n3)cccc21 ; CC1(C)c2ccccc2-c2c(-c3nc(-c4ccc5c(c4)oc4cccc(-c6ccccc6)c45)nc(-c4ccc5c(c4)sc4ccccc45)n3)cccc21 ; CC1(C)c2ccccc2-c2c(-c3nc(-c4ccc5c(c4)oc4cccc(-c6ccccc6)c45)nc(-c4cccc5c4sc4ccccc45)n3)cccc21] 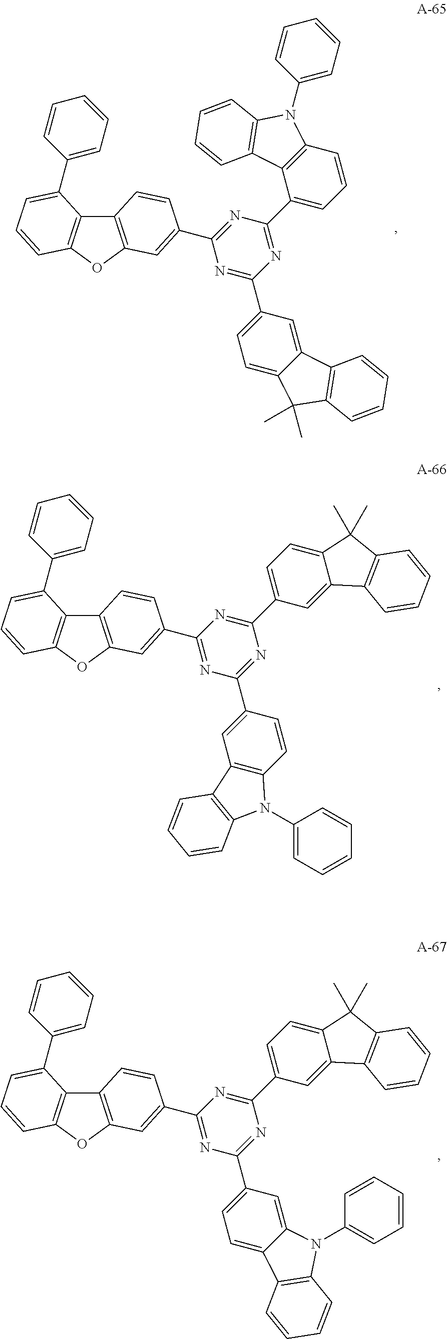 [SMILES: CC1(C)c2ccccc2-c2cc(-c3nc(-c4ccc5c(c4)oc4cccc(-c6ccccc6)c45)nc(-c4cccc5c4c4ccccc4n5-c4ccccc4)n3)ccc21 ; CC1(C)c2ccccc2-c2cc(-c3nc(-c4ccc5c(c4)oc4cccc(-c6ccccc6)c45)nc(-c4ccc5c(c4)c4ccccc4n5-c4ccccc4)n3)ccc21 ; CC1(C)c2ccccc2-c2cc(-c3nc(-c4ccc5c(c4)oc4cccc(-c6ccccc6)c45)nc(-c4ccc5c6ccccc6n(-c6ccccc6)c5c4)n3)ccc21] 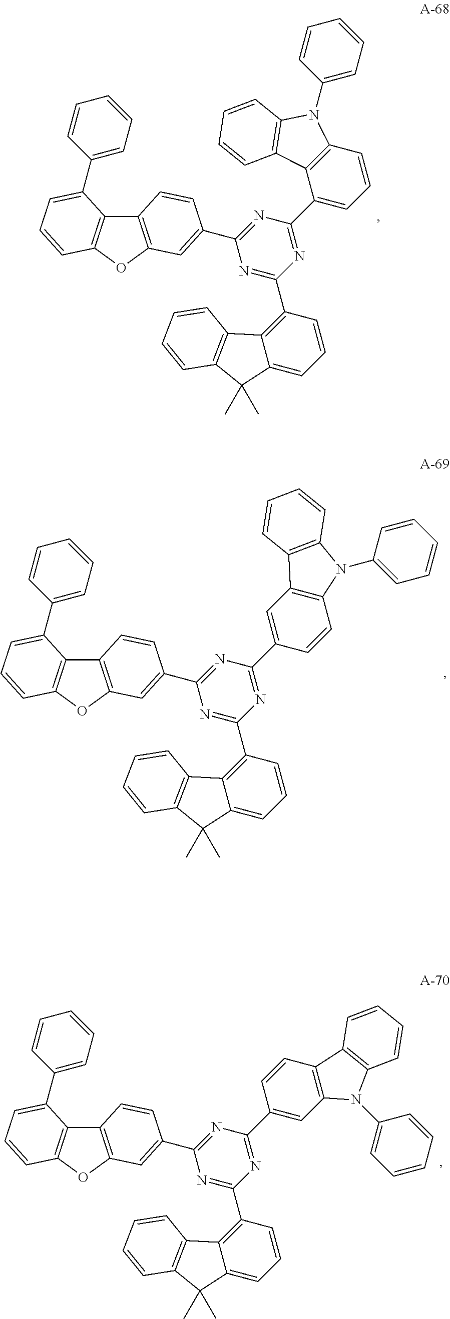 [SMILES: CC1(C)c2ccccc2-c2c(-c3nc(-c4ccc5c(c4)oc4cccc(-c6ccccc6)c45)nc(-c4cccc5c4c4ccccc4n5-c4ccccc4)n3)cccc21 ; CC1(C)c2ccccc2-c2c(-c3nc(-c4ccc5c(c4)oc4cccc(-c6ccccc6)c45)nc(-c4ccc5c(c4)c4ccccc4n5-c4ccccc4)n3)cccc21 ; CC1(C)c2ccccc2-c2c(-c3nc(-c4ccc5c(c4)oc4cccc(-c6ccccc6)c45)nc(-c4ccc5c6ccccc6n(-c6ccccc6)c5c4)n3)cccc21] 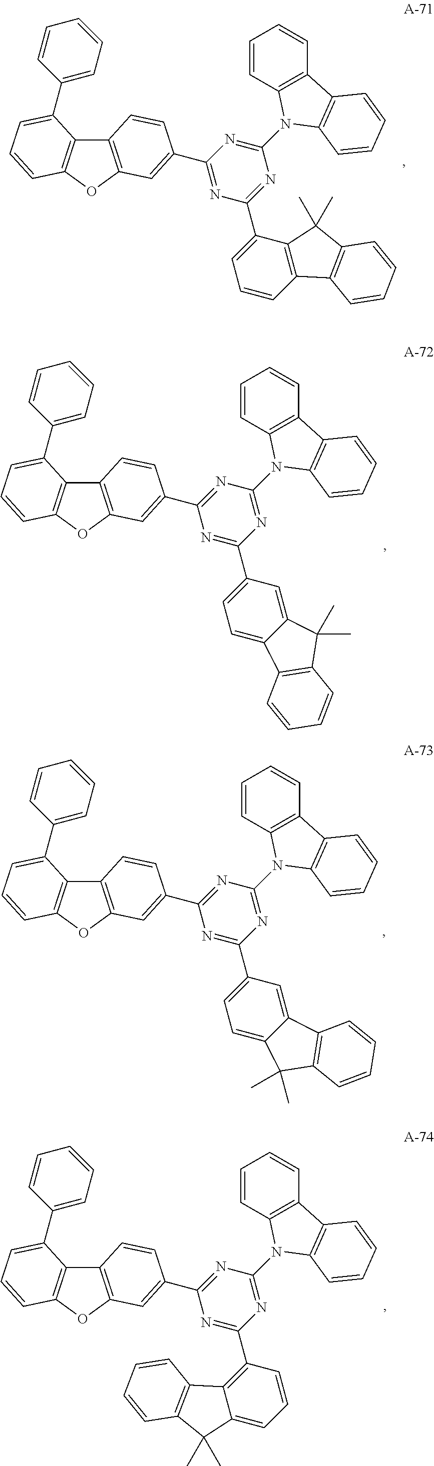 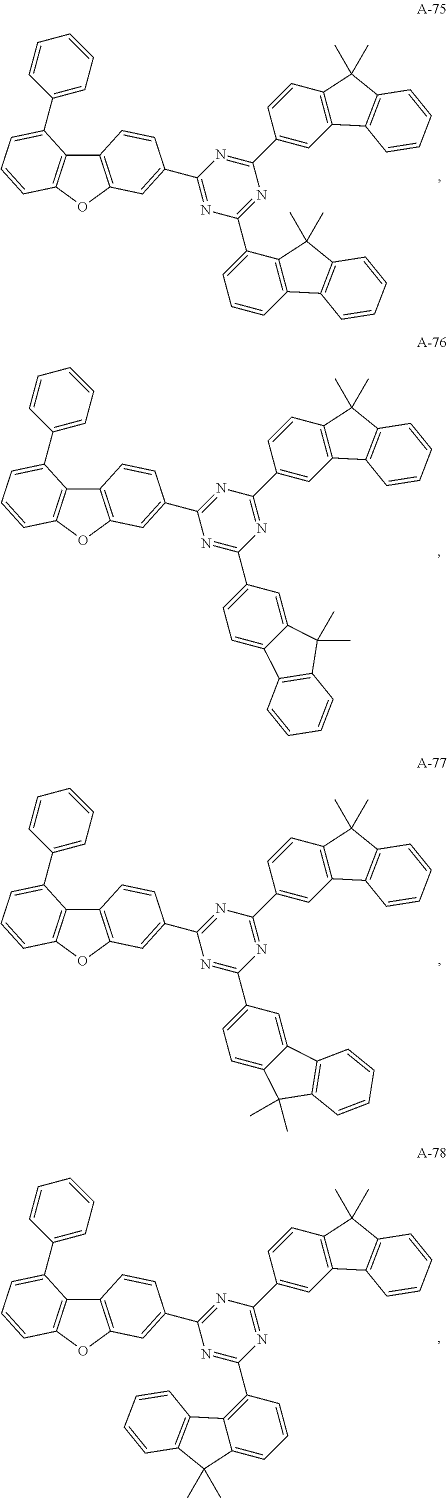 [SMILES: CC1(C)c2ccccc2-c2cc(-c3nc(-c4ccc5c(c4)oc4cccc(-c6ccccc6)c45)nc(-c4cccc5c4C(C)(C)c4ccccc4-5)n3)ccc21 ; CC1(C)c2ccccc2-c2cc(-c3nc(-c4ccc5c(c4)C(C)(C)c4ccccc4-5)nc(-c4ccc5c(c4)oc4cccc(-c6ccccc6)c45)n3)ccc21 ; CC1(C)c2ccccc2-c2cc(-c3nc(-c4ccc5c(c4)-c4ccccc4C5(C)C)nc(-c4ccc5c(c4)oc4cccc(-c6ccccc6)c45)n3)ccc21 ; CC1(C)c2ccccc2-c2cc(-c3nc(-c4ccc5c(c4)oc4cccc(-c6ccccc6)c45)nc(-c4cccc5c4-c4ccccc4C5(C)C)n3)ccc21] 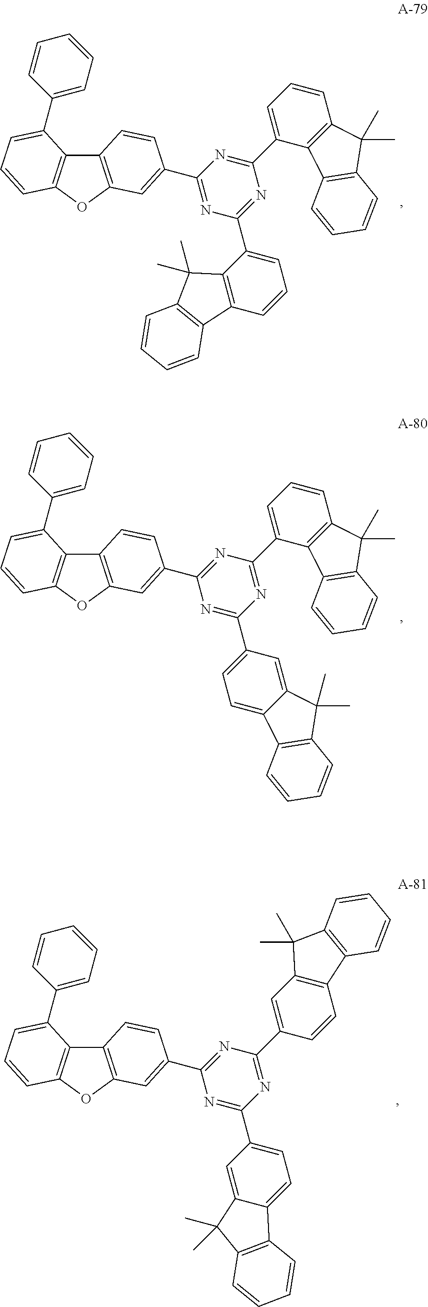 [SMILES: CC1(C)c2ccccc2-c2c(-c3nc(-c4ccc5c(c4)oc4cccc(-c6ccccc6)c45)nc(-c4cccc5c4C(C)(C)c4ccccc4-5)n3)cccc21 ; CC1(C)c2ccccc2-c2ccc(-c3nc(-c4ccc5c(c4)oc4cccc(-c6ccccc6)c45)nc(-c4cccc5c4-c4ccccc4C5(C)C)n3)cc21 ; CC1(C)c2ccccc2-c2ccc(-c3nc(-c4ccc5c(c4)C(C)(C)c4ccccc4-5)nc(-c4ccc5c(c4)oc4cccc(-c6ccccc6)c45)n3)cc21] 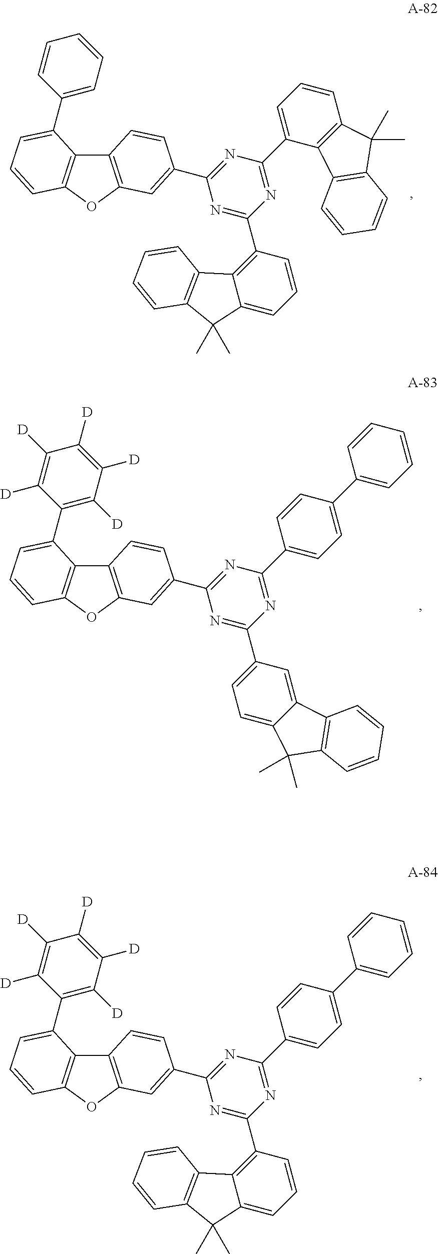 [SMILES: CC1(C)c2ccccc2-c2c(-c3nc(-c4ccc5c(c4)oc4cccc(-c6ccccc6)c45)nc(-c4cccc5c4-c4ccccc4C5(C)C)n3)cccc21 ; [2H]c1c([2H])c([2H])c(-c2cccc3oc4cc(-c5nc(-c6ccc(-c7ccccc7)cc6)nc(-c6ccc7c(c6)-c6ccccc6C7(C)C)n5)ccc4c23)c([2H])c1[2H] ; [2H]c1c([2H])c([2H])c(-c2cccc3oc4cc(-c5nc(-c6ccc(-c7ccccc7)cc6)nc(-c6cccc7c6-c6ccccc6C7(C)C)n5)ccc4c23)c([2H])c1[2H]] 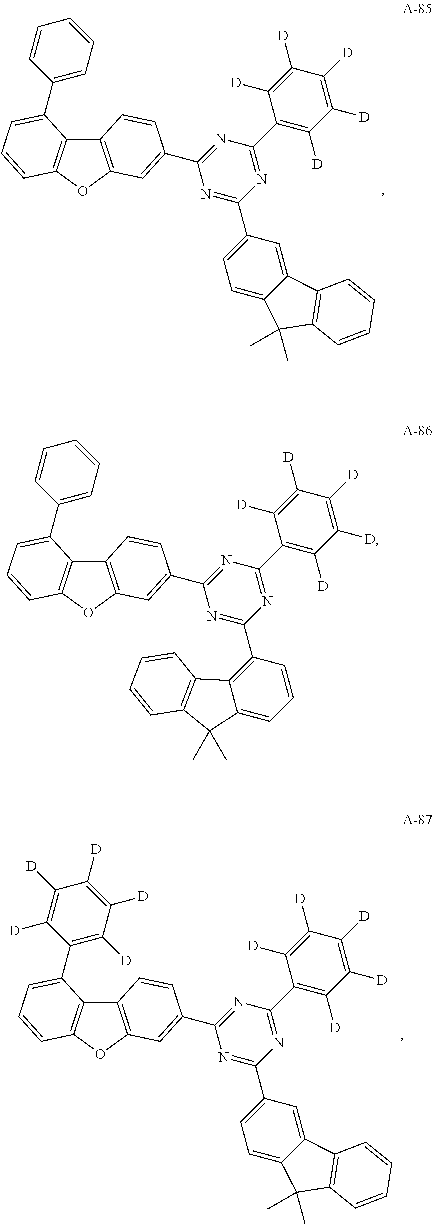 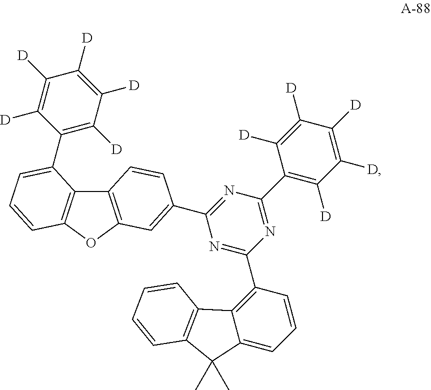 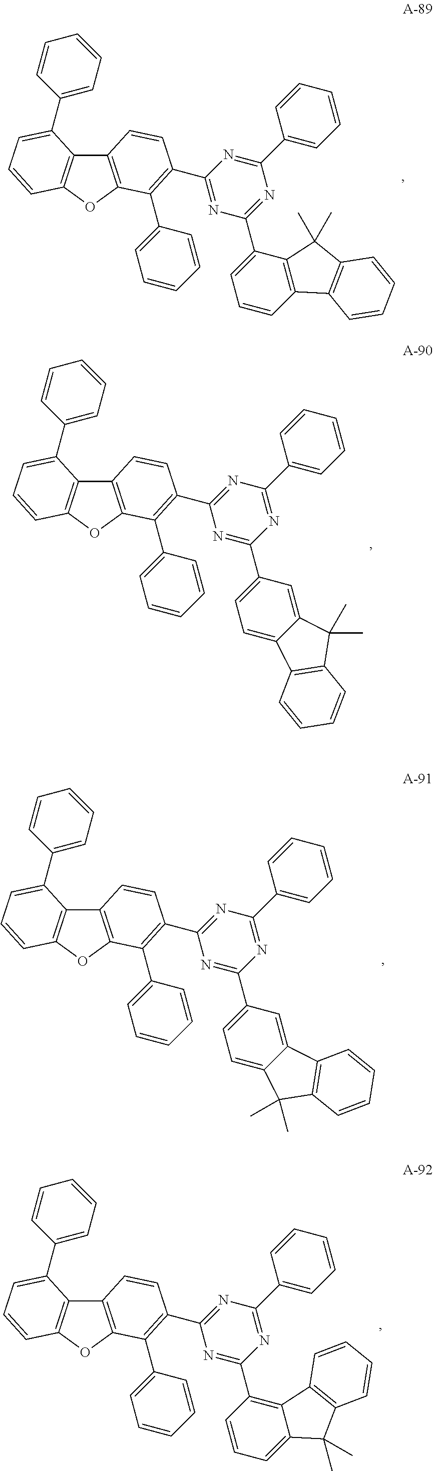 [SMILES: CC1(C)c2ccccc2-c2cccc(-c3nc(-c4ccccc4)nc(-c4ccc5c(oc6cccc(-c7ccccc7)c65)c4-c4ccccc4)n3)c21 ; CC1(C)c2ccccc2-c2ccc(-c3nc(-c4ccccc4)nc(-c4ccc5c(oc6cccc(-c7ccccc7)c65)c4-c4ccccc4)n3)cc21 ; CC1(C)c2ccccc2-c2cc(-c3nc(-c4ccccc4)nc(-c4ccc5c(oc6cccc(-c7ccccc7)c65)c4-c4ccccc4)n3)ccc21 ; CC1(C)c2ccccc2-c2c(-c3nc(-c4ccccc4)nc(-c4ccc5c(oc6cccc(-c7ccccc7)c65)c4-c4ccccc4)n3)cccc21] 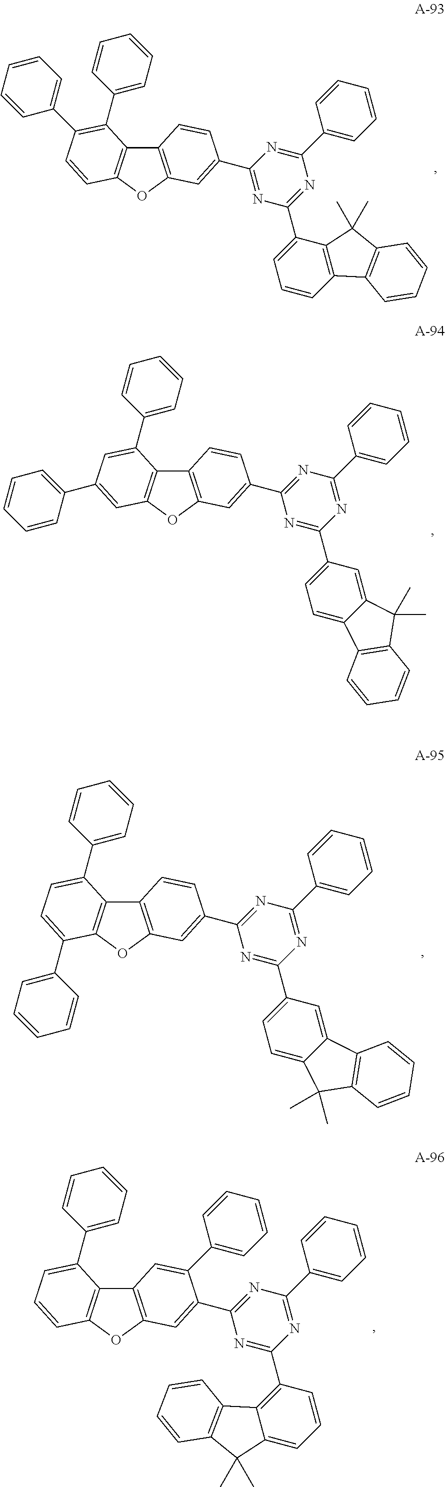 [SMILES: CC1(C)c2ccccc2-c2cccc(-c3nc(-c4ccccc4)nc(-c4ccc5c(c4)oc4ccc(-c6ccccc6)c(-c6ccccc6)c45)n3)c21 ; CC1(C)c2ccccc2-c2ccc(-c3nc(-c4ccccc4)nc(-c4ccc5c(c4)oc4cc(-c6ccccc6)cc(-c6ccccc6)c45)n3)cc21 ; CC1(C)c2ccccc2-c2cc(-c3nc(-c4ccccc4)nc(-c4ccc5c(c4)oc4c(-c6ccccc6)ccc(-c6ccccc6)c45)n3)ccc21 ; CC1(C)c2ccccc2-c2c(-c3nc(-c4ccccc4)nc(-c4cc5oc6cccc(-c7ccccc7)c6c5cc4-c4ccccc4)n3)cccc21] 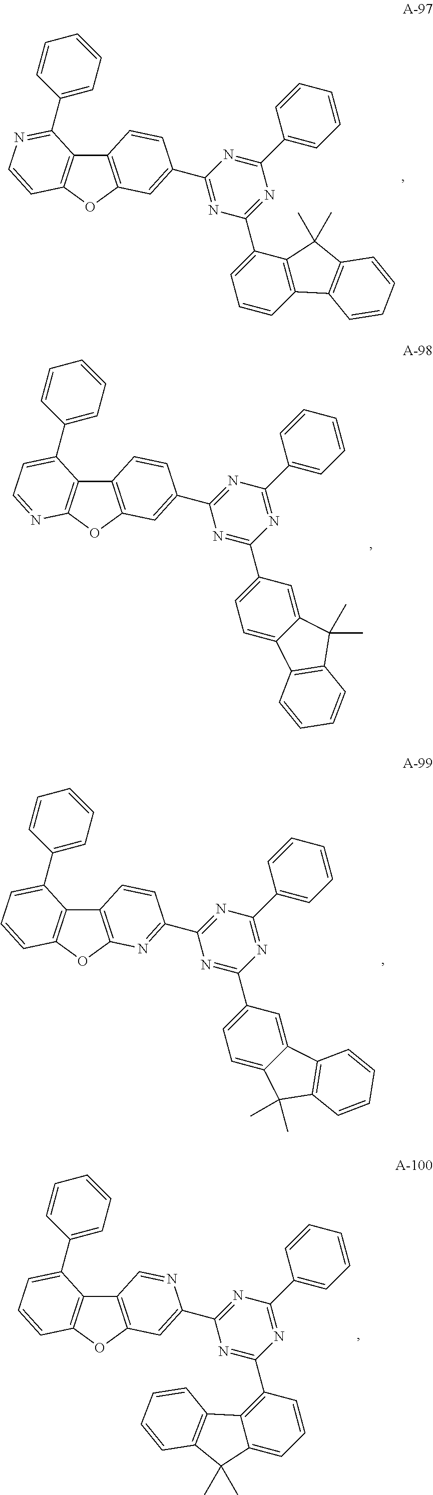 [SMILES: CC1(C)c2ccccc2-c2cccc(-c3nc(-c4ccccc4)nc(-c4ccc5c(c4)oc4ccnc(-c6ccccc6)c45)n3)c21 ; CC1(C)c2ccccc2-c2ccc(-c3nc(-c4ccccc4)nc(-c4ccc5c(c4)oc4nccc(-c6ccccc6)c45)n3)cc21 ; CC1(C)c2ccccc2-c2cc(-c3nc(-c4ccccc4)nc(-c4ccc5c(n4)oc4cccc(-c6ccccc6)c45)n3)ccc21 ; CC1(C)c2ccccc2-c2c(-c3nc(-c4ccccc4)nc(-c4cc5oc6cccc(-c7ccccc7)c6c5cn4)n3)cccc21] 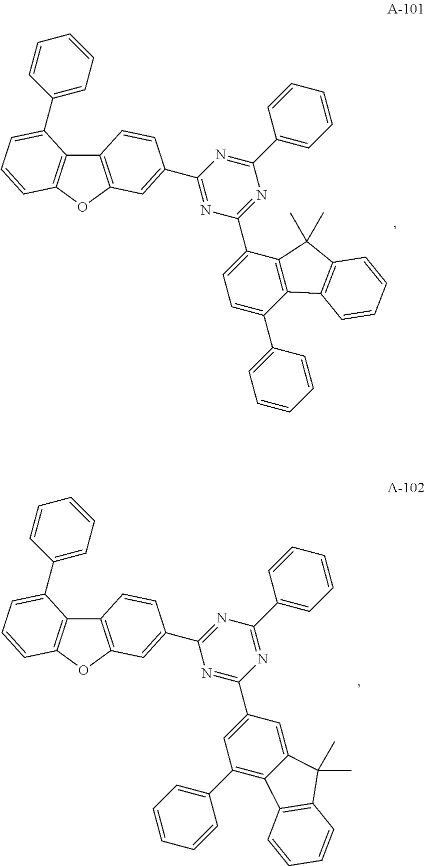 [SMILES: CC1(C)c2ccccc2-c2c(-c3ccccc3)ccc(-c3nc(-c4ccccc4)nc(-c4ccc5c(c4)oc4cccc(-c6ccccc6)c45)n3)c21 ; CC1(C)c2ccccc2-c2c(-c3ccccc3)cc(-c3nc(-c4ccccc4)nc(-c4ccc5c(c4)oc4cccc(-c6ccccc6)c45)n3)cc21] 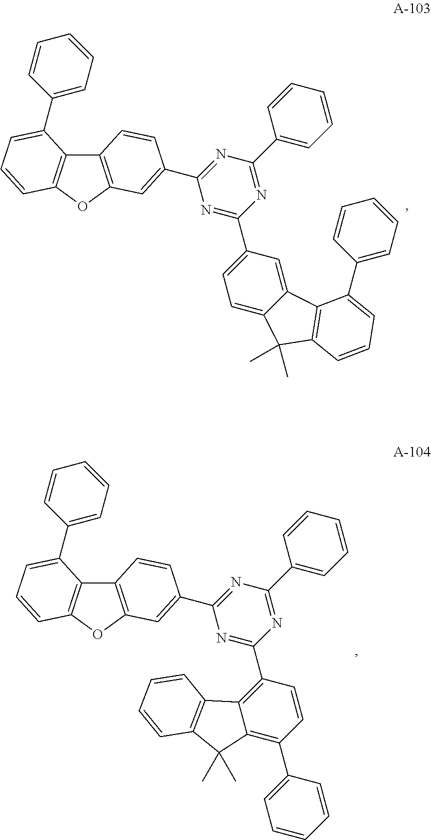 [SMILES: CC1(C)c2ccc(-c3nc(-c4ccccc4)nc(-c4ccc5c(c4)oc4cccc(-c6ccccc6)c45)n3)cc2-c2c(-c3ccccc3)cccc21 ; CC1(C)c2ccccc2-c2c(-c3nc(-c4ccccc4)nc(-c4ccc5c(c4)oc4cccc(-c6ccccc6)c45)n3)ccc(-c3ccccc3)c21] 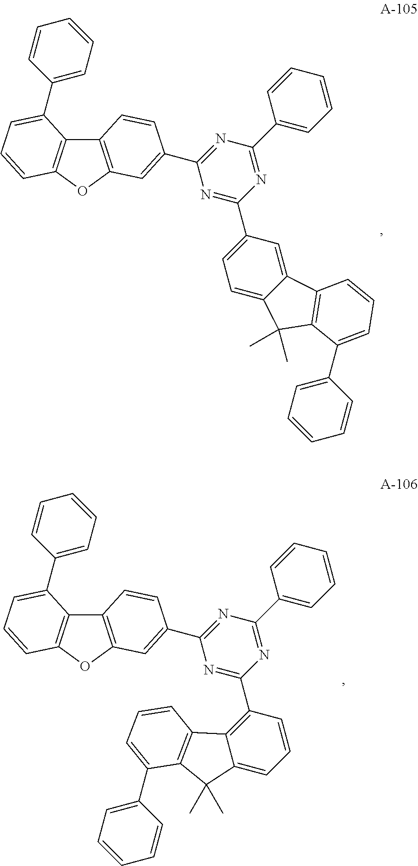 [SMILES: CC1(C)c2ccc(-c3nc(-c4ccccc4)nc(-c4ccc5c(c4)oc4cccc(-c6ccccc6)c45)n3)cc2-c2cccc(-c3ccccc3)c21 ; CC1(C)c2cccc(-c3nc(-c4ccccc4)nc(-c4ccc5c(c4)oc4cccc(-c6ccccc6)c45)n3)c2-c2cccc(-c3ccccc3)c21] 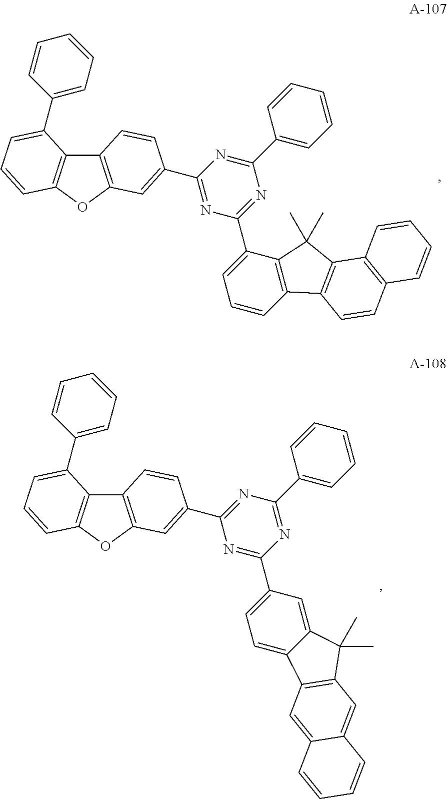 [SMILES: CC1(C)c2c(-c3nc(-c4ccccc4)nc(-c4ccc5c(c4)oc4cccc(-c6ccccc6)c45)n3)cccc2-c2ccc3ccccc3c21 ; CC1(C)c2cc(-c3nc(-c4ccccc4)nc(-c4ccc5c(c4)oc4cccc(-c6ccccc6)c45)n3)ccc2-c2cc3ccccc3cc21] 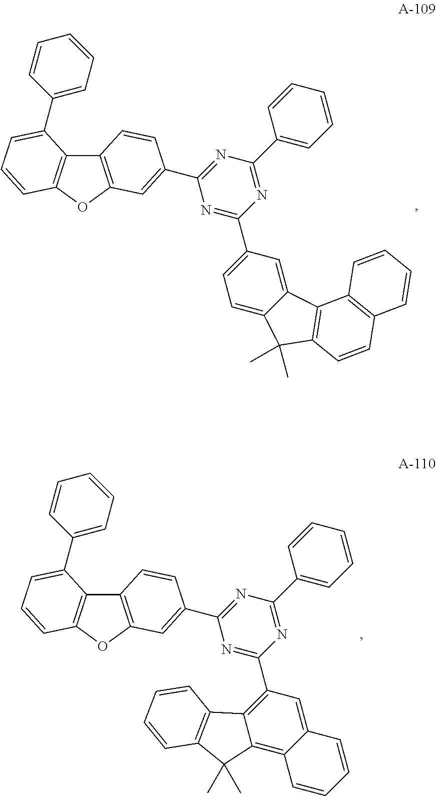 [SMILES: CC1(C)c2ccc(-c3nc(-c4ccccc4)nc(-c4ccc5c(c4)oc4cccc(-c6ccccc6)c45)n3)cc2-c2c1ccc1ccccc21 ; CC1(C)c2ccccc2-c2c(-c3nc(-c4ccccc4)nc(-c4ccc5c(c4)oc4cccc(-c6ccccc6)c45)n3)cc3ccccc3c21] 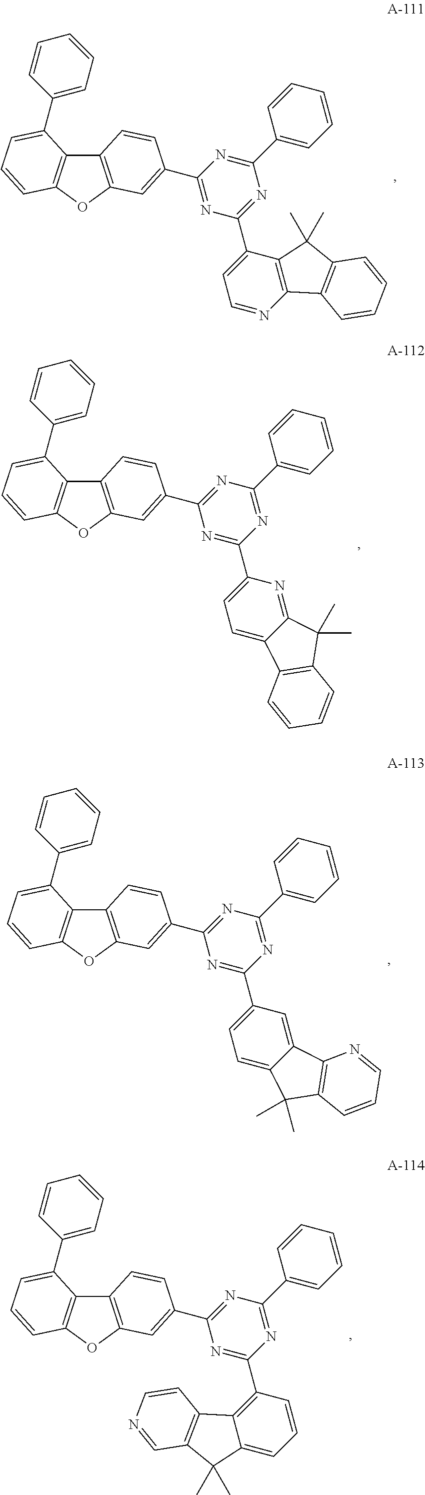 [SMILES: CC1(C)c2ccccc2-c2nccc(-c3nc(-c4ccccc4)nc(-c4ccc5c(c4)oc4cccc(-c6ccccc6)c45)n3)c21 ; CC1(C)c2ccccc2-c2ccc(-c3nc(-c4ccccc4)nc(-c4ccc5c(c4)oc4cccc(-c6ccccc6)c45)n3)nc21 ; CC1(C)c2ccc(-c3nc(-c4ccccc4)nc(-c4ccc5c(c4)oc4cccc(-c6ccccc6)c45)n3)cc2-c2ncccc21 ; CC1(C)c2cnccc2-c2c(-c3nc(-c4ccccc4)nc(-c4ccc5c(c4)oc4cccc(-c6ccccc6)c45)n3)cccc21] 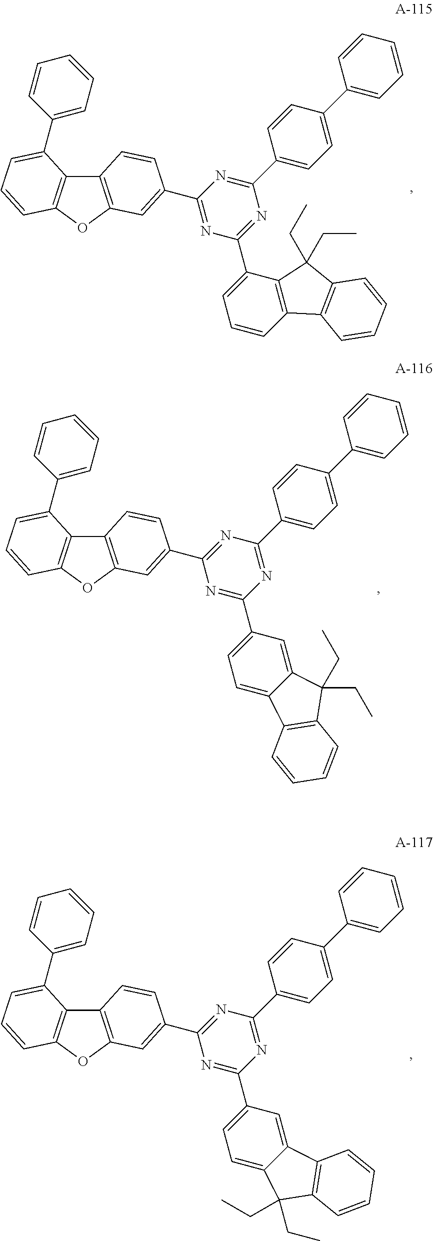 [SMILES: CCC1(CC)c2ccccc2-c2cccc(-c3nc(-c4ccc(-c5ccccc5)cc4)nc(-c4ccc5c(c4)oc4cccc(-c6ccccc6)c45)n3)c21 ; CCC1(CC)c2ccccc2-c2ccc(-c3nc(-c4ccc(-c5ccccc5)cc4)nc(-c4ccc5c(c4)oc4cccc(-c6ccccc6)c45)n3)cc21 ; CCC1(CC)c2ccccc2-c2cc(-c3nc(-c4ccc(-c5ccccc5)cc4)nc(-c4ccc5c(c4)oc4cccc(-c6ccccc6)c45)n3)ccc21] 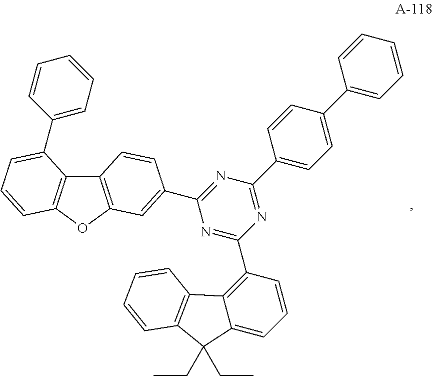 [SMILES: CCC1(CC)c2ccccc2-c2c(-c3nc(-c4ccc(-c5ccccc5)cc4)nc(-c4ccc5c(c4)oc4cccc(-c6ccccc6)c45)n3)cccc21] 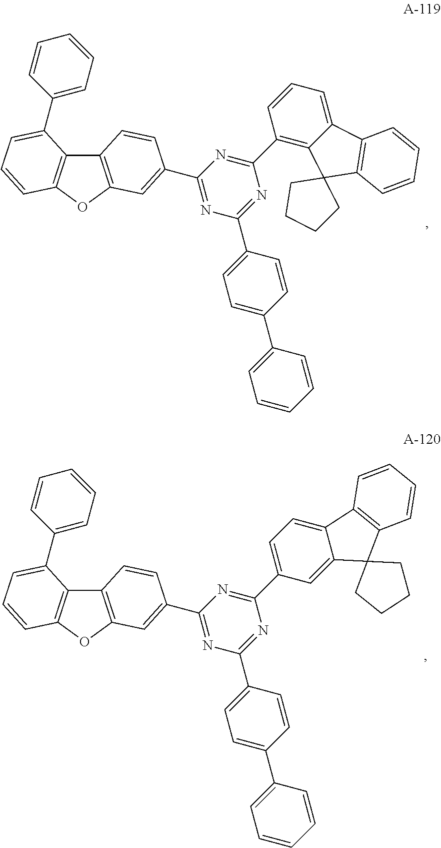 [SMILES: c1ccc(-c2ccc(-c3nc(-c4ccc5c(c4)oc4cccc(-c6ccccc6)c45)nc(-c4cccc5c4C4(CCCC4)c4ccccc4-5)n3)cc2)cc1 ; c1ccc(-c2ccc(-c3nc(-c4ccc5c(c4)C4(CCCC4)c4ccccc4-5)nc(-c4ccc5c(c4)oc4cccc(-c6ccccc6)c45)n3)cc2)cc1] 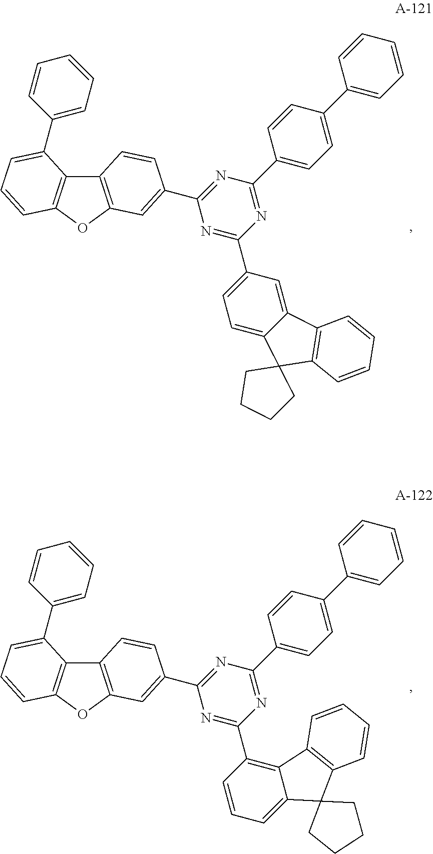 [SMILES: c1ccc(-c2ccc(-c3nc(-c4ccc5c(c4)-c4ccccc4C54CCCC4)nc(-c4ccc5c(c4)oc4cccc(-c6ccccc6)c45)n3)cc2)cc1 ; c1ccc(-c2ccc(-c3nc(-c4ccc5c(c4)oc4cccc(-c6ccccc6)c45)nc(-c4cccc5c4-c4ccccc4C54CCCC4)n3)cc2)cc1] 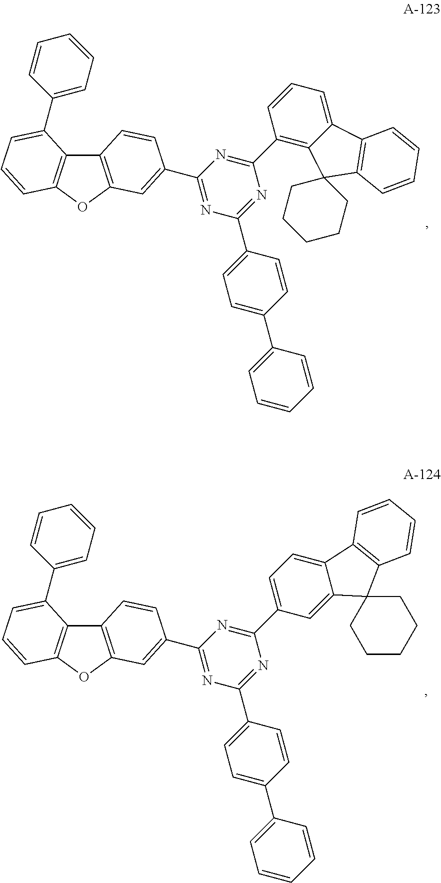 [SMILES: c1ccc(-c2ccc(-c3nc(-c4ccc5c(c4)oc4cccc(-c6ccccc6)c45)nc(-c4cccc5c4C4(CCCCC4)c4ccccc4-5)n3)cc2)cc1 ; c1ccc(-c2ccc(-c3nc(-c4ccc5c(c4)C4(CCCCC4)c4ccccc4-5)nc(-c4ccc5c(c4)oc4cccc(-c6ccccc6)c45)n3)cc2)cc1] 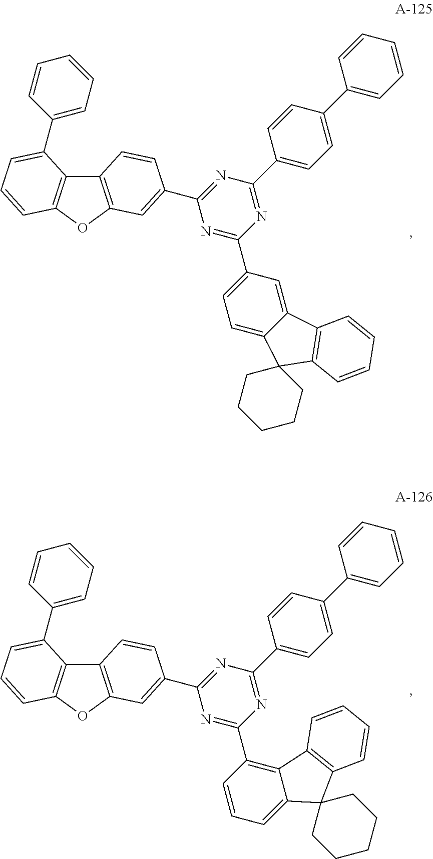 [SMILES: c1ccc(-c2ccc(-c3nc(-c4ccc5c(c4)-c4ccccc4C54CCCCC4)nc(-c4ccc5c(c4)oc4cccc(-c6ccccc6)c45)n3)cc2)cc1 ; c1ccc(-c2ccc(-c3nc(-c4ccc5c(c4)oc4cccc(-c6ccccc6)c45)nc(-c4cccc5c4-c4ccccc4C54CCCCC4)n3)cc2)cc1] 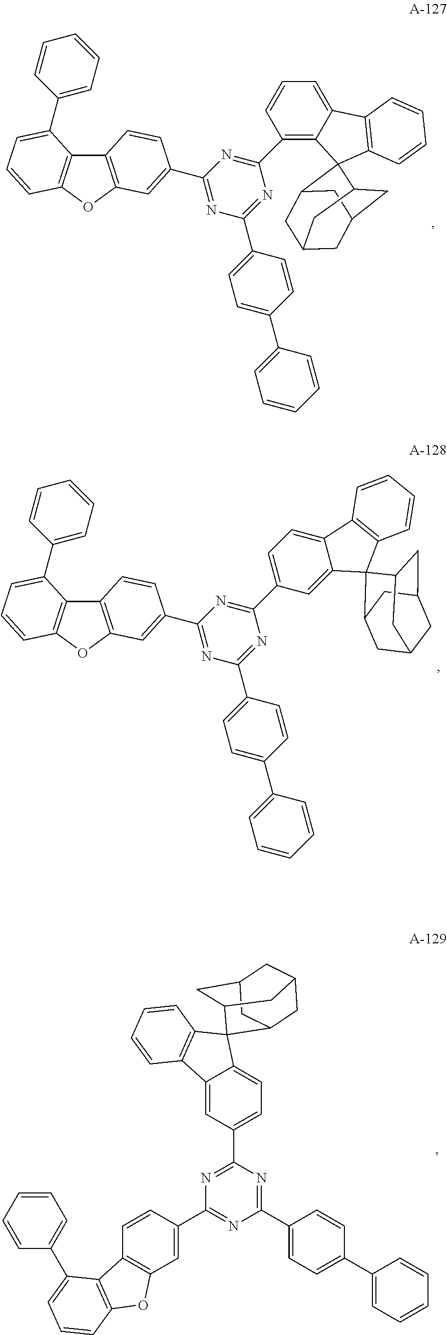 [SMILES: c1ccc(-c2ccc(-c3nc(-c4ccc5c(c4)oc4cccc(-c6ccccc6)c45)nc(-c4cccc5c4C4(c6ccccc6-5)C5CC6CC(C5)CC4C6)n3)cc2)cc1 ; c1ccc(-c2ccc(-c3nc(-c4ccc5c(c4)C4(c6ccccc6-5)C5CC6CC(C5)CC4C6)nc(-c4ccc5c(c4)oc4cccc(-c6ccccc6)c45)n3)cc2)cc1 ; c1ccc(-c2ccc(-c3nc(-c4ccc5c(c4)-c4ccccc4C54C5CC6CC(C5)CC4C6)nc(-c4ccc5c(c4)oc4cccc(-c6ccccc6)c45)n3)cc2)cc1] 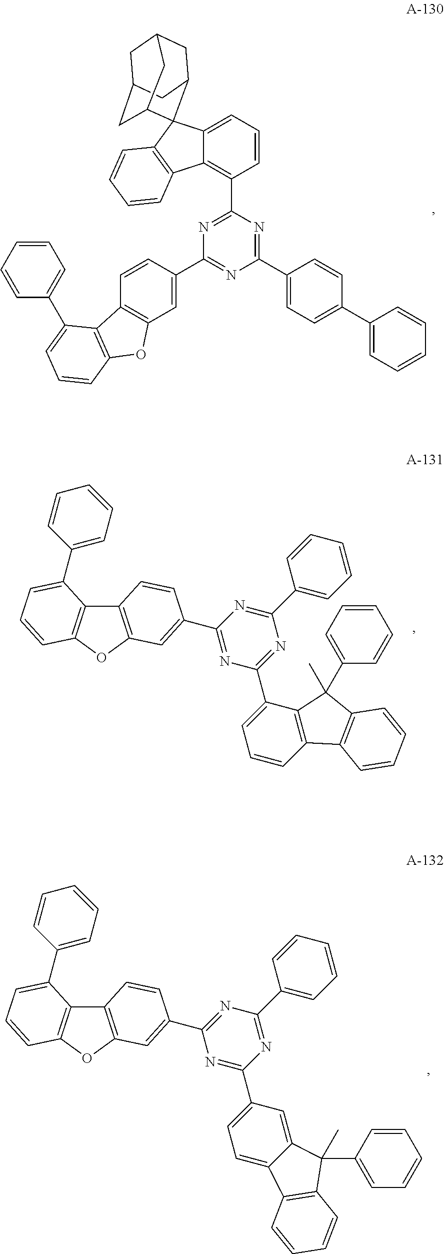 [SMILES: c1ccc(-c2ccc(-c3nc(-c4ccc5c(c4)oc4cccc(-c6ccccc6)c45)nc(-c4cccc5c4-c4ccccc4C54C5CC6CC(C5)CC4C6)n3)cc2)cc1 ; CC1(c2ccccc2)c2ccccc2-c2cccc(-c3nc(-c4ccccc4)nc(-c4ccc5c(c4)oc4cccc(-c6ccccc6)c45)n3)c21 ; CC1(c2ccccc2)c2ccccc2-c2ccc(-c3nc(-c4ccccc4)nc(-c4ccc5c(c4)oc4cccc(-c6ccccc6)c45)n3)cc21] 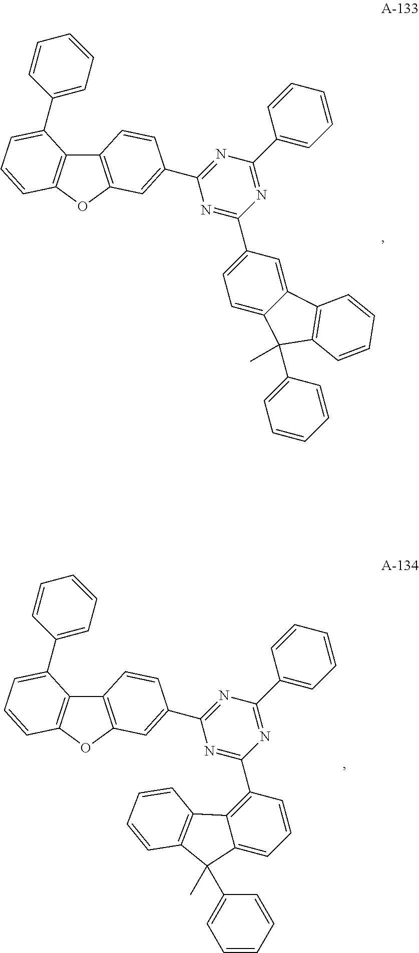 [SMILES: CC1(c2ccccc2)c2ccccc2-c2cc(-c3nc(-c4ccccc4)nc(-c4ccc5c(c4)oc4cccc(-c6ccccc6)c45)n3)ccc21 ; CC1(c2ccccc2)c2ccccc2-c2c(-c3nc(-c4ccccc4)nc(-c4ccc5c(c4)oc4cccc(-c6ccccc6)c45)n3)cccc21] 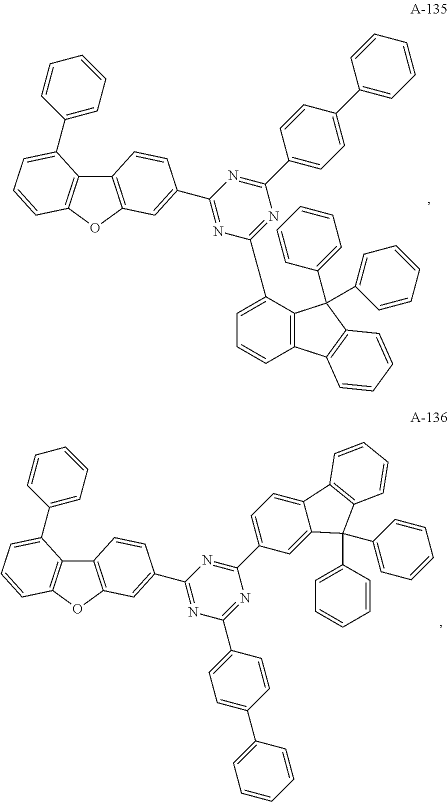 [SMILES: c1ccc(-c2ccc(-c3nc(-c4ccc5c(c4)oc4cccc(-c6ccccc6)c45)nc(-c4cccc5c4C(c4ccccc4)(c4ccccc4)c4ccccc4-5)n3)cc2)cc1 ; c1ccc(-c2ccc(-c3nc(-c4ccc5c(c4)C(c4ccccc4)(c4ccccc4)c4ccccc4-5)nc(-c4ccc5c(c4)oc4cccc(-c6ccccc6)c45)n3)cc2)cc1] 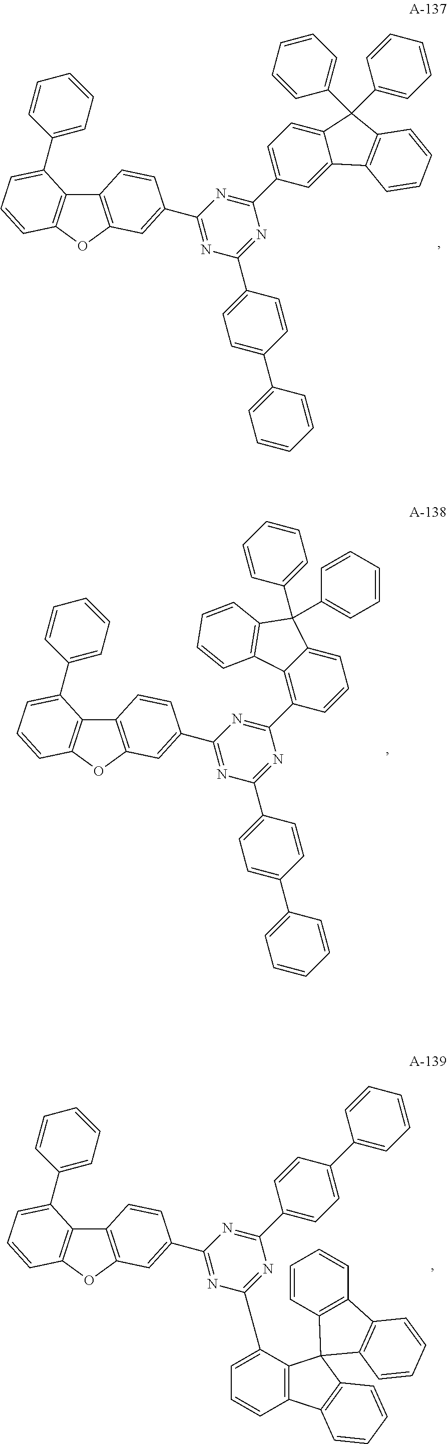 [SMILES: c1ccc(-c2ccc(-c3nc(-c4ccc5c(c4)-c4ccccc4C5(c4ccccc4)c4ccccc4)nc(-c4ccc5c(c4)oc4cccc(-c6ccccc6)c45)n3)cc2)cc1 ; c1ccc(-c2ccc(-c3nc(-c4ccc5c(c4)oc4cccc(-c6ccccc6)c45)nc(-c4cccc5c4-c4ccccc4C5(c4ccccc4)c4ccccc4)n3)cc2)cc1 ; c1ccc(-c2ccc(-c3nc(-c4ccc5c(c4)oc4cccc(-c6ccccc6)c45)nc(-c4cccc5c4C4(c6ccccc6-c6ccccc64)c4ccccc4-5)n3)cc2)cc1] 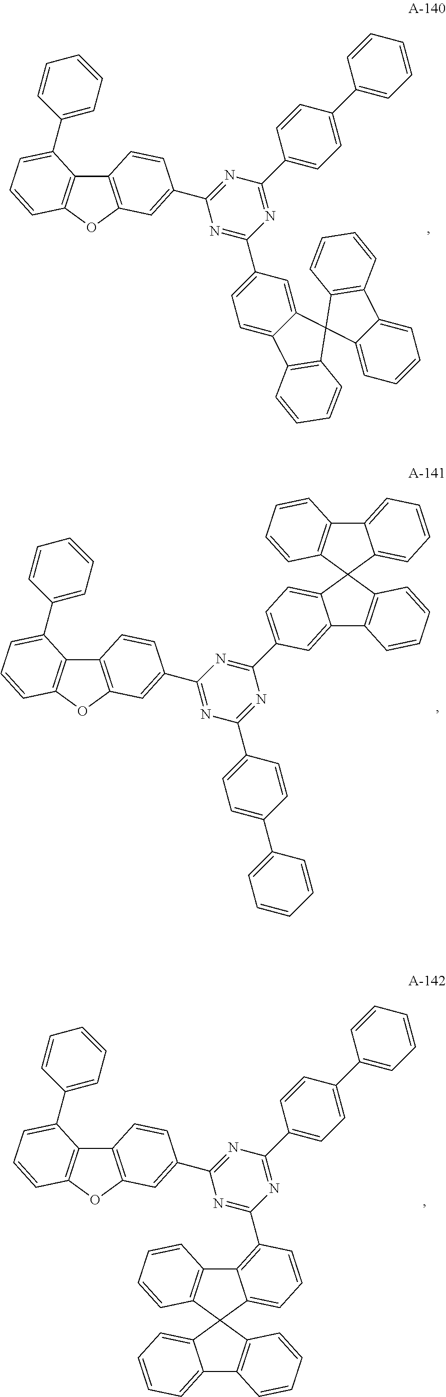 [SMILES: c1ccc(-c2ccc(-c3nc(-c4ccc5c(c4)C4(c6ccccc6-c6ccccc64)c4ccccc4-5)nc(-c4ccc5c(c4)oc4cccc(-c6ccccc6)c45)n3)cc2)cc1 ; c1ccc(-c2ccc(-c3nc(-c4ccc5c(c4)-c4ccccc4C54c5ccccc5-c5ccccc54)nc(-c4ccc5c(c4)oc4cccc(-c6ccccc6)c45)n3)cc2)cc1 ; c1ccc(-c2ccc(-c3nc(-c4ccc5c(c4)oc4cccc(-c6ccccc6)c45)nc(-c4cccc5c4-c4ccccc4C54c5ccccc5-c5ccccc54)n3)cc2)cc1] 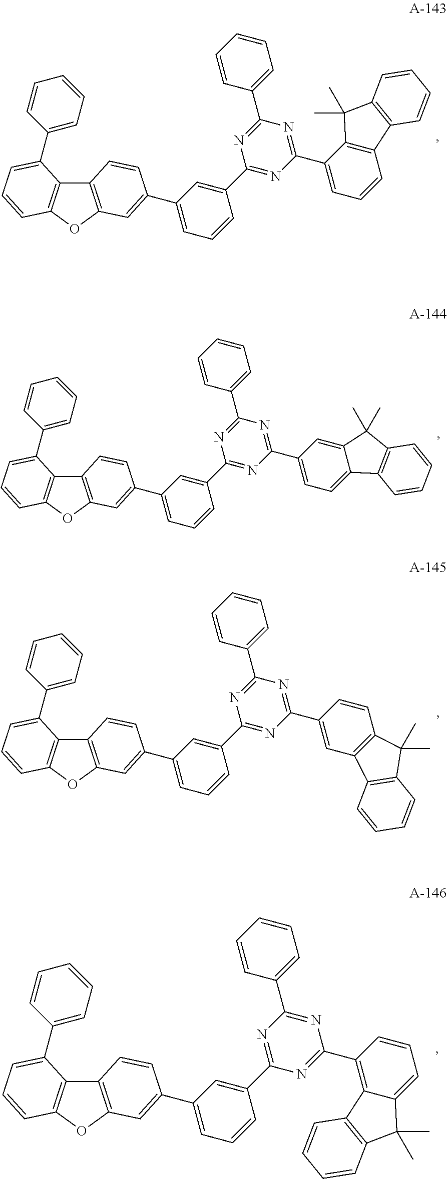 [SMILES: CC1(C)c2ccccc2-c2cccc(-c3nc(-c4ccccc4)nc(-c4cccc(-c5ccc6c(c5)oc5cccc(-c7ccccc7)c56)c4)n3)c21 ; CC1(C)c2ccccc2-c2ccc(-c3nc(-c4ccccc4)nc(-c4cccc(-c5ccc6c(c5)oc5cccc(-c7ccccc7)c56)c4)n3)cc21 ; CC1(C)c2ccccc2-c2cc(-c3nc(-c4ccccc4)nc(-c4cccc(-c5ccc6c(c5)oc5cccc(-c7ccccc7)c56)c4)n3)ccc21 ; CC1(C)c2ccccc2-c2c(-c3nc(-c4ccccc4)nc(-c4cccc(-c5ccc6c(c5)oc5cccc(-c7ccccc7)c56)c4)n3)cccc21] 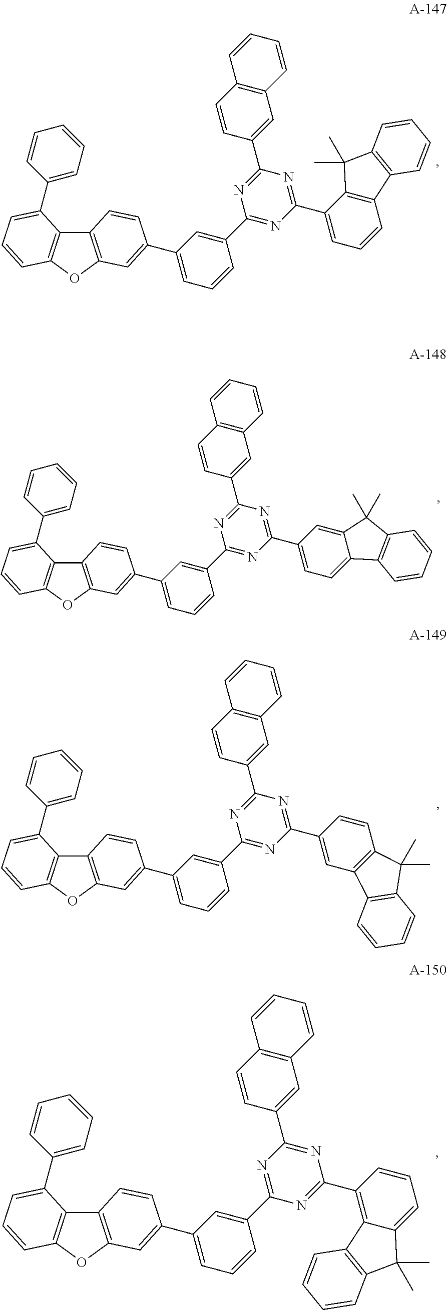 [SMILES: CC1(C)c2ccccc2-c2cccc(-c3nc(-c4cccc(-c5ccc6c(c5)oc5cccc(-c7ccccc7)c56)c4)nc(-c4ccc5ccccc5c4)n3)c21 ; CC1(C)c2ccccc2-c2ccc(-c3nc(-c4cccc(-c5ccc6c(c5)oc5cccc(-c7ccccc7)c56)c4)nc(-c4ccc5ccccc5c4)n3)cc21 ; CC1(C)c2ccccc2-c2cc(-c3nc(-c4cccc(-c5ccc6c(c5)oc5cccc(-c7ccccc7)c56)c4)nc(-c4ccc5ccccc5c4)n3)ccc21 ; CC1(C)c2ccccc2-c2c(-c3nc(-c4cccc(-c5ccc6c(c5)oc5cccc(-c7ccccc7)c56)c4)nc(-c4ccc5ccccc5c4)n3)cccc21] 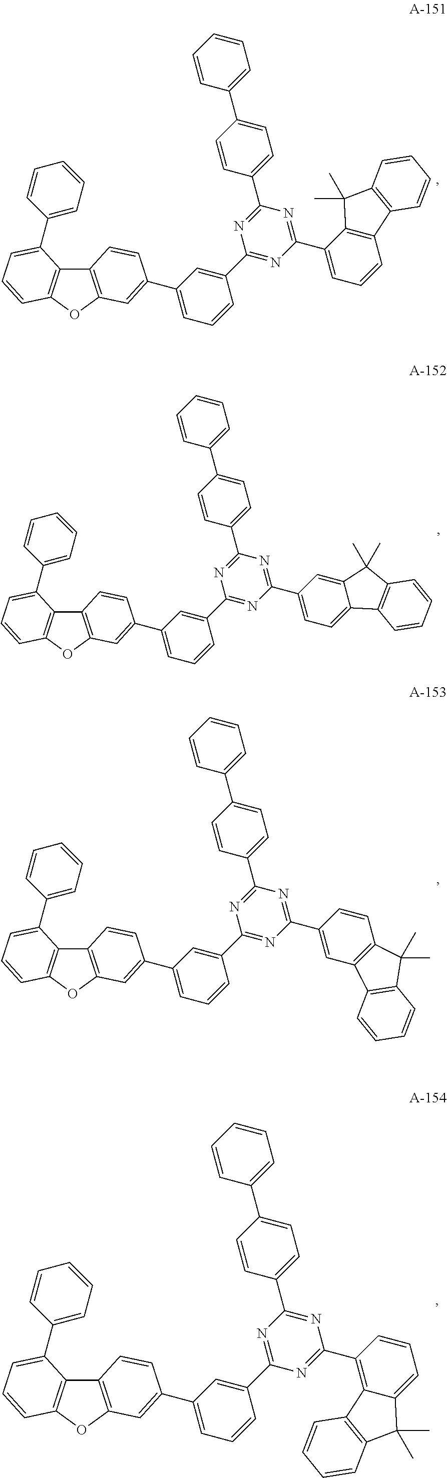 [SMILES: CC1(C)c2ccccc2-c2cccc(-c3nc(-c4ccc(-c5ccccc5)cc4)nc(-c4cccc(-c5ccc6c(c5)oc5cccc(-c7ccccc7)c56)c4)n3)c21 ; CC1(C)c2ccccc2-c2ccc(-c3nc(-c4ccc(-c5ccccc5)cc4)nc(-c4cccc(-c5ccc6c(c5)oc5cccc(-c7ccccc7)c56)c4)n3)cc21 ; CC1(C)c2ccccc2-c2cc(-c3nc(-c4ccc(-c5ccccc5)cc4)nc(-c4cccc(-c5ccc6c(c5)oc5cccc(-c7ccccc7)c56)c4)n3)ccc21 ; CC1(C)c2ccccc2-c2c(-c3nc(-c4ccc(-c5ccccc5)cc4)nc(-c4cccc(-c5ccc6c(c5)oc5cccc(-c7ccccc7)c56)c4)n3)cccc21] 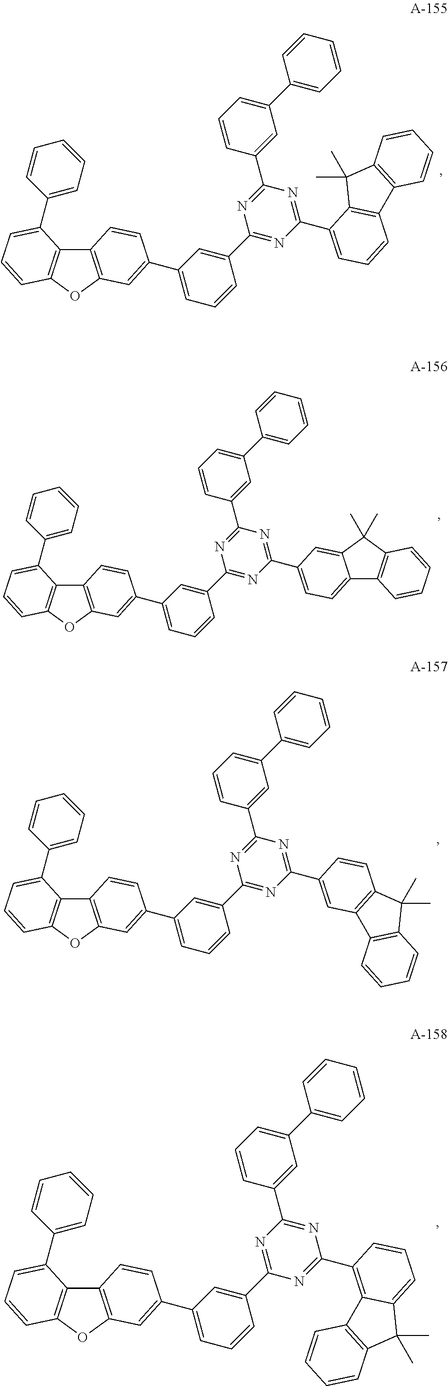 [SMILES: CC1(C)c2ccccc2-c2cccc(-c3nc(-c4cccc(-c5ccccc5)c4)nc(-c4cccc(-c5ccc6c(c5)oc5cccc(-c7ccccc7)c56)c4)n3)c21 ; CC1(C)c2ccccc2-c2ccc(-c3nc(-c4cccc(-c5ccccc5)c4)nc(-c4cccc(-c5ccc6c(c5)oc5cccc(-c7ccccc7)c56)c4)n3)cc21 ; CC1(C)c2ccccc2-c2cc(-c3nc(-c4cccc(-c5ccccc5)c4)nc(-c4cccc(-c5ccc6c(c5)oc5cccc(-c7ccccc7)c56)c4)n3)ccc21 ; CC1(C)c2ccccc2-c2c(-c3nc(-c4cccc(-c5ccccc5)c4)nc(-c4cccc(-c5ccc6c(c5)oc5cccc(-c7ccccc7)c56)c4)n3)cccc21] 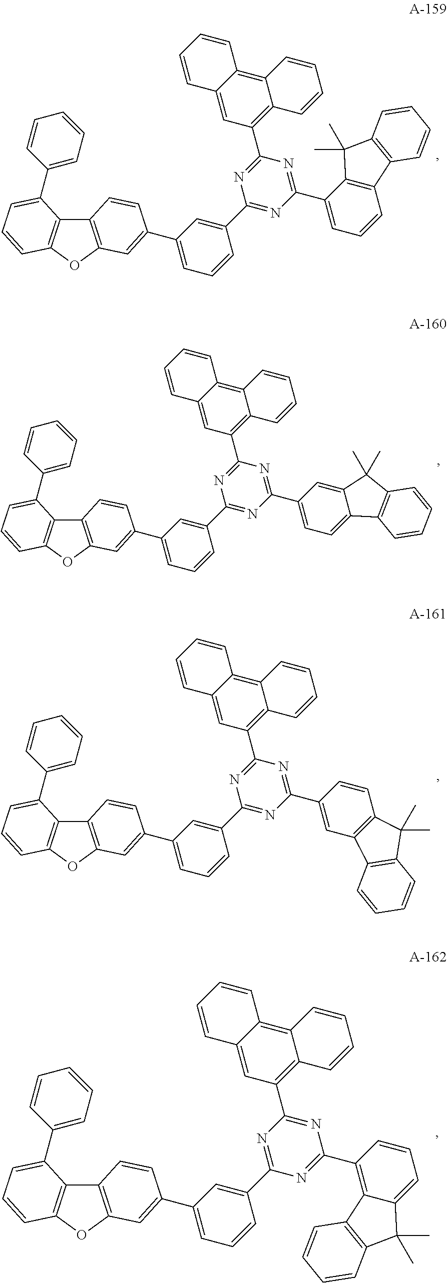 [SMILES: CC1(C)c2ccccc2-c2cccc(-c3nc(-c4cccc(-c5ccc6c(c5)oc5cccc(-c7ccccc7)c56)c4)nc(-c4cc5ccccc5c5ccccc45)n3)c21 ; CC1(C)c2ccccc2-c2ccc(-c3nc(-c4cccc(-c5ccc6c(c5)oc5cccc(-c7ccccc7)c56)c4)nc(-c4cc5ccccc5c5ccccc45)n3)cc21 ; CC1(C)c2ccccc2-c2cc(-c3nc(-c4cccc(-c5ccc6c(c5)oc5cccc(-c7ccccc7)c56)c4)nc(-c4cc5ccccc5c5ccccc45)n3)ccc21 ; CC1(C)c2ccccc2-c2c(-c3nc(-c4cccc(-c5ccc6c(c5)oc5cccc(-c7ccccc7)c56)c4)nc(-c4cc5ccccc5c5ccccc45)n3)cccc21] 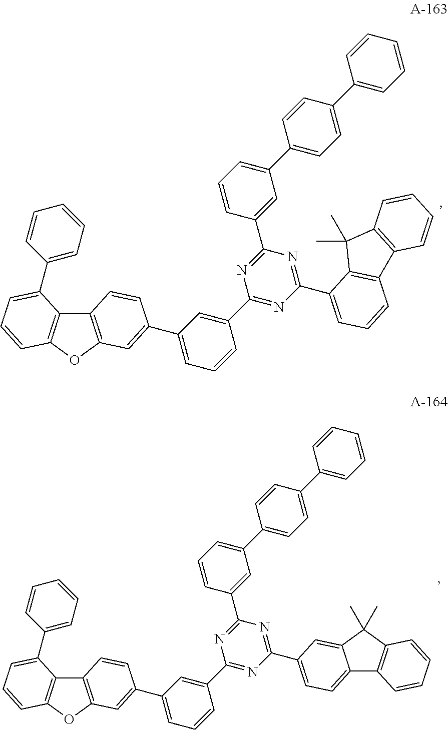 [SMILES: CC1(C)c2ccccc2-c2cccc(-c3nc(-c4cccc(-c5ccc(-c6ccccc6)cc5)c4)nc(-c4cccc(-c5ccc6c(c5)oc5cccc(-c7ccccc7)c56)c4)n3)c21 ; CC1(C)c2ccccc2-c2ccc(-c3nc(-c4cccc(-c5ccc(-c6ccccc6)cc5)c4)nc(-c4cccc(-c5ccc6c(c5)oc5cccc(-c7ccccc7)c56)c4)n3)cc21] 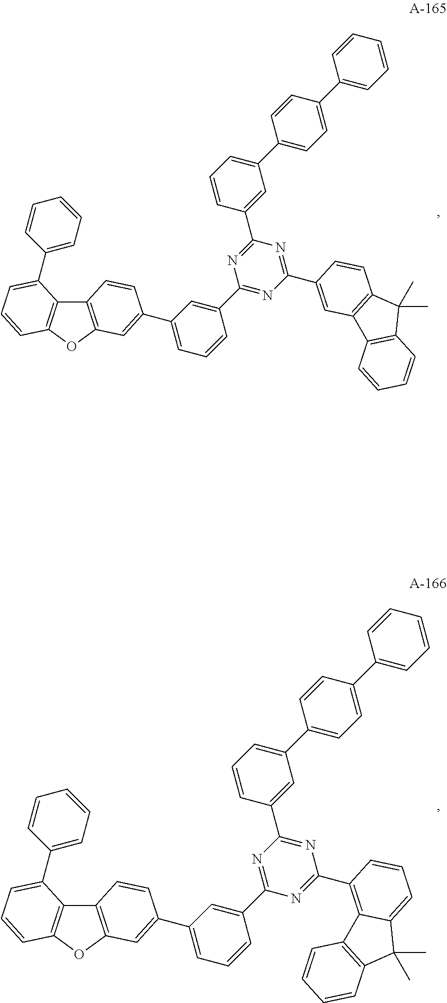 [SMILES: CC1(C)c2ccccc2-c2cc(-c3nc(-c4cccc(-c5ccc(-c6ccccc6)cc5)c4)nc(-c4cccc(-c5ccc6c(c5)oc5cccc(-c7ccccc7)c56)c4)n3)ccc21 ; CC1(C)c2ccccc2-c2c(-c3nc(-c4cccc(-c5ccc(-c6ccccc6)cc5)c4)nc(-c4cccc(-c5ccc6c(c5)oc5cccc(-c7ccccc7)c56)c4)n3)cccc21] 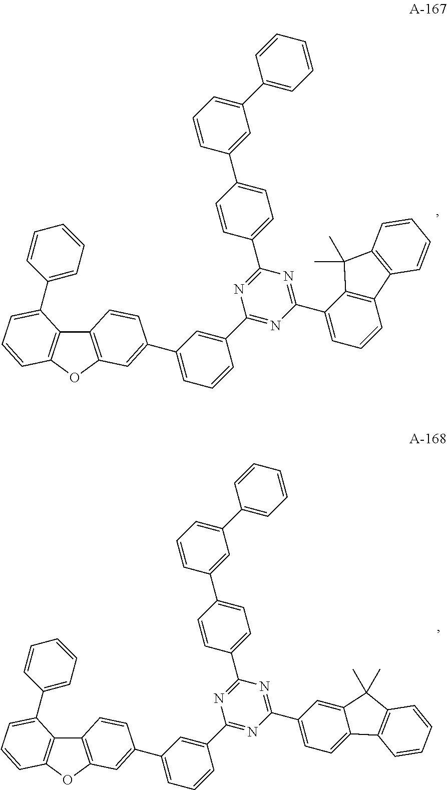 [SMILES: CC1(C)c2ccccc2-c2cccc(-c3nc(-c4ccc(-c5cccc(-c6ccccc6)c5)cc4)nc(-c4cccc(-c5ccc6c(c5)oc5cccc(-c7ccccc7)c56)c4)n3)c21 ; CC1(C)c2ccccc2-c2ccc(-c3nc(-c4ccc(-c5cccc(-c6ccccc6)c5)cc4)nc(-c4cccc(-c5ccc6c(c5)oc5cccc(-c7ccccc7)c56)c4)n3)cc21] 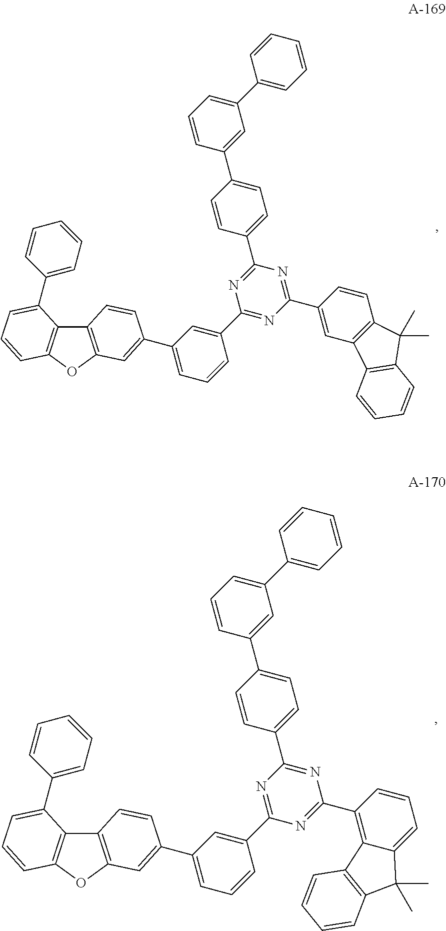 [SMILES: CC1(C)c2ccccc2-c2cc(-c3nc(-c4ccc(-c5cccc(-c6ccccc6)c5)cc4)nc(-c4cccc(-c5ccc6c(c5)oc5cccc(-c7ccccc7)c56)c4)n3)ccc21 ; CC1(C)c2ccccc2-c2c(-c3nc(-c4ccc(-c5cccc(-c6ccccc6)c5)cc4)nc(-c4cccc(-c5ccc6c(c5)oc5cccc(-c7ccccc7)c56)c4)n3)cccc21] 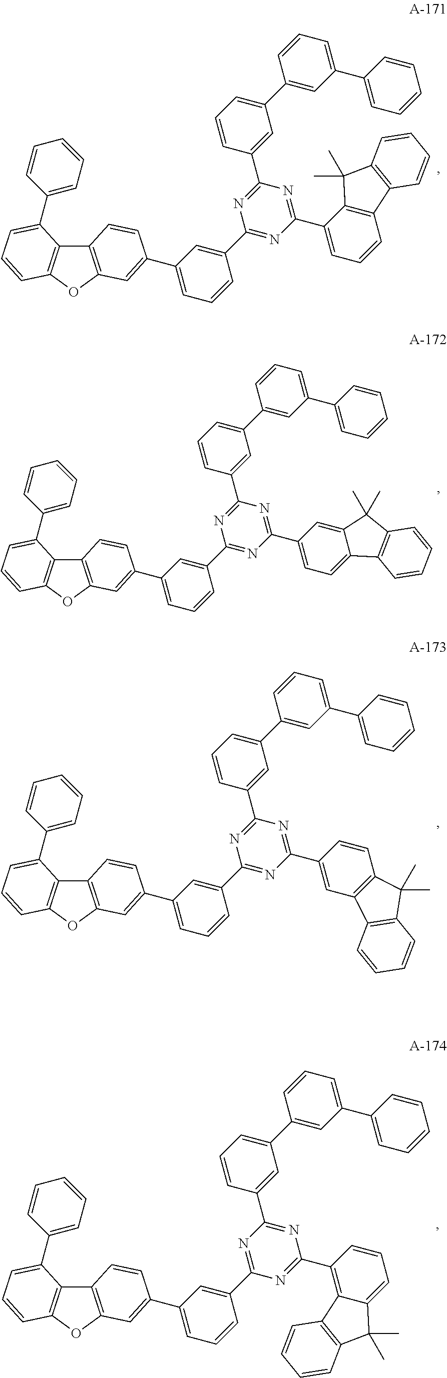 [SMILES: CC1(C)c2ccccc2-c2cccc(-c3nc(-c4cccc(-c5cccc(-c6ccccc6)c5)c4)nc(-c4cccc(-c5ccc6c(c5)oc5cccc(-c7ccccc7)c56)c4)n3)c21 ; CC1(C)c2ccccc2-c2ccc(-c3nc(-c4cccc(-c5cccc(-c6ccccc6)c5)c4)nc(-c4cccc(-c5ccc6c(c5)oc5cccc(-c7ccccc7)c56)c4)n3)cc21 ; CC1(C)c2ccccc2-c2cc(-c3nc(-c4cccc(-c5cccc(-c6ccccc6)c5)c4)nc(-c4cccc(-c5ccc6c(c5)oc5cccc(-c7ccccc7)c56)c4)n3)ccc21 ; CC1(C)c2ccccc2-c2c(-c3nc(-c4cccc(-c5cccc(-c6ccccc6)c5)c4)nc(-c4cccc(-c5ccc6c(c5)oc5cccc(-c7ccccc7)c56)c4)n3)cccc21] 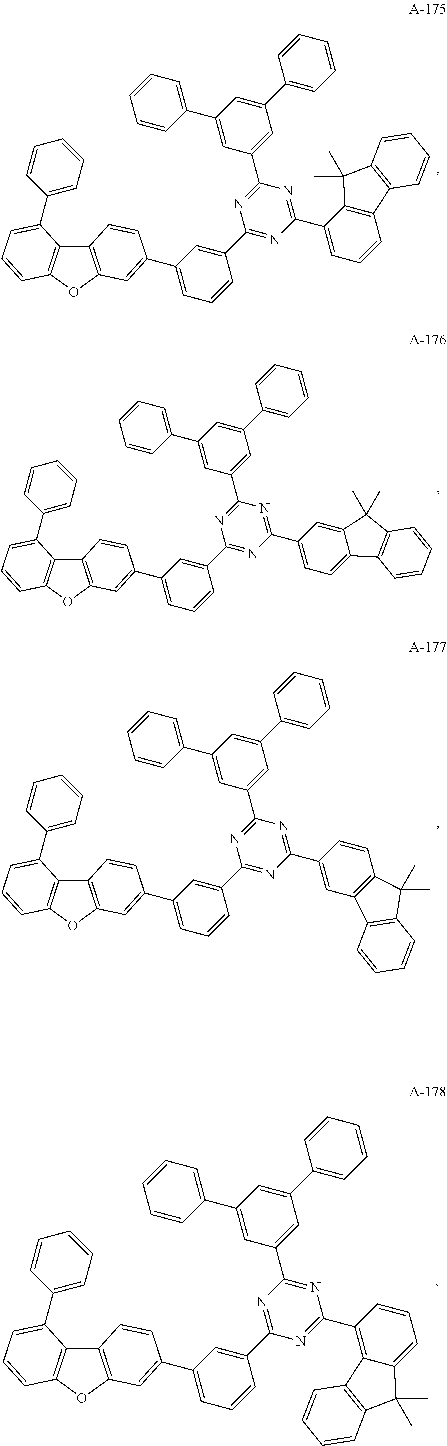 [SMILES: CC1(C)c2ccccc2-c2cccc(-c3nc(-c4cccc(-c5ccc6c(c5)oc5cccc(-c7ccccc7)c56)c4)nc(-c4cc(-c5ccccc5)cc(-c5ccccc5)c4)n3)c21 ; CC1(C)c2ccccc2-c2ccc(-c3nc(-c4cccc(-c5ccc6c(c5)oc5cccc(-c7ccccc7)c56)c4)nc(-c4cc(-c5ccccc5)cc(-c5ccccc5)c4)n3)cc21 ; CC1(C)c2ccccc2-c2cc(-c3nc(-c4cccc(-c5ccc6c(c5)oc5cccc(-c7ccccc7)c56)c4)nc(-c4cc(-c5ccccc5)cc(-c5ccccc5)c4)n3)ccc21 ; CC1(C)c2ccccc2-c2c(-c3nc(-c4cccc(-c5ccc6c(c5)oc5cccc(-c7ccccc7)c56)c4)nc(-c4cc(-c5ccccc5)cc(-c5ccccc5)c4)n3)cccc21] 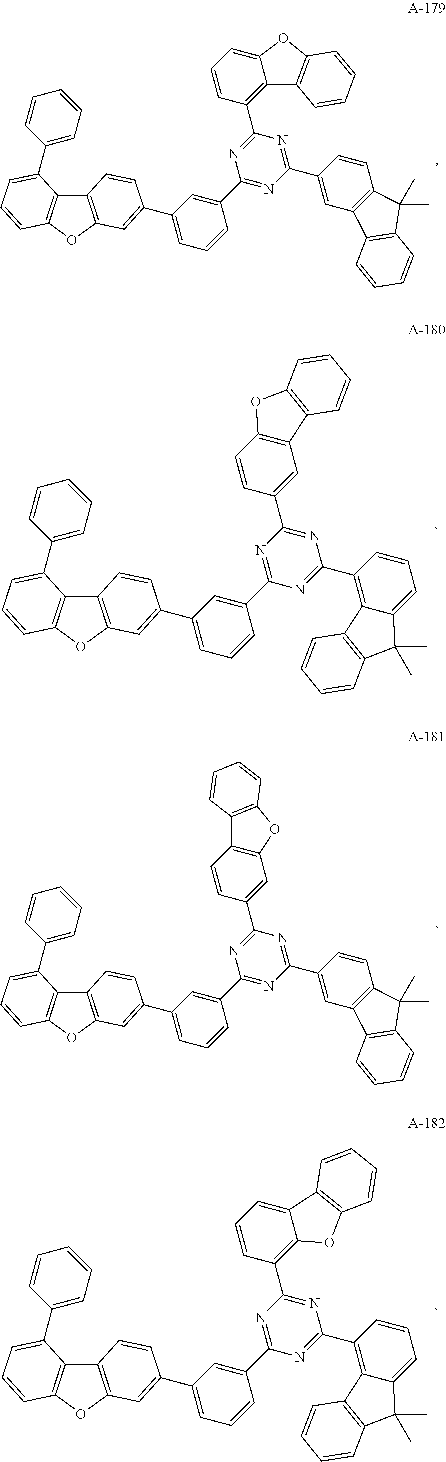 [SMILES: CC1(C)c2ccccc2-c2cc(-c3nc(-c4cccc(-c5ccc6c(c5)oc5cccc(-c7ccccc7)c56)c4)nc(-c4cccc5oc6ccccc6c45)n3)ccc21 ; CC1(C)c2ccccc2-c2c(-c3nc(-c4cccc(-c5ccc6c(c5)oc5cccc(-c7ccccc7)c56)c4)nc(-c4ccc5oc6ccccc6c5c4)n3)cccc21 ; CC1(C)c2ccccc2-c2cc(-c3nc(-c4cccc(-c5ccc6c(c5)oc5cccc(-c7ccccc7)c56)c4)nc(-c4ccc5c(c4)oc4ccccc45)n3)ccc21 ; CC1(C)c2ccccc2-c2c(-c3nc(-c4cccc(-c5ccc6c(c5)oc5cccc(-c7ccccc7)c56)c4)nc(-c4cccc5c4oc4ccccc45)n3)cccc21] 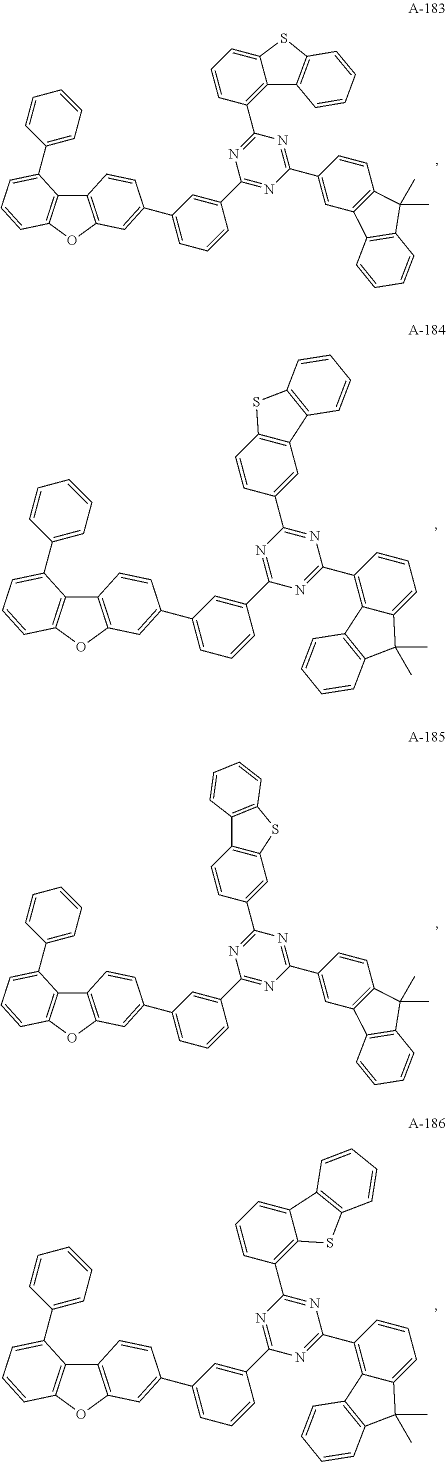 [SMILES: CC1(C)c2ccccc2-c2cc(-c3nc(-c4cccc(-c5ccc6c(c5)oc5cccc(-c7ccccc7)c56)c4)nc(-c4cccc5sc6ccccc6c45)n3)ccc21 ; CC1(C)c2ccccc2-c2c(-c3nc(-c4cccc(-c5ccc6c(c5)oc5cccc(-c7ccccc7)c56)c4)nc(-c4ccc5sc6ccccc6c5c4)n3)cccc21 ; CC1(C)c2ccccc2-c2cc(-c3nc(-c4cccc(-c5ccc6c(c5)oc5cccc(-c7ccccc7)c56)c4)nc(-c4ccc5c(c4)sc4ccccc45)n3)ccc21 ; CC1(C)c2ccccc2-c2c(-c3nc(-c4cccc(-c5ccc6c(c5)oc5cccc(-c7ccccc7)c56)c4)nc(-c4cccc5c4sc4ccccc45)n3)cccc21] 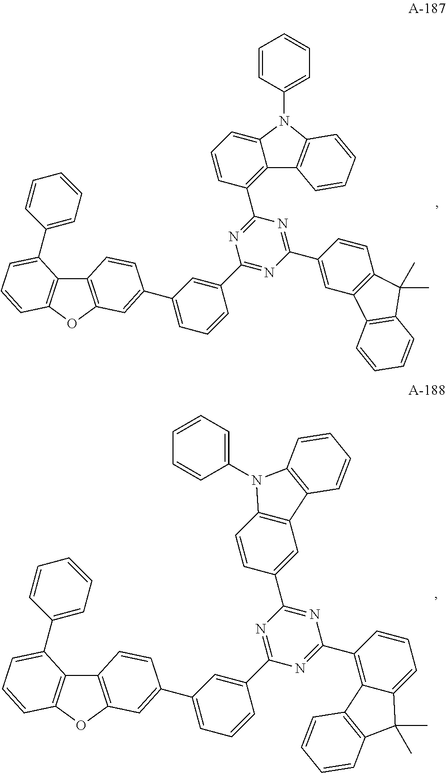 [SMILES: CC1(C)c2ccccc2-c2cc(-c3nc(-c4cccc(-c5ccc6c(c5)oc5cccc(-c7ccccc7)c56)c4)nc(-c4cccc5c4c4ccccc4n5-c4ccccc4)n3)ccc21 ; CC1(C)c2ccccc2-c2c(-c3nc(-c4cccc(-c5ccc6c(c5)oc5cccc(-c7ccccc7)c56)c4)nc(-c4ccc5c(c4)c4ccccc4n5-c4ccccc4)n3)cccc21] 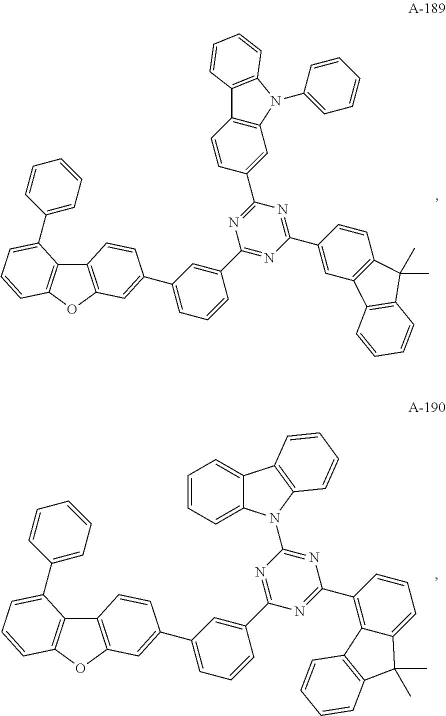 [SMILES: CC1(C)c2ccccc2-c2cc(-c3nc(-c4cccc(-c5ccc6c(c5)oc5cccc(-c7ccccc7)c56)c4)nc(-c4ccc5c6ccccc6n(-c6ccccc6)c5c4)n3)ccc21 ; CC1(C)c2ccccc2-c2c(-c3nc(-c4cccc(-c5ccc6c(c5)oc5cccc(-c7ccccc7)c56)c4)nc(-n4c5ccccc5c5ccccc54)n3)cccc21] 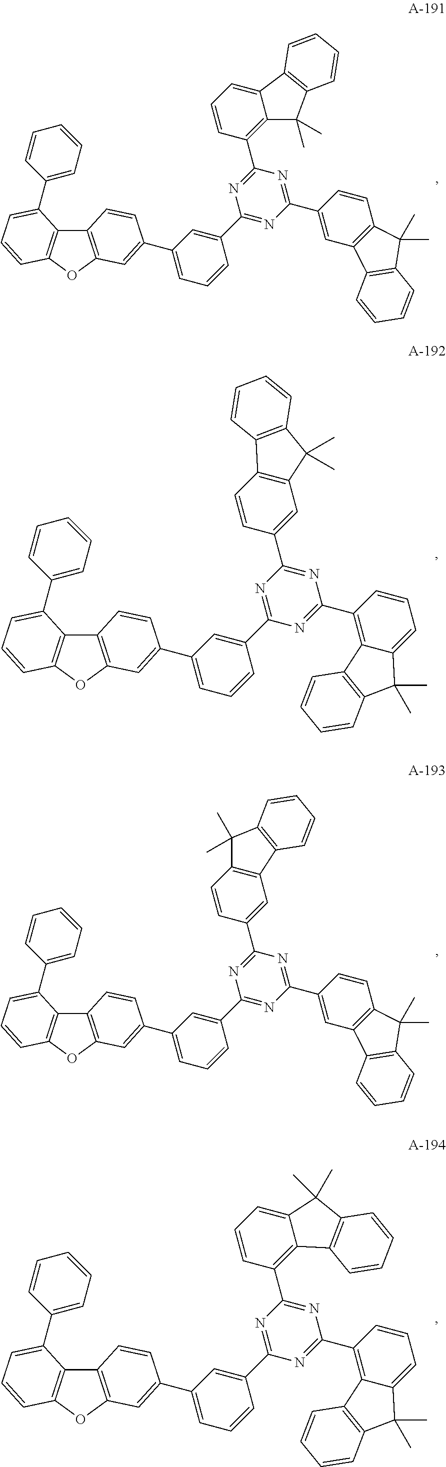 [SMILES: CC1(C)c2ccccc2-c2cc(-c3nc(-c4cccc(-c5ccc6c(c5)oc5cccc(-c7ccccc7)c56)c4)nc(-c4cccc5c4C(C)(C)c4ccccc4-5)n3)ccc21 ; CC1(C)c2ccccc2-c2ccc(-c3nc(-c4cccc(-c5ccc6c(c5)oc5cccc(-c7ccccc7)c56)c4)nc(-c4cccc5c4-c4ccccc4C5(C)C)n3)cc21 ; CC1(C)c2ccccc2-c2cc(-c3nc(-c4cccc(-c5ccc6c(c5)oc5cccc(-c7ccccc7)c56)c4)nc(-c4ccc5c(c4)-c4ccccc4C5(C)C)n3)ccc21 ; CC1(C)c2ccccc2-c2c(-c3nc(-c4cccc(-c5ccc6c(c5)oc5cccc(-c7ccccc7)c56)c4)nc(-c4cccc5c4-c4ccccc4C5(C)C)n3)cccc21] 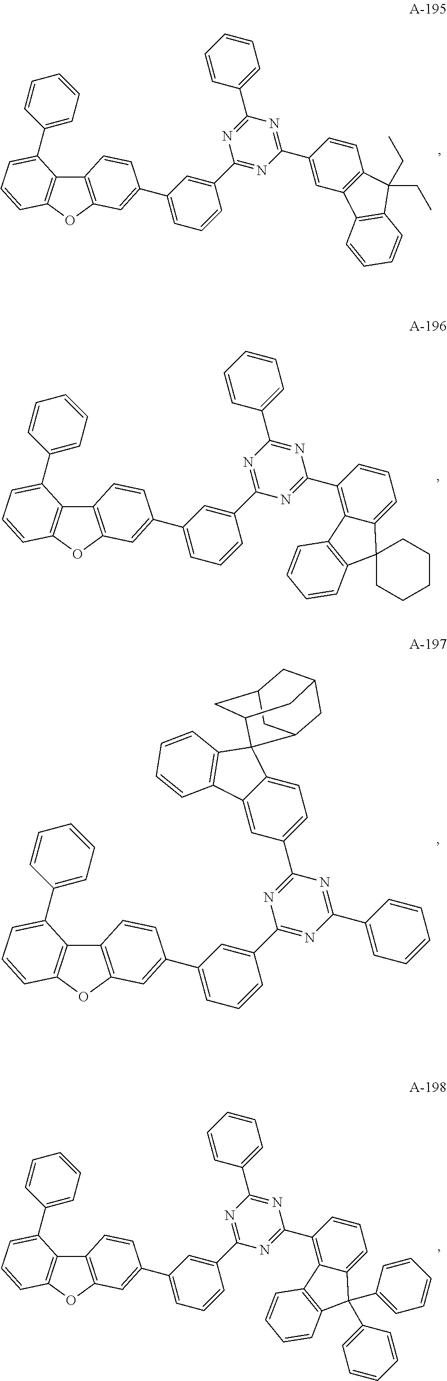 [SMILES: CCC1(CC)c2ccccc2-c2cc(-c3nc(-c4ccccc4)nc(-c4cccc(-c5ccc6c(c5)oc5cccc(-c7ccccc7)c56)c4)n3)ccc21 ; c1ccc(-c2nc(-c3cccc(-c4ccc5c(c4)oc4cccc(-c6ccccc6)c45)c3)nc(-c3cccc4c3-c3ccccc3C43CCCCC3)n2)cc1 ; c1ccc(-c2nc(-c3cccc(-c4ccc5c(c4)oc4cccc(-c6ccccc6)c45)c3)nc(-c3ccc4c(c3)-c3ccccc3C43C4CC5CC(C4)CC3C5)n2)cc1 ; c1ccc(-c2nc(-c3cccc(-c4ccc5c(c4)oc4cccc(-c6ccccc6)c45)c3)nc(-c3cccc4c3-c3ccccc3C4(c3ccccc3)c3ccccc3)n2)cc1] 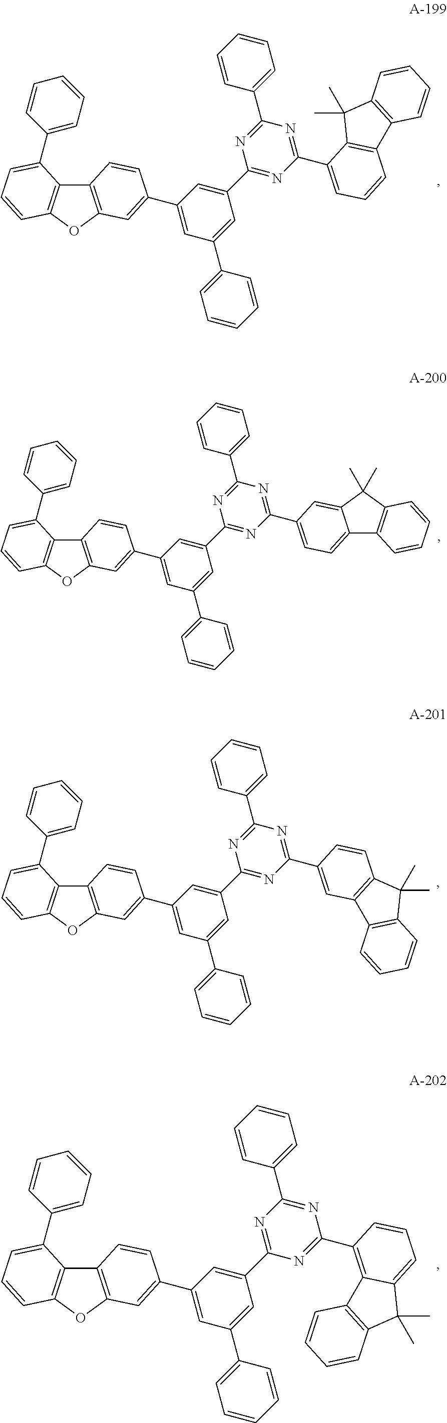 [SMILES: CC1(C)c2ccccc2-c2cccc(-c3nc(-c4ccccc4)nc(-c4cc(-c5ccccc5)cc(-c5ccc6c(c5)oc5cccc(-c7ccccc7)c56)c4)n3)c21 ; CC1(C)c2ccccc2-c2ccc(-c3nc(-c4ccccc4)nc(-c4cc(-c5ccccc5)cc(-c5ccc6c(c5)oc5cccc(-c7ccccc7)c56)c4)n3)cc21 ; CC1(C)c2ccccc2-c2cc(-c3nc(-c4ccccc4)nc(-c4cc(-c5ccccc5)cc(-c5ccc6c(c5)oc5cccc(-c7ccccc7)c56)c4)n3)ccc21 ; CC1(C)c2ccccc2-c2c(-c3nc(-c4ccccc4)nc(-c4cc(-c5ccccc5)cc(-c5ccc6c(c5)oc5cccc(-c7ccccc7)c56)c4)n3)cccc21] 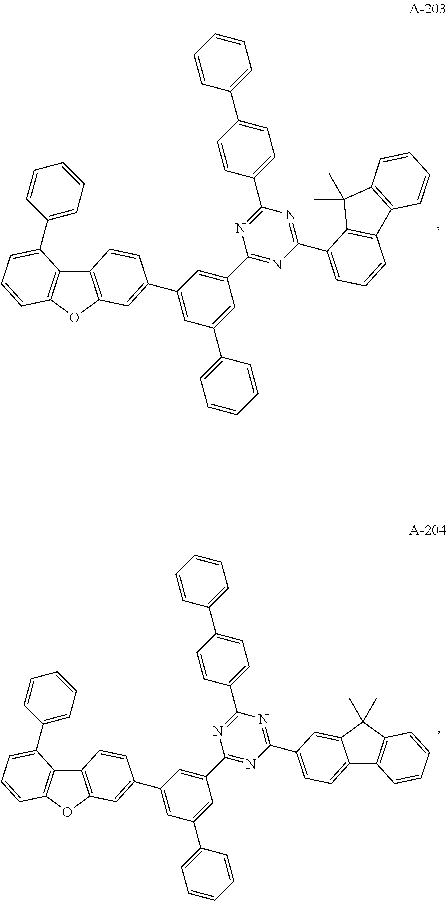 [SMILES: CC1(C)c2ccccc2-c2cccc(-c3nc(-c4ccc(-c5ccccc5)cc4)nc(-c4cc(-c5ccccc5)cc(-c5ccc6c(c5)oc5cccc(-c7ccccc7)c56)c4)n3)c21 ; CC1(C)c2ccccc2-c2ccc(-c3nc(-c4ccc(-c5ccccc5)cc4)nc(-c4cc(-c5ccccc5)cc(-c5ccc6c(c5)oc5cccc(-c7ccccc7)c56)c4)n3)cc21] 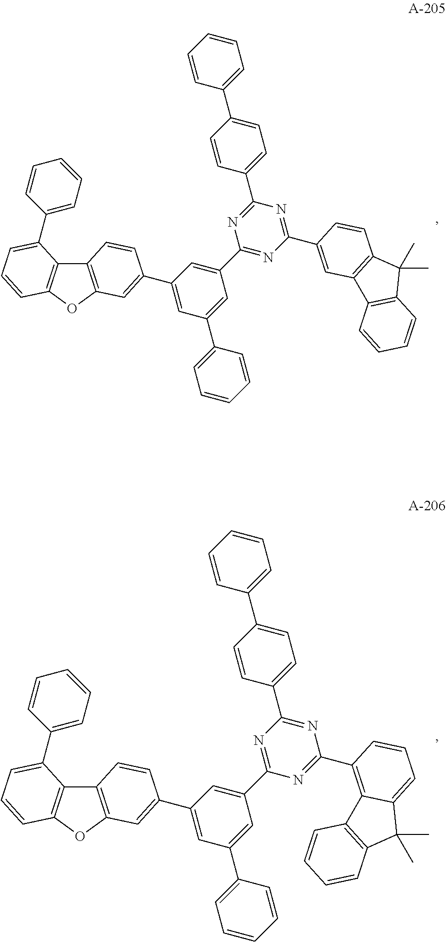 [SMILES: CC1(C)c2ccccc2-c2cc(-c3nc(-c4ccc(-c5ccccc5)cc4)nc(-c4cc(-c5ccccc5)cc(-c5ccc6c(c5)oc5cccc(-c7ccccc7)c56)c4)n3)ccc21 ; CC1(C)c2ccccc2-c2c(-c3nc(-c4ccc(-c5ccccc5)cc4)nc(-c4cc(-c5ccccc5)cc(-c5ccc6c(c5)oc5cccc(-c7ccccc7)c56)c4)n3)cccc21] 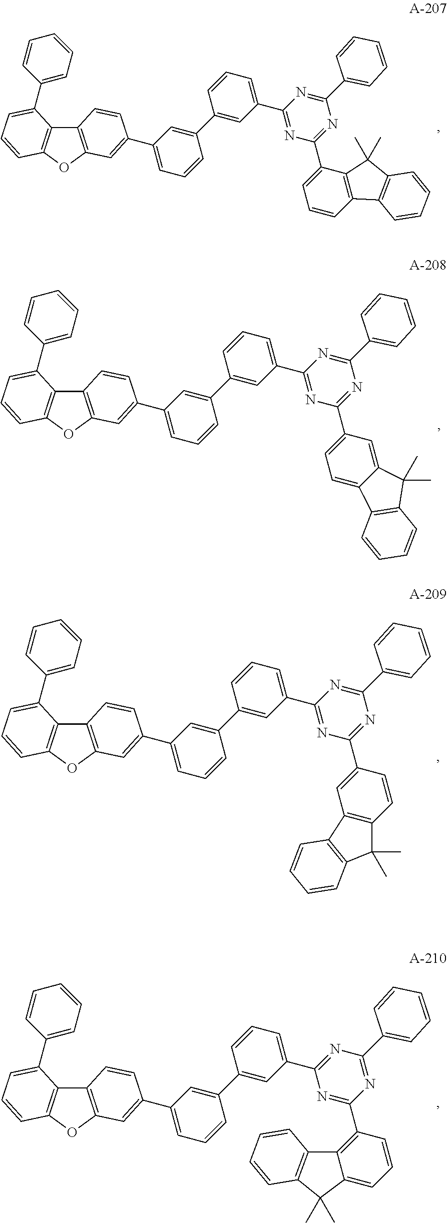 [SMILES: CC1(C)c2ccccc2-c2cccc(-c3nc(-c4ccccc4)nc(-c4cccc(-c5cccc(-c6ccc7c(c6)oc6cccc(-c8ccccc8)c67)c5)c4)n3)c21 ; CC1(C)c2ccccc2-c2ccc(-c3nc(-c4ccccc4)nc(-c4cccc(-c5cccc(-c6ccc7c(c6)oc6cccc(-c8ccccc8)c67)c5)c4)n3)cc21 ; CC1(C)c2ccccc2-c2cc(-c3nc(-c4ccccc4)nc(-c4cccc(-c5cccc(-c6ccc7c(c6)oc6cccc(-c8ccccc8)c67)c5)c4)n3)ccc21 ; CC1(C)c2ccccc2-c2c(-c3nc(-c4ccccc4)nc(-c4cccc(-c5cccc(-c6ccc7c(c6)oc6cccc(-c8ccccc8)c67)c5)c4)n3)cccc21] 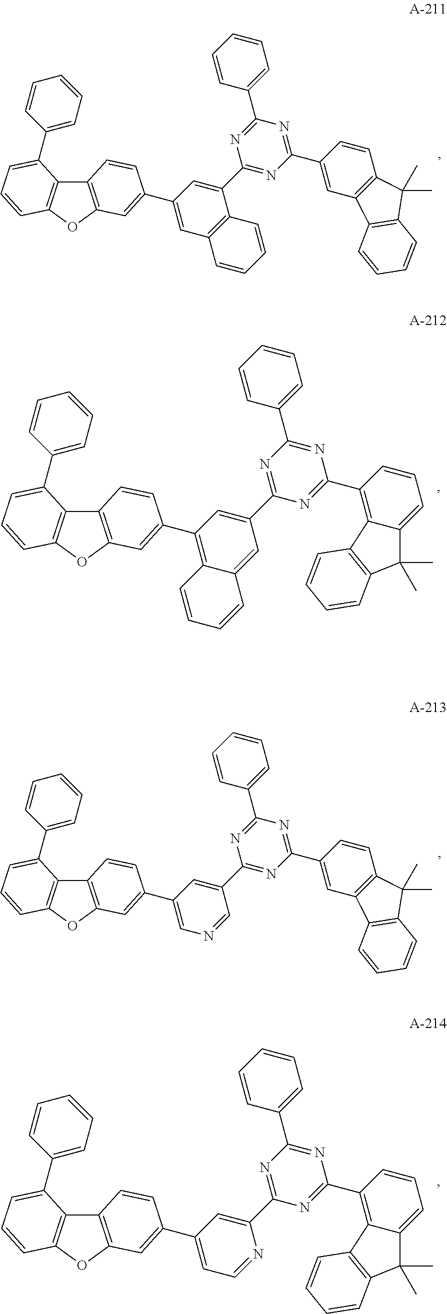 [SMILES: CC1(C)c2ccccc2-c2cc(-c3nc(-c4ccccc4)nc(-c4cc(-c5ccc6c(c5)oc5cccc(-c7ccccc7)c56)cc5ccccc45)n3)ccc21 ; CC1(C)c2ccccc2-c2c(-c3nc(-c4ccccc4)nc(-c4cc(-c5ccc6c(c5)oc5cccc(-c7ccccc7)c56)c5ccccc5c4)n3)cccc21 ; CC1(C)c2ccccc2-c2cc(-c3nc(-c4ccccc4)nc(-c4cncc(-c5ccc6c(c5)oc5cccc(-c7ccccc7)c56)c4)n3)ccc21 ; CC1(C)c2ccccc2-c2c(-c3nc(-c4ccccc4)nc(-c4cc(-c5ccc6c(c5)oc5cccc(-c7ccccc7)c56)ccn4)n3)cccc21] 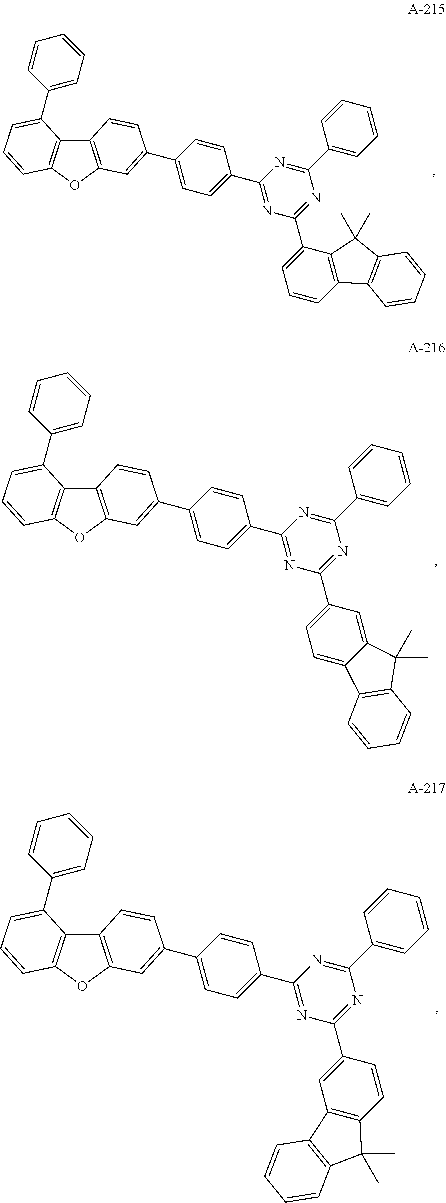 [SMILES: CC1(C)c2ccccc2-c2cccc(-c3nc(-c4ccccc4)nc(-c4ccc(-c5ccc6c(c5)oc5cccc(-c7ccccc7)c56)cc4)n3)c21 ; CC1(C)c2ccccc2-c2ccc(-c3nc(-c4ccccc4)nc(-c4ccc(-c5ccc6c(c5)oc5cccc(-c7ccccc7)c56)cc4)n3)cc21 ; CC1(C)c2ccccc2-c2cc(-c3nc(-c4ccccc4)nc(-c4ccc(-c5ccc6c(c5)oc5cccc(-c7ccccc7)c56)cc4)n3)ccc21] 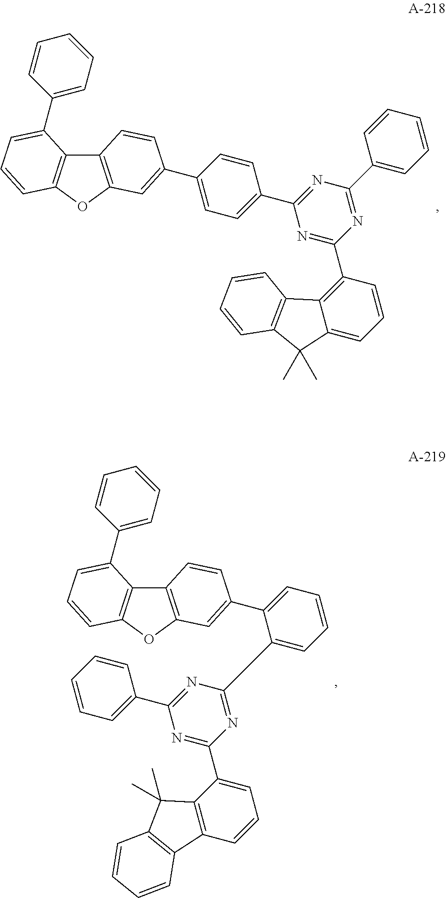 [SMILES: CC1(C)c2ccccc2-c2c(-c3nc(-c4ccccc4)nc(-c4ccc(-c5ccc6c(c5)oc5cccc(-c7ccccc7)c56)cc4)n3)cccc21 ; CC1(C)c2ccccc2-c2cccc(-c3nc(-c4ccccc4)nc(-c4ccccc4-c4ccc5c(c4)oc4cccc(-c6ccccc6)c45)n3)c21] 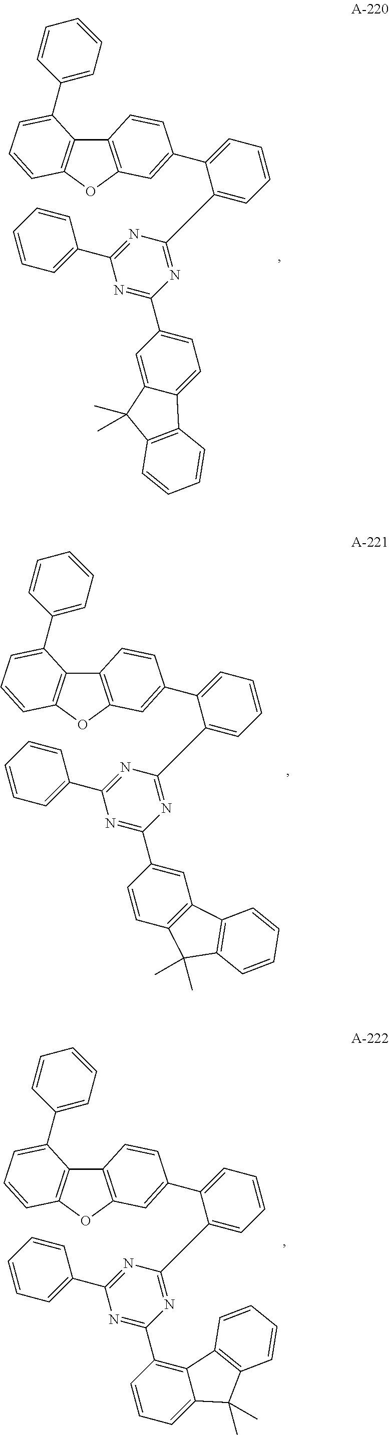 [SMILES: CC1(C)c2ccccc2-c2ccc(-c3nc(-c4ccccc4)nc(-c4ccccc4-c4ccc5c(c4)oc4cccc(-c6ccccc6)c45)n3)cc21 ; CC1(C)c2ccccc2-c2cc(-c3nc(-c4ccccc4)nc(-c4ccccc4-c4ccc5c(c4)oc4cccc(-c6ccccc6)c45)n3)ccc21 ; CC1(C)c2ccccc2-c2c(-c3nc(-c4ccccc4)nc(-c4ccccc4-c4ccc5c(c4)oc4cccc(-c6ccccc6)c45)n3)cccc21] 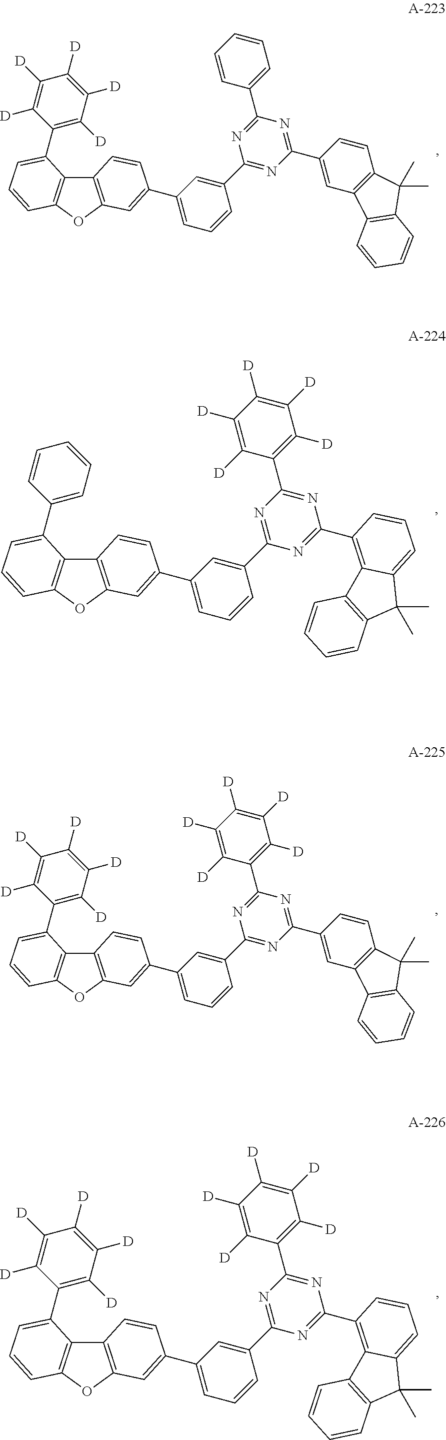 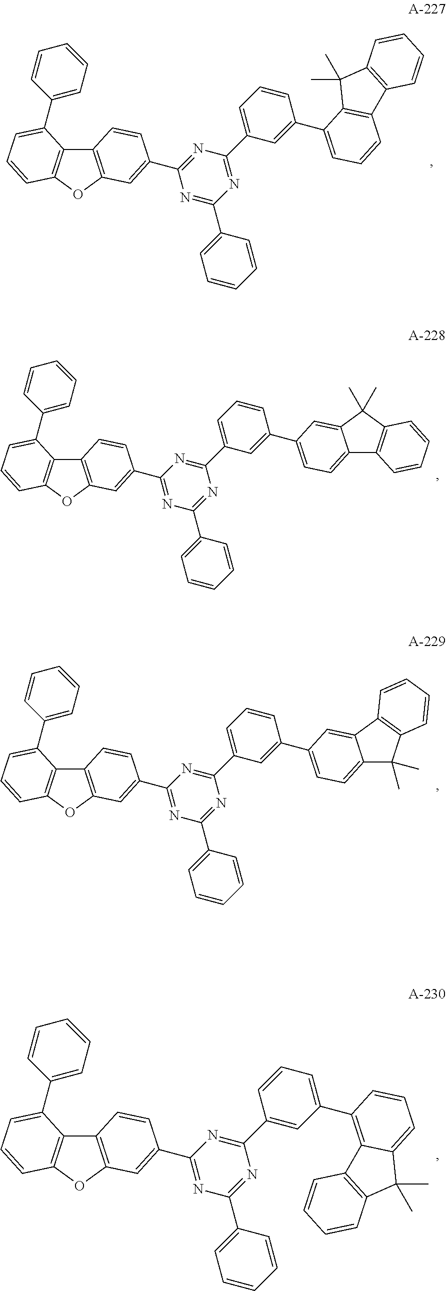 [SMILES: CC1(C)c2ccccc2-c2cccc(-c3cccc(-c4nc(-c5ccccc5)nc(-c5ccc6c(c5)oc5cccc(-c7ccccc7)c56)n4)c3)c21 ; CC1(C)c2ccccc2-c2ccc(-c3cccc(-c4nc(-c5ccccc5)nc(-c5ccc6c(c5)oc5cccc(-c7ccccc7)c56)n4)c3)cc21 ; CC1(C)c2ccccc2-c2cc(-c3cccc(-c4nc(-c5ccccc5)nc(-c5ccc6c(c5)oc5cccc(-c7ccccc7)c56)n4)c3)ccc21 ; CC1(C)c2ccccc2-c2c(-c3cccc(-c4nc(-c5ccccc5)nc(-c5ccc6c(c5)oc5cccc(-c7ccccc7)c56)n4)c3)cccc21] 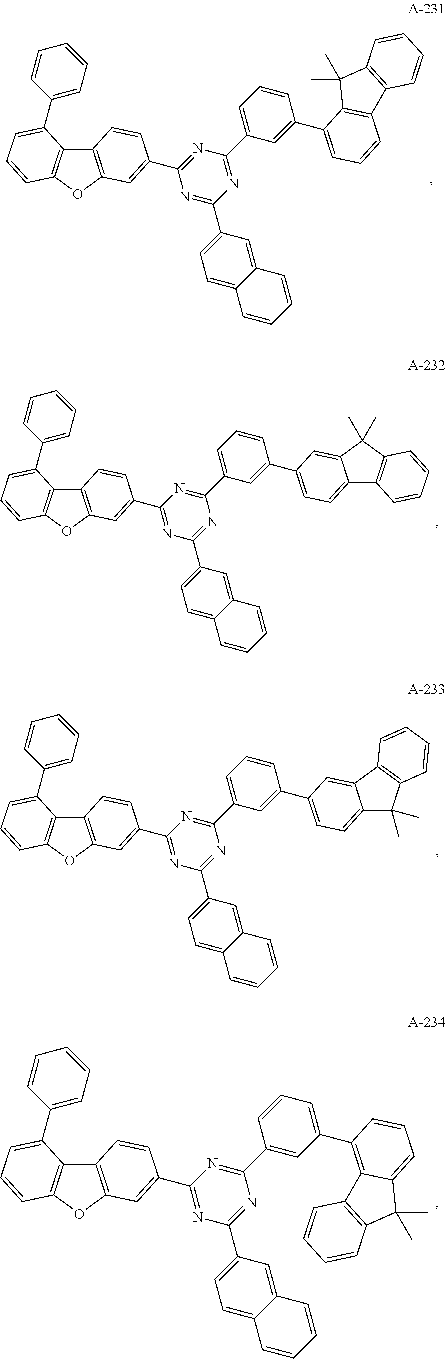 [SMILES: CC1(C)c2ccccc2-c2cccc(-c3cccc(-c4nc(-c5ccc6ccccc6c5)nc(-c5ccc6c(c5)oc5cccc(-c7ccccc7)c56)n4)c3)c21 ; CC1(C)c2ccccc2-c2ccc(-c3cccc(-c4nc(-c5ccc6ccccc6c5)nc(-c5ccc6c(c5)oc5cccc(-c7ccccc7)c56)n4)c3)cc21 ; CC1(C)c2ccccc2-c2cc(-c3cccc(-c4nc(-c5ccc6ccccc6c5)nc(-c5ccc6c(c5)oc5cccc(-c7ccccc7)c56)n4)c3)ccc21 ; CC1(C)c2ccccc2-c2c(-c3cccc(-c4nc(-c5ccc6ccccc6c5)nc(-c5ccc6c(c5)oc5cccc(-c7ccccc7)c56)n4)c3)cccc21] 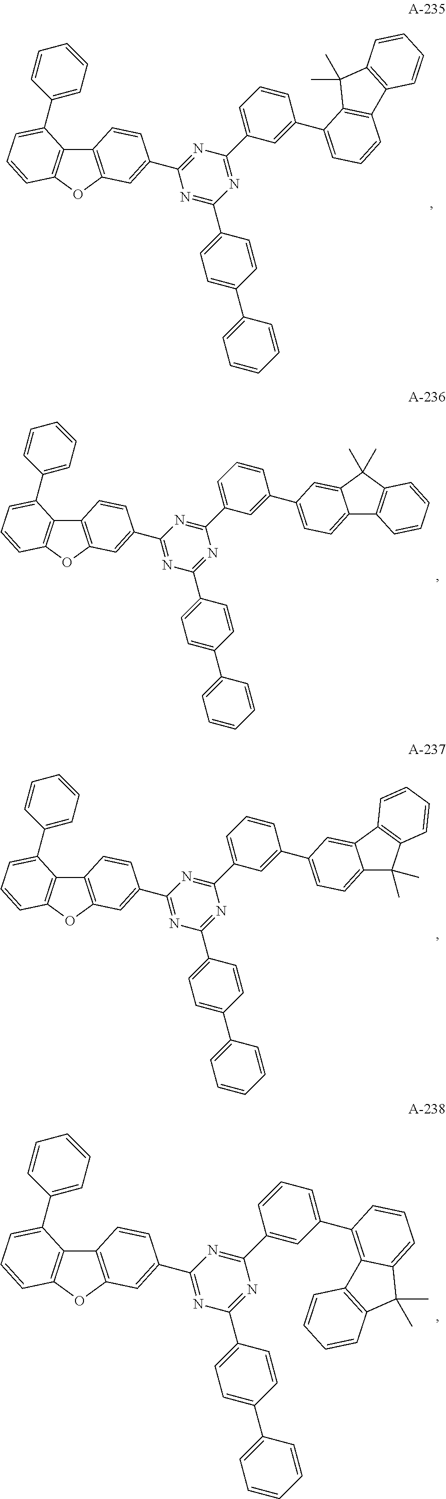 [SMILES: CC1(C)c2ccccc2-c2cccc(-c3cccc(-c4nc(-c5ccc(-c6ccccc6)cc5)nc(-c5ccc6c(c5)oc5cccc(-c7ccccc7)c56)n4)c3)c21 ; CC1(C)c2ccccc2-c2ccc(-c3cccc(-c4nc(-c5ccc(-c6ccccc6)cc5)nc(-c5ccc6c(c5)oc5cccc(-c7ccccc7)c56)n4)c3)cc21 ; CC1(C)c2ccccc2-c2cc(-c3cccc(-c4nc(-c5ccc(-c6ccccc6)cc5)nc(-c5ccc6c(c5)oc5cccc(-c7ccccc7)c56)n4)c3)ccc21 ; CC1(C)c2ccccc2-c2c(-c3cccc(-c4nc(-c5ccc(-c6ccccc6)cc5)nc(-c5ccc6c(c5)oc5cccc(-c7ccccc7)c56)n4)c3)cccc21] 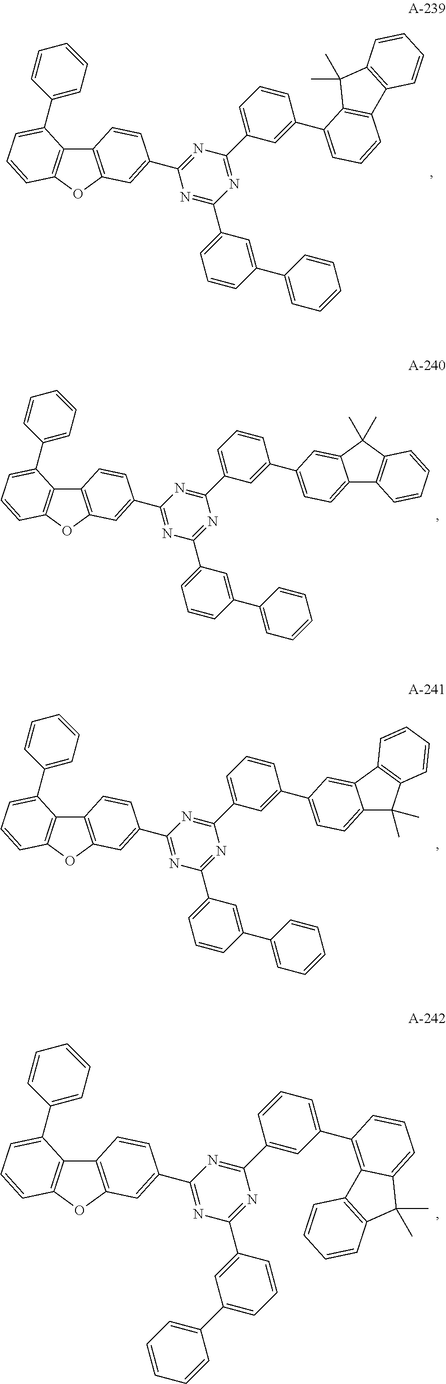 [SMILES: CC1(C)c2ccccc2-c2cccc(-c3cccc(-c4nc(-c5cccc(-c6ccccc6)c5)nc(-c5ccc6c(c5)oc5cccc(-c7ccccc7)c56)n4)c3)c21 ; CC1(C)c2ccccc2-c2ccc(-c3cccc(-c4nc(-c5cccc(-c6ccccc6)c5)nc(-c5ccc6c(c5)oc5cccc(-c7ccccc7)c56)n4)c3)cc21 ; CC1(C)c2ccccc2-c2cc(-c3cccc(-c4nc(-c5cccc(-c6ccccc6)c5)nc(-c5ccc6c(c5)oc5cccc(-c7ccccc7)c56)n4)c3)ccc21 ; CC1(C)c2ccccc2-c2c(-c3cccc(-c4nc(-c5cccc(-c6ccccc6)c5)nc(-c5ccc6c(c5)oc5cccc(-c7ccccc7)c56)n4)c3)cccc21] 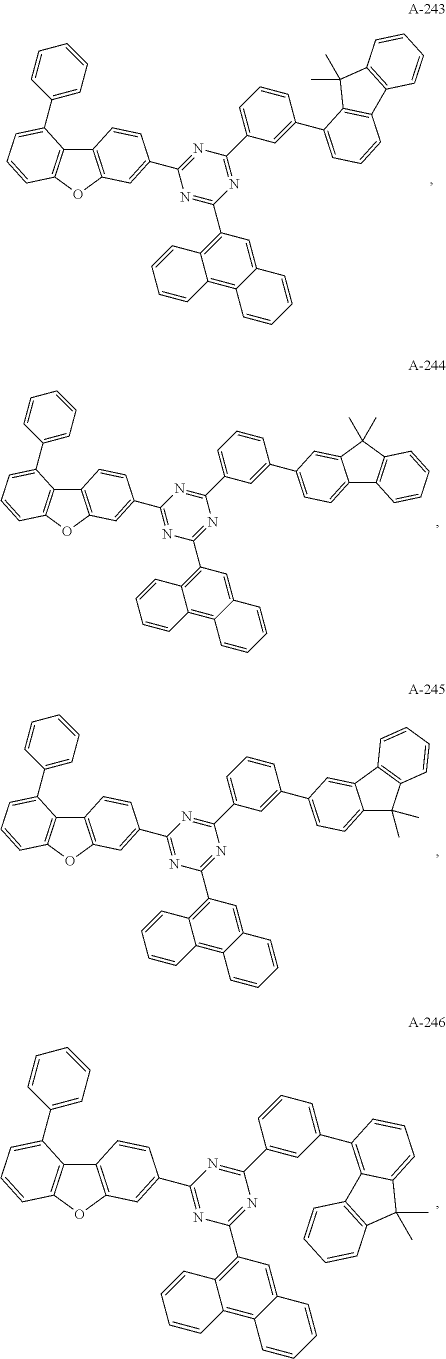 [SMILES: CC1(C)c2ccccc2-c2cccc(-c3cccc(-c4nc(-c5ccc6c(c5)oc5cccc(-c7ccccc7)c56)nc(-c5cc6ccccc6c6ccccc56)n4)c3)c21 ; CC1(C)c2ccccc2-c2ccc(-c3cccc(-c4nc(-c5ccc6c(c5)oc5cccc(-c7ccccc7)c56)nc(-c5cc6ccccc6c6ccccc56)n4)c3)cc21 ; CC1(C)c2ccccc2-c2cc(-c3cccc(-c4nc(-c5ccc6c(c5)oc5cccc(-c7ccccc7)c56)nc(-c5cc6ccccc6c6ccccc56)n4)c3)ccc21 ; CC1(C)c2ccccc2-c2c(-c3cccc(-c4nc(-c5ccc6c(c5)oc5cccc(-c7ccccc7)c56)nc(-c5cc6ccccc6c6ccccc56)n4)c3)cccc21] 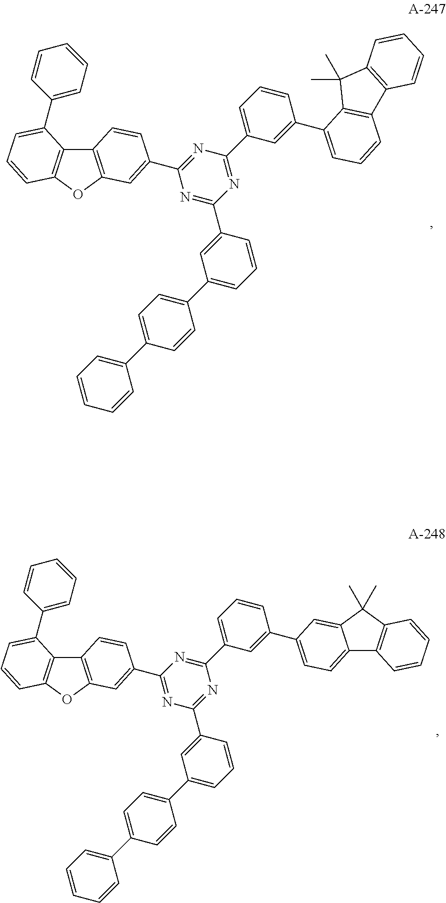 [SMILES: CC1(C)c2ccccc2-c2cccc(-c3cccc(-c4nc(-c5cccc(-c6ccc(-c7ccccc7)cc6)c5)nc(-c5ccc6c(c5)oc5cccc(-c7ccccc7)c56)n4)c3)c21 ; CC1(C)c2ccccc2-c2ccc(-c3cccc(-c4nc(-c5cccc(-c6ccc(-c7ccccc7)cc6)c5)nc(-c5ccc6c(c5)oc5cccc(-c7ccccc7)c56)n4)c3)cc21] 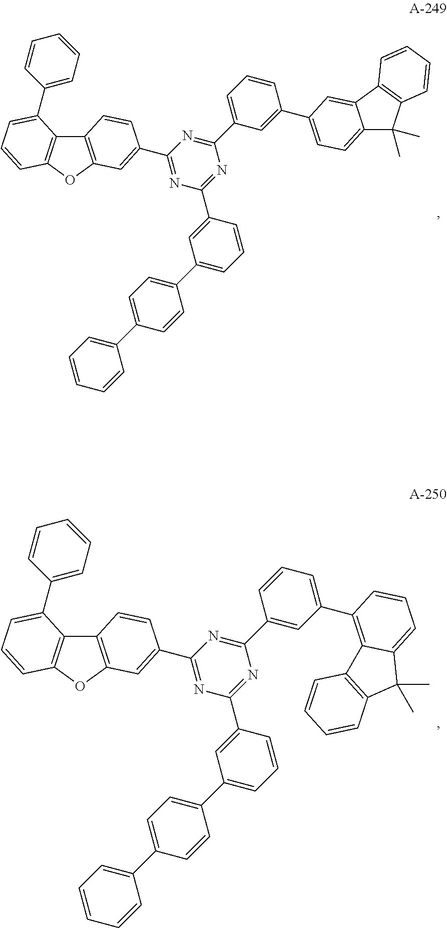 [SMILES: CC1(C)c2ccccc2-c2cc(-c3cccc(-c4nc(-c5cccc(-c6ccc(-c7ccccc7)cc6)c5)nc(-c5ccc6c(c5)oc5cccc(-c7ccccc7)c56)n4)c3)ccc21 ; CC1(C)c2ccccc2-c2c(-c3cccc(-c4nc(-c5cccc(-c6ccc(-c7ccccc7)cc6)c5)nc(-c5ccc6c(c5)oc5cccc(-c7ccccc7)c56)n4)c3)cccc21] 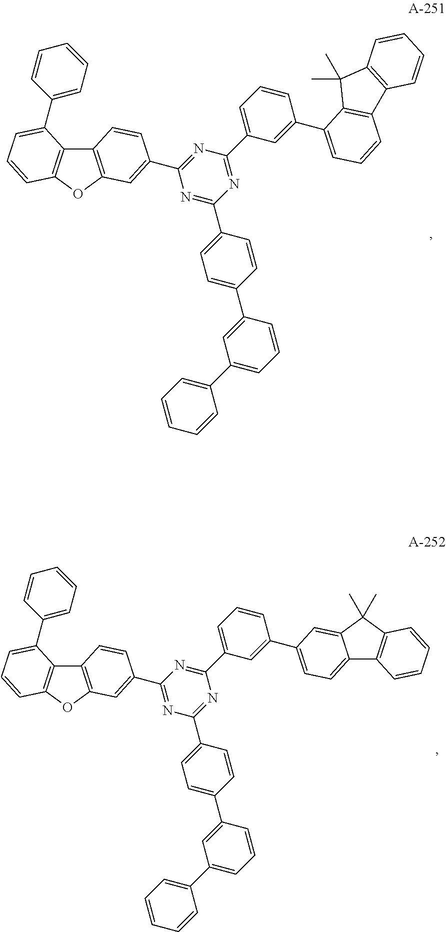 [SMILES: CC1(C)c2ccccc2-c2cccc(-c3cccc(-c4nc(-c5ccc(-c6cccc(-c7ccccc7)c6)cc5)nc(-c5ccc6c(c5)oc5cccc(-c7ccccc7)c56)n4)c3)c21 ; CC1(C)c2ccccc2-c2ccc(-c3cccc(-c4nc(-c5ccc(-c6cccc(-c7ccccc7)c6)cc5)nc(-c5ccc6c(c5)oc5cccc(-c7ccccc7)c56)n4)c3)cc21] 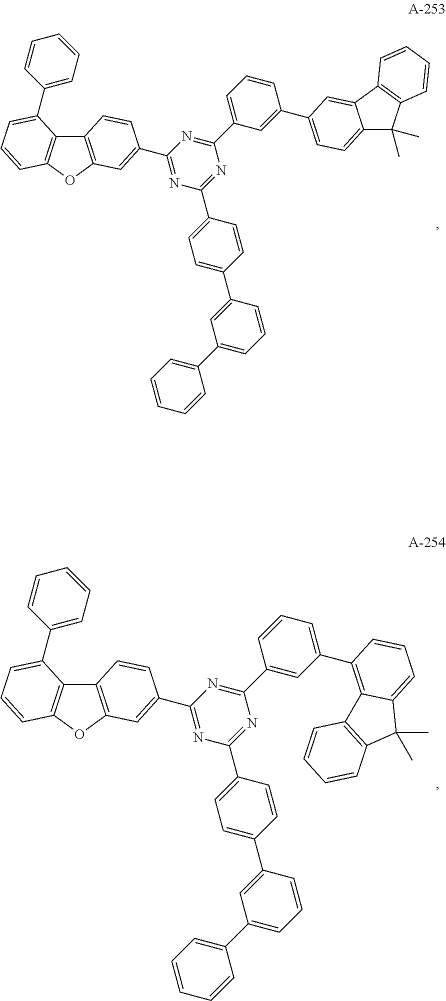 [SMILES: CC1(C)c2ccccc2-c2cc(-c3cccc(-c4nc(-c5ccc(-c6cccc(-c7ccccc7)c6)cc5)nc(-c5ccc6c(c5)oc5cccc(-c7ccccc7)c56)n4)c3)ccc21 ; CC1(C)c2ccccc2-c2c(-c3cccc(-c4nc(-c5ccc(-c6cccc(-c7ccccc7)c6)cc5)nc(-c5ccc6c(c5)oc5cccc(-c7ccccc7)c56)n4)c3)cccc21] 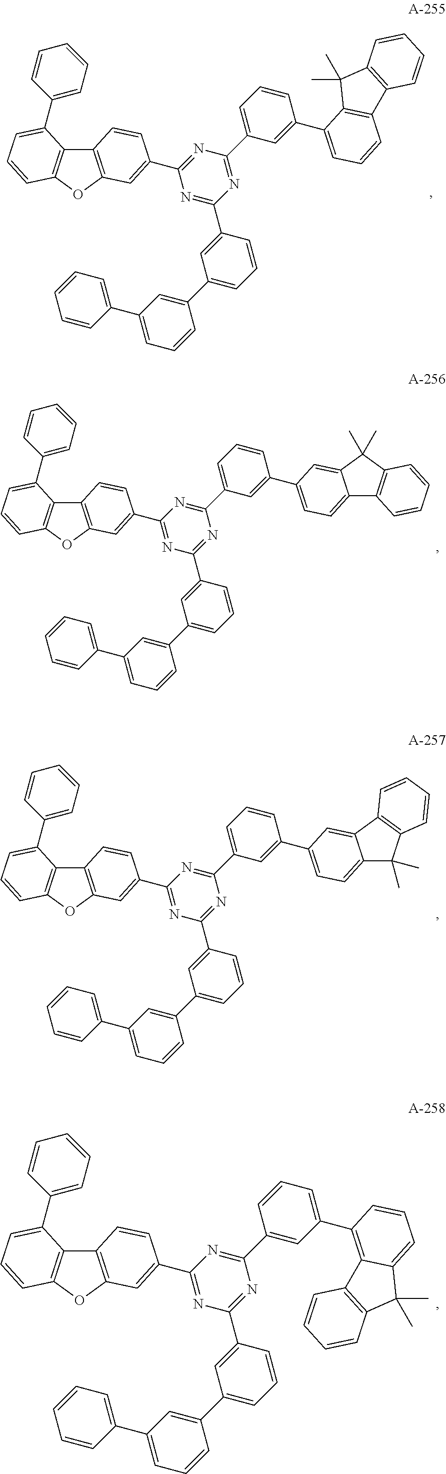 [SMILES: CC1(C)c2ccccc2-c2cccc(-c3cccc(-c4nc(-c5cccc(-c6cccc(-c7ccccc7)c6)c5)nc(-c5ccc6c(c5)oc5cccc(-c7ccccc7)c56)n4)c3)c21 ; CC1(C)c2ccccc2-c2ccc(-c3cccc(-c4nc(-c5cccc(-c6cccc(-c7ccccc7)c6)c5)nc(-c5ccc6c(c5)oc5cccc(-c7ccccc7)c56)n4)c3)cc21 ; CC1(C)c2ccccc2-c2cc(-c3cccc(-c4nc(-c5cccc(-c6cccc(-c7ccccc7)c6)c5)nc(-c5ccc6c(c5)oc5cccc(-c7ccccc7)c56)n4)c3)ccc21 ; CC1(C)c2ccccc2-c2c(-c3cccc(-c4nc(-c5cccc(-c6cccc(-c7ccccc7)c6)c5)nc(-c5ccc6c(c5)oc5cccc(-c7ccccc7)c56)n4)c3)cccc21] 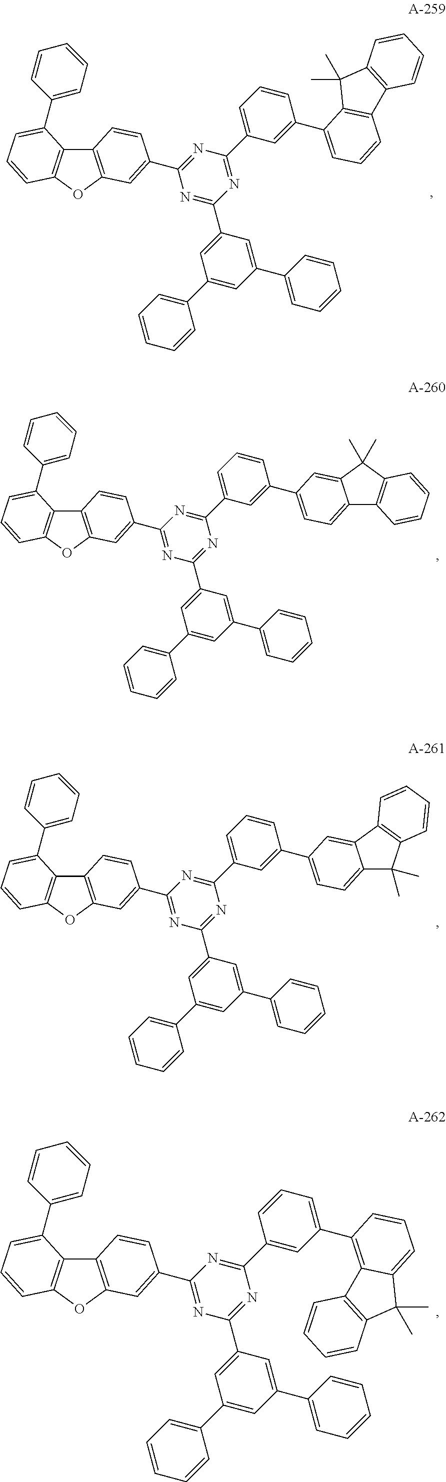 [SMILES: CC1(C)c2ccccc2-c2cccc(-c3cccc(-c4nc(-c5cc(-c6ccccc6)cc(-c6ccccc6)c5)nc(-c5ccc6c(c5)oc5cccc(-c7ccccc7)c56)n4)c3)c21 ; CC1(C)c2ccccc2-c2ccc(-c3cccc(-c4nc(-c5cc(-c6ccccc6)cc(-c6ccccc6)c5)nc(-c5ccc6c(c5)oc5cccc(-c7ccccc7)c56)n4)c3)cc21 ; CC1(C)c2ccccc2-c2cc(-c3cccc(-c4nc(-c5cc(-c6ccccc6)cc(-c6ccccc6)c5)nc(-c5ccc6c(c5)oc5cccc(-c7ccccc7)c56)n4)c3)ccc21 ; CC1(C)c2ccccc2-c2c(-c3cccc(-c4nc(-c5cc(-c6ccccc6)cc(-c6ccccc6)c5)nc(-c5ccc6c(c5)oc5cccc(-c7ccccc7)c56)n4)c3)cccc21] 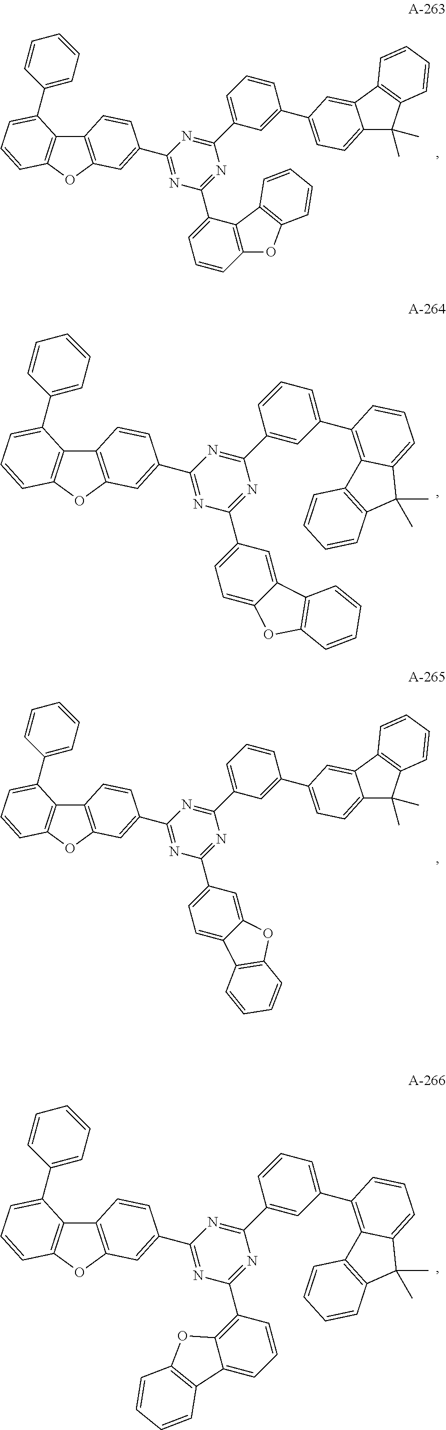 [SMILES: CC1(C)c2ccccc2-c2cc(-c3cccc(-c4nc(-c5ccc6c(c5)oc5cccc(-c7ccccc7)c56)nc(-c5cccc6oc7ccccc7c56)n4)c3)ccc21 ; CC1(C)c2ccccc2-c2c(-c3cccc(-c4nc(-c5ccc6c(c5)oc5cccc(-c7ccccc7)c56)nc(-c5ccc6oc7ccccc7c6c5)n4)c3)cccc21 ; CC1(C)c2ccccc2-c2cc(-c3cccc(-c4nc(-c5ccc6c(c5)oc5ccccc56)nc(-c5ccc6c(c5)oc5cccc(-c7ccccc7)c56)n4)c3)ccc21 ; CC1(C)c2ccccc2-c2c(-c3cccc(-c4nc(-c5ccc6c(c5)oc5cccc(-c7ccccc7)c56)nc(-c5cccc6c5oc5ccccc56)n4)c3)cccc21] 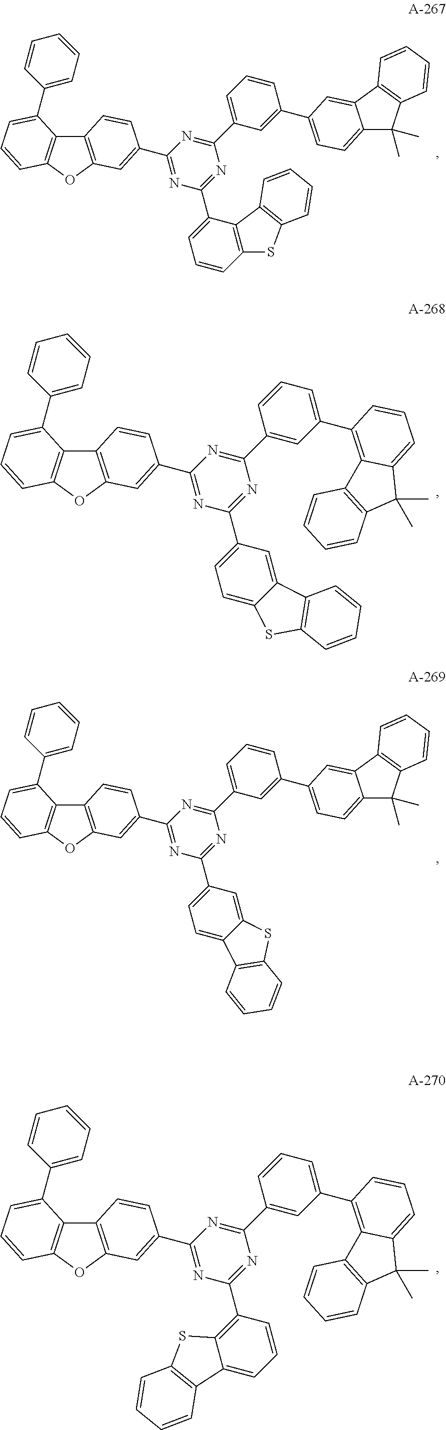 [SMILES: CC1(C)c2ccccc2-c2cc(-c3cccc(-c4nc(-c5ccc6c(c5)oc5cccc(-c7ccccc7)c56)nc(-c5cccc6sc7ccccc7c56)n4)c3)ccc21 ; CC1(C)c2ccccc2-c2c(-c3cccc(-c4nc(-c5ccc6c(c5)oc5cccc(-c7ccccc7)c56)nc(-c5ccc6sc7ccccc7c6c5)n4)c3)cccc21 ; CC1(C)c2ccccc2-c2cc(-c3cccc(-c4nc(-c5ccc6c(c5)oc5cccc(-c7ccccc7)c56)nc(-c5ccc6c(c5)sc5ccccc56)n4)c3)ccc21 ; CC1(C)c2ccccc2-c2c(-c3cccc(-c4nc(-c5ccc6c(c5)oc5cccc(-c7ccccc7)c56)nc(-c5cccc6c5sc5ccccc56)n4)c3)cccc21] 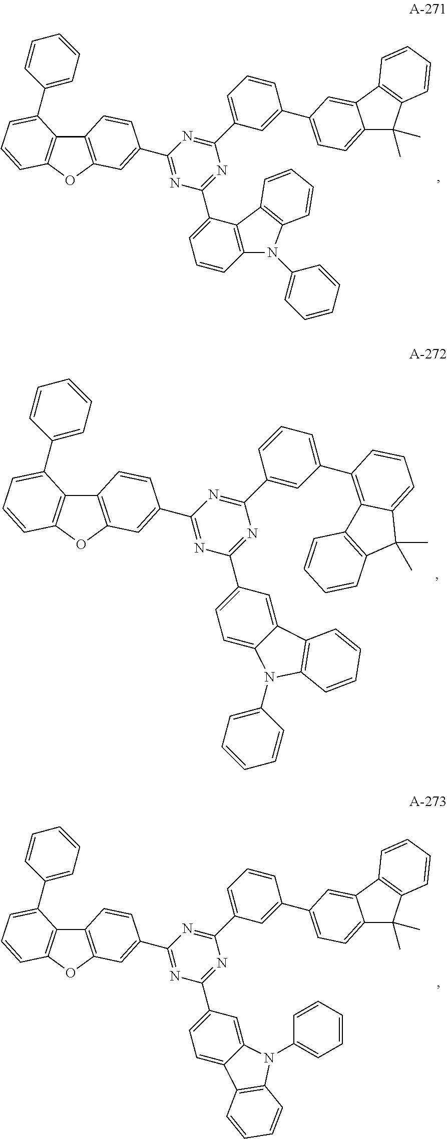 [SMILES: CC1(C)c2ccccc2-c2cc(-c3cccc(-c4nc(-c5ccc6c(c5)oc5cccc(-c7ccccc7)c56)nc(-c5cccc6c5c5ccccc5n6-c5ccccc5)n4)c3)ccc21 ; CC1(C)c2ccccc2-c2c(-c3cccc(-c4nc(-c5ccc6c(c5)oc5cccc(-c7ccccc7)c56)nc(-c5ccc6c(c5)c5ccccc5n6-c5ccccc5)n4)c3)cccc21 ; CC1(C)c2ccccc2-c2cc(-c3cccc(-c4nc(-c5ccc6c(c5)oc5cccc(-c7ccccc7)c56)nc(-c5ccc6c7ccccc7n(-c7ccccc7)c6c5)n4)c3)ccc21] 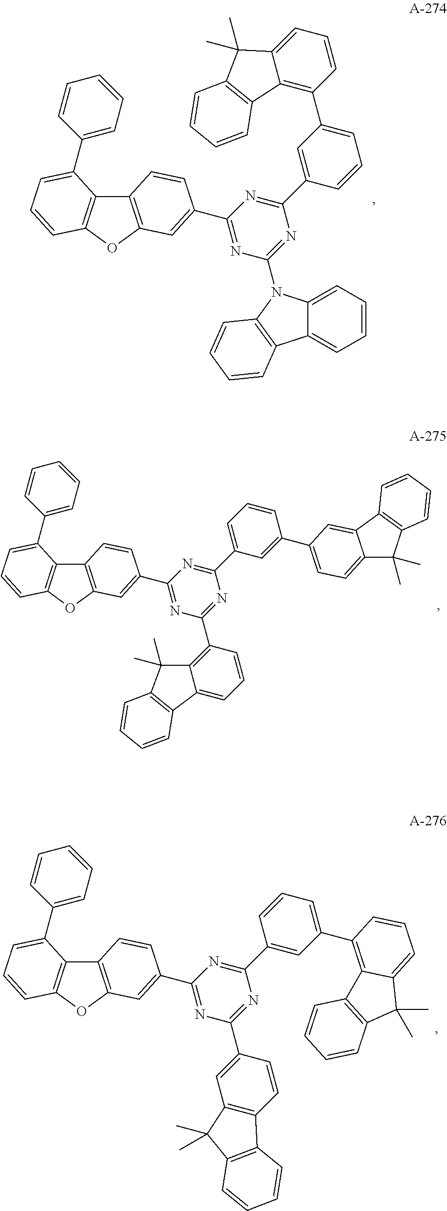 [SMILES: CC1(C)c2ccccc2-c2c(-c3cccc(-c4nc(-c5ccc6c(c5)oc5cccc(-c7ccccc7)c56)nc(-n5c6ccccc6c6ccccc65)n4)c3)cccc21 ; CC1(C)c2ccccc2-c2cc(-c3cccc(-c4nc(-c5ccc6c(c5)oc5cccc(-c7ccccc7)c56)nc(-c5cccc6c5C(C)(C)c5ccccc5-6)n4)c3)ccc21 ; CC1(C)c2ccccc2-c2ccc(-c3nc(-c4cccc(-c5cccc6c5-c5ccccc5C6(C)C)c4)nc(-c4ccc5c(c4)oc4cccc(-c6ccccc6)c45)n3)cc21] 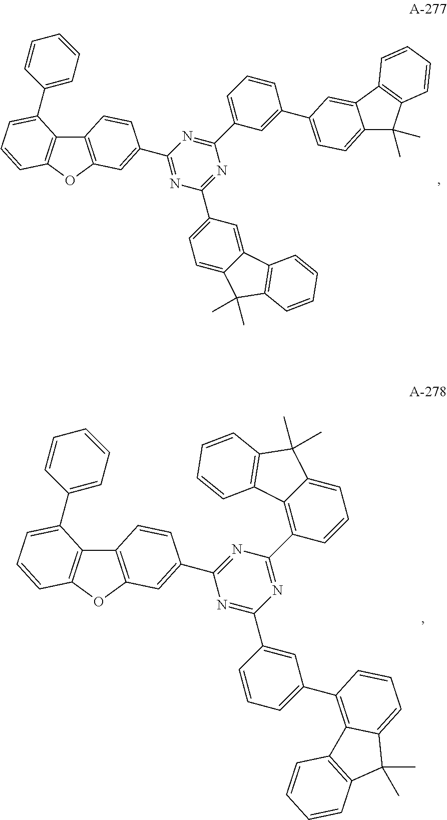 [SMILES: CC1(C)c2ccccc2-c2cc(-c3cccc(-c4nc(-c5ccc6c(c5)-c5ccccc5C6(C)C)nc(-c5ccc6c(c5)oc5cccc(-c7ccccc7)c56)n4)c3)ccc21 ; CC1(C)c2ccccc2-c2c(-c3cccc(-c4nc(-c5ccc6c(c5)oc5cccc(-c7ccccc7)c56)nc(-c5cccc6c5-c5ccccc5C6(C)C)n4)c3)cccc21] 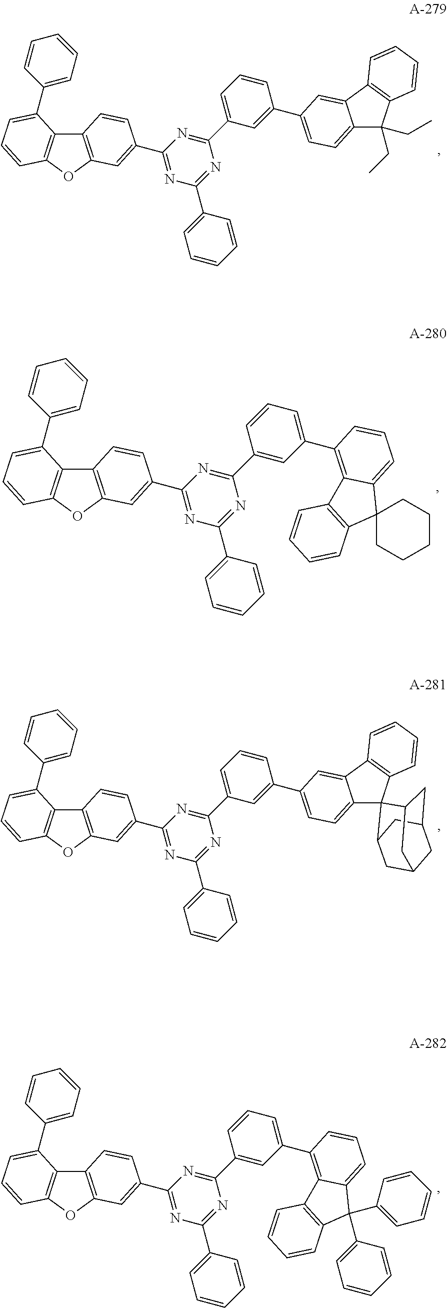 [SMILES: CCC1(CC)c2ccccc2-c2cc(-c3cccc(-c4nc(-c5ccccc5)nc(-c5ccc6c(c5)oc5cccc(-c7ccccc7)c56)n4)c3)ccc21 ; c1ccc(-c2nc(-c3cccc(-c4cccc5c4-c4ccccc4C54CCCCC4)c3)nc(-c3ccc4c(c3)oc3cccc(-c5ccccc5)c34)n2)cc1 ; c1ccc(-c2nc(-c3cccc(-c4ccc5c(c4)-c4ccccc4C54C5CC6CC(C5)CC4C6)c3)nc(-c3ccc4c(c3)oc3cccc(-c5ccccc5)c34)n2)cc1 ; c1ccc(-c2nc(-c3cccc(-c4cccc5c4-c4ccccc4C5(c4ccccc4)c4ccccc4)c3)nc(-c3ccc4c(c3)oc3cccc(-c5ccccc5)c34)n2)cc1] 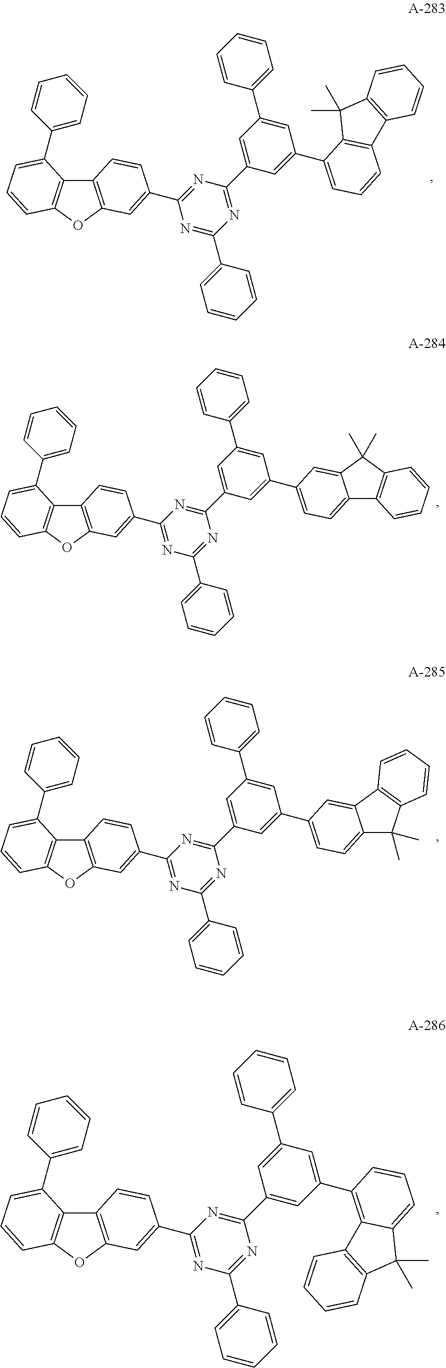 [SMILES: CC1(C)c2ccccc2-c2cccc(-c3cc(-c4ccccc4)cc(-c4nc(-c5ccccc5)nc(-c5ccc6c(c5)oc5cccc(-c7ccccc7)c56)n4)c3)c21 ; CC1(C)c2ccccc2-c2ccc(-c3cc(-c4ccccc4)cc(-c4nc(-c5ccccc5)nc(-c5ccc6c(c5)oc5cccc(-c7ccccc7)c56)n4)c3)cc21 ; CC1(C)c2ccccc2-c2cc(-c3cc(-c4ccccc4)cc(-c4nc(-c5ccccc5)nc(-c5ccc6c(c5)oc5cccc(-c7ccccc7)c56)n4)c3)ccc21 ; CC1(C)c2ccccc2-c2c(-c3cc(-c4ccccc4)cc(-c4nc(-c5ccccc5)nc(-c5ccc6c(c5)oc5cccc(-c7ccccc7)c56)n4)c3)cccc21] 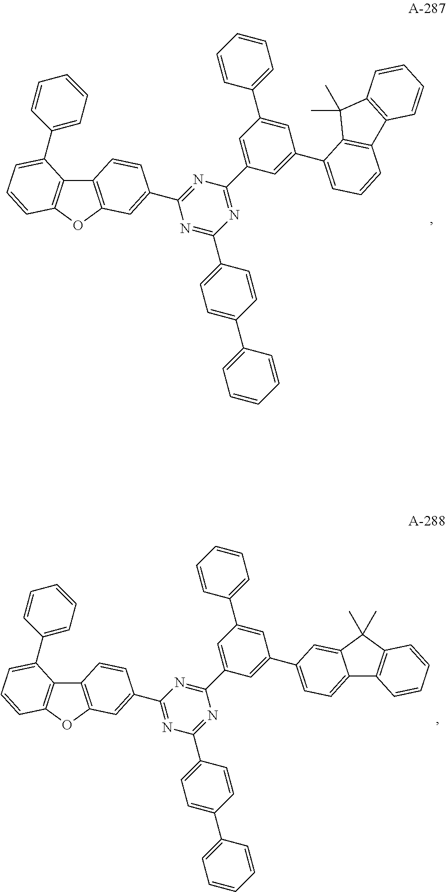 [SMILES: CC1(C)c2ccccc2-c2cccc(-c3cc(-c4ccccc4)cc(-c4nc(-c5ccc(-c6ccccc6)cc5)nc(-c5ccc6c(c5)oc5cccc(-c7ccccc7)c56)n4)c3)c21 ; CC1(C)c2ccccc2-c2ccc(-c3cc(-c4ccccc4)cc(-c4nc(-c5ccc(-c6ccccc6)cc5)nc(-c5ccc6c(c5)oc5cccc(-c7ccccc7)c56)n4)c3)cc21] 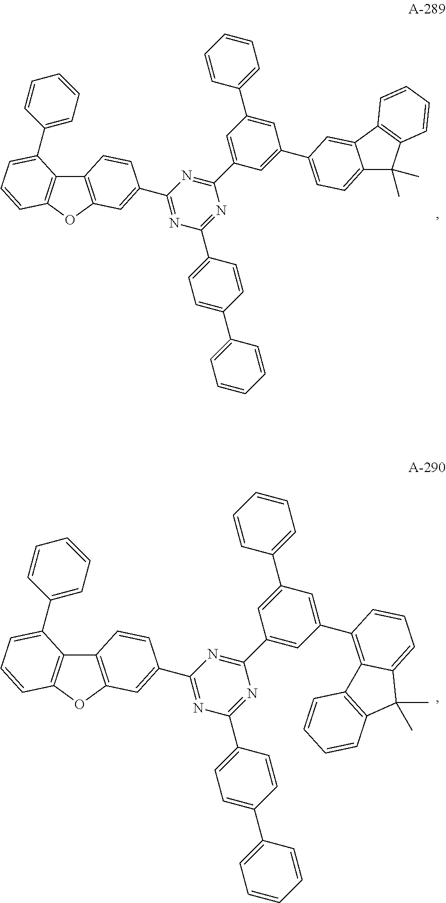 [SMILES: CC1(C)c2ccccc2-c2cc(-c3cc(-c4ccccc4)cc(-c4nc(-c5ccc(-c6ccccc6)cc5)nc(-c5ccc6c(c5)oc5cccc(-c7ccccc7)c56)n4)c3)ccc21 ; CC1(C)c2ccccc2-c2c(-c3cc(-c4ccccc4)cc(-c4nc(-c5ccc(-c6ccccc6)cc5)nc(-c5ccc6c(c5)oc5cccc(-c7ccccc7)c56)n4)c3)cccc21] 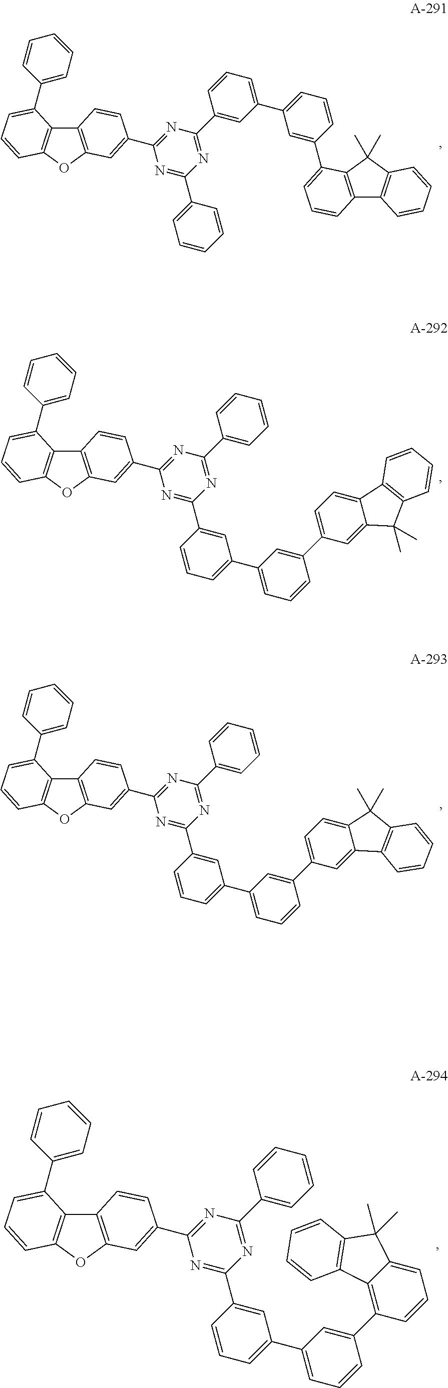 [SMILES: CC1(C)c2ccccc2-c2cccc(-c3cccc(-c4cccc(-c5nc(-c6ccccc6)nc(-c6ccc7c(c6)oc6cccc(-c8ccccc8)c67)n5)c4)c3)c21 ; CC1(C)c2ccccc2-c2ccc(-c3cccc(-c4cccc(-c5nc(-c6ccccc6)nc(-c6ccc7c(c6)oc6cccc(-c8ccccc8)c67)n5)c4)c3)cc21 ; CC1(C)c2ccccc2-c2cc(-c3cccc(-c4cccc(-c5nc(-c6ccccc6)nc(-c6ccc7c(c6)oc6cccc(-c8ccccc8)c67)n5)c4)c3)ccc21 ; CC1(C)c2ccccc2-c2c(-c3cccc(-c4cccc(-c5nc(-c6ccccc6)nc(-c6ccc7c(c6)oc6cccc(-c8ccccc8)c67)n5)c4)c3)cccc21] 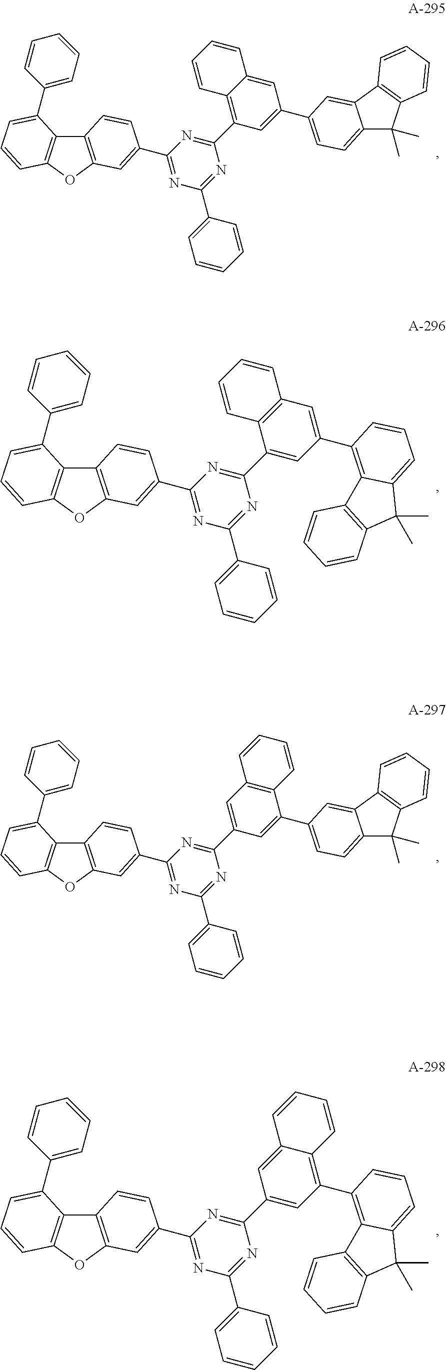 [SMILES: CC1(C)c2ccccc2-c2cc(-c3cc(-c4nc(-c5ccccc5)nc(-c5ccc6c(c5)oc5cccc(-c7ccccc7)c56)n4)c4ccccc4c3)ccc21 ; CC1(C)c2ccccc2-c2c(-c3cc(-c4nc(-c5ccccc5)nc(-c5ccc6c(c5)oc5cccc(-c7ccccc7)c56)n4)c4ccccc4c3)cccc21 ; CC1(C)c2ccccc2-c2cc(-c3cc(-c4nc(-c5ccccc5)nc(-c5ccc6c(c5)oc5cccc(-c7ccccc7)c56)n4)cc4ccccc34)ccc21 ; CC1(C)c2ccccc2-c2c(-c3cc(-c4nc(-c5ccccc5)nc(-c5ccc6c(c5)oc5cccc(-c7ccccc7)c56)n4)cc4ccccc34)cccc21] 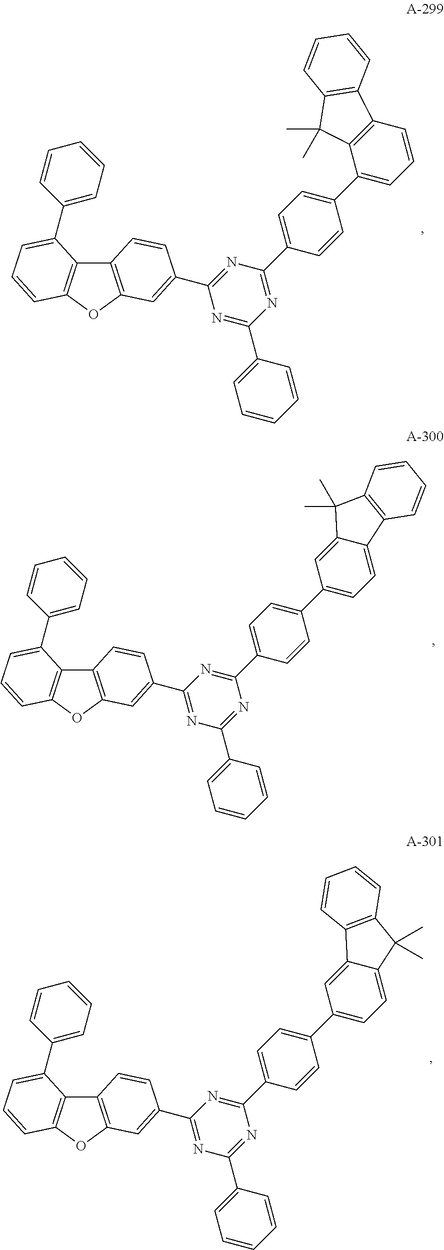 [SMILES: CC1(C)c2ccccc2-c2cccc(-c3ccc(-c4nc(-c5ccccc5)nc(-c5ccc6c(c5)oc5cccc(-c7ccccc7)c56)n4)cc3)c21 ; CC1(C)c2ccccc2-c2ccc(-c3ccc(-c4nc(-c5ccccc5)nc(-c5ccc6c(c5)oc5cccc(-c7ccccc7)c56)n4)cc3)cc21 ; CC1(C)c2ccccc2-c2cc(-c3ccc(-c4nc(-c5ccccc5)nc(-c5ccc6c(c5)oc5cccc(-c7ccccc7)c56)n4)cc3)ccc21] 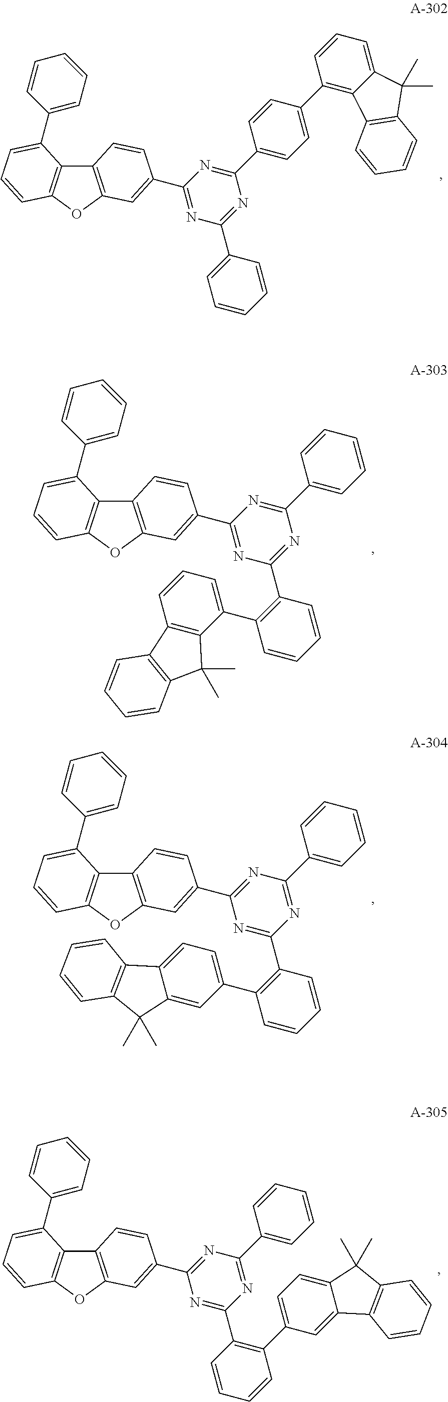 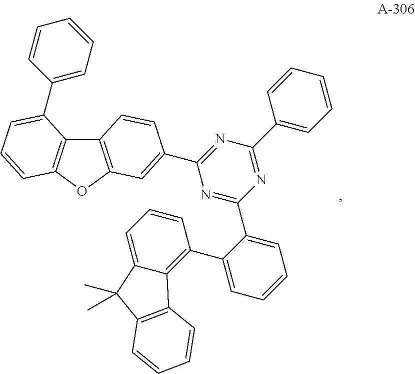 [SMILES: CC1(C)c2ccccc2-c2c(-c3ccccc3-c3nc(-c4ccccc4)nc(-c4ccc5c(c4)oc4cccc(-c6ccccc6)c45)n3)cccc21] 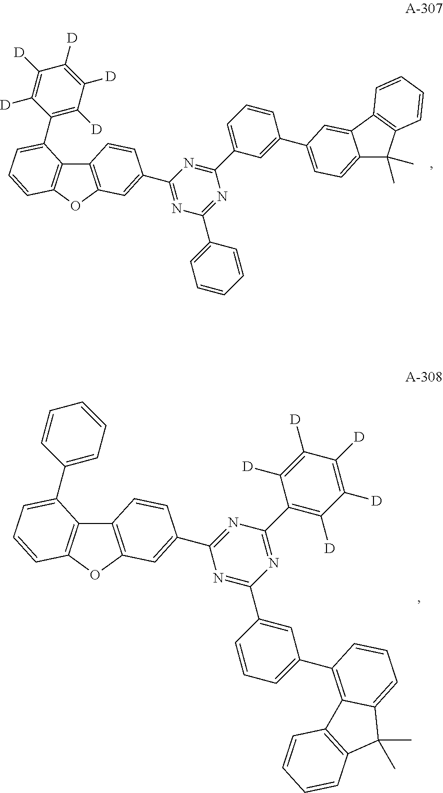 [SMILES: [2H]c1c([2H])c([2H])c(-c2cccc3oc4cc(-c5nc(-c6ccccc6)nc(-c6cccc(-c7ccc8c(c7)-c7ccccc7C8(C)C)c6)n5)ccc4c23)c([2H])c1[2H] ; [2H]c1c([2H])c([2H])c(-c2nc(-c3cccc(-c4cccc5c4-c4ccccc4C5(C)C)c3)nc(-c3ccc4c(c3)oc3cccc(-c5ccccc5)c34)n2)c([2H])c1[2H]] 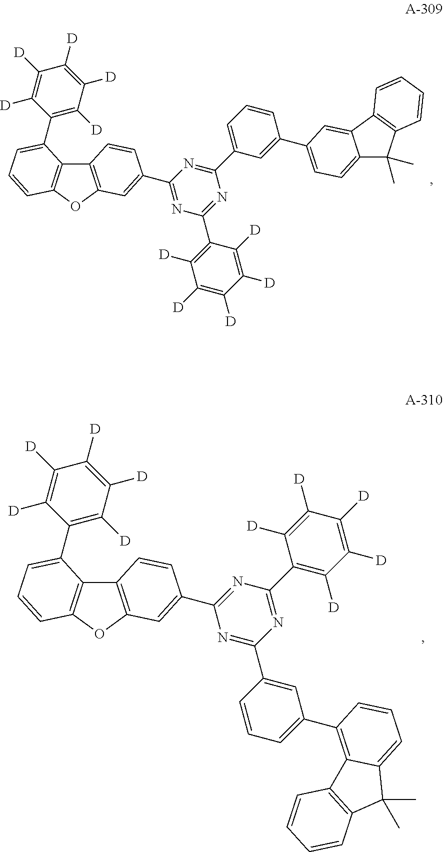 [SMILES: [2H]c1c([2H])c([2H])c(-c2nc(-c3cccc(-c4ccc5c(c4)-c4ccccc4C5(C)C)c3)nc(-c3ccc4c(c3)oc3cccc(-c5c([2H])c([2H])c([2H])c([2H])c5[2H])c34)n2)c([2H])c1[2H] ; [2H]c1c([2H])c([2H])c(-c2nc(-c3cccc(-c4cccc5c4-c4ccccc4C5(C)C)c3)nc(-c3ccc4c(c3)oc3cccc(-c5c([2H])c([2H])c([2H])c([2H])c5[2H])c34)n2)c([2H])c1[2H]] 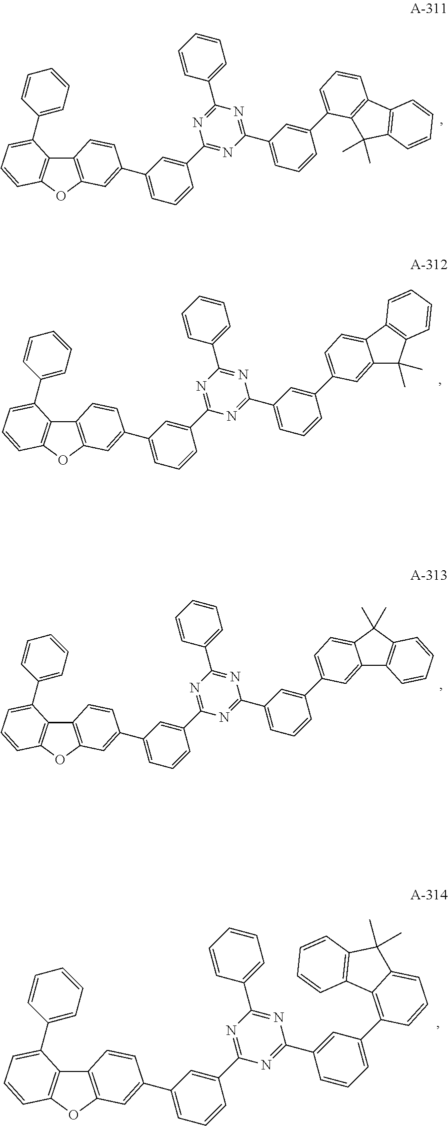 [SMILES: CC1(C)c2ccccc2-c2cccc(-c3cccc(-c4nc(-c5ccccc5)nc(-c5cccc(-c6ccc7c(c6)oc6cccc(-c8ccccc8)c67)c5)n4)c3)c21 ; CC1(C)c2ccccc2-c2ccc(-c3cccc(-c4nc(-c5ccccc5)nc(-c5cccc(-c6ccc7c(c6)oc6cccc(-c8ccccc8)c67)c5)n4)c3)cc21 ; CC1(C)c2ccccc2-c2cc(-c3cccc(-c4nc(-c5ccccc5)nc(-c5cccc(-c6ccc7c(c6)oc6cccc(-c8ccccc8)c67)c5)n4)c3)ccc21 ; CC1(C)c2ccccc2-c2c(-c3cccc(-c4nc(-c5ccccc5)nc(-c5cccc(-c6ccc7c(c6)oc6cccc(-c8ccccc8)c67)c5)n4)c3)cccc21] 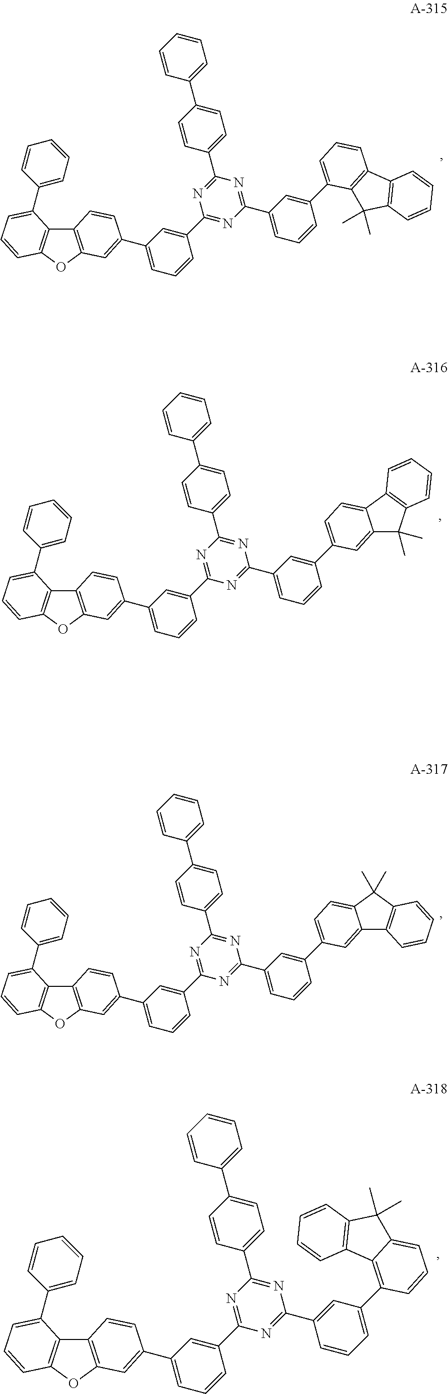 [SMILES: CC1(C)c2ccccc2-c2cccc(-c3cccc(-c4nc(-c5ccc(-c6ccccc6)cc5)nc(-c5cccc(-c6ccc7c(c6)oc6cccc(-c8ccccc8)c67)c5)n4)c3)c21 ; CC1(C)c2ccccc2-c2ccc(-c3cccc(-c4nc(-c5ccc(-c6ccccc6)cc5)nc(-c5cccc(-c6ccc7c(c6)oc6cccc(-c8ccccc8)c67)c5)n4)c3)cc21 ; CC1(C)c2ccccc2-c2cc(-c3cccc(-c4nc(-c5ccc(-c6ccccc6)cc5)nc(-c5cccc(-c6ccc7c(c6)oc6cccc(-c8ccccc8)c67)c5)n4)c3)ccc21 ; CC1(C)c2ccccc2-c2c(-c3cccc(-c4nc(-c5ccc(-c6ccccc6)cc5)nc(-c5cccc(-c6ccc7c(c6)oc6cccc(-c8ccccc8)c67)c5)n4)c3)cccc21] 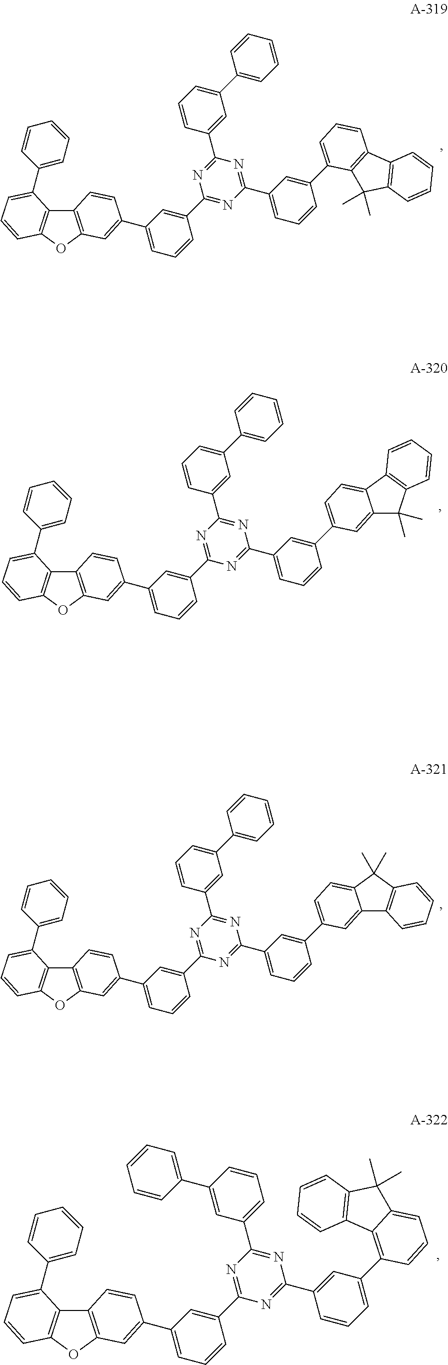 [SMILES: CC1(C)c2ccccc2-c2cccc(-c3cccc(-c4nc(-c5cccc(-c6ccccc6)c5)nc(-c5cccc(-c6ccc7c(c6)oc6cccc(-c8ccccc8)c67)c5)n4)c3)c21 ; CC1(C)c2ccccc2-c2ccc(-c3cccc(-c4nc(-c5cccc(-c6ccccc6)c5)nc(-c5cccc(-c6ccc7c(c6)oc6cccc(-c8ccccc8)c67)c5)n4)c3)cc21 ; CC1(C)c2ccccc2-c2cc(-c3cccc(-c4nc(-c5cccc(-c6ccccc6)c5)nc(-c5cccc(-c6ccc7c(c6)oc6cccc(-c8ccccc8)c67)c5)n4)c3)ccc21 ; CC1(C)c2ccccc2-c2c(-c3cccc(-c4nc(-c5cccc(-c6ccccc6)c5)nc(-c5cccc(-c6ccc7c(c6)oc6cccc(-c8ccccc8)c67)c5)n4)c3)cccc21] 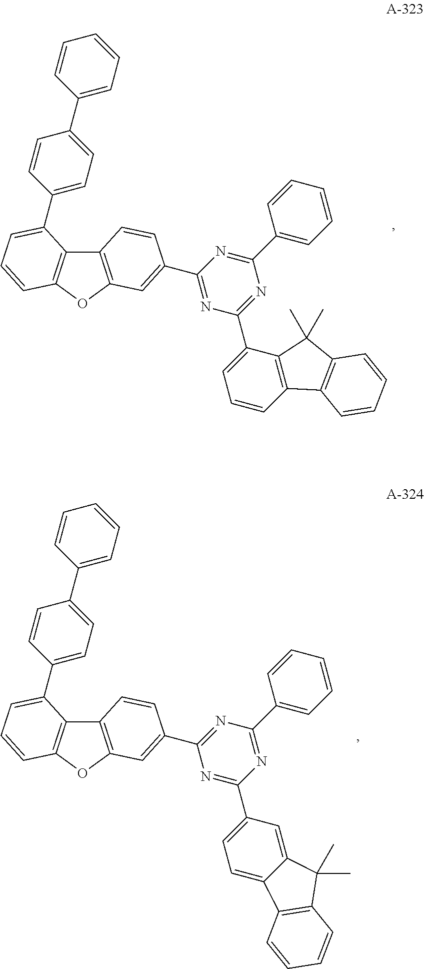 [SMILES: CC1(C)c2ccccc2-c2cccc(-c3nc(-c4ccccc4)nc(-c4ccc5c(c4)oc4cccc(-c6ccc(-c7ccccc7)cc6)c45)n3)c21 ; CC1(C)c2ccccc2-c2ccc(-c3nc(-c4ccccc4)nc(-c4ccc5c(c4)oc4cccc(-c6ccc(-c7ccccc7)cc6)c45)n3)cc21] 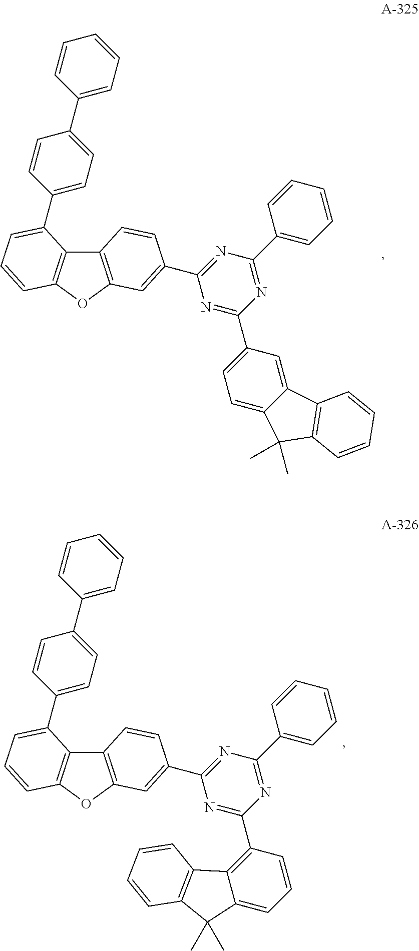 [SMILES: CC1(C)c2ccccc2-c2cc(-c3nc(-c4ccccc4)nc(-c4ccc5c(c4)oc4cccc(-c6ccc(-c7ccccc7)cc6)c45)n3)ccc21 ; CC1(C)c2ccccc2-c2c(-c3nc(-c4ccccc4)nc(-c4ccc5c(c4)oc4cccc(-c6ccc(-c7ccccc7)cc6)c45)n3)cccc21] 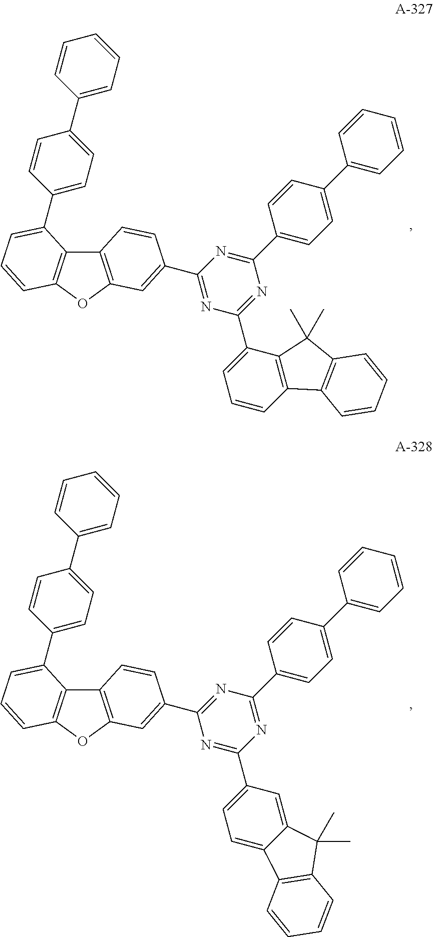 [SMILES: CC1(C)c2ccccc2-c2cccc(-c3nc(-c4ccc(-c5ccccc5)cc4)nc(-c4ccc5c(c4)oc4cccc(-c6ccc(-c7ccccc7)cc6)c45)n3)c21 ; CC1(C)c2ccccc2-c2ccc(-c3nc(-c4ccc(-c5ccccc5)cc4)nc(-c4ccc5c(c4)oc4cccc(-c6ccc(-c7ccccc7)cc6)c45)n3)cc21] 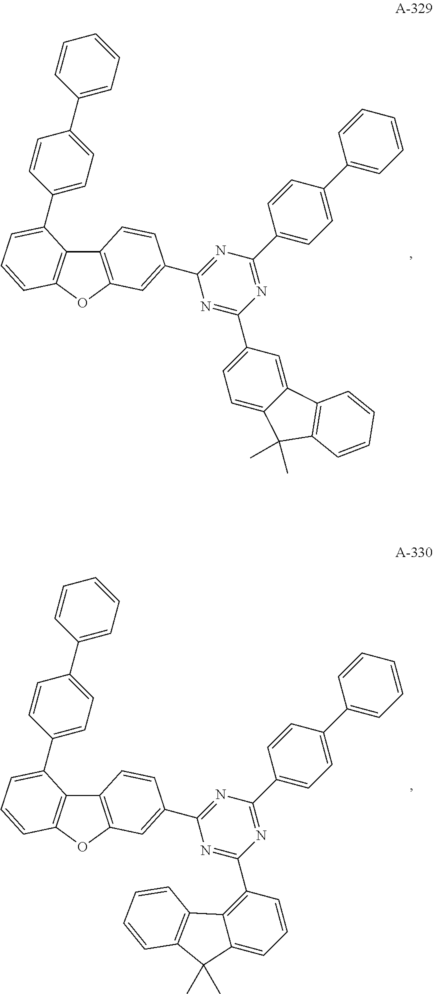 [SMILES: CC1(C)c2ccccc2-c2cc(-c3nc(-c4ccc(-c5ccccc5)cc4)nc(-c4ccc5c(c4)oc4cccc(-c6ccc(-c7ccccc7)cc6)c45)n3)ccc21 ; CC1(C)c2ccccc2-c2c(-c3nc(-c4ccc(-c5ccccc5)cc4)nc(-c4ccc5c(c4)oc4cccc(-c6ccc(-c7ccccc7)cc6)c45)n3)cccc21] 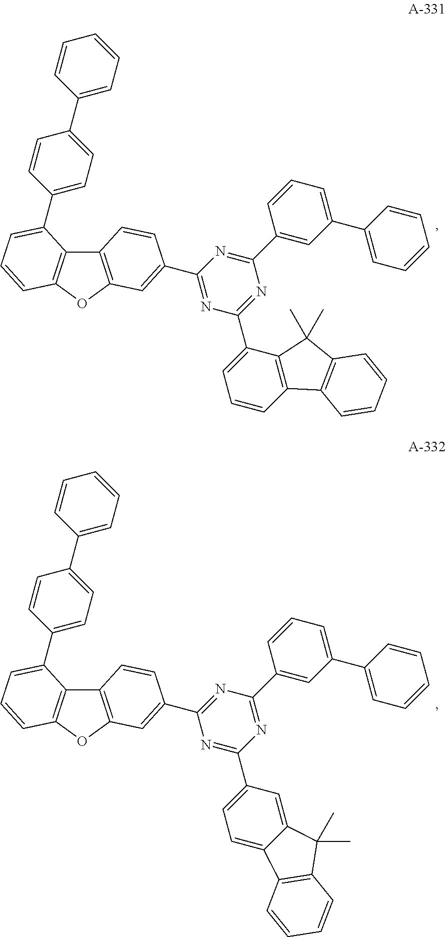 [SMILES: CC1(C)c2ccccc2-c2cccc(-c3nc(-c4cccc(-c5ccccc5)c4)nc(-c4ccc5c(c4)oc4cccc(-c6ccc(-c7ccccc7)cc6)c45)n3)c21 ; CC1(C)c2ccccc2-c2ccc(-c3nc(-c4cccc(-c5ccccc5)c4)nc(-c4ccc5c(c4)oc4cccc(-c6ccc(-c7ccccc7)cc6)c45)n3)cc21] 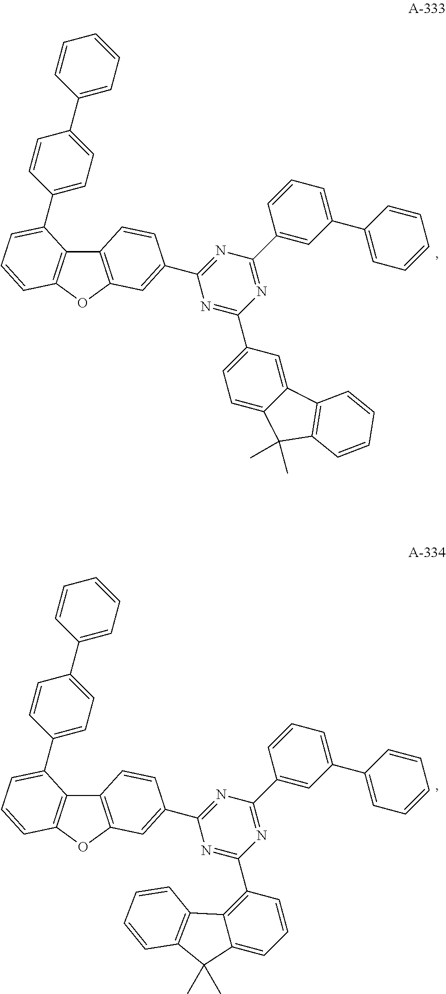 [SMILES: CC1(C)c2ccccc2-c2cc(-c3nc(-c4cccc(-c5ccccc5)c4)nc(-c4ccc5c(c4)oc4cccc(-c6ccc(-c7ccccc7)cc6)c45)n3)ccc21 ; CC1(C)c2ccccc2-c2c(-c3nc(-c4cccc(-c5ccccc5)c4)nc(-c4ccc5c(c4)oc4cccc(-c6ccc(-c7ccccc7)cc6)c45)n3)cccc21] 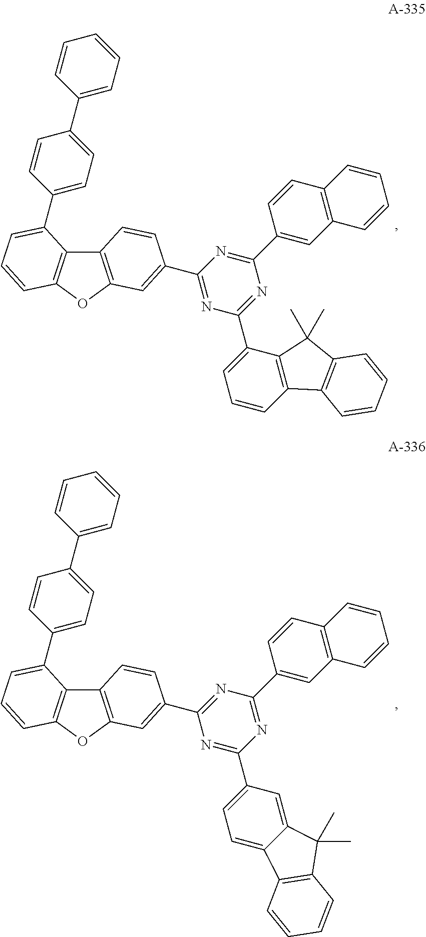 [SMILES: CC1(C)c2ccccc2-c2cccc(-c3nc(-c4ccc5ccccc5c4)nc(-c4ccc5c(c4)oc4cccc(-c6ccc(-c7ccccc7)cc6)c45)n3)c21 ; CC1(C)c2ccccc2-c2ccc(-c3nc(-c4ccc5ccccc5c4)nc(-c4ccc5c(c4)oc4cccc(-c6ccc(-c7ccccc7)cc6)c45)n3)cc21] 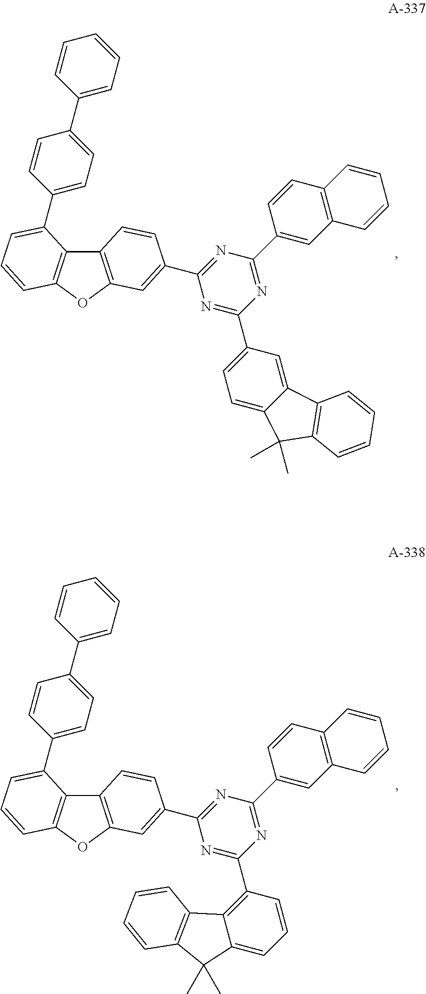 [SMILES: CC1(C)c2ccccc2-c2cc(-c3nc(-c4ccc5ccccc5c4)nc(-c4ccc5c(c4)oc4cccc(-c6ccc(-c7ccccc7)cc6)c45)n3)ccc21 ; CC1(C)c2ccccc2-c2c(-c3nc(-c4ccc5ccccc5c4)nc(-c4ccc5c(c4)oc4cccc(-c6ccc(-c7ccccc7)cc6)c45)n3)cccc21] 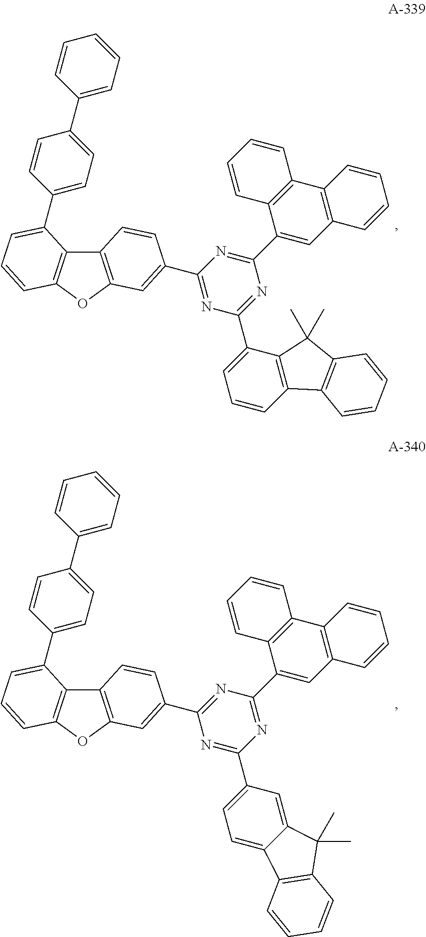 [SMILES: CC1(C)c2ccccc2-c2cccc(-c3nc(-c4ccc5c(c4)oc4cccc(-c6ccc(-c7ccccc7)cc6)c45)nc(-c4cc5ccccc5c5ccccc45)n3)c21 ; CC1(C)c2ccccc2-c2ccc(-c3nc(-c4ccc5c(c4)oc4cccc(-c6ccc(-c7ccccc7)cc6)c45)nc(-c4cc5ccccc5c5ccccc45)n3)cc21] 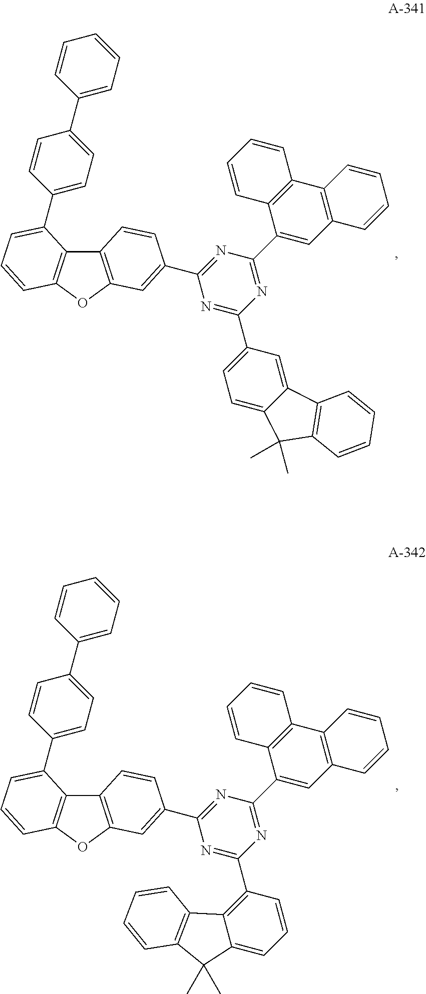 [SMILES: CC1(C)c2ccccc2-c2cc(-c3nc(-c4ccc5c(c4)oc4cccc(-c6ccc(-c7ccccc7)cc6)c45)nc(-c4cc5ccccc5c5ccccc45)n3)ccc21 ; CC1(C)c2ccccc2-c2c(-c3nc(-c4ccc5c(c4)oc4cccc(-c6ccc(-c7ccccc7)cc6)c45)nc(-c4cc5ccccc5c5ccccc45)n3)cccc21] 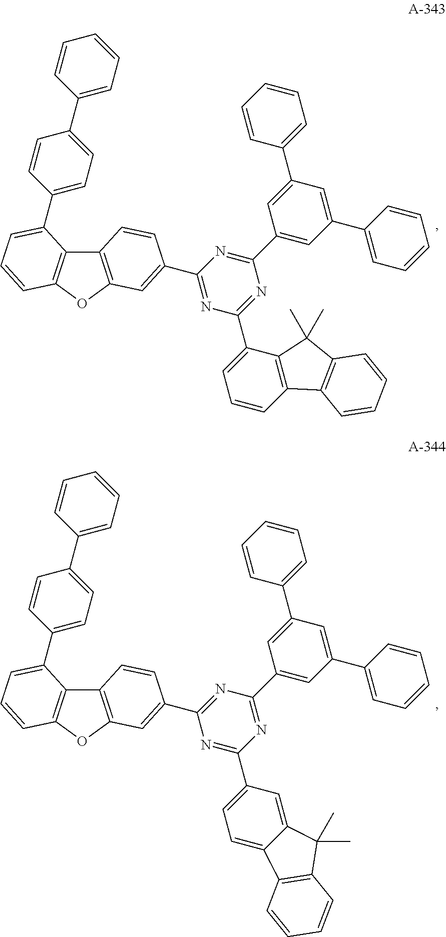 [SMILES: CC1(C)c2ccccc2-c2cccc(-c3nc(-c4cc(-c5ccccc5)cc(-c5ccccc5)c4)nc(-c4ccc5c(c4)oc4cccc(-c6ccc(-c7ccccc7)cc6)c45)n3)c21 ; CC1(C)c2ccccc2-c2ccc(-c3nc(-c4cc(-c5ccccc5)cc(-c5ccccc5)c4)nc(-c4ccc5c(c4)oc4cccc(-c6ccc(-c7ccccc7)cc6)c45)n3)cc21] 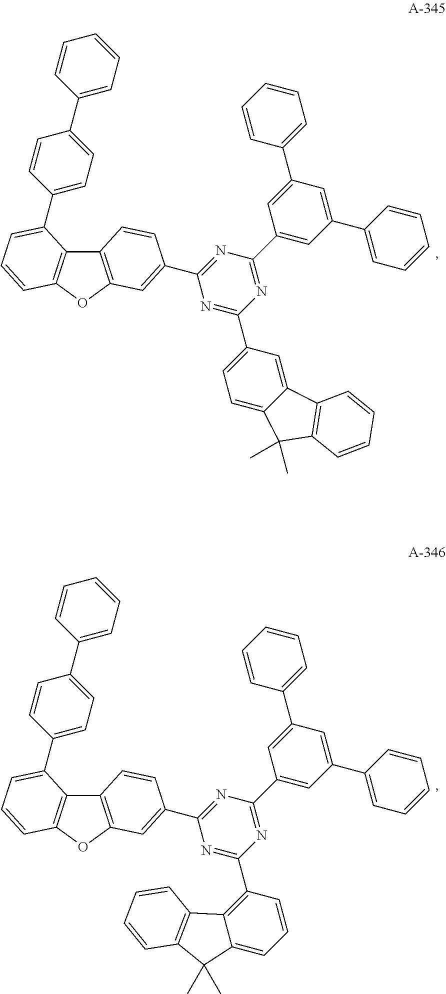 [SMILES: CC1(C)c2ccccc2-c2cc(-c3nc(-c4cc(-c5ccccc5)cc(-c5ccccc5)c4)nc(-c4ccc5c(c4)oc4cccc(-c6ccc(-c7ccccc7)cc6)c45)n3)ccc21 ; CC1(C)c2ccccc2-c2c(-c3nc(-c4cc(-c5ccccc5)cc(-c5ccccc5)c4)nc(-c4ccc5c(c4)oc4cccc(-c6ccc(-c7ccccc7)cc6)c45)n3)cccc21] 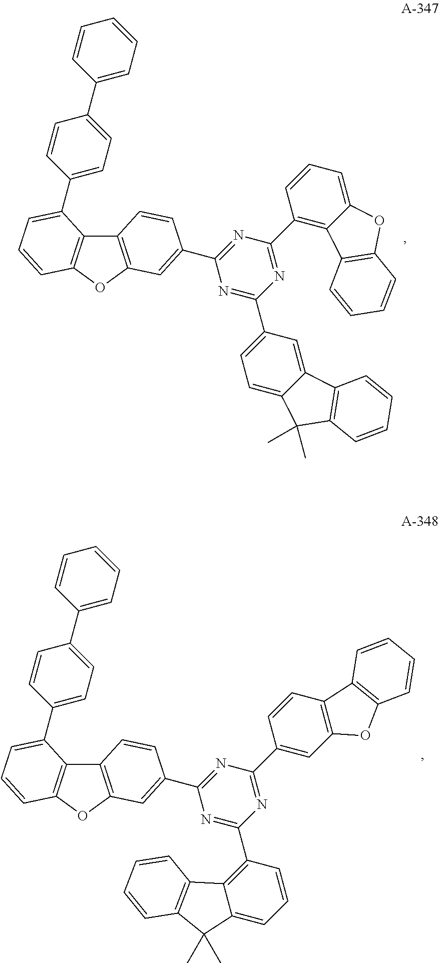 [SMILES: CC1(C)c2ccccc2-c2cc(-c3nc(-c4ccc5c(c4)oc4cccc(-c6ccc(-c7ccccc7)cc6)c45)nc(-c4cccc5oc6ccccc6c45)n3)ccc21 ; CC1(C)c2ccccc2-c2c(-c3nc(-c4ccc5c(c4)oc4ccccc45)nc(-c4ccc5c(c4)oc4cccc(-c6ccc(-c7ccccc7)cc6)c45)n3)cccc21] 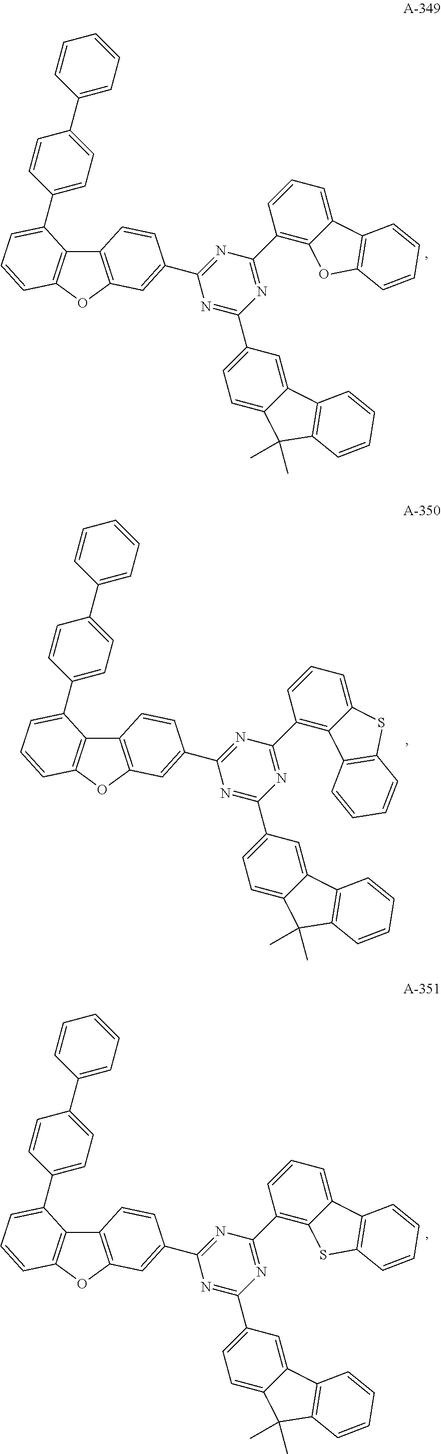 [SMILES: CC1(C)c2ccccc2-c2cc(-c3nc(-c4ccc5c(c4)oc4cccc(-c6ccc(-c7ccccc7)cc6)c45)nc(-c4cccc5c4oc4ccccc45)n3)ccc21 ; CC1(C)c2ccccc2-c2cc(-c3nc(-c4ccc5c(c4)oc4cccc(-c6ccc(-c7ccccc7)cc6)c45)nc(-c4cccc5sc6ccccc6c45)n3)ccc21 ; CC1(C)c2ccccc2-c2cc(-c3nc(-c4ccc5c(c4)oc4cccc(-c6ccc(-c7ccccc7)cc6)c45)nc(-c4cccc5c4sc4ccccc45)n3)ccc21] 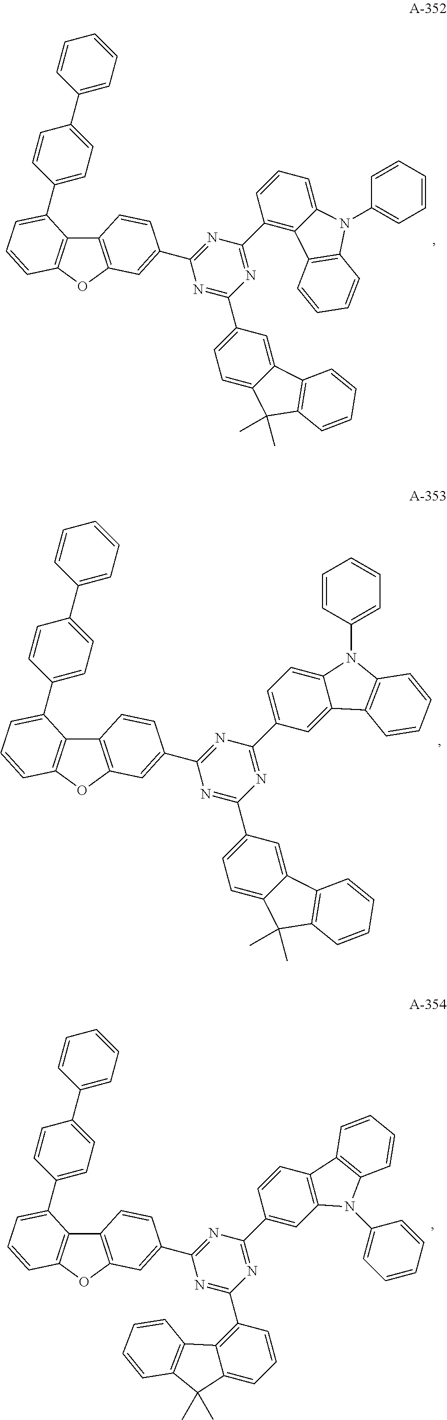 [SMILES: CC1(C)c2ccccc2-c2cc(-c3nc(-c4ccc5c(c4)oc4cccc(-c6ccc(-c7ccccc7)cc6)c45)nc(-c4cccc5c4c4ccccc4n5-c4ccccc4)n3)ccc21 ; CC1(C)c2ccccc2-c2cc(-c3nc(-c4ccc5c(c4)oc4cccc(-c6ccc(-c7ccccc7)cc6)c45)nc(-c4ccc5c(c4)c4ccccc4n5-c4ccccc4)n3)ccc21 ; CC1(C)c2ccccc2-c2c(-c3nc(-c4ccc5c(c4)oc4cccc(-c6ccc(-c7ccccc7)cc6)c45)nc(-c4ccc5c6ccccc6n(-c6ccccc6)c5c4)n3)cccc21] 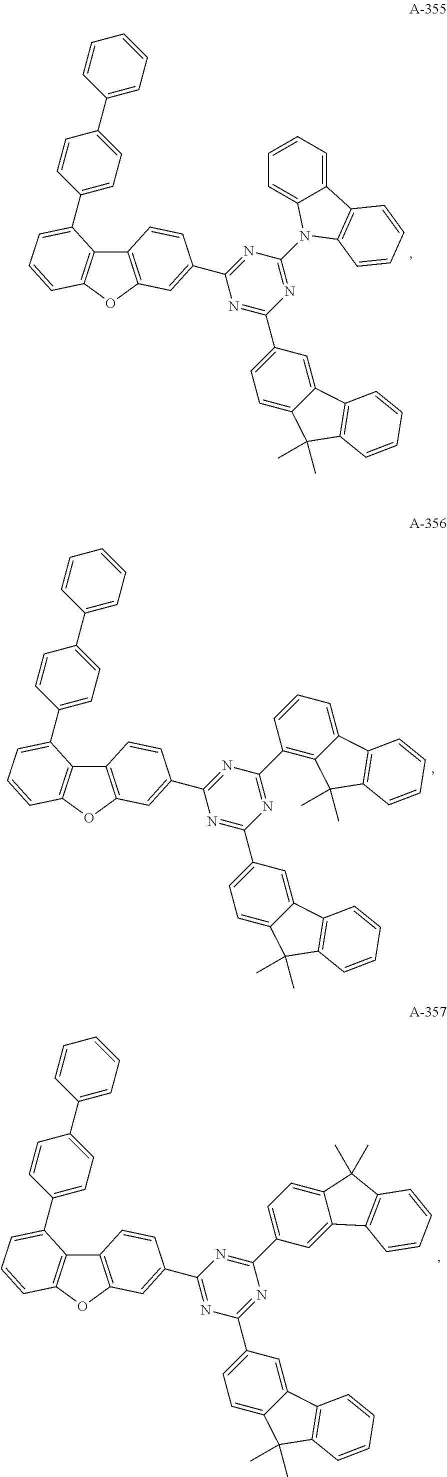 [SMILES: CC1(C)c2ccccc2-c2cc(-c3nc(-c4ccc5c(c4)oc4cccc(-c6ccc(-c7ccccc7)cc6)c45)nc(-n4c5ccccc5c5ccccc54)n3)ccc21 ; CC1(C)c2ccccc2-c2cc(-c3nc(-c4ccc5c(c4)oc4cccc(-c6ccc(-c7ccccc7)cc6)c45)nc(-c4cccc5c4C(C)(C)c4ccccc4-5)n3)ccc21 ; CC1(C)c2ccccc2-c2cc(-c3nc(-c4ccc5c(c4)-c4ccccc4C5(C)C)nc(-c4ccc5c(c4)oc4cccc(-c6ccc(-c7ccccc7)cc6)c45)n3)ccc21] 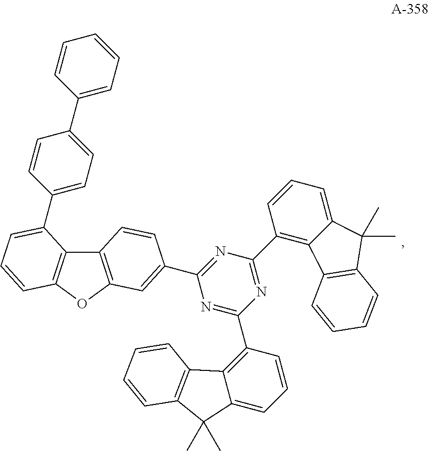 [SMILES: CC1(C)c2ccccc2-c2c(-c3nc(-c4ccc5c(c4)oc4cccc(-c6ccc(-c7ccccc7)cc6)c45)nc(-c4cccc5c4-c4ccccc4C5(C)C)n3)cccc21] 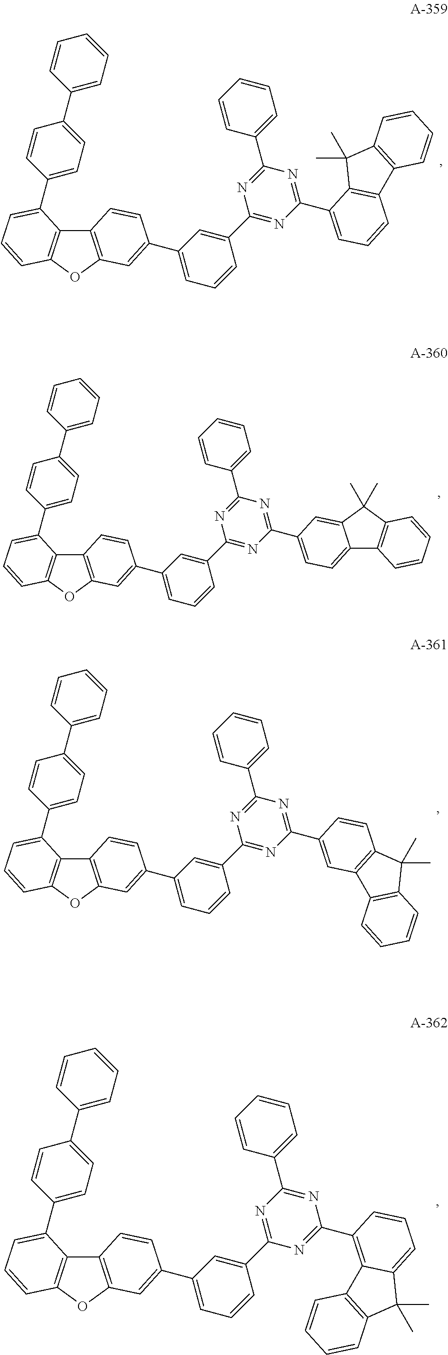 [SMILES: CC1(C)c2ccccc2-c2cccc(-c3nc(-c4ccccc4)nc(-c4cccc(-c5ccc6c(c5)oc5cccc(-c7ccc(-c8ccccc8)cc7)c56)c4)n3)c21 ; CC1(C)c2ccccc2-c2ccc(-c3nc(-c4ccccc4)nc(-c4cccc(-c5ccc6c(c5)oc5cccc(-c7ccc(-c8ccccc8)cc7)c56)c4)n3)cc21 ; CC1(C)c2ccccc2-c2cc(-c3nc(-c4ccccc4)nc(-c4cccc(-c5ccc6c(c5)oc5cccc(-c7ccc(-c8ccccc8)cc7)c56)c4)n3)ccc21 ; CC1(C)c2ccccc2-c2c(-c3nc(-c4ccccc4)nc(-c4cccc(-c5ccc6c(c5)oc5cccc(-c7ccc(-c8ccccc8)cc7)c56)c4)n3)cccc21] 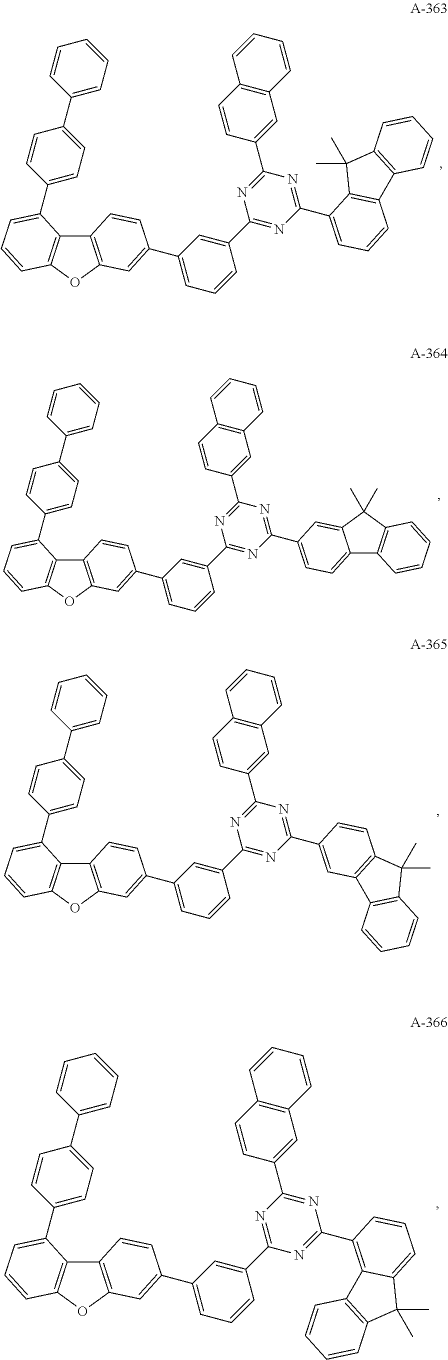 [SMILES: CC1(C)c2ccccc2-c2cccc(-c3nc(-c4cccc(-c5ccc6c(c5)oc5cccc(-c7ccc(-c8ccccc8)cc7)c56)c4)nc(-c4ccc5ccccc5c4)n3)c21 ; CC1(C)c2ccccc2-c2ccc(-c3nc(-c4cccc(-c5ccc6c(c5)oc5cccc(-c7ccc(-c8ccccc8)cc7)c56)c4)nc(-c4ccc5ccccc5c4)n3)cc21 ; CC1(C)c2ccccc2-c2cc(-c3nc(-c4cccc(-c5ccc6c(c5)oc5cccc(-c7ccc(-c8ccccc8)cc7)c56)c4)nc(-c4ccc5ccccc5c4)n3)ccc21 ; CC1(C)c2ccccc2-c2c(-c3nc(-c4cccc(-c5ccc6c(c5)oc5cccc(-c7ccc(-c8ccccc8)cc7)c56)c4)nc(-c4ccc5ccccc5c4)n3)cccc21] 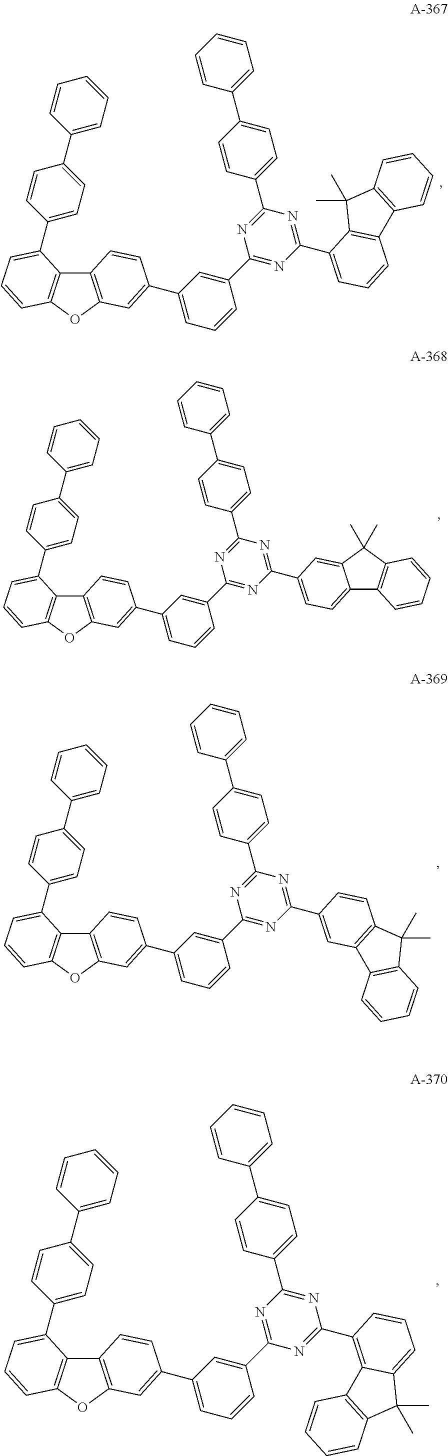 [SMILES: CC1(C)c2ccccc2-c2cccc(-c3nc(-c4ccc(-c5ccccc5)cc4)nc(-c4cccc(-c5ccc6c(c5)oc5cccc(-c7ccc(-c8ccccc8)cc7)c56)c4)n3)c21 ; CC1(C)c2ccccc2-c2ccc(-c3nc(-c4ccc(-c5ccccc5)cc4)nc(-c4cccc(-c5ccc6c(c5)oc5cccc(-c7ccc(-c8ccccc8)cc7)c56)c4)n3)cc21 ; CC1(C)c2ccccc2-c2cc(-c3nc(-c4ccc(-c5ccccc5)cc4)nc(-c4cccc(-c5ccc6c(c5)oc5cccc(-c7ccc(-c8ccccc8)cc7)c56)c4)n3)ccc21 ; CC1(C)c2ccccc2-c2c(-c3nc(-c4ccc(-c5ccccc5)cc4)nc(-c4cccc(-c5ccc6c(c5)oc5cccc(-c7ccc(-c8ccccc8)cc7)c56)c4)n3)cccc21] 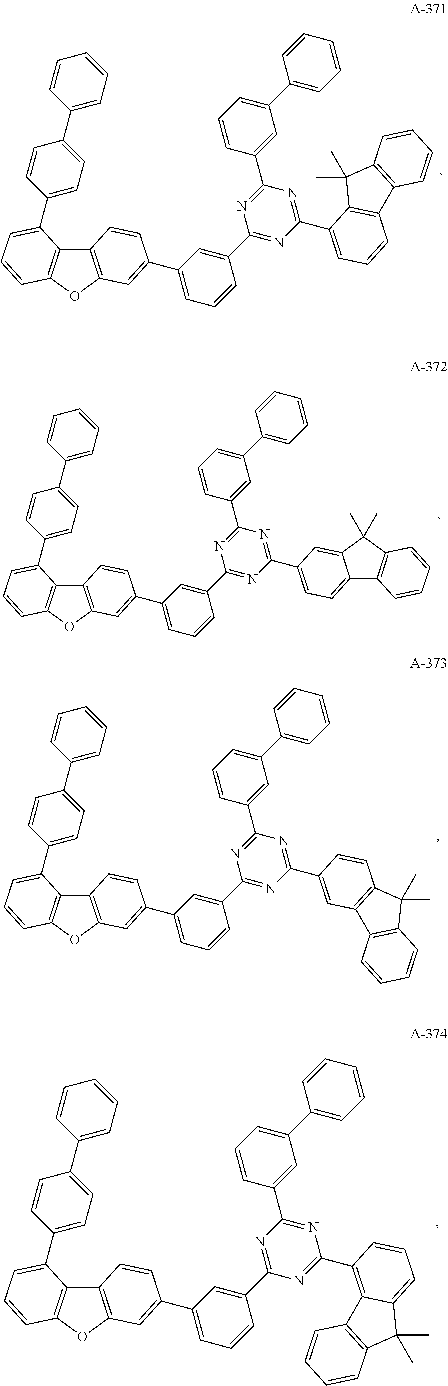 [SMILES: CC1(C)c2ccccc2-c2cccc(-c3nc(-c4cccc(-c5ccccc5)c4)nc(-c4cccc(-c5ccc6c(c5)oc5cccc(-c7ccc(-c8ccccc8)cc7)c56)c4)n3)c21 ; CC1(C)c2ccccc2-c2ccc(-c3nc(-c4cccc(-c5ccccc5)c4)nc(-c4cccc(-c5ccc6c(c5)oc5cccc(-c7ccc(-c8ccccc8)cc7)c56)c4)n3)cc21 ; CC1(C)c2ccccc2-c2cc(-c3nc(-c4cccc(-c5ccccc5)c4)nc(-c4cccc(-c5ccc6c(c5)oc5cccc(-c7ccc(-c8ccccc8)cc7)c56)c4)n3)ccc21 ; CC1(C)c2ccccc2-c2c(-c3nc(-c4cccc(-c5ccccc5)c4)nc(-c4cccc(-c5ccc6c(c5)oc5cccc(-c7ccc(-c8ccccc8)cc7)c56)c4)n3)cccc21] 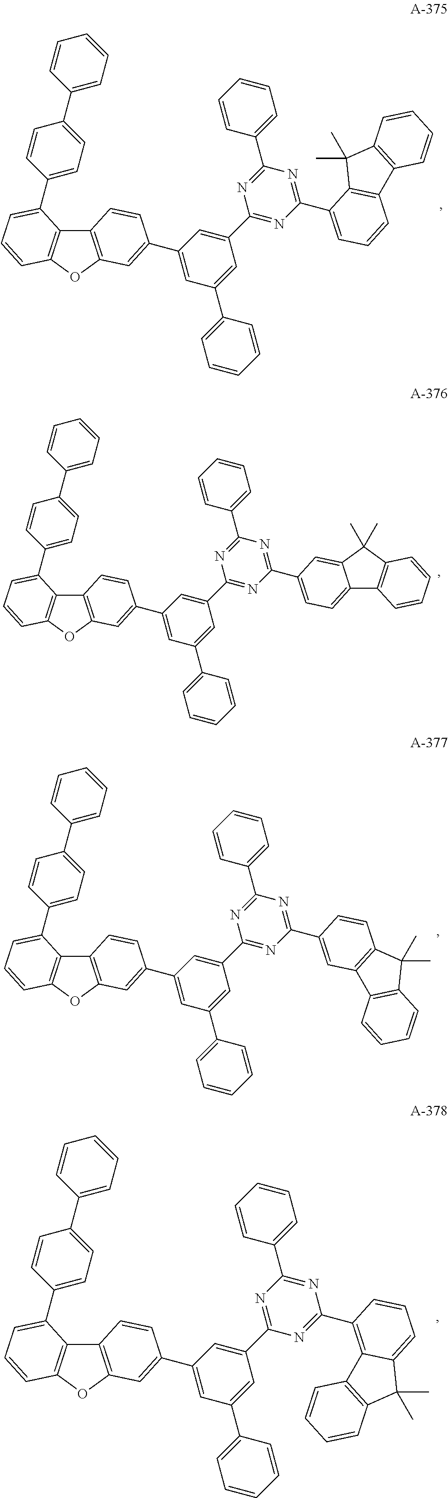 [SMILES: CC1(C)c2ccccc2-c2cccc(-c3nc(-c4ccccc4)nc(-c4cc(-c5ccccc5)cc(-c5ccc6c(c5)oc5cccc(-c7ccc(-c8ccccc8)cc7)c56)c4)n3)c21 ; CC1(C)c2ccccc2-c2ccc(-c3nc(-c4ccccc4)nc(-c4cc(-c5ccccc5)cc(-c5ccc6c(c5)oc5cccc(-c7ccc(-c8ccccc8)cc7)c56)c4)n3)cc21 ; CC1(C)c2ccccc2-c2cc(-c3nc(-c4ccccc4)nc(-c4cc(-c5ccccc5)cc(-c5ccc6c(c5)oc5cccc(-c7ccc(-c8ccccc8)cc7)c56)c4)n3)ccc21 ; CC1(C)c2ccccc2-c2c(-c3nc(-c4ccccc4)nc(-c4cc(-c5ccccc5)cc(-c5ccc6c(c5)oc5cccc(-c7ccc(-c8ccccc8)cc7)c56)c4)n3)cccc21] 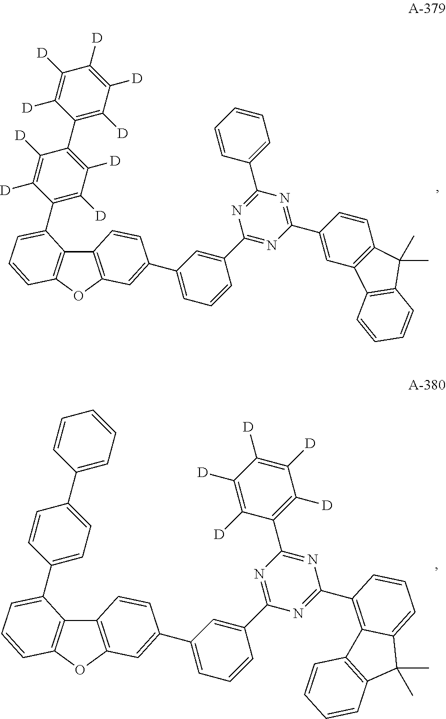 [SMILES: [2H]c1c([2H])c([2H])c(-c2c([2H])c([2H])c(-c3cccc4oc5cc(-c6cccc(-c7nc(-c8ccccc8)nc(-c8ccc9c(c8)-c8ccccc8C9(C)C)n7)c6)ccc5c34)c([2H])c2[2H])c([2H])c1[2H] ; [2H]c1c([2H])c([2H])c(-c2nc(-c3cccc(-c4ccc5c(c4)oc4cccc(-c6ccc(-c7ccccc7)cc6)c45)c3)nc(-c3cccc4c3-c3ccccc3C4(C)C)n2)c([2H])c1[2H]] 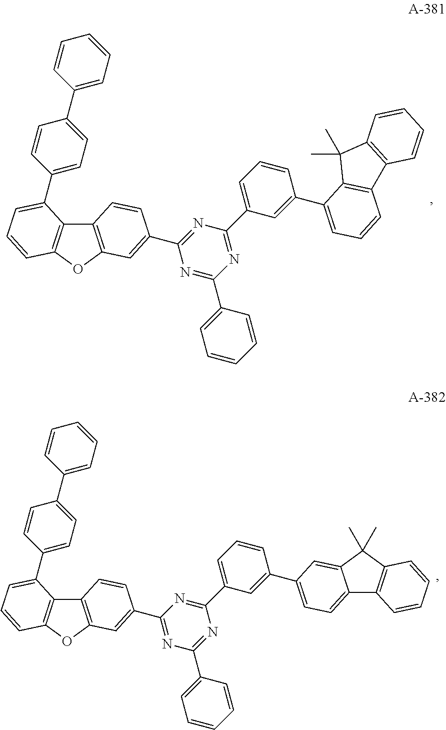 [SMILES: CC1(C)c2ccccc2-c2cccc(-c3cccc(-c4nc(-c5ccccc5)nc(-c5ccc6c(c5)oc5cccc(-c7ccc(-c8ccccc8)cc7)c56)n4)c3)c21 ; CC1(C)c2ccccc2-c2ccc(-c3cccc(-c4nc(-c5ccccc5)nc(-c5ccc6c(c5)oc5cccc(-c7ccc(-c8ccccc8)cc7)c56)n4)c3)cc21] 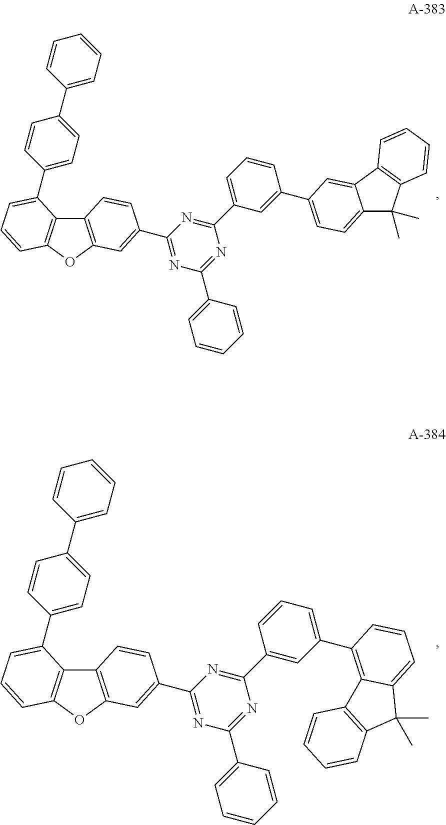 [SMILES: CC1(C)c2ccccc2-c2cc(-c3cccc(-c4nc(-c5ccccc5)nc(-c5ccc6c(c5)oc5cccc(-c7ccc(-c8ccccc8)cc7)c56)n4)c3)ccc21 ; CC1(C)c2ccccc2-c2c(-c3cccc(-c4nc(-c5ccccc5)nc(-c5ccc6c(c5)oc5cccc(-c7ccc(-c8ccccc8)cc7)c56)n4)c3)cccc21] 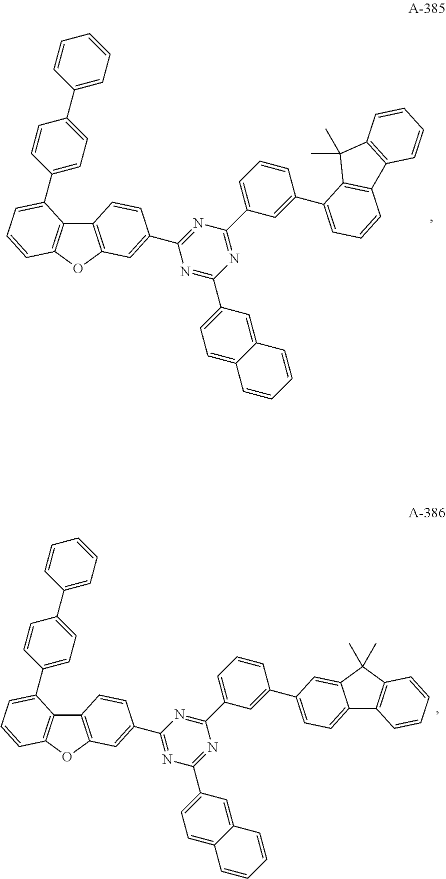 [SMILES: CC1(C)c2ccccc2-c2cccc(-c3cccc(-c4nc(-c5ccc6ccccc6c5)nc(-c5ccc6c(c5)oc5cccc(-c7ccc(-c8ccccc8)cc7)c56)n4)c3)c21 ; CC1(C)c2ccccc2-c2ccc(-c3cccc(-c4nc(-c5ccc6ccccc6c5)nc(-c5ccc6c(c5)oc5cccc(-c7ccc(-c8ccccc8)cc7)c56)n4)c3)cc21] 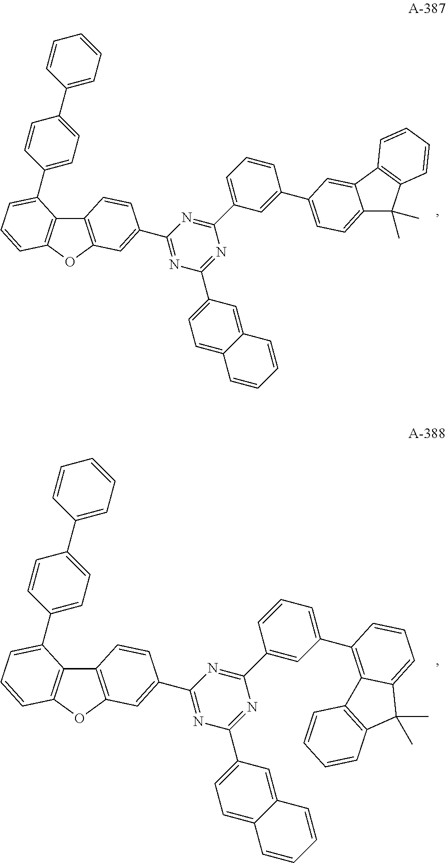 [SMILES: CC1(C)c2ccccc2-c2cc(-c3cccc(-c4nc(-c5ccc6ccccc6c5)nc(-c5ccc6c(c5)oc5cccc(-c7ccc(-c8ccccc8)cc7)c56)n4)c3)ccc21 ; CC1(C)c2ccccc2-c2c(-c3cccc(-c4nc(-c5ccc6ccccc6c5)nc(-c5ccc6c(c5)oc5cccc(-c7ccc(-c8ccccc8)cc7)c56)n4)c3)cccc21] 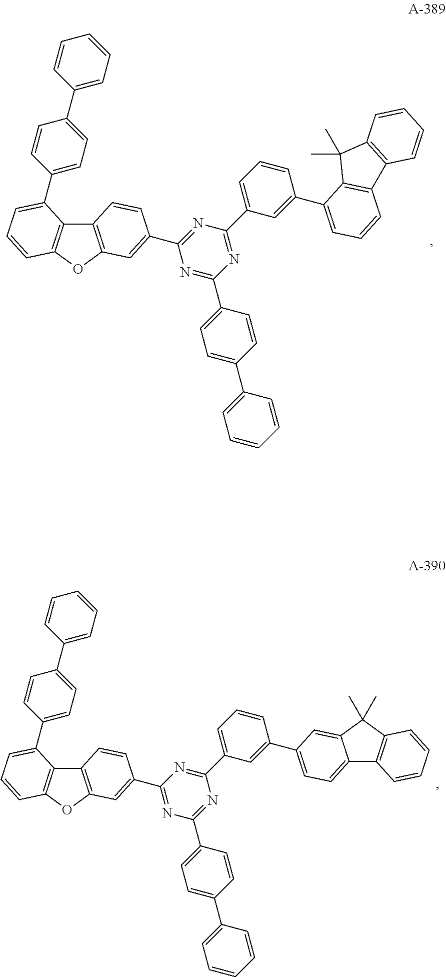 [SMILES: CC1(C)c2ccccc2-c2cccc(-c3cccc(-c4nc(-c5ccc(-c6ccccc6)cc5)nc(-c5ccc6c(c5)oc5cccc(-c7ccc(-c8ccccc8)cc7)c56)n4)c3)c21 ; CC1(C)c2ccccc2-c2ccc(-c3cccc(-c4nc(-c5ccc(-c6ccccc6)cc5)nc(-c5ccc6c(c5)oc5cccc(-c7ccc(-c8ccccc8)cc7)c56)n4)c3)cc21] 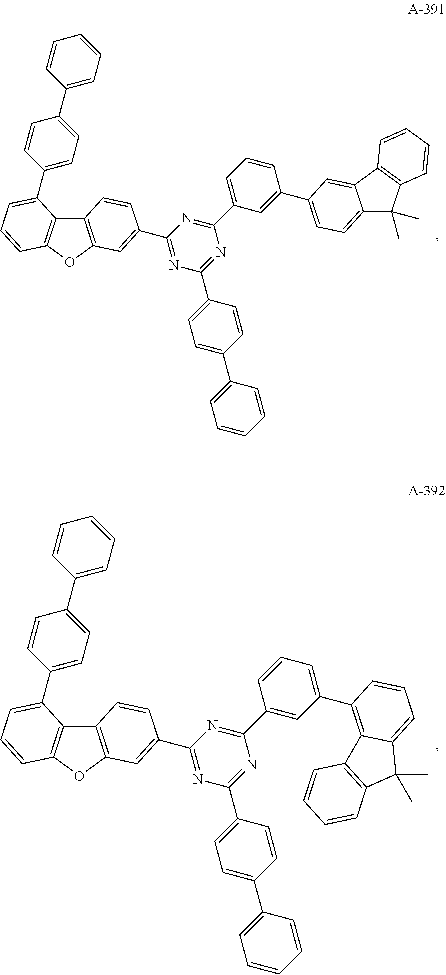 [SMILES: CC1(C)c2ccccc2-c2cc(-c3cccc(-c4nc(-c5ccc(-c6ccccc6)cc5)nc(-c5ccc6c(c5)oc5cccc(-c7ccc(-c8ccccc8)cc7)c56)n4)c3)ccc21 ; CC1(C)c2ccccc2-c2c(-c3cccc(-c4nc(-c5ccc(-c6ccccc6)cc5)nc(-c5ccc6c(c5)oc5cccc(-c7ccc(-c8ccccc8)cc7)c56)n4)c3)cccc21] 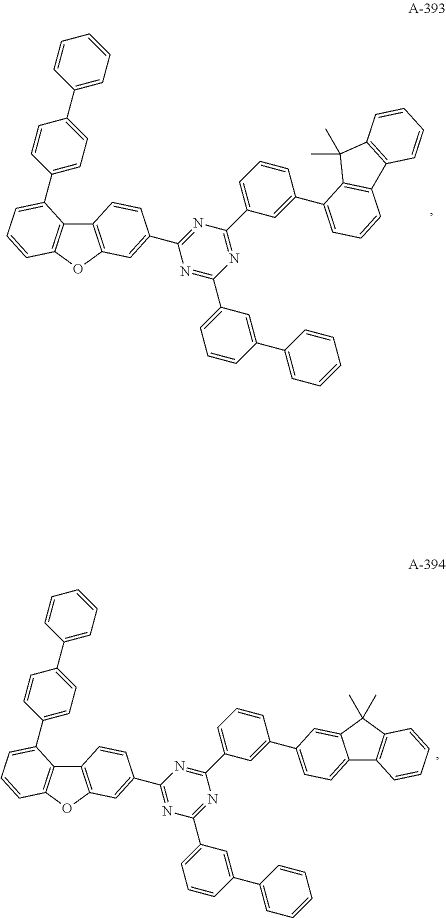 [SMILES: CC1(C)c2ccccc2-c2cccc(-c3cccc(-c4nc(-c5cccc(-c6ccccc6)c5)nc(-c5ccc6c(c5)oc5cccc(-c7ccc(-c8ccccc8)cc7)c56)n4)c3)c21 ; CC1(C)c2ccccc2-c2ccc(-c3cccc(-c4nc(-c5cccc(-c6ccccc6)c5)nc(-c5ccc6c(c5)oc5cccc(-c7ccc(-c8ccccc8)cc7)c56)n4)c3)cc21] 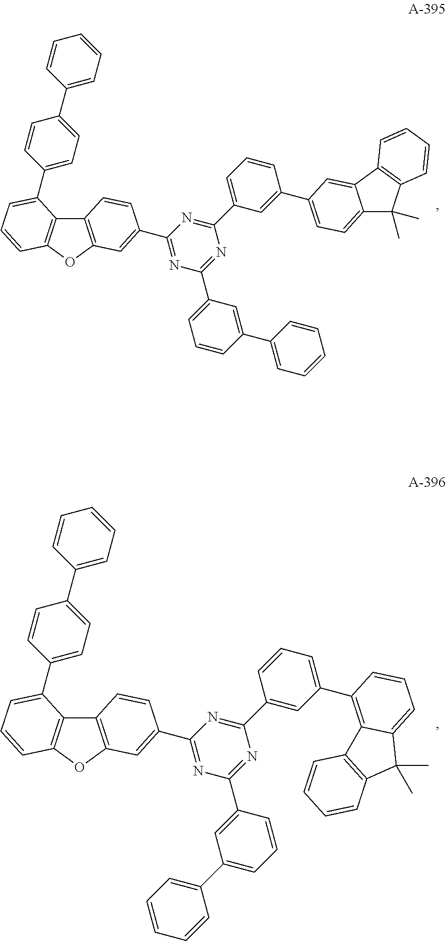 [SMILES: CC1(C)c2ccccc2-c2cc(-c3cccc(-c4nc(-c5cccc(-c6ccccc6)c5)nc(-c5ccc6c(c5)oc5cccc(-c7ccc(-c8ccccc8)cc7)c56)n4)c3)ccc21 ; CC1(C)c2ccccc2-c2c(-c3cccc(-c4nc(-c5cccc(-c6ccccc6)c5)nc(-c5ccc6c(c5)oc5cccc(-c7ccc(-c8ccccc8)cc7)c56)n4)c3)cccc21] 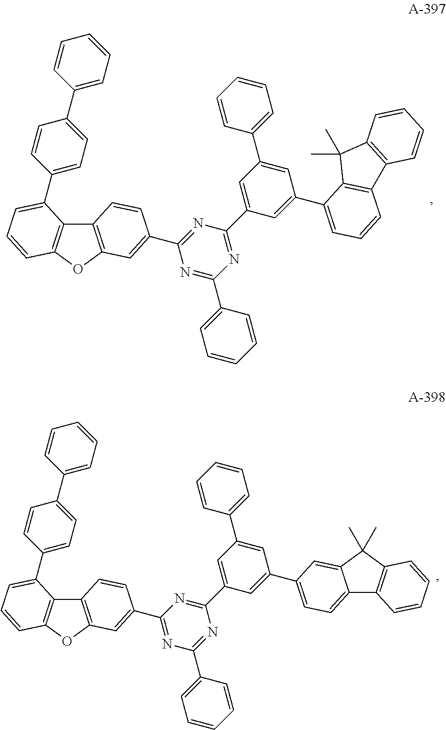 [SMILES: CC1(C)c2ccccc2-c2cccc(-c3cc(-c4ccccc4)cc(-c4nc(-c5ccccc5)nc(-c5ccc6c(c5)oc5cccc(-c7ccc(-c8ccccc8)cc7)c56)n4)c3)c21 ; CC1(C)c2ccccc2-c2ccc(-c3cc(-c4ccccc4)cc(-c4nc(-c5ccccc5)nc(-c5ccc6c(c5)oc5cccc(-c7ccc(-c8ccccc8)cc7)c56)n4)c3)cc21] 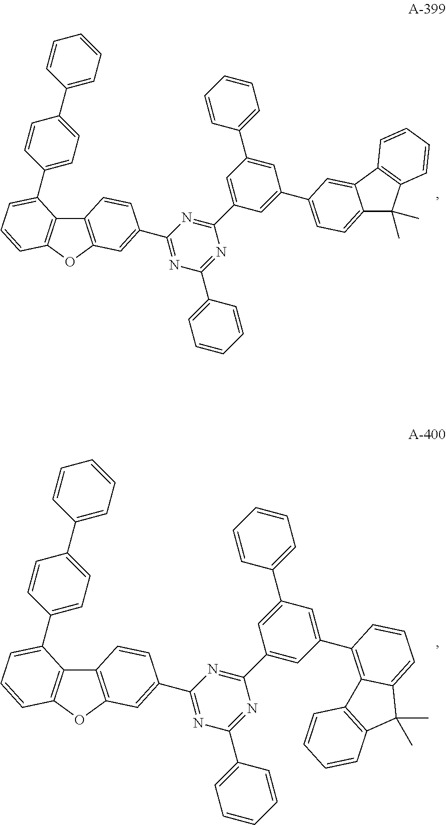 [SMILES: CC1(C)c2ccccc2-c2cc(-c3cc(-c4ccccc4)cc(-c4nc(-c5ccccc5)nc(-c5ccc6c(c5)oc5cccc(-c7ccc(-c8ccccc8)cc7)c56)n4)c3)ccc21 ; CC1(C)c2ccccc2-c2c(-c3cc(-c4ccccc4)cc(-c4nc(-c5ccccc5)nc(-c5ccc6c(c5)oc5cccc(-c7ccc(-c8ccccc8)cc7)c56)n4)c3)cccc21] 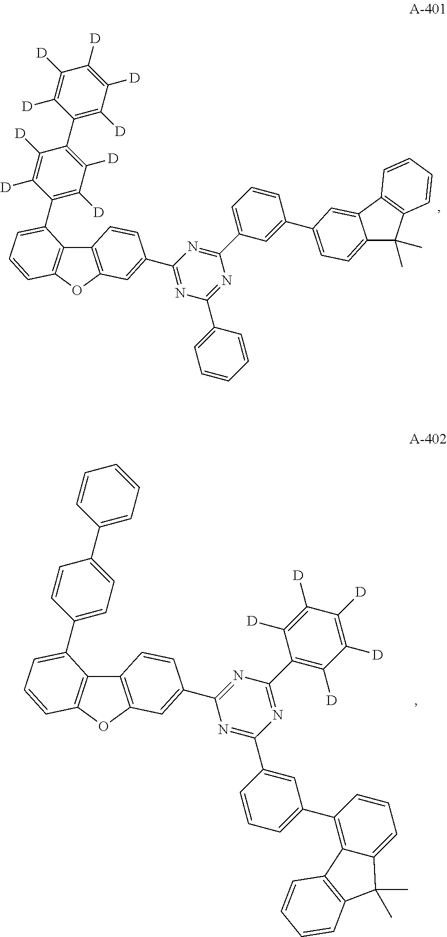 [SMILES: [2H]c1c([2H])c([2H])c(-c2c([2H])c([2H])c(-c3cccc4oc5cc(-c6nc(-c7ccccc7)nc(-c7cccc(-c8ccc9c(c8)-c8ccccc8C9(C)C)c7)n6)ccc5c34)c([2H])c2[2H])c([2H])c1[2H] ; [2H]c1c([2H])c([2H])c(-c2nc(-c3cccc(-c4cccc5c4-c4ccccc4C5(C)C)c3)nc(-c3ccc4c(c3)oc3cccc(-c5ccc(-c6ccccc6)cc5)c34)n2)c([2H])c1[2H]] 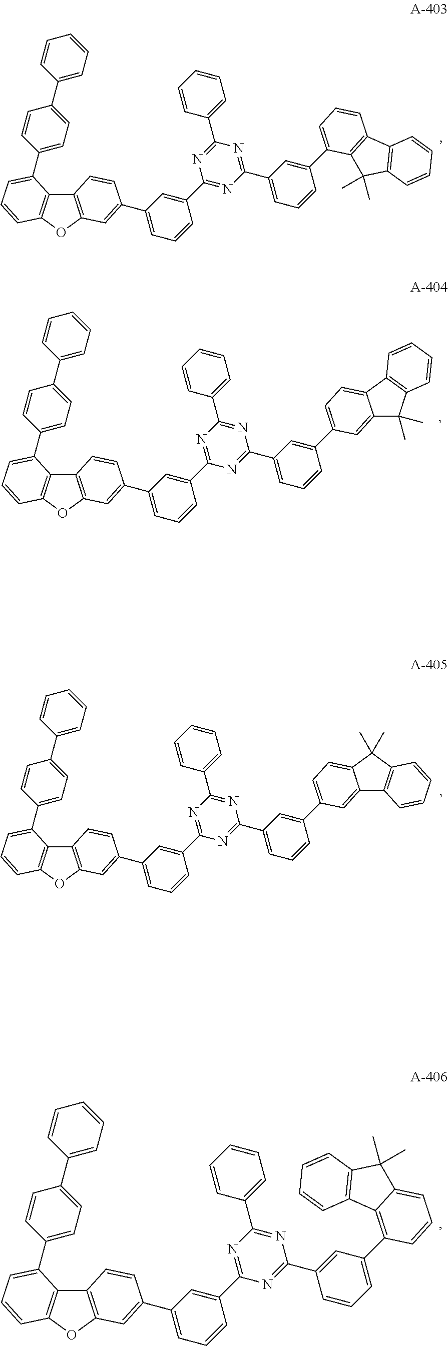 [SMILES: CC1(C)c2ccccc2-c2cccc(-c3cccc(-c4nc(-c5ccccc5)nc(-c5cccc(-c6ccc7c(c6)oc6cccc(-c8ccc(-c9ccccc9)cc8)c67)c5)n4)c3)c21 ; CC1(C)c2ccccc2-c2ccc(-c3cccc(-c4nc(-c5ccccc5)nc(-c5cccc(-c6ccc7c(c6)oc6cccc(-c8ccc(-c9ccccc9)cc8)c67)c5)n4)c3)cc21 ; CC1(C)c2ccccc2-c2cc(-c3cccc(-c4nc(-c5ccccc5)nc(-c5cccc(-c6ccc7c(c6)oc6cccc(-c8ccc(-c9ccccc9)cc8)c67)c5)n4)c3)ccc21 ; CC1(C)c2ccccc2-c2c(-c3cccc(-c4nc(-c5ccccc5)nc(-c5cccc(-c6ccc7c(c6)oc6cccc(-c8ccc(-c9ccccc9)cc8)c67)c5)n4)c3)cccc21] 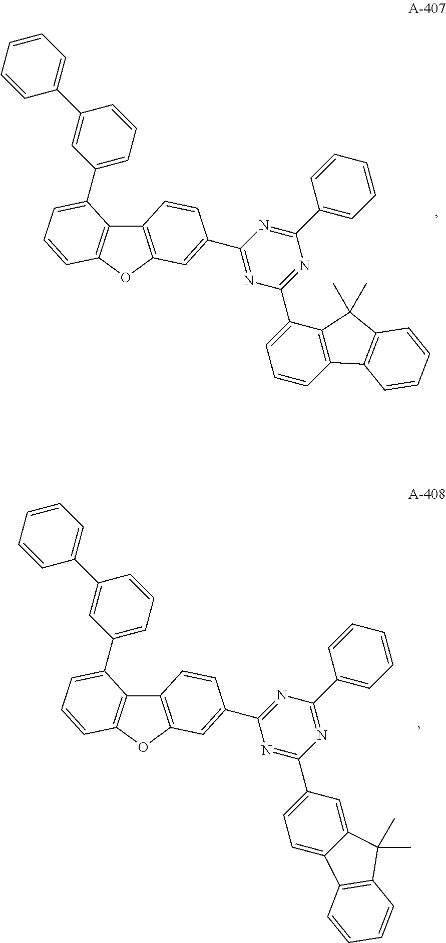 [SMILES: CC1(C)c2ccccc2-c2cccc(-c3nc(-c4ccccc4)nc(-c4ccc5c(c4)oc4cccc(-c6cccc(-c7ccccc7)c6)c45)n3)c21 ; CC1(C)c2ccccc2-c2ccc(-c3nc(-c4ccccc4)nc(-c4ccc5c(c4)oc4cccc(-c6cccc(-c7ccccc7)c6)c45)n3)cc21] 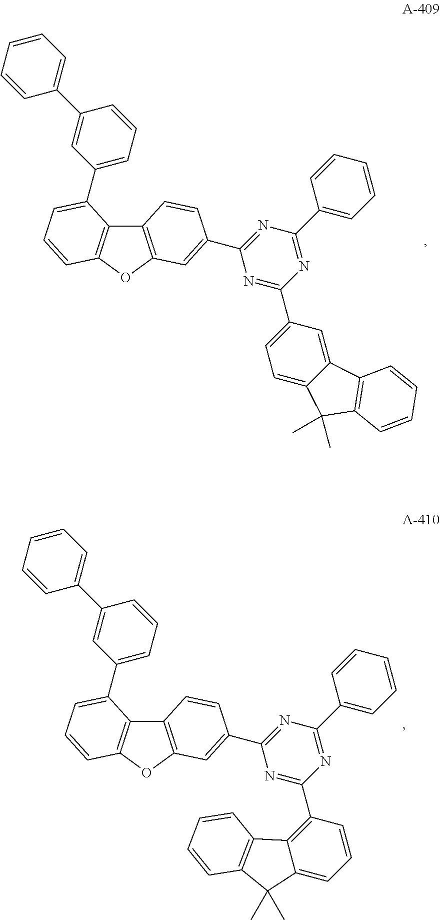 [SMILES: CC1(C)c2ccccc2-c2cc(-c3nc(-c4ccccc4)nc(-c4ccc5c(c4)oc4cccc(-c6cccc(-c7ccccc7)c6)c45)n3)ccc21 ; CC1(C)c2ccccc2-c2c(-c3nc(-c4ccccc4)nc(-c4ccc5c(c4)oc4cccc(-c6cccc(-c7ccccc7)c6)c45)n3)cccc21] 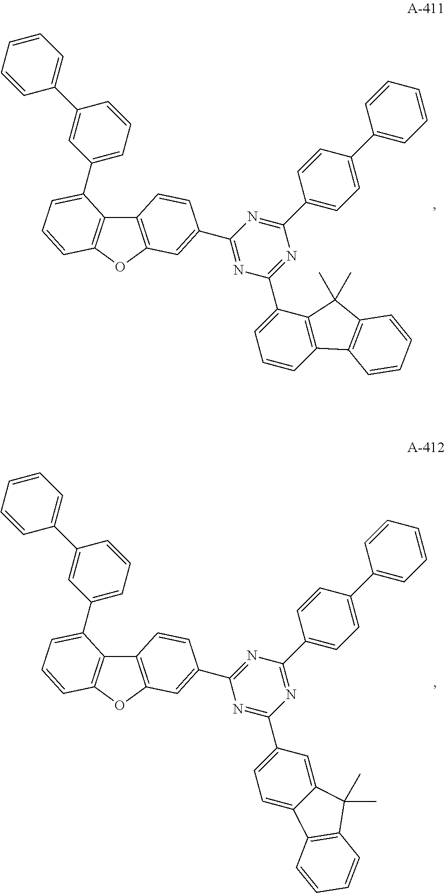 [SMILES: CC1(C)c2ccccc2-c2cccc(-c3nc(-c4ccc(-c5ccccc5)cc4)nc(-c4ccc5c(c4)oc4cccc(-c6cccc(-c7ccccc7)c6)c45)n3)c21 ; CC1(C)c2ccccc2-c2ccc(-c3nc(-c4ccc(-c5ccccc5)cc4)nc(-c4ccc5c(c4)oc4cccc(-c6cccc(-c7ccccc7)c6)c45)n3)cc21] 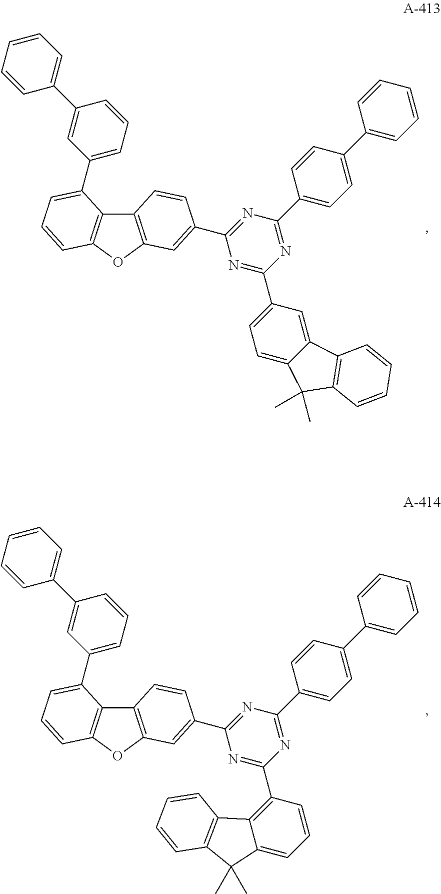 [SMILES: CC1(C)c2ccccc2-c2cc(-c3nc(-c4ccc(-c5ccccc5)cc4)nc(-c4ccc5c(c4)oc4cccc(-c6cccc(-c7ccccc7)c6)c45)n3)ccc21 ; CC1(C)c2ccccc2-c2c(-c3nc(-c4ccc(-c5ccccc5)cc4)nc(-c4ccc5c(c4)oc4cccc(-c6cccc(-c7ccccc7)c6)c45)n3)cccc21] 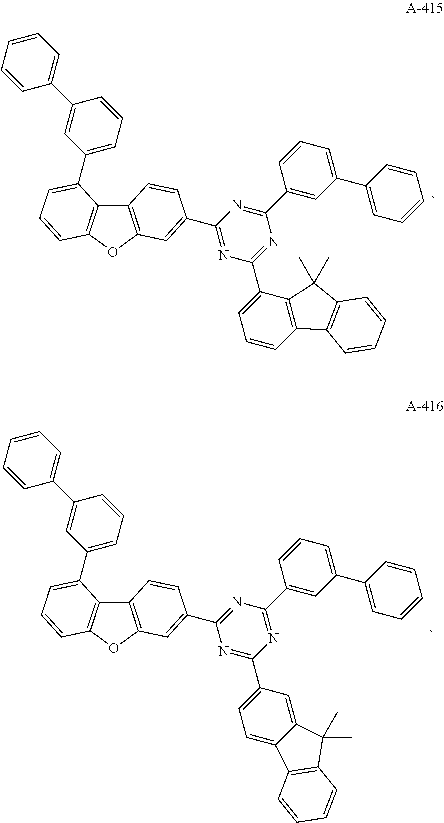 [SMILES: CC1(C)c2ccccc2-c2cccc(-c3nc(-c4cccc(-c5ccccc5)c4)nc(-c4ccc5c(c4)oc4cccc(-c6cccc(-c7ccccc7)c6)c45)n3)c21 ; CC1(C)c2ccccc2-c2ccc(-c3nc(-c4cccc(-c5ccccc5)c4)nc(-c4ccc5c(c4)oc4cccc(-c6cccc(-c7ccccc7)c6)c45)n3)cc21] 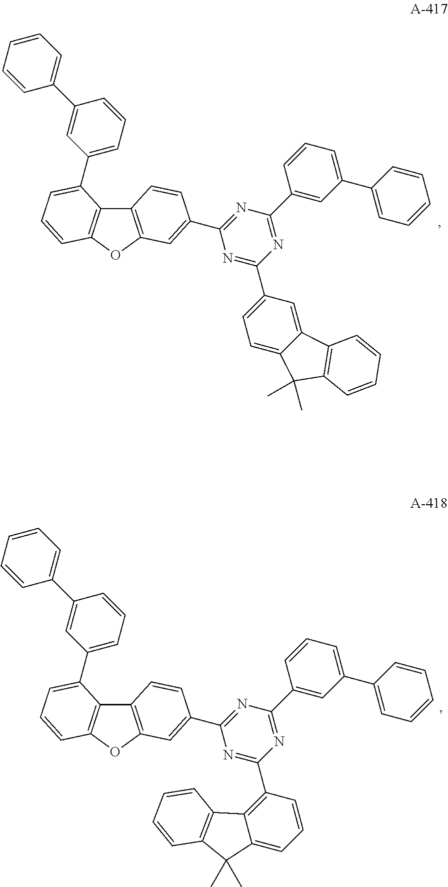 [SMILES: CC1(C)c2ccccc2-c2cc(-c3nc(-c4cccc(-c5ccccc5)c4)nc(-c4ccc5c(c4)oc4cccc(-c6cccc(-c7ccccc7)c6)c45)n3)ccc21 ; CC1(C)c2ccccc2-c2c(-c3nc(-c4cccc(-c5ccccc5)c4)nc(-c4ccc5c(c4)oc4cccc(-c6cccc(-c7ccccc7)c6)c45)n3)cccc21] 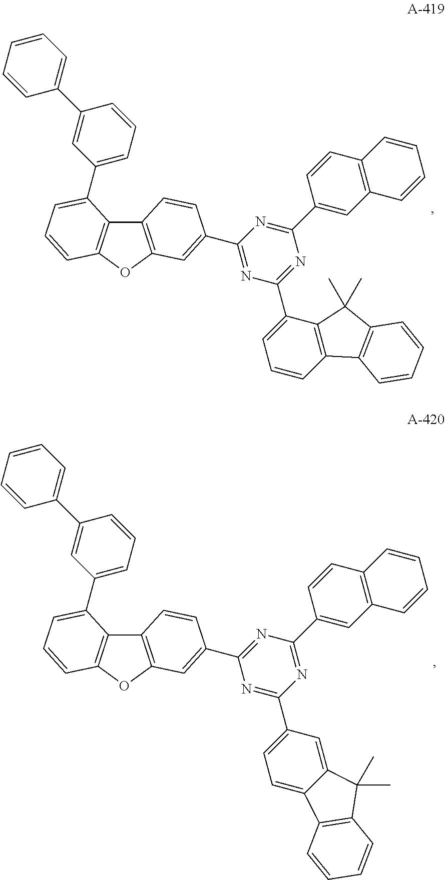 [SMILES: CC1(C)c2ccccc2-c2cccc(-c3nc(-c4ccc5ccccc5c4)nc(-c4ccc5c(c4)oc4cccc(-c6cccc(-c7ccccc7)c6)c45)n3)c21 ; CC1(C)c2ccccc2-c2ccc(-c3nc(-c4ccc5ccccc5c4)nc(-c4ccc5c(c4)oc4cccc(-c6cccc(-c7ccccc7)c6)c45)n3)cc21] 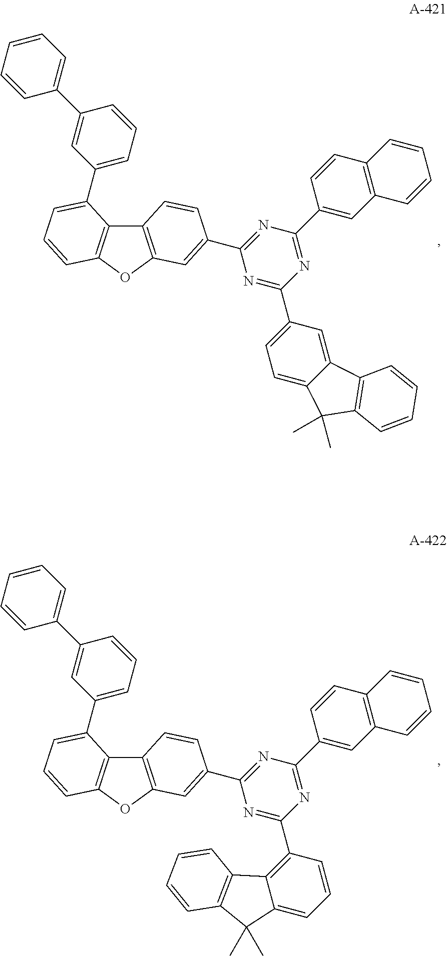 [SMILES: CC1(C)c2ccccc2-c2cc(-c3nc(-c4ccc5ccccc5c4)nc(-c4ccc5c(c4)oc4cccc(-c6cccc(-c7ccccc7)c6)c45)n3)ccc21 ; CC1(C)c2ccccc2-c2c(-c3nc(-c4ccc5ccccc5c4)nc(-c4ccc5c(c4)oc4cccc(-c6cccc(-c7ccccc7)c6)c45)n3)cccc21] 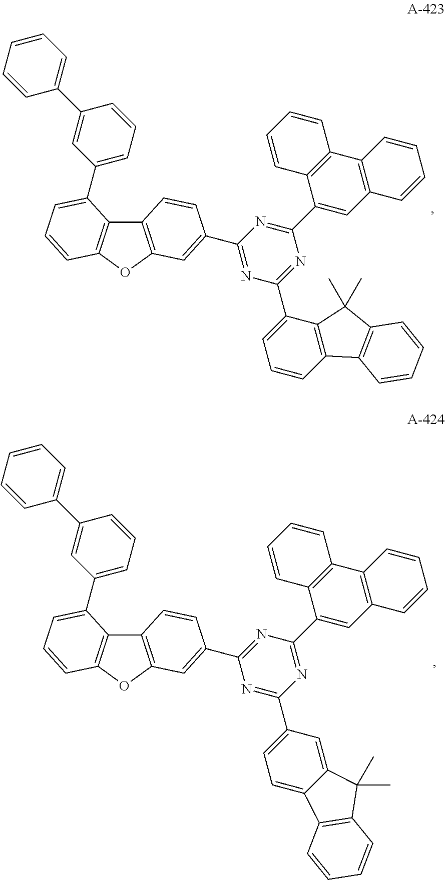 [SMILES: CC1(C)c2ccccc2-c2cccc(-c3nc(-c4ccc5c(c4)oc4cccc(-c6cccc(-c7ccccc7)c6)c45)nc(-c4cc5ccccc5c5ccccc45)n3)c21 ; CC1(C)c2ccccc2-c2ccc(-c3nc(-c4ccc5c(c4)oc4cccc(-c6cccc(-c7ccccc7)c6)c45)nc(-c4cc5ccccc5c5ccccc45)n3)cc21] 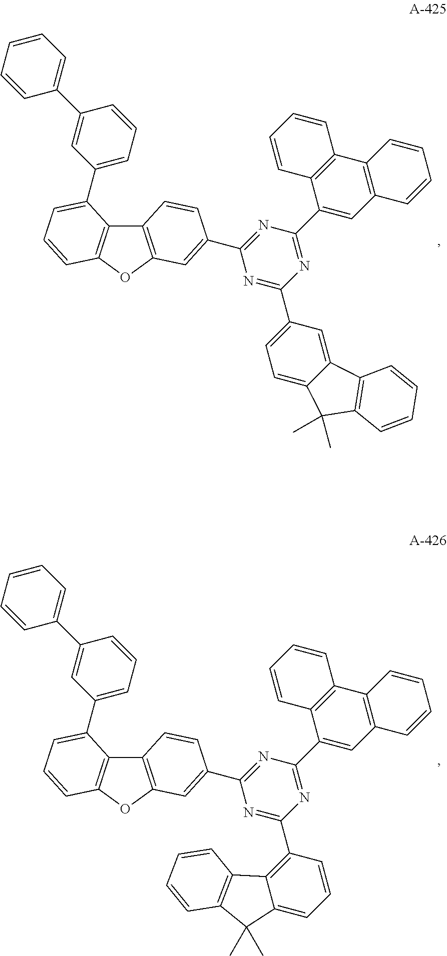 [SMILES: CC1(C)c2ccccc2-c2cc(-c3nc(-c4ccc5c(c4)oc4cccc(-c6cccc(-c7ccccc7)c6)c45)nc(-c4cc5ccccc5c5ccccc45)n3)ccc21 ; CC1(C)c2ccccc2-c2c(-c3nc(-c4ccc5c(c4)oc4cccc(-c6cccc(-c7ccccc7)c6)c45)nc(-c4cc5ccccc5c5ccccc45)n3)cccc21] 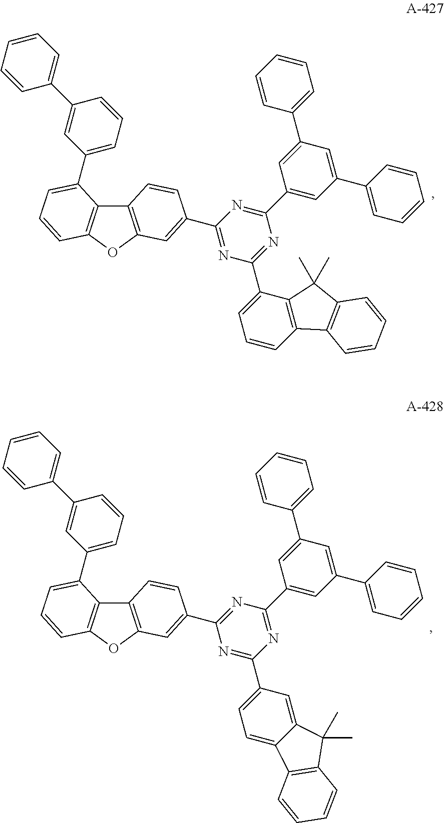 [SMILES: CC1(C)c2ccccc2-c2cccc(-c3nc(-c4cc(-c5ccccc5)cc(-c5ccccc5)c4)nc(-c4ccc5c(c4)oc4cccc(-c6cccc(-c7ccccc7)c6)c45)n3)c21 ; CC1(C)c2ccccc2-c2ccc(-c3nc(-c4cc(-c5ccccc5)cc(-c5ccccc5)c4)nc(-c4ccc5c(c4)oc4cccc(-c6cccc(-c7ccccc7)c6)c45)n3)cc21] 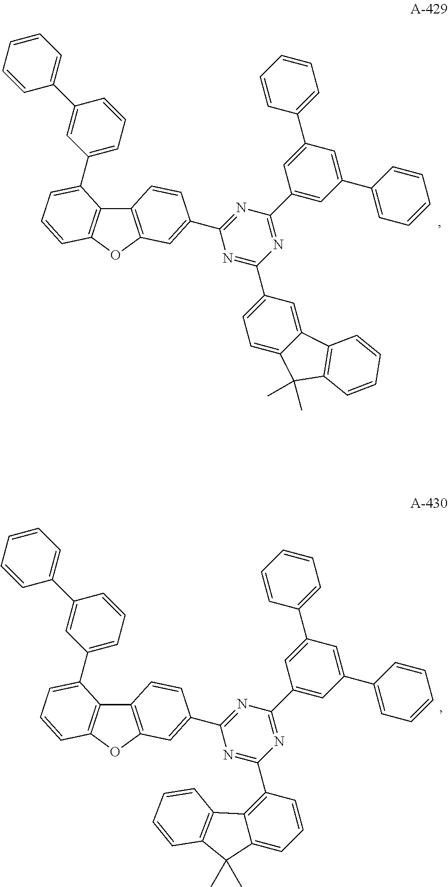 [SMILES: CC1(C)c2ccccc2-c2cc(-c3nc(-c4cc(-c5ccccc5)cc(-c5ccccc5)c4)nc(-c4ccc5c(c4)oc4cccc(-c6cccc(-c7ccccc7)c6)c45)n3)ccc21 ; CC1(C)c2ccccc2-c2c(-c3nc(-c4cc(-c5ccccc5)cc(-c5ccccc5)c4)nc(-c4ccc5c(c4)oc4cccc(-c6cccc(-c7ccccc7)c6)c45)n3)cccc21] 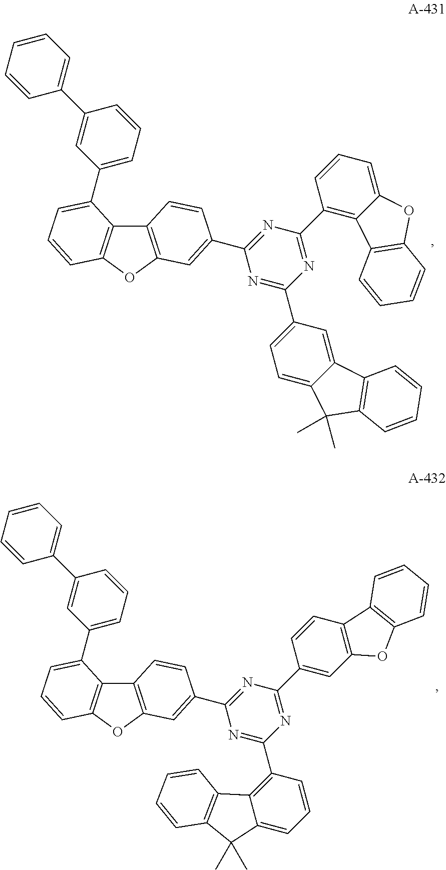 [SMILES: CC1(C)c2ccccc2-c2cc(-c3nc(-c4ccc5c(c4)oc4cccc(-c6cccc(-c7ccccc7)c6)c45)nc(-c4cccc5oc6ccccc6c45)n3)ccc21 ; CC1(C)c2ccccc2-c2c(-c3nc(-c4ccc5c(c4)oc4ccccc45)nc(-c4ccc5c(c4)oc4cccc(-c6cccc(-c7ccccc7)c6)c45)n3)cccc21] 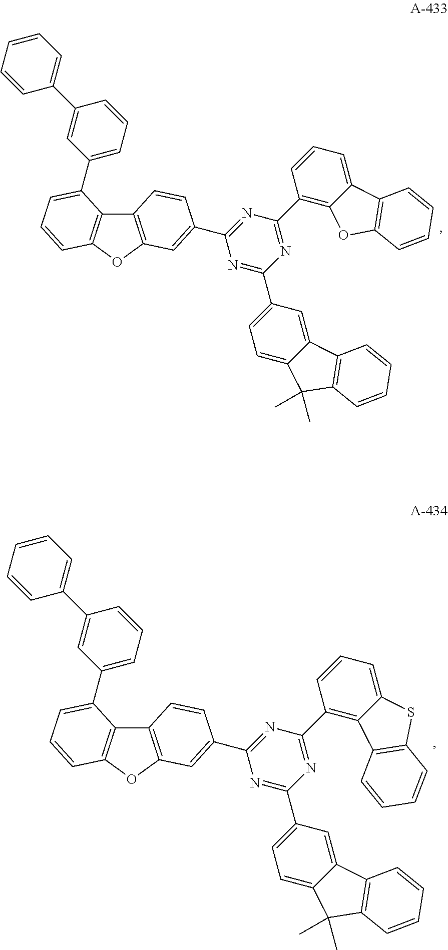 [SMILES: CC1(C)c2ccccc2-c2cc(-c3nc(-c4ccc5c(c4)oc4cccc(-c6cccc(-c7ccccc7)c6)c45)nc(-c4cccc5c4oc4ccccc45)n3)ccc21 ; CC1(C)c2ccccc2-c2cc(-c3nc(-c4ccc5c(c4)oc4cccc(-c6cccc(-c7ccccc7)c6)c45)nc(-c4cccc5sc6ccccc6c45)n3)ccc21] 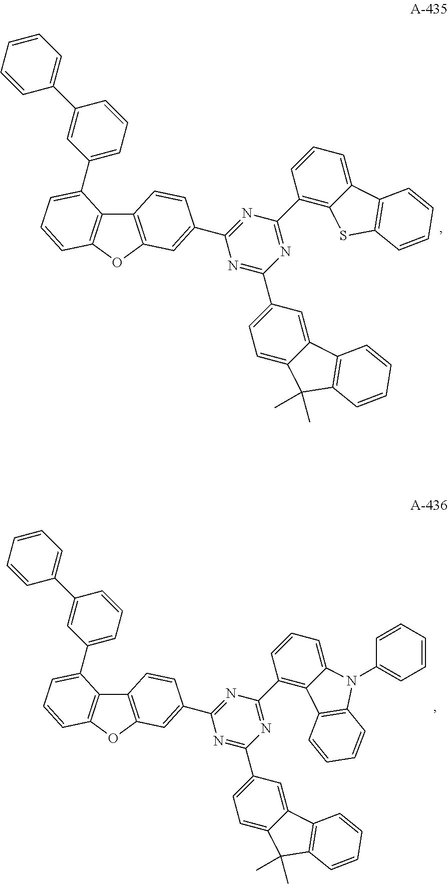 [SMILES: CC1(C)c2ccccc2-c2cc(-c3nc(-c4ccc5c(c4)oc4cccc(-c6cccc(-c7ccccc7)c6)c45)nc(-c4cccc5c4sc4ccccc45)n3)ccc21 ; CC1(C)c2ccccc2-c2cc(-c3nc(-c4ccc5c(c4)oc4cccc(-c6cccc(-c7ccccc7)c6)c45)nc(-c4cccc5c4c4ccccc4n5-c4ccccc4)n3)ccc21] 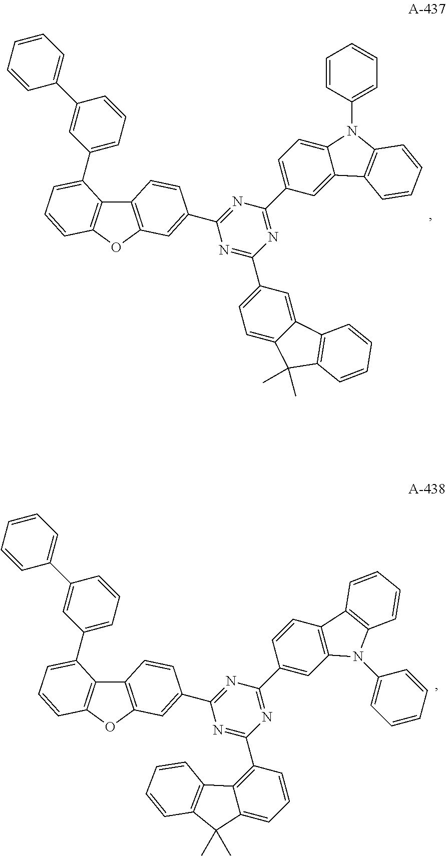 [SMILES: CC1(C)c2ccccc2-c2cc(-c3nc(-c4ccc5c(c4)oc4cccc(-c6cccc(-c7ccccc7)c6)c45)nc(-c4ccc5c(c4)c4ccccc4n5-c4ccccc4)n3)ccc21 ; CC1(C)c2ccccc2-c2c(-c3nc(-c4ccc5c(c4)oc4cccc(-c6cccc(-c7ccccc7)c6)c45)nc(-c4ccc5c6ccccc6n(-c6ccccc6)c5c4)n3)cccc21] 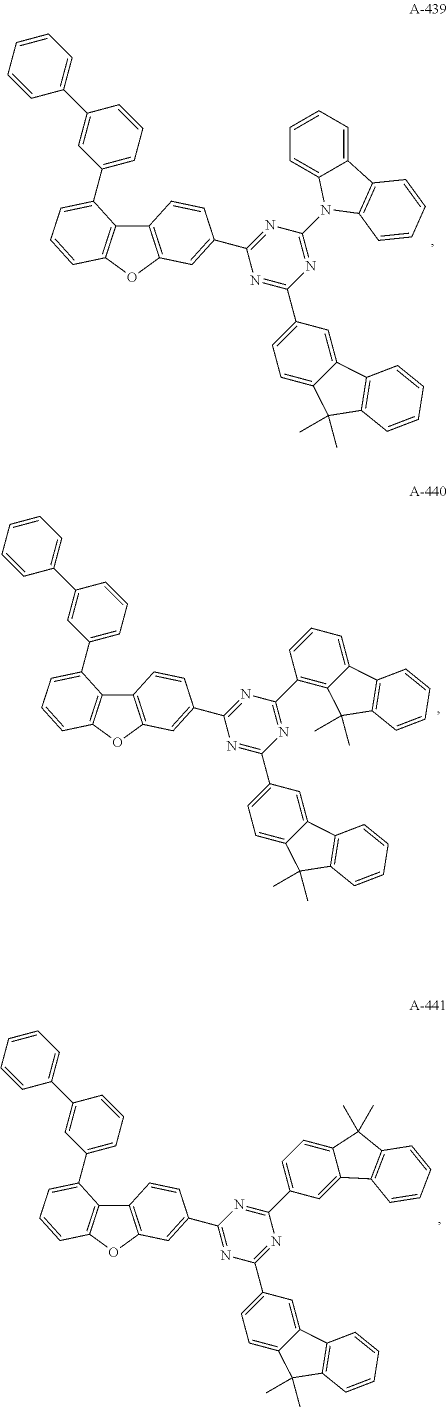 [SMILES: CC1(C)c2ccccc2-c2cc(-c3nc(-c4ccc5c(c4)oc4cccc(-c6cccc(-c7ccccc7)c6)c45)nc(-n4c5ccccc5c5ccccc54)n3)ccc21 ; CC1(C)c2ccccc2-c2cc(-c3nc(-c4ccc5c(c4)oc4cccc(-c6cccc(-c7ccccc7)c6)c45)nc(-c4cccc5c4C(C)(C)c4ccccc4-5)n3)ccc21 ; CC1(C)c2ccccc2-c2cc(-c3nc(-c4ccc5c(c4)-c4ccccc4C5(C)C)nc(-c4ccc5c(c4)oc4cccc(-c6cccc(-c7ccccc7)c6)c45)n3)ccc21] 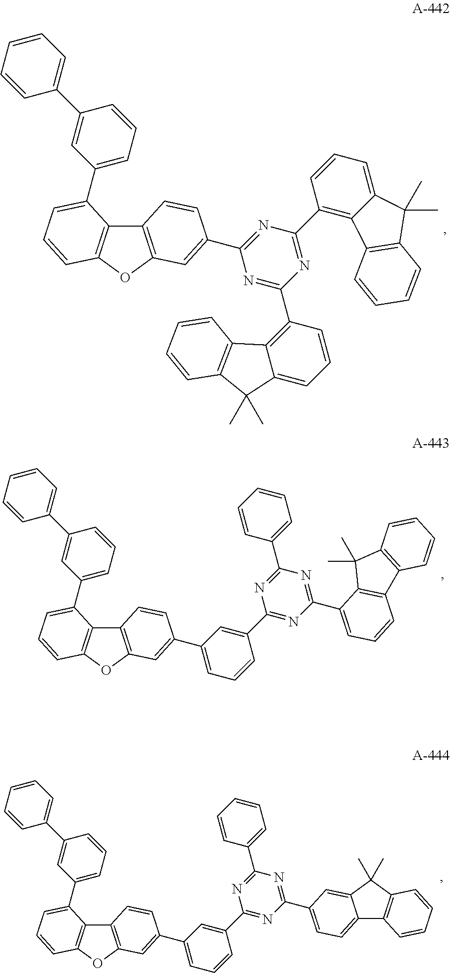 [SMILES: CC1(C)c2ccccc2-c2c(-c3nc(-c4ccc5c(c4)oc4cccc(-c6cccc(-c7ccccc7)c6)c45)nc(-c4cccc5c4-c4ccccc4C5(C)C)n3)cccc21 ; CC1(C)c2ccccc2-c2cccc(-c3nc(-c4ccccc4)nc(-c4cccc(-c5ccc6c(c5)oc5cccc(-c7cccc(-c8ccccc8)c7)c56)c4)n3)c21 ; CC1(C)c2ccccc2-c2ccc(-c3nc(-c4ccccc4)nc(-c4cccc(-c5ccc6c(c5)oc5cccc(-c7cccc(-c8ccccc8)c7)c56)c4)n3)cc21] 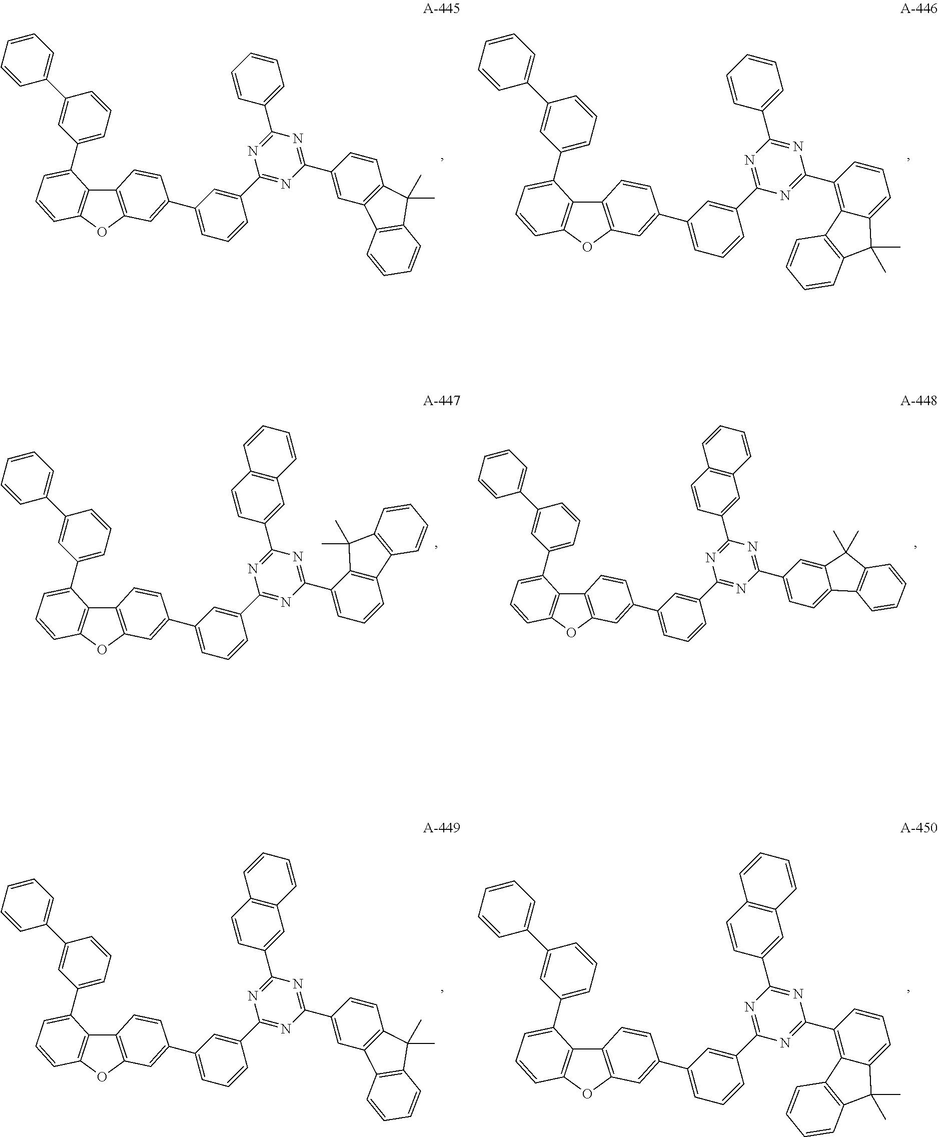 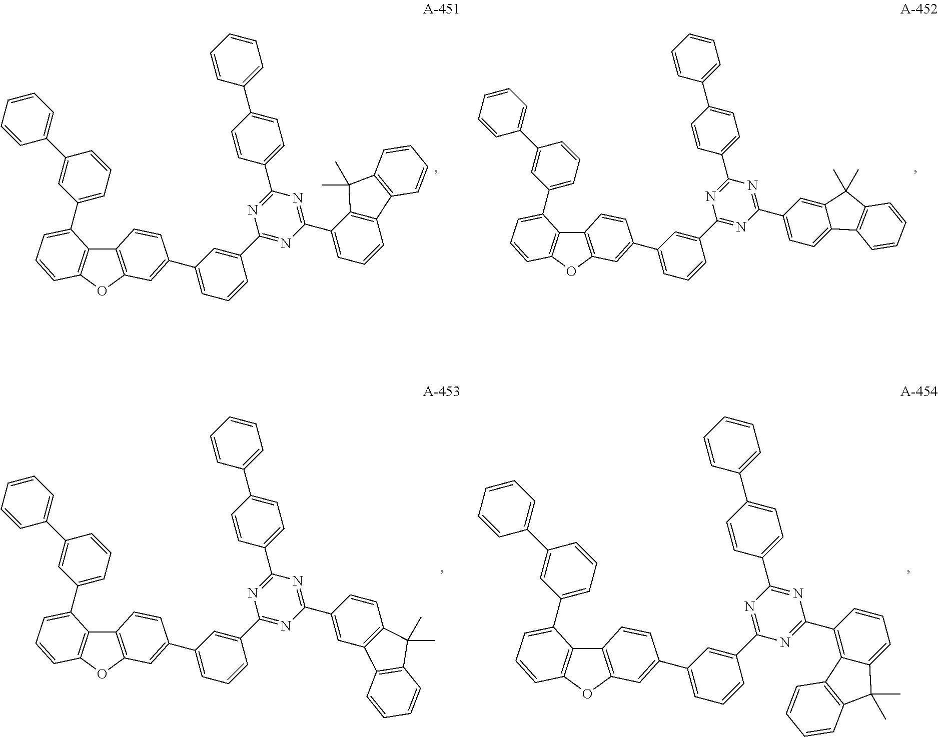 [SMILES: CC1(C)c2ccccc2-c2cccc(-c3nc(-c4ccc(-c5ccccc5)cc4)nc(-c4cccc(-c5ccc6c(c5)oc5cccc(-c7cccc(-c8ccccc8)c7)c56)c4)n3)c21 ; CC1(C)c2ccccc2-c2ccc(-c3nc(-c4ccc(-c5ccccc5)cc4)nc(-c4cccc(-c5ccc6c(c5)oc5cccc(-c7cccc(-c8ccccc8)c7)c56)c4)n3)cc21 ; CC1(C)c2ccccc2-c2cc(-c3nc(-c4ccc(-c5ccccc5)cc4)nc(-c4cccc(-c5ccc6c(c5)oc5cccc(-c7cccc(-c8ccccc8)c7)c56)c4)n3)ccc21 ; CC1(C)c2ccccc2-c2c(-c3nc(-c4ccc(-c5ccccc5)cc4)nc(-c4cccc(-c5ccc6c(c5)oc5cccc(-c7cccc(-c8ccccc8)c7)c56)c4)n3)cccc21] 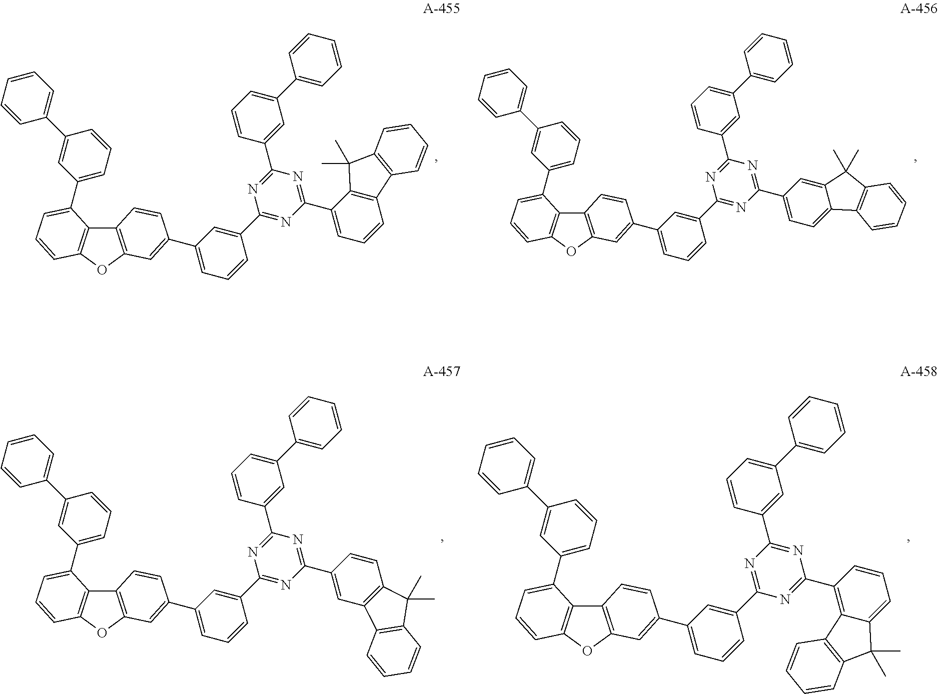 [SMILES: CC1(C)c2ccccc2-c2cccc(-c3nc(-c4cccc(-c5ccccc5)c4)nc(-c4cccc(-c5ccc6c(c5)oc5cccc(-c7cccc(-c8ccccc8)c7)c56)c4)n3)c21 ; CC1(C)c2ccccc2-c2ccc(-c3nc(-c4cccc(-c5ccccc5)c4)nc(-c4cccc(-c5ccc6c(c5)oc5cccc(-c7cccc(-c8ccccc8)c7)c56)c4)n3)cc21 ; CC1(C)c2ccccc2-c2cc(-c3nc(-c4cccc(-c5ccccc5)c4)nc(-c4cccc(-c5ccc6c(c5)oc5cccc(-c7cccc(-c8ccccc8)c7)c56)c4)n3)ccc21 ; CC1(C)c2ccccc2-c2c(-c3nc(-c4cccc(-c5ccccc5)c4)nc(-c4cccc(-c5ccc6c(c5)oc5cccc(-c7cccc(-c8ccccc8)c7)c56)c4)n3)cccc21] 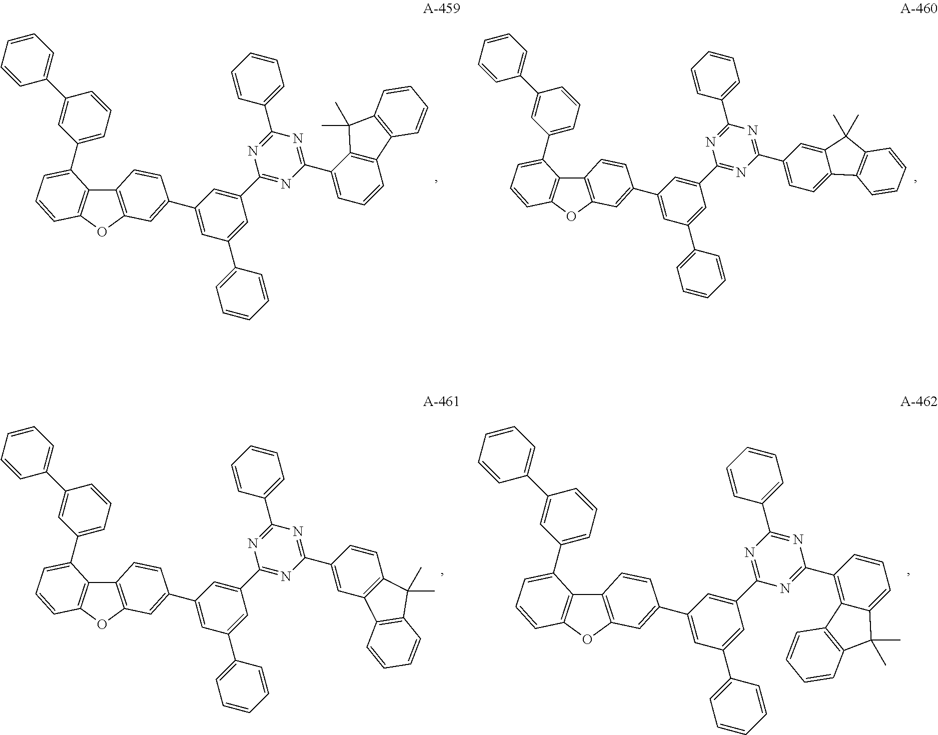 [SMILES: CC1(C)c2ccccc2-c2cccc(-c3nc(-c4ccccc4)nc(-c4cc(-c5ccccc5)cc(-c5ccc6c(c5)oc5cccc(-c7cccc(-c8ccccc8)c7)c56)c4)n3)c21 ; CC1(C)c2ccccc2-c2ccc(-c3nc(-c4ccccc4)nc(-c4cc(-c5ccccc5)cc(-c5ccc6c(c5)oc5cccc(-c7cccc(-c8ccccc8)c7)c56)c4)n3)cc21 ; CC1(C)c2ccccc2-c2cc(-c3nc(-c4ccccc4)nc(-c4cc(-c5ccccc5)cc(-c5ccc6c(c5)oc5cccc(-c7cccc(-c8ccccc8)c7)c56)c4)n3)ccc21 ; CC1(C)c2ccccc2-c2c(-c3nc(-c4ccccc4)nc(-c4cc(-c5ccccc5)cc(-c5ccc6c(c5)oc5cccc(-c7cccc(-c8ccccc8)c7)c56)c4)n3)cccc21] 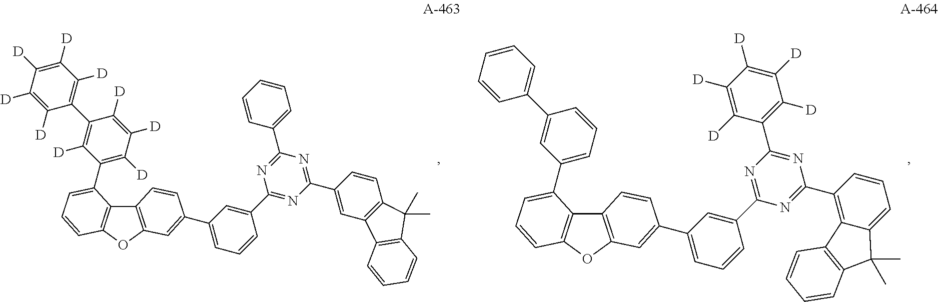 [SMILES: [2H]c1c([2H])c([2H])c(-c2c([2H])c([2H])c([2H])c(-c3cccc4oc5cc(-c6cccc(-c7nc(-c8ccccc8)nc(-c8ccc9c(c8)-c8ccccc8C9(C)C)n7)c6)ccc5c34)c2[2H])c([2H])c1[2H] ; [2H]c1c([2H])c([2H])c(-c2nc(-c3cccc(-c4ccc5c(c4)oc4cccc(-c6cccc(-c7ccccc7)c6)c45)c3)nc(-c3cccc4c3-c3ccccc3C4(C)C)n2)c([2H])c1[2H]] 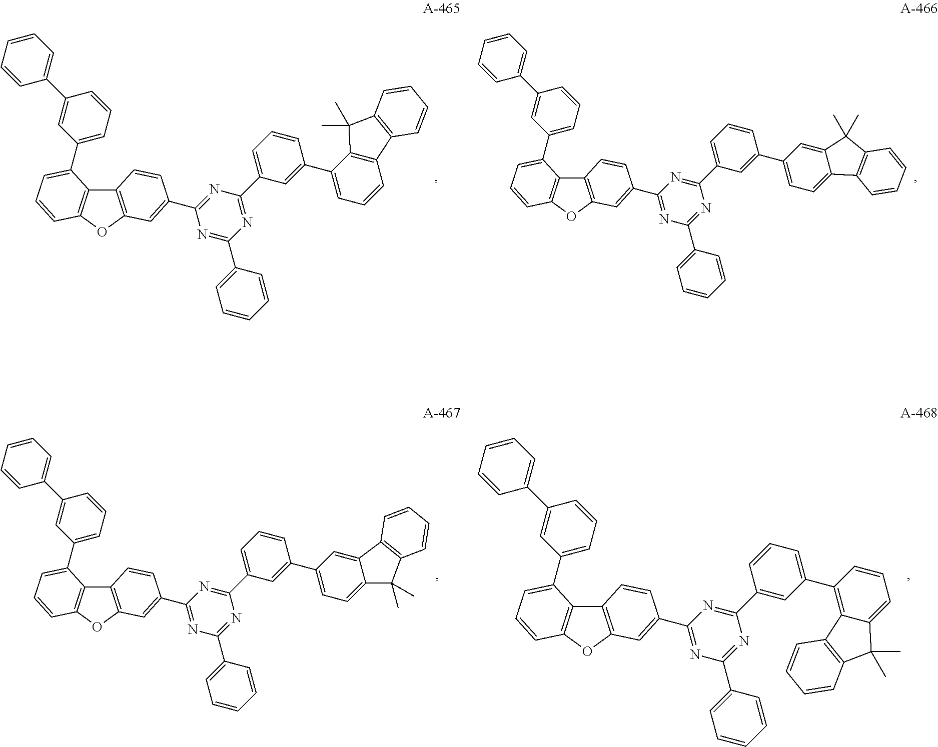 [SMILES: CC1(C)c2ccccc2-c2cccc(-c3cccc(-c4nc(-c5ccccc5)nc(-c5ccc6c(c5)oc5cccc(-c7cccc(-c8ccccc8)c7)c56)n4)c3)c21 ; CC1(C)c2ccccc2-c2ccc(-c3cccc(-c4nc(-c5ccccc5)nc(-c5ccc6c(c5)oc5cccc(-c7cccc(-c8ccccc8)c7)c56)n4)c3)cc21 ; CC1(C)c2ccccc2-c2cc(-c3cccc(-c4nc(-c5ccccc5)nc(-c5ccc6c(c5)oc5cccc(-c7cccc(-c8ccccc8)c7)c56)n4)c3)ccc21 ; CC1(C)c2ccccc2-c2c(-c3cccc(-c4nc(-c5ccccc5)nc(-c5ccc6c(c5)oc5cccc(-c7cccc(-c8ccccc8)c7)c56)n4)c3)cccc21] 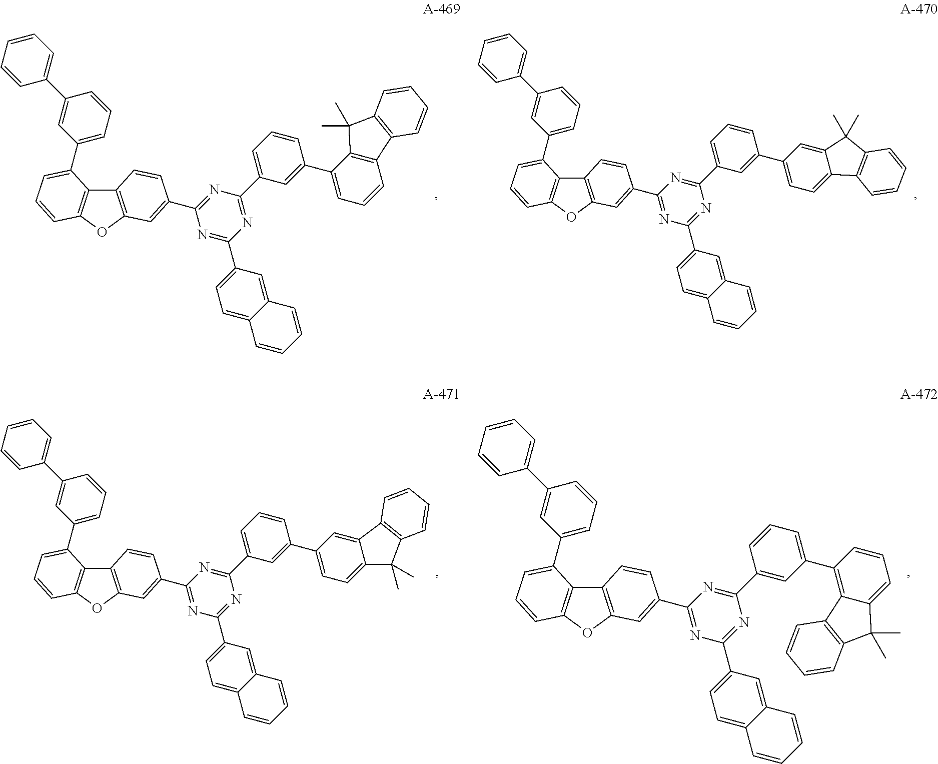 [SMILES: CC1(C)c2ccccc2-c2cccc(-c3cccc(-c4nc(-c5ccc6ccccc6c5)nc(-c5ccc6c(c5)oc5cccc(-c7cccc(-c8ccccc8)c7)c56)n4)c3)c21 ; CC1(C)c2ccccc2-c2ccc(-c3cccc(-c4nc(-c5ccc6ccccc6c5)nc(-c5ccc6c(c5)oc5cccc(-c7cccc(-c8ccccc8)c7)c56)n4)c3)cc21 ; CC1(C)c2ccccc2-c2cc(-c3cccc(-c4nc(-c5ccc6ccccc6c5)nc(-c5ccc6c(c5)oc5cccc(-c7cccc(-c8ccccc8)c7)c56)n4)c3)ccc21 ; CC1(C)c2ccccc2-c2c(-c3cccc(-c4nc(-c5ccc6ccccc6c5)nc(-c5ccc6c(c5)oc5cccc(-c7cccc(-c8ccccc8)c7)c56)n4)c3)cccc21] 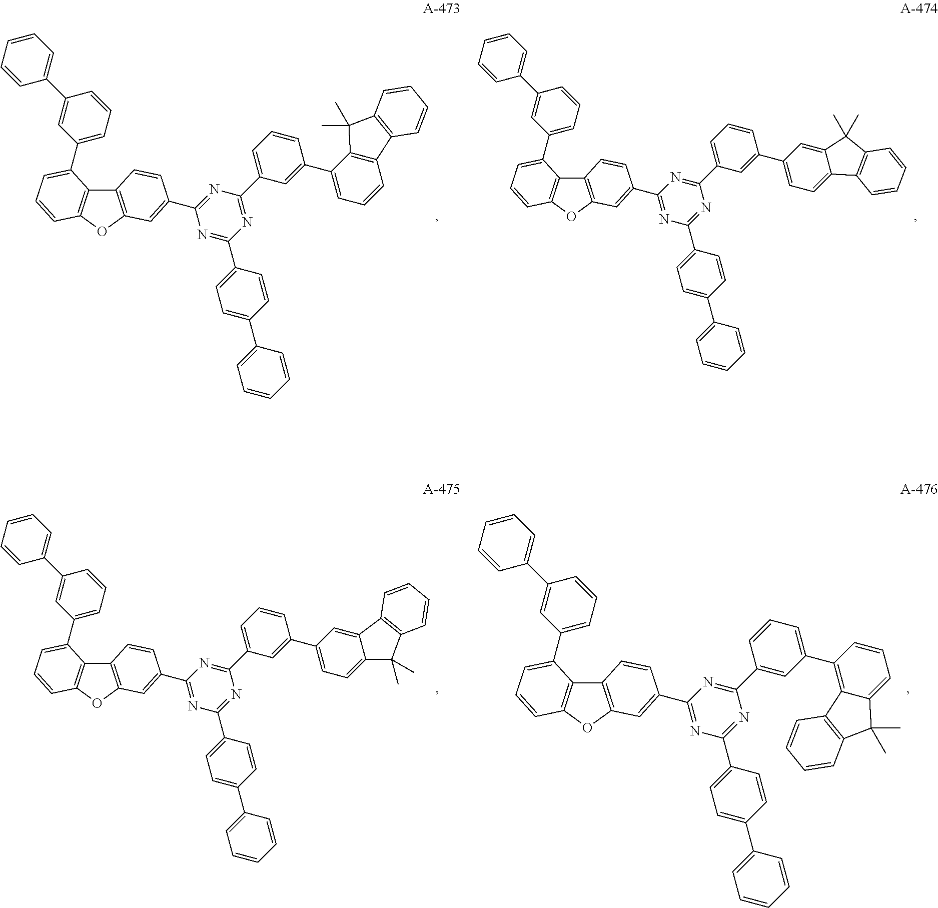 [SMILES: CC1(C)c2ccccc2-c2cccc(-c3cccc(-c4nc(-c5ccc(-c6ccccc6)cc5)nc(-c5ccc6c(c5)oc5cccc(-c7cccc(-c8ccccc8)c7)c56)n4)c3)c21 ; CC1(C)c2ccccc2-c2ccc(-c3cccc(-c4nc(-c5ccc(-c6ccccc6)cc5)nc(-c5ccc6c(c5)oc5cccc(-c7cccc(-c8ccccc8)c7)c56)n4)c3)cc21 ; CC1(C)c2ccccc2-c2cc(-c3cccc(-c4nc(-c5ccc(-c6ccccc6)cc5)nc(-c5ccc6c(c5)oc5cccc(-c7cccc(-c8ccccc8)c7)c56)n4)c3)ccc21 ; CC1(C)c2ccccc2-c2c(-c3cccc(-c4nc(-c5ccc(-c6ccccc6)cc5)nc(-c5ccc6c(c5)oc5cccc(-c7cccc(-c8ccccc8)c7)c56)n4)c3)cccc21] 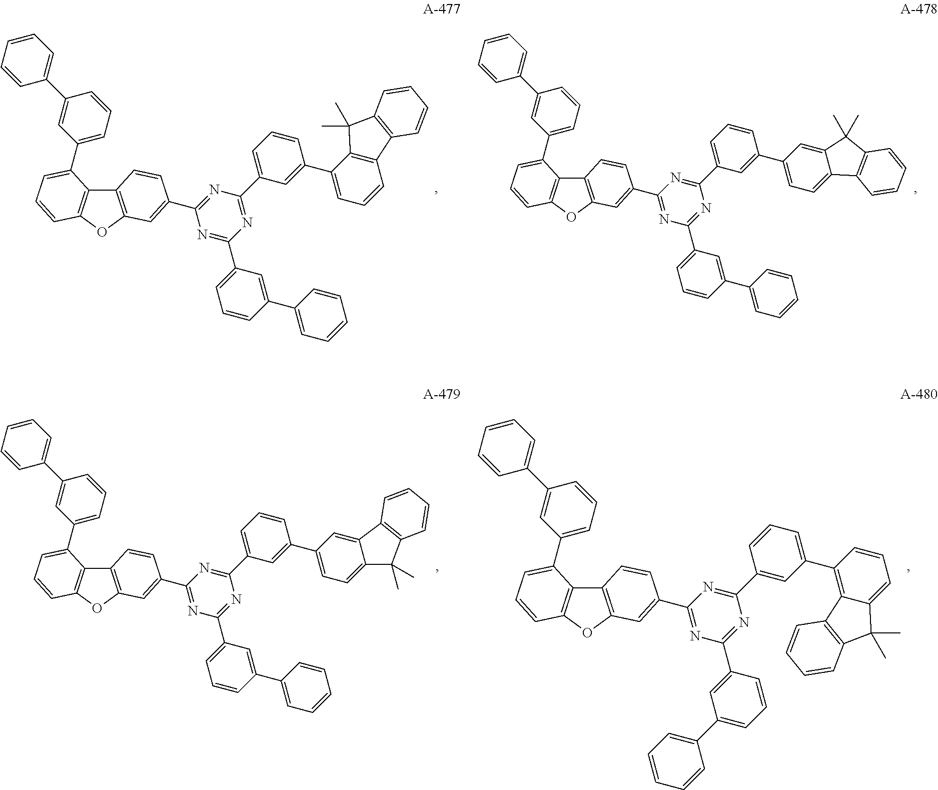 [SMILES: CC1(C)c2ccccc2-c2cccc(-c3cccc(-c4nc(-c5cccc(-c6ccccc6)c5)nc(-c5ccc6c(c5)oc5cccc(-c7cccc(-c8ccccc8)c7)c56)n4)c3)c21 ; CC1(C)c2ccccc2-c2ccc(-c3cccc(-c4nc(-c5cccc(-c6ccccc6)c5)nc(-c5ccc6c(c5)oc5cccc(-c7cccc(-c8ccccc8)c7)c56)n4)c3)cc21 ; CC1(C)c2ccccc2-c2cc(-c3cccc(-c4nc(-c5cccc(-c6ccccc6)c5)nc(-c5ccc6c(c5)oc5cccc(-c7cccc(-c8ccccc8)c7)c56)n4)c3)ccc21 ; CC1(C)c2ccccc2-c2c(-c3cccc(-c4nc(-c5cccc(-c6ccccc6)c5)nc(-c5ccc6c(c5)oc5cccc(-c7cccc(-c8ccccc8)c7)c56)n4)c3)cccc21] 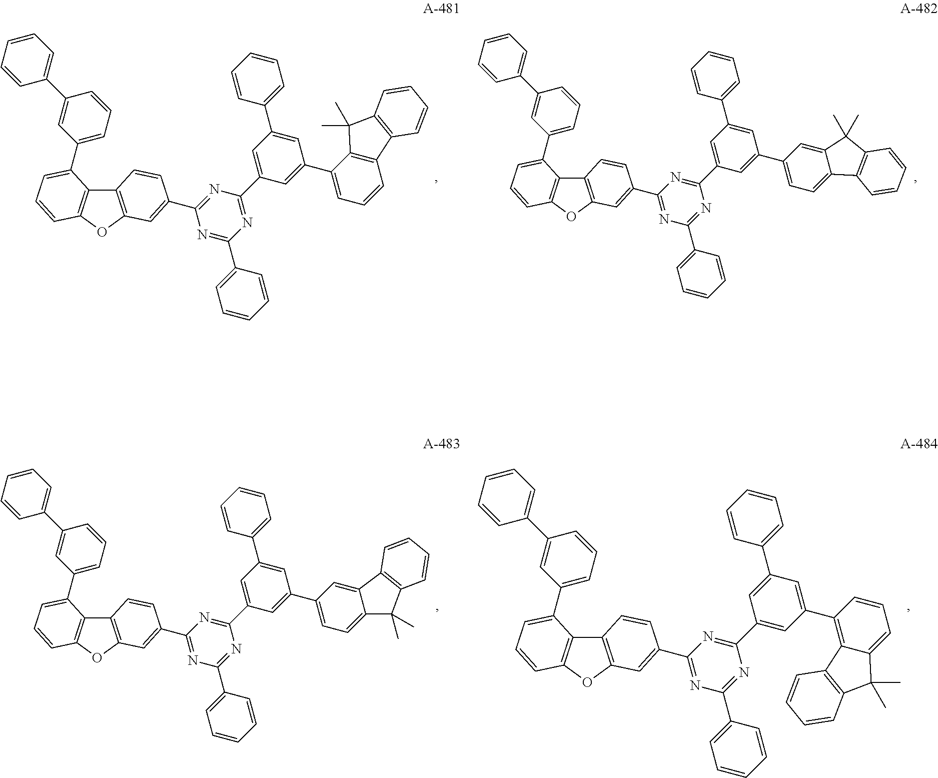 [SMILES: CC1(C)c2ccccc2-c2cccc(-c3cc(-c4ccccc4)cc(-c4nc(-c5ccccc5)nc(-c5ccc6c(c5)oc5cccc(-c7cccc(-c8ccccc8)c7)c56)n4)c3)c21 ; CC1(C)c2ccccc2-c2ccc(-c3cc(-c4ccccc4)cc(-c4nc(-c5ccccc5)nc(-c5ccc6c(c5)oc5cccc(-c7cccc(-c8ccccc8)c7)c56)n4)c3)cc21 ; CC1(C)c2ccccc2-c2cc(-c3cc(-c4ccccc4)cc(-c4nc(-c5ccccc5)nc(-c5ccc6c(c5)oc5cccc(-c7cccc(-c8ccccc8)c7)c56)n4)c3)ccc21 ; CC1(C)c2ccccc2-c2c(-c3cc(-c4ccccc4)cc(-c4nc(-c5ccccc5)nc(-c5ccc6c(c5)oc5cccc(-c7cccc(-c8ccccc8)c7)c56)n4)c3)cccc21] 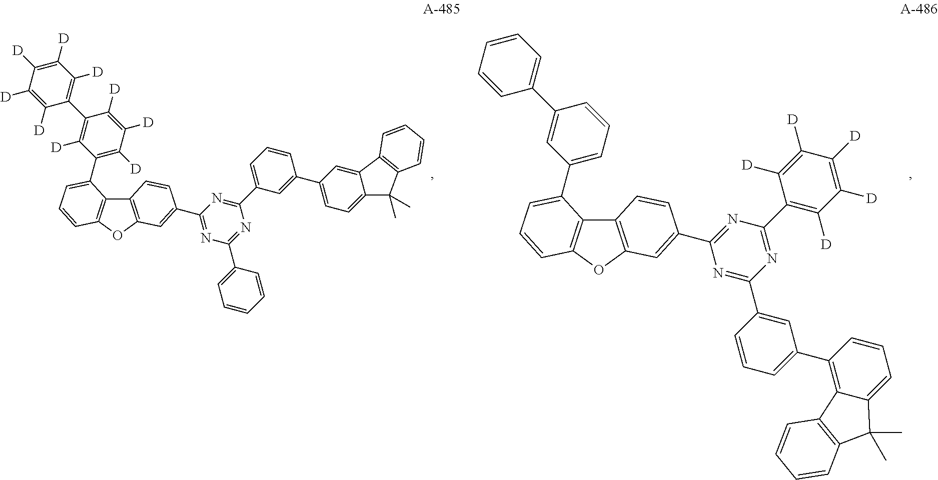 [SMILES: [2H]c1c([2H])c([2H])c(-c2c([2H])c([2H])c([2H])c(-c3cccc4oc5cc(-c6nc(-c7ccccc7)nc(-c7cccc(-c8ccc9c(c8)-c8ccccc8C9(C)C)c7)n6)ccc5c34)c2[2H])c([2H])c1[2H] ; [2H]c1c([2H])c([2H])c(-c2nc(-c3cccc(-c4cccc5c4-c4ccccc4C5(C)C)c3)nc(-c3ccc4c(c3)oc3cccc(-c5cccc(-c6ccccc6)c5)c34)n2)c([2H])c1[2H]] 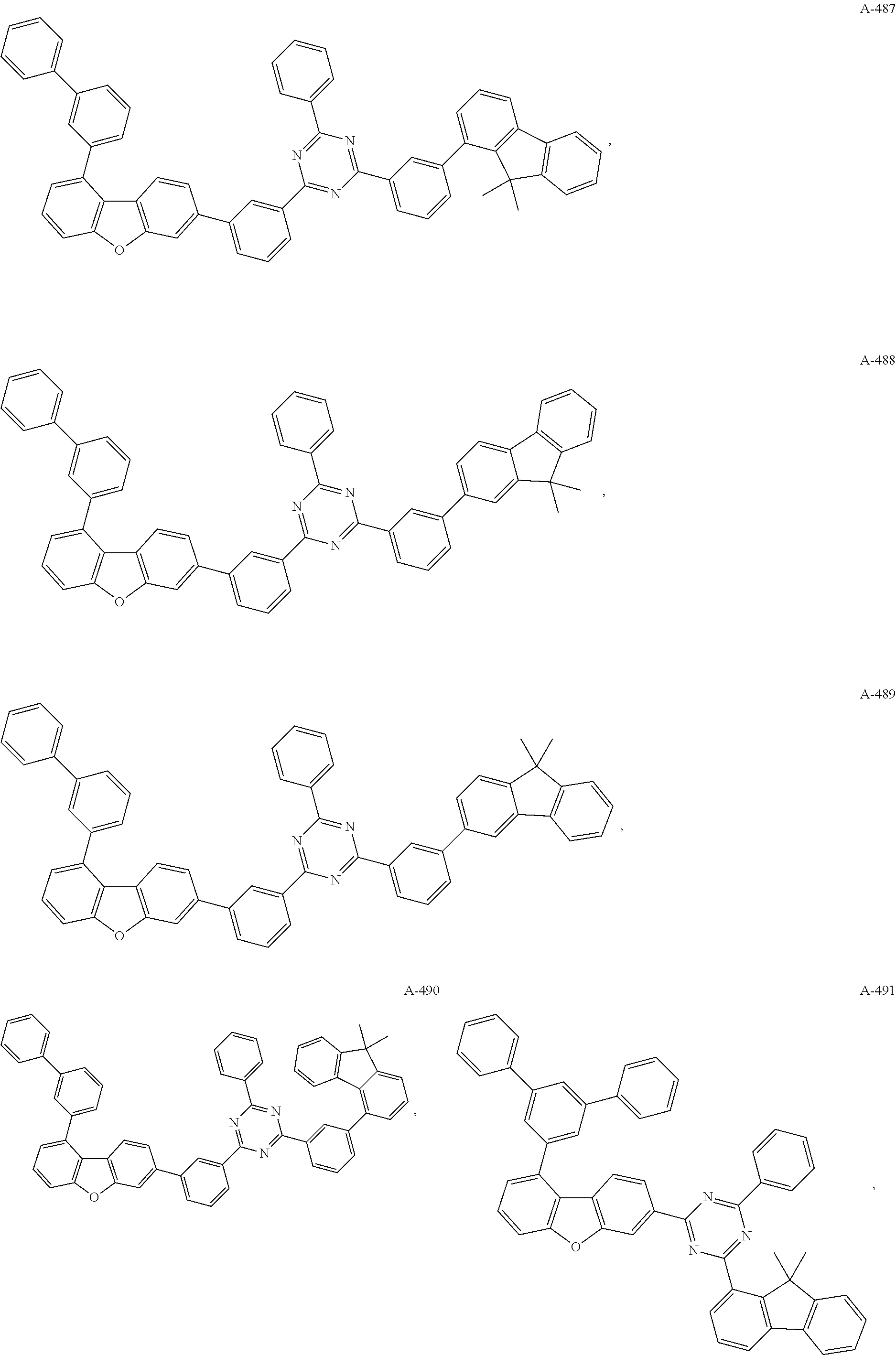 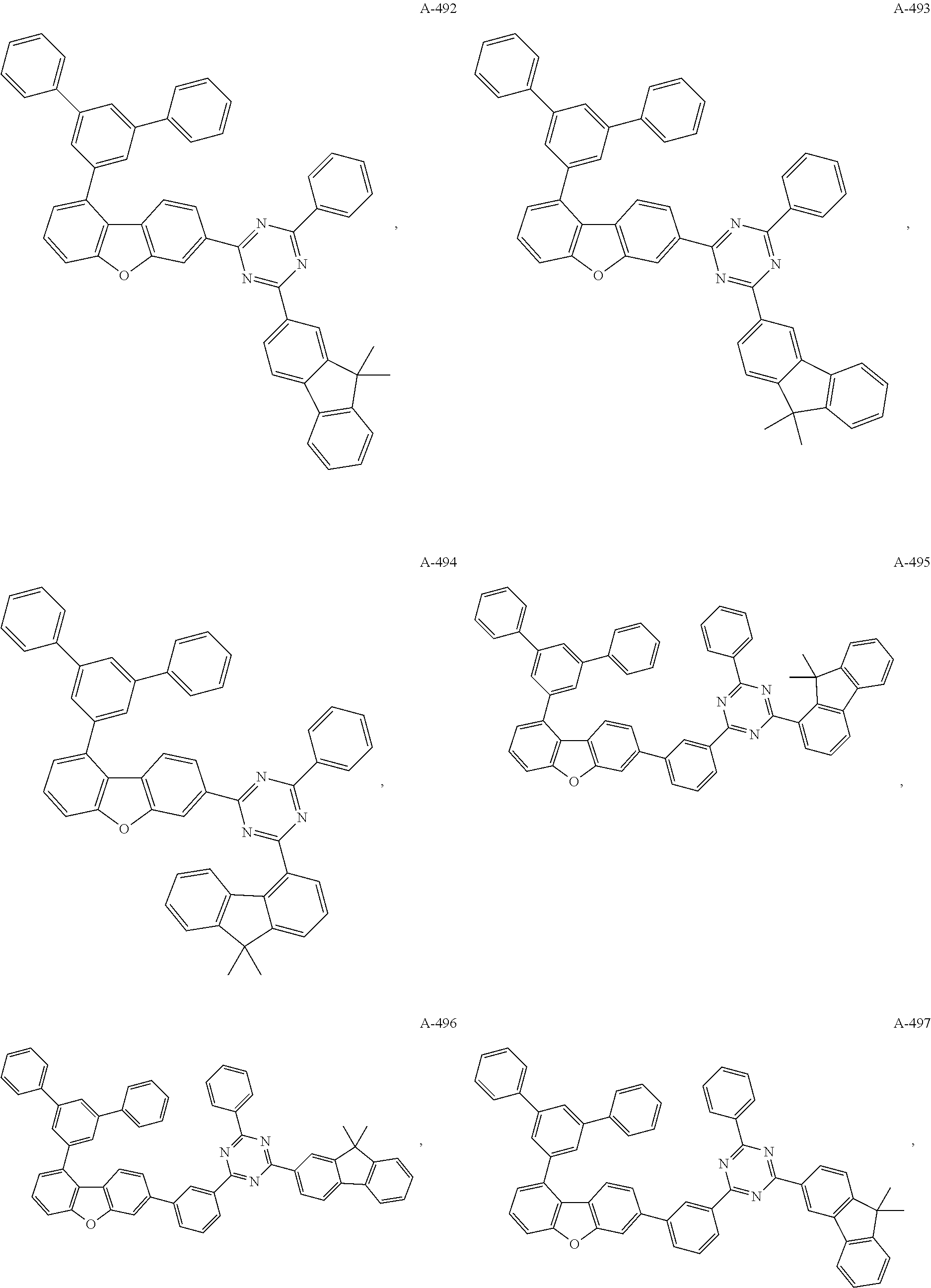 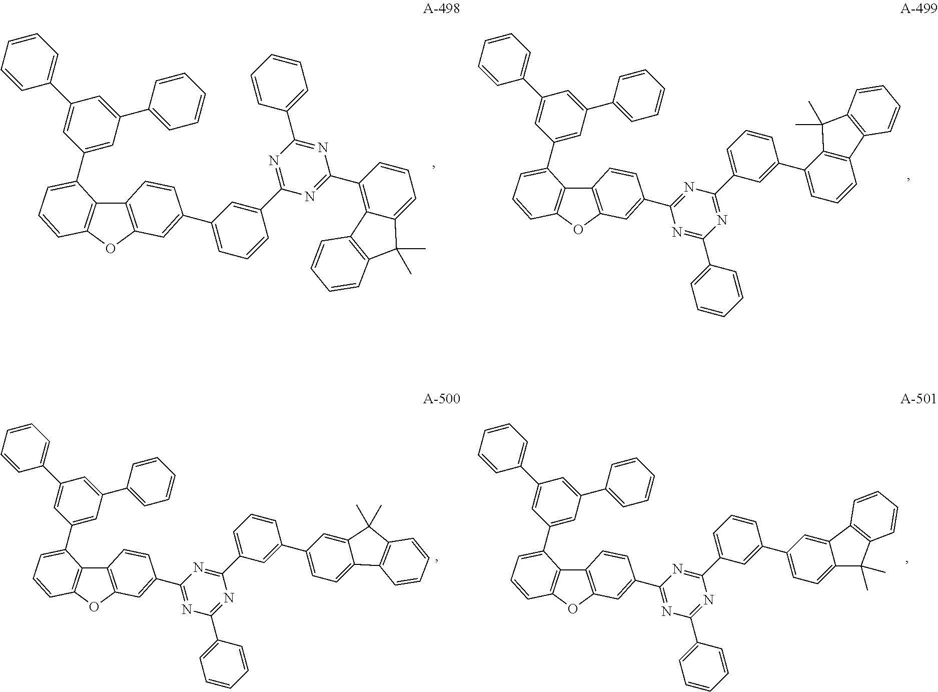 [SMILES: CC1(C)c2ccccc2-c2c(-c3nc(-c4ccccc4)nc(-c4cccc(-c5ccc6c(c5)oc5cccc(-c7cc(-c8ccccc8)cc(-c8ccccc8)c7)c56)c4)n3)cccc21 ; CC1(C)c2ccccc2-c2cccc(-c3cccc(-c4nc(-c5ccccc5)nc(-c5ccc6c(c5)oc5cccc(-c7cc(-c8ccccc8)cc(-c8ccccc8)c7)c56)n4)c3)c21 ; CC1(C)c2ccccc2-c2ccc(-c3cccc(-c4nc(-c5ccccc5)nc(-c5ccc6c(c5)oc5cccc(-c7cc(-c8ccccc8)cc(-c8ccccc8)c7)c56)n4)c3)cc21 ; CC1(C)c2ccccc2-c2cc(-c3cccc(-c4nc(-c5ccccc5)nc(-c5ccc6c(c5)oc5cccc(-c7cc(-c8ccccc8)cc(-c8ccccc8)c7)c56)n4)c3)ccc21] 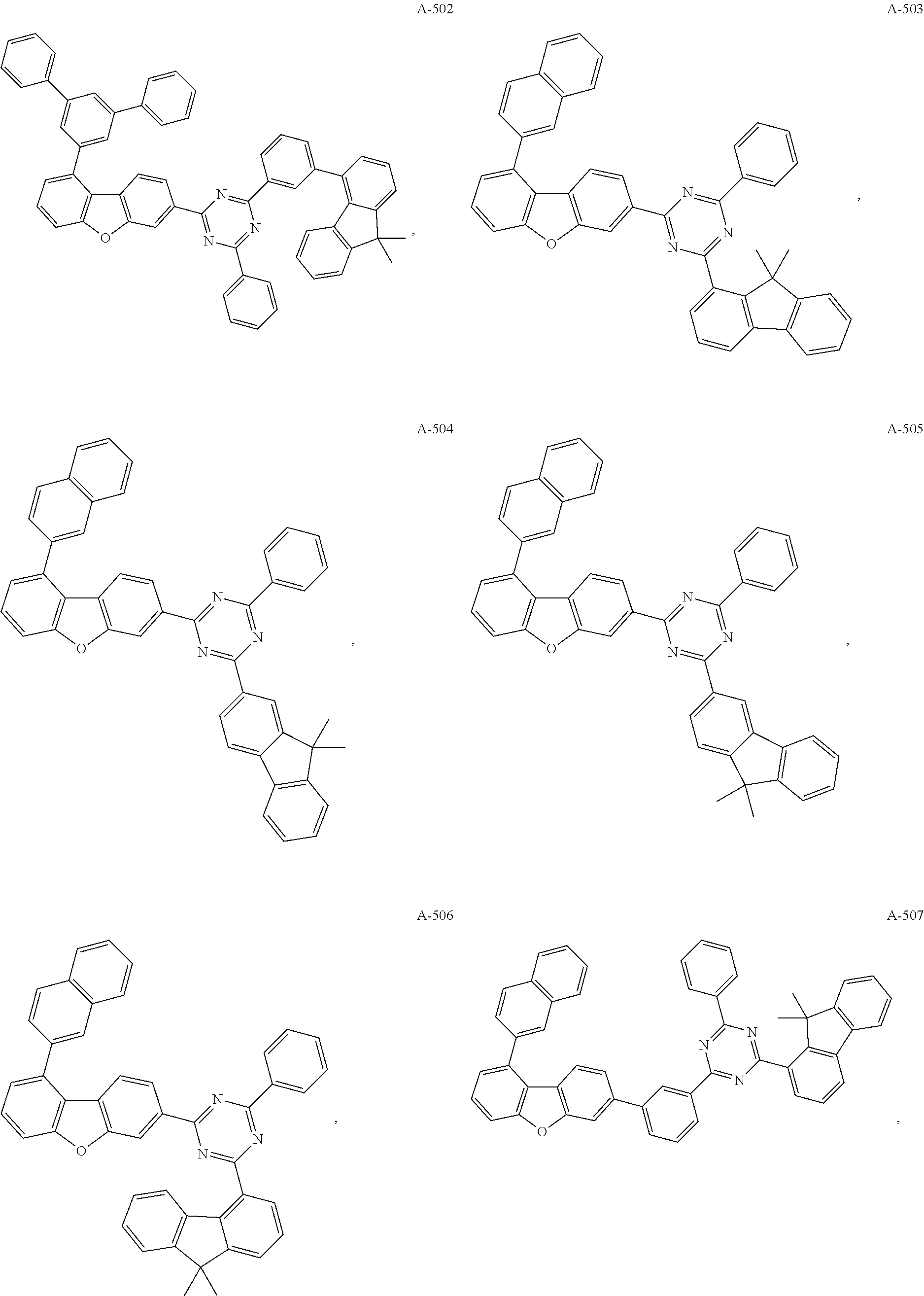 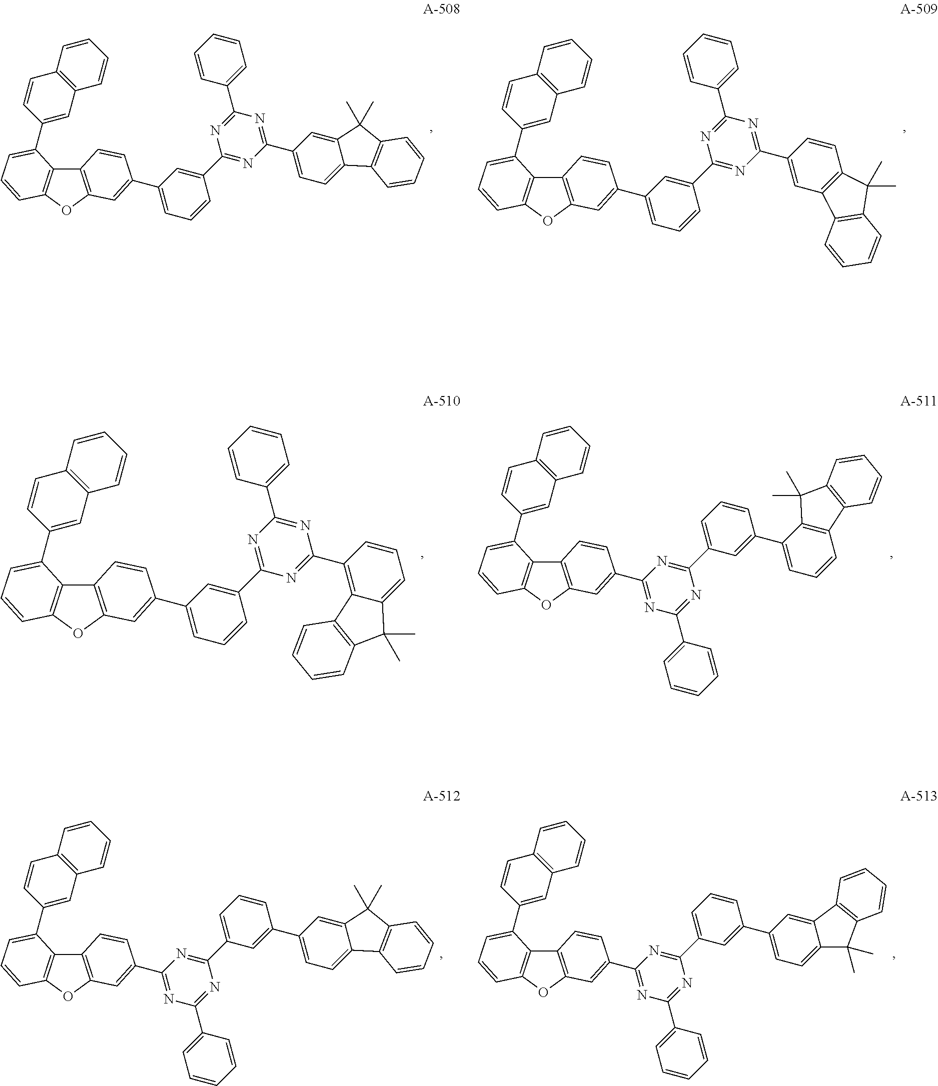 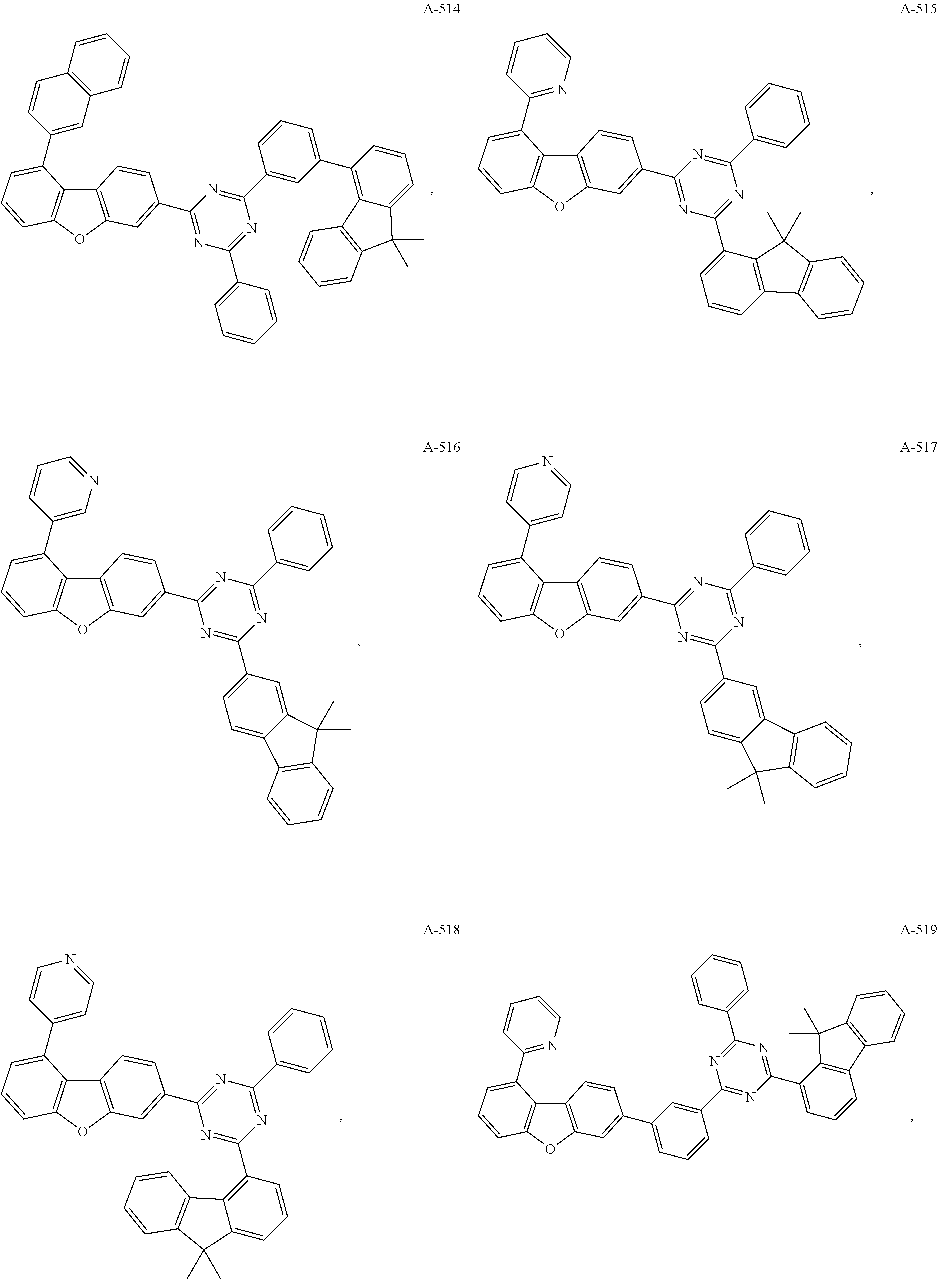 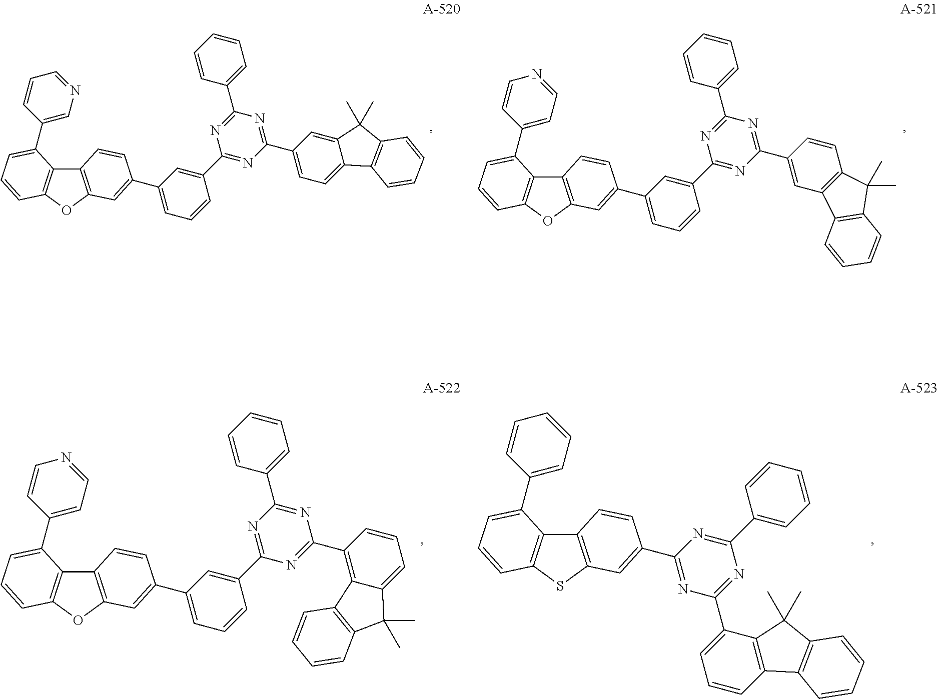 [SMILES: CC1(C)c2ccccc2-c2ccc(-c3nc(-c4ccccc4)nc(-c4cccc(-c5ccc6c(c5)oc5cccc(-c7cccnc7)c56)c4)n3)cc21 ; CC1(C)c2ccccc2-c2cc(-c3nc(-c4ccccc4)nc(-c4cccc(-c5ccc6c(c5)oc5cccc(-c7ccncc7)c56)c4)n3)ccc21 ; CC1(C)c2ccccc2-c2c(-c3nc(-c4ccccc4)nc(-c4cccc(-c5ccc6c(c5)oc5cccc(-c7ccncc7)c56)c4)n3)cccc21 ; CC1(C)c2ccccc2-c2cccc(-c3nc(-c4ccccc4)nc(-c4ccc5c(c4)sc4cccc(-c6ccccc6)c45)n3)c21] 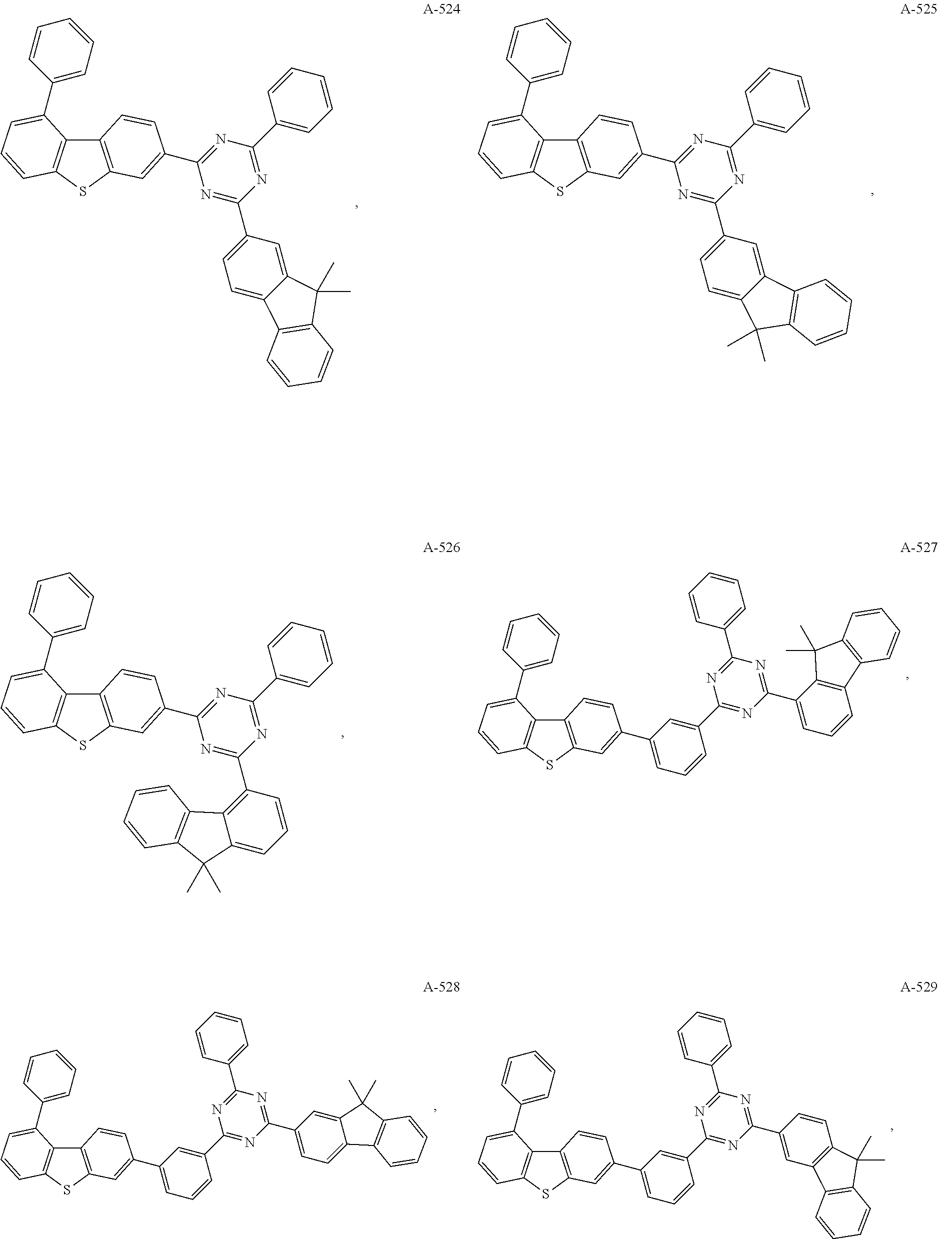 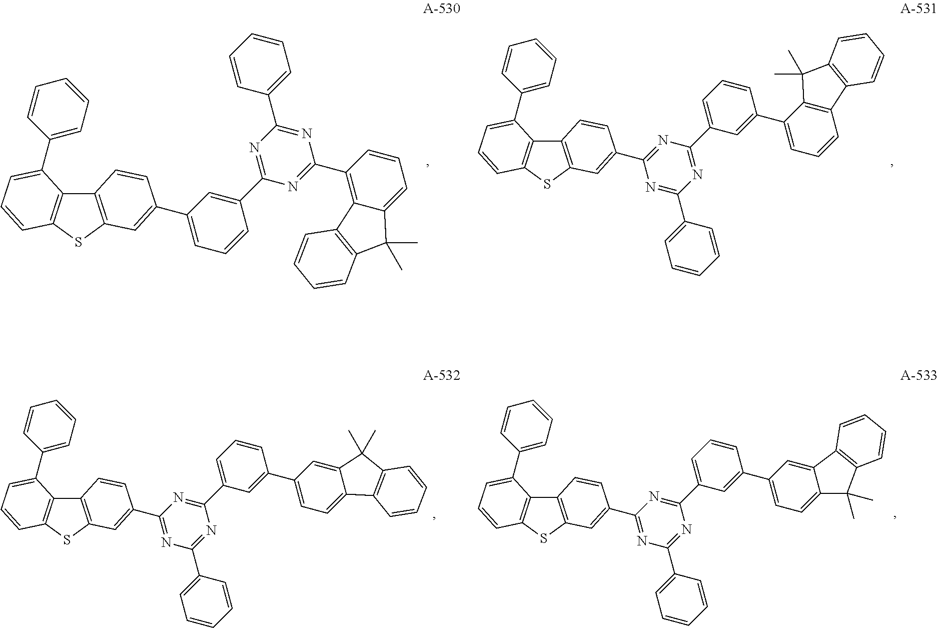 [SMILES: CC1(C)c2ccccc2-c2c(-c3nc(-c4ccccc4)nc(-c4cccc(-c5ccc6c(c5)sc5cccc(-c7ccccc7)c56)c4)n3)cccc21 ; CC1(C)c2ccccc2-c2cccc(-c3cccc(-c4nc(-c5ccccc5)nc(-c5ccc6c(c5)sc5cccc(-c7ccccc7)c56)n4)c3)c21 ; CC1(C)c2ccccc2-c2ccc(-c3cccc(-c4nc(-c5ccccc5)nc(-c5ccc6c(c5)sc5cccc(-c7ccccc7)c56)n4)c3)cc21 ; CC1(C)c2ccccc2-c2cc(-c3cccc(-c4nc(-c5ccccc5)nc(-c5ccc6c(c5)sc5cccc(-c7ccccc7)c56)n4)c3)ccc21] 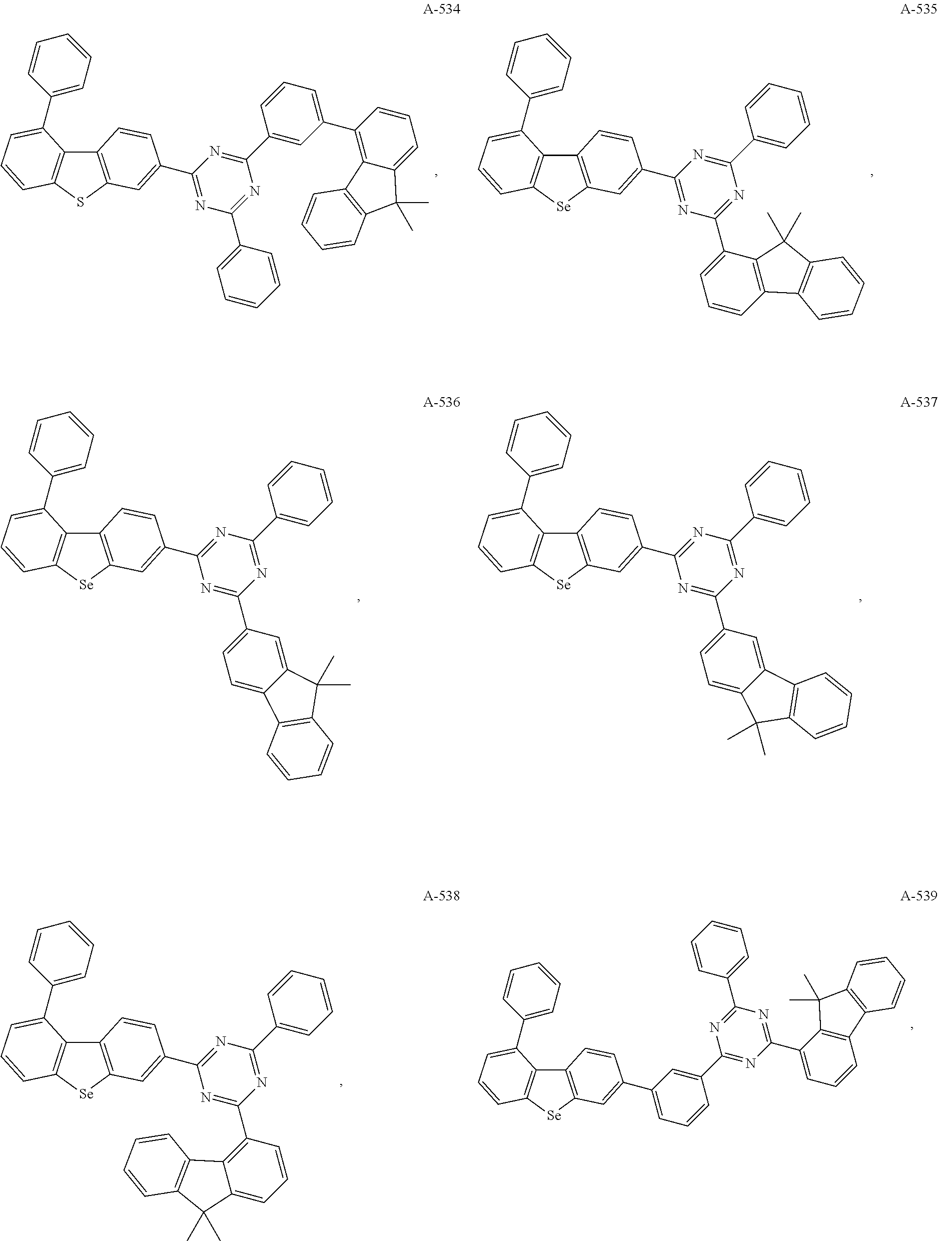 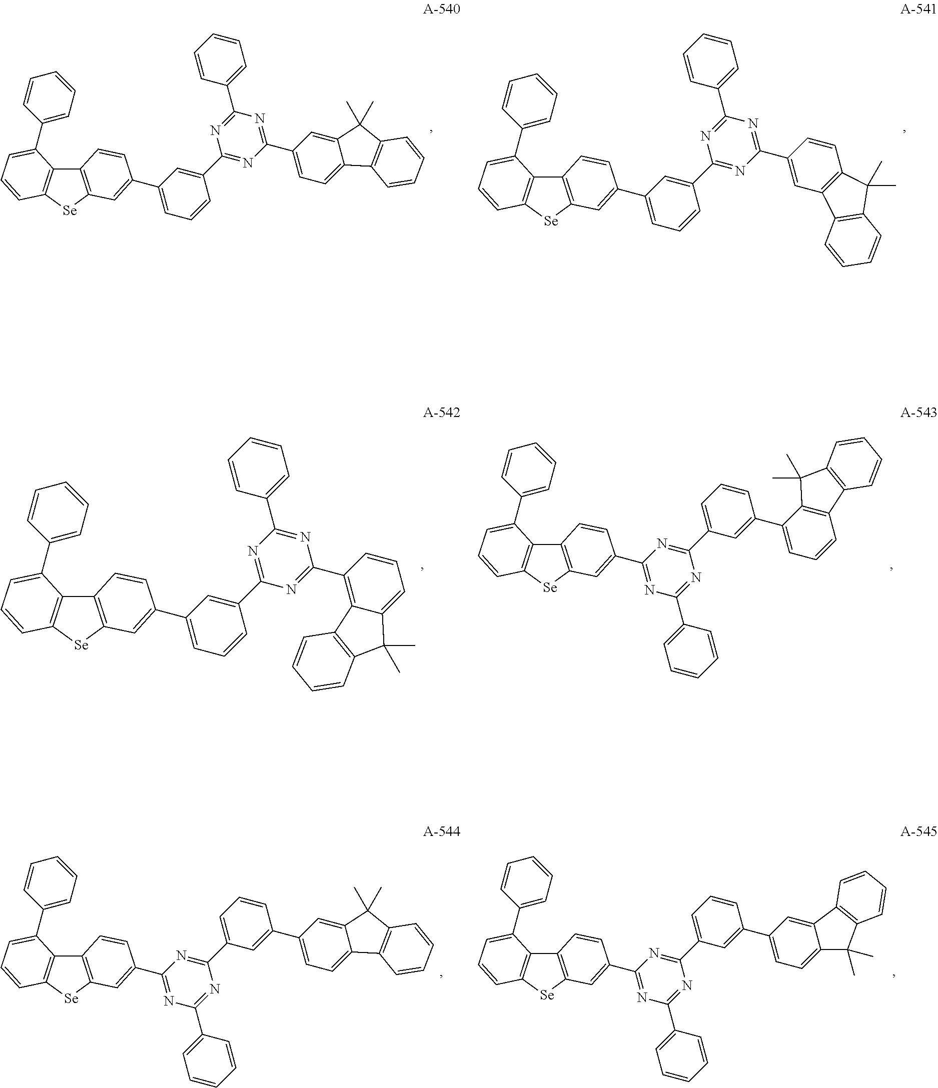 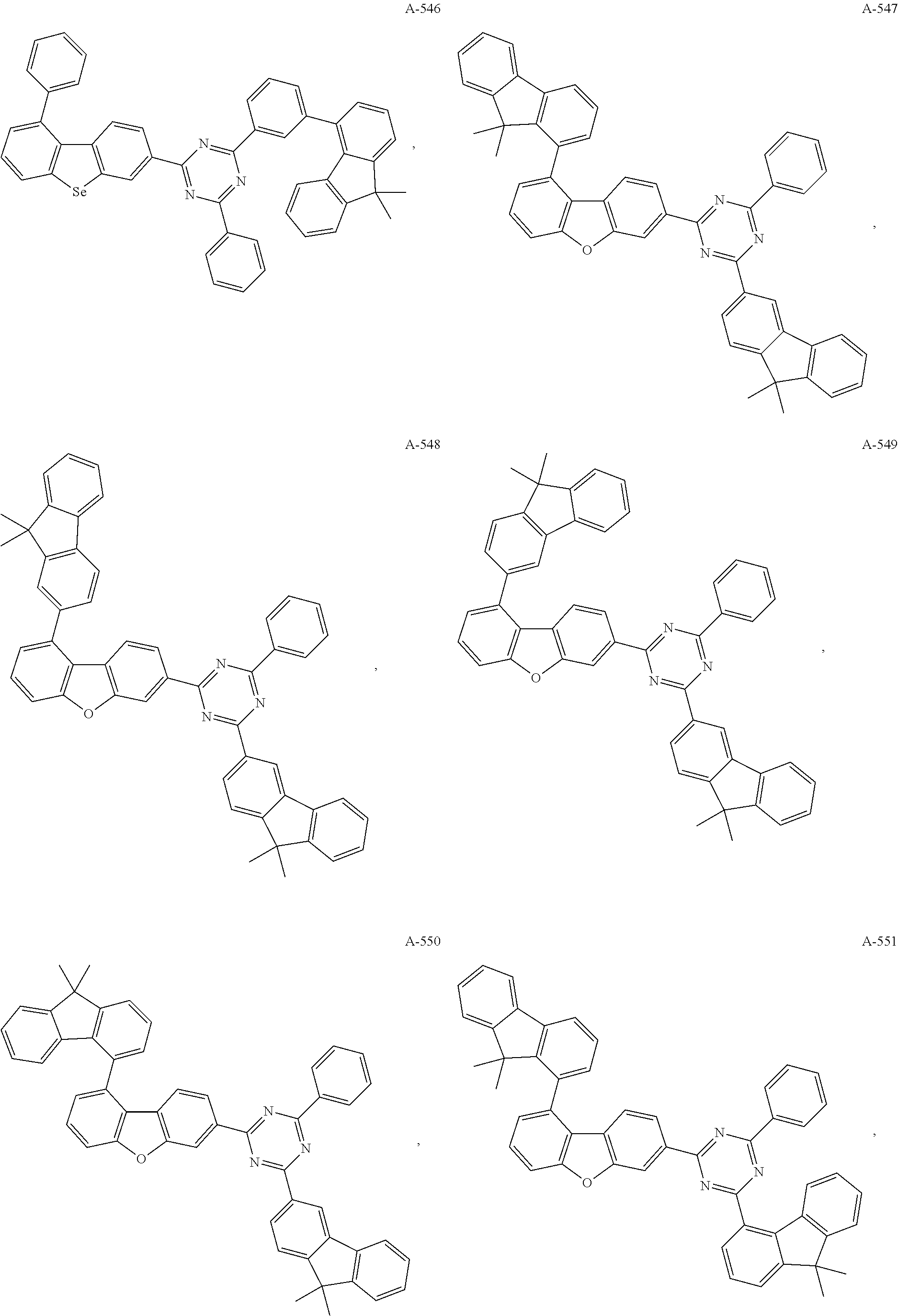 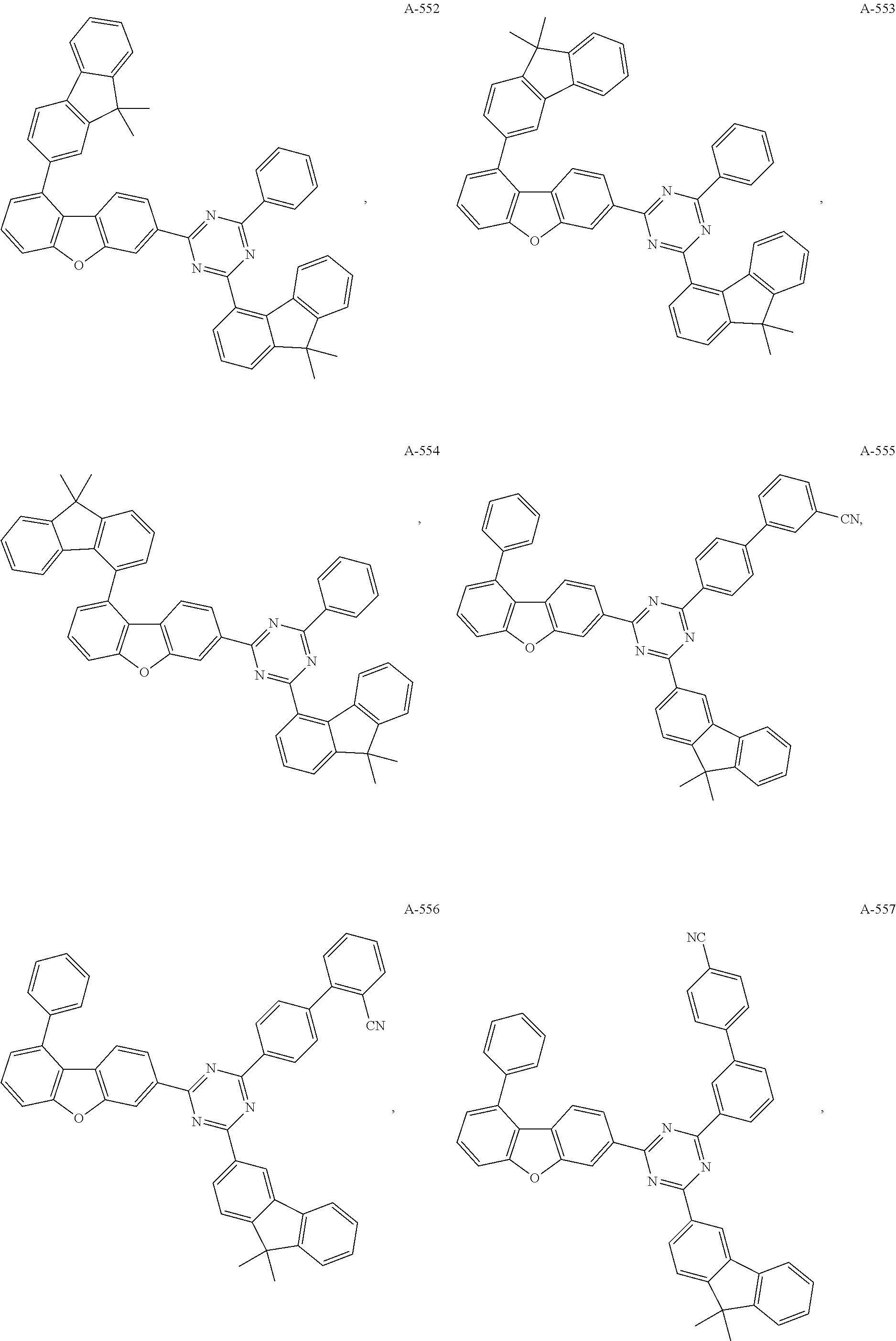 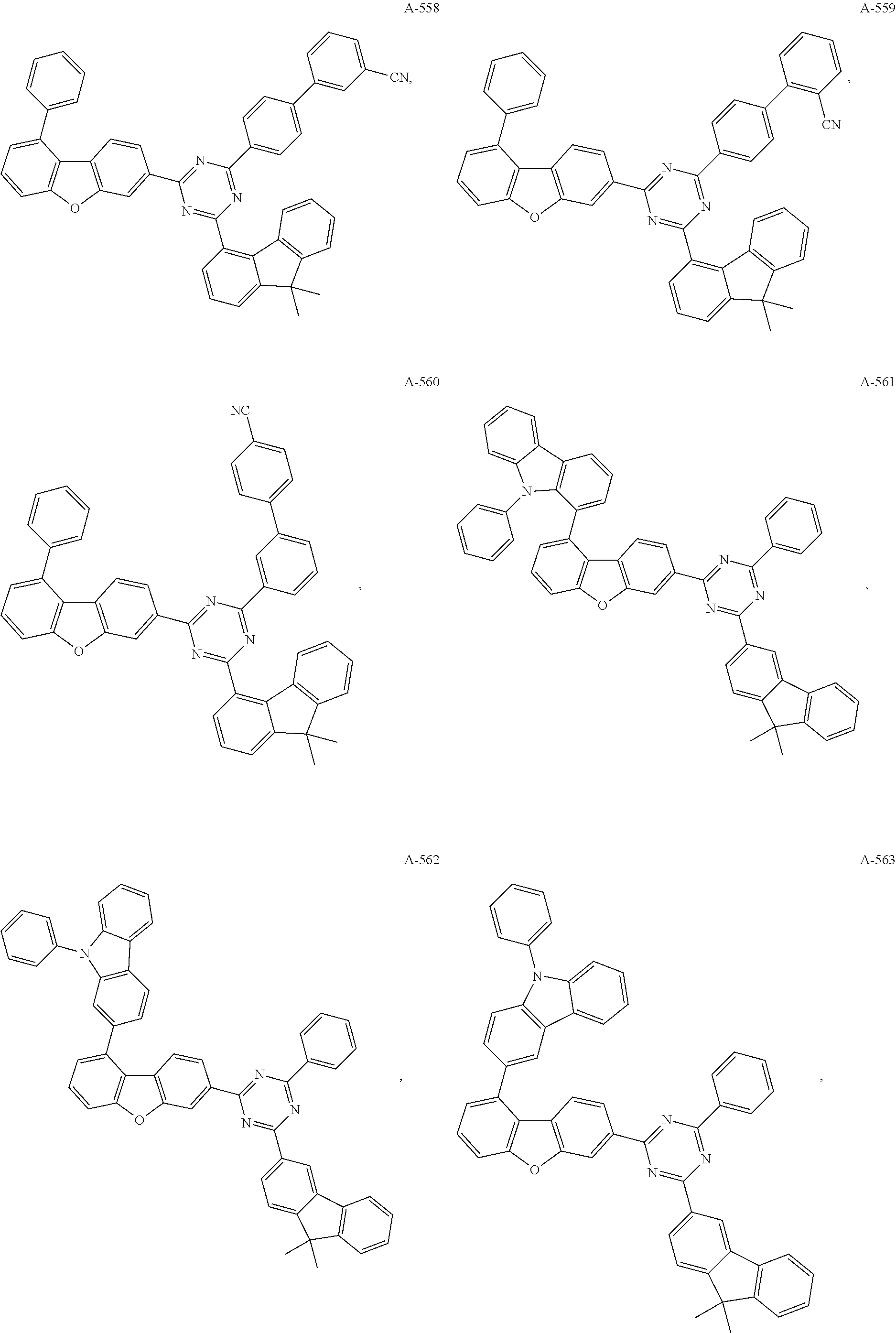 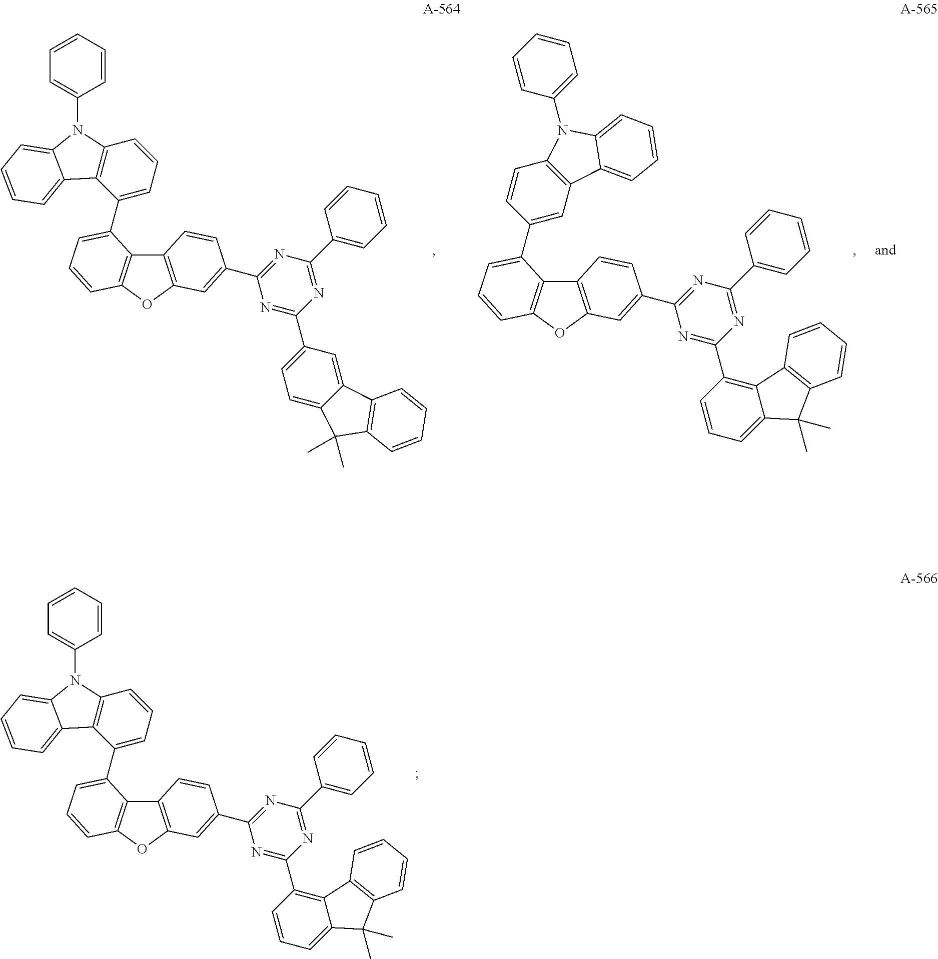 [SMILES: CC1(C)c2ccccc2-c2cc(-c3nc(-c4ccccc4)nc(-c4ccc5c(c4)oc4cccc(-c6cccc7c6c6ccccc6n7-c6ccccc6)c45)n3)ccc21 ; CC1(C)c2ccccc2-c2c(-c3nc(-c4ccccc4)nc(-c4ccc5c(c4)oc4cccc(-c6ccc7c(c6)c6ccccc6n7-c6ccccc6)c45)n3)cccc21 ; CC1(C)c2ccccc2-c2c(-c3nc(-c4ccccc4)nc(-c4ccc5c(c4)oc4cccc(-c6cccc7c6c6ccccc6n7-c6ccccc6)c45)n3)cccc21]
optionally, hydrogens in Compound A-1 to Compound A-566 can be partially or fully substituted with deuterium.

10. An organic electroluminescent device, comprising an anode, a cathode, and an organic layer disposed between the anode and the cathode, wherein at least one layer of the organic layer comprises the compound according to claim 1.

11. The organic electroluminescent device according to claim 10, wherein the organic layer comprising the compound is an emissive layer, and the compound is a host compound; or the organic layer comprising the compound is an electron transport layer, and the compound is an electron transporting compound.

12. The organic electroluminescent device according to claim 11, wherein the emissive layer at least comprises a first metal complex, and the first metal complex has a general formula of M(Lₐ)ₘ(L_{b})ₙ(L_{c})_{q};
the metal M is selected from a metal with a relative atomic mass greater than 40;
ligands Lₐ, L_{b} and L_{c} are a first ligand, a second ligand and a third ligand coordinated to the metal M, respectively; ligands Lₐ, L_{b} and L_{c} may be the same or different;
ligands Lₐ, L_{b} and L_{c} can be optionally joined to form a multidentate ligand;
m is 1, 2 or 3, n is 0, 1 or 2, q is 0, 1 or 2, and the sum of m, n and q is equal to an oxidation state of the metal M; when m is greater than or equal to 2, a plurality of Lₐ may be the same or different; when n is 2, two L_{b} may be the same or different; when q is 2, two L_{c} may be the same or different;
the ligand Lₐ has a structure represented by Formula 2: 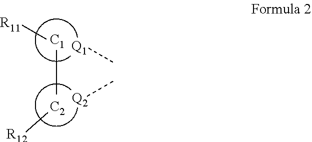
wherein the ring C₁ and the ring C₂ are, at each occurrence identically or differently, selected from an aromatic ring having 5 to 30 ring atoms, a heteroaromatic ring having 5 to 30 ring atoms or combinations thereof,
Q₁ and Q₂ are, at each occurrence identically or differently, selected from C or N;
R₁₁ and R₁₂ represent, at each occurrence identically or differently, mono-substitution, multiple substitutions or non-substitution;
R₁₁ and R₁₂ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof,
adjacent substituents R₁₁ and R₁₂ can be optionally joined to form a ring;
ligands L_{b} and L_{c} are, at each occurrence identically or differently, selected from a monoanionic bidentate ligand;
preferably, ligands L_{b} and L_{c} are, at each occurrence identically or differently, selected from the group consisting of the following structures: 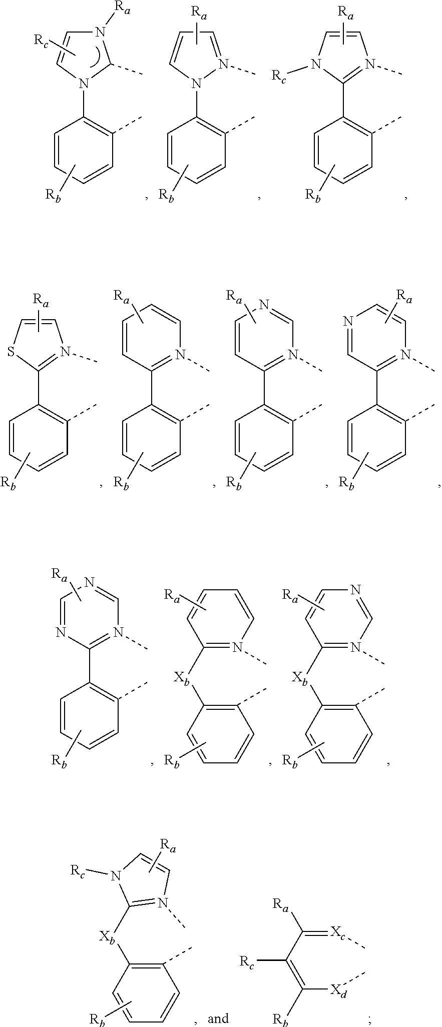
wherein
Rₐ and R_{b} represent, at each occurrence identically or differently, mono-substitution, multiple substitutions or non-substitution;
X_{b} is, at each occurrence identically or differently, selected from the group consisting of: O, S, Se, NR_{N1}, and CR_{C1}R_{C2};
X_{c} and X_{d} are, at each occurrence identically or differently, selected from the group consisting of: O, S, Se, and NR_{N2};
Rₐ, R_{b}, R_{c}, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof,
adjacent substituents Rₐ, R_{b}, R_{c}, R_{N1}, R_{N2}, R_{C1} and R_{C2} can be optionally joined to form a ring.

13. The organic electroluminescent device according to claim 11, wherein the emissive layer further comprises a second host compound, wherein the second host compound comprises at least one chemical group selected from the group consisting of: benzene, pyridine, pyrimidine, triazine, carbazole, azacarbazole, indolocarbazole, dibenzothiophene, azadibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene, and combinations thereof,
preferably, the second host compound comprises at least one chemical group selected from the group consisting of: benzene, carbazole, indolocarbazole, fluorene, silafluorene, and combinations thereof.

14. The organic electroluminescent device according to claim 13, wherein the second host compound has a structure represented by Formula 3 or Formula 4: 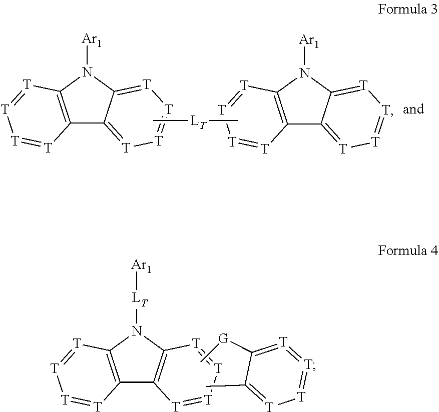
wherein
G is, at each occurrence identically or differently, selected from C(R_{g})₂, NR_{g}, O or S;
L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or combinations thereof,
T is, at each occurrence identically or differently, selected from C, CRₜ or N;
Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof,
Ar₁ is, at each occurrence identically or differently, selected from substituted or unsubstituted aryl having 6 to 30 carbon atoms, substituted or unsubstituted heteroaryl having 3 to 30 carbon atoms or combinations thereof,
adjacent substituents Rₜ, R_{g} can be optionally joined to form a ring;
preferably, the second host compound has a structure represented by one of Formulas 3-a to 3-j and Formulas 4-a to 4-f: 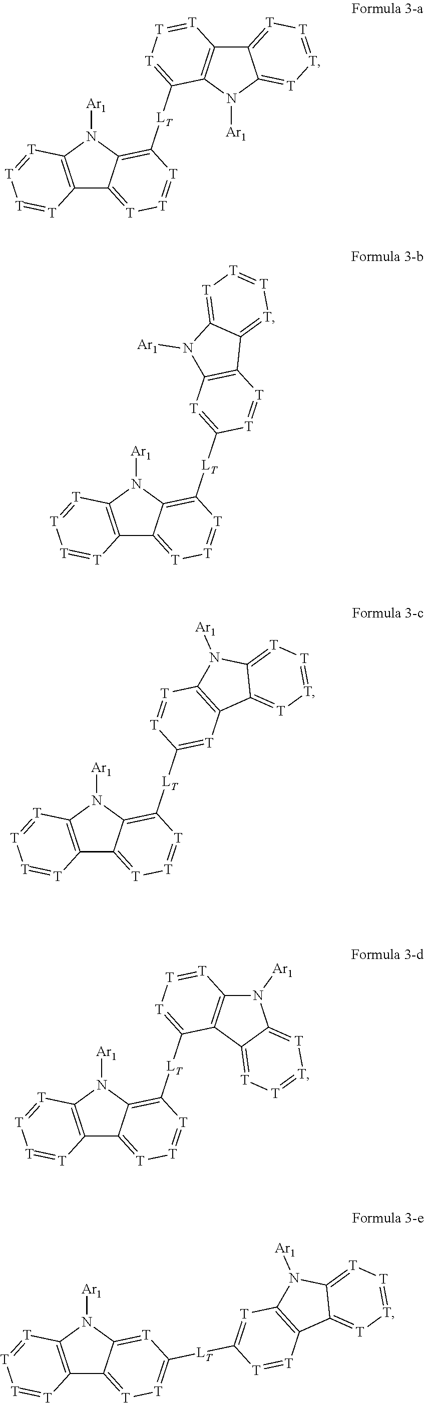 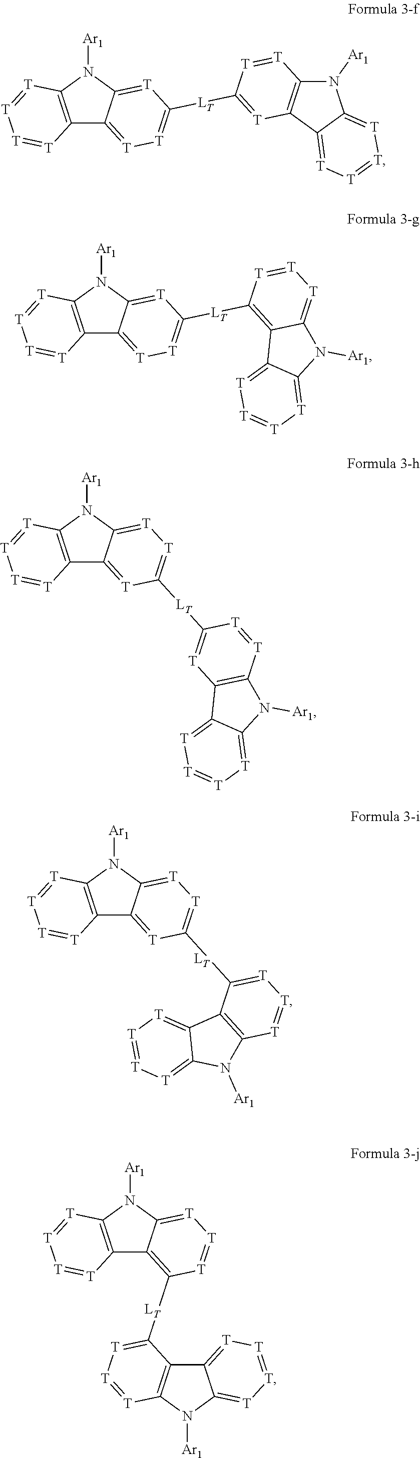 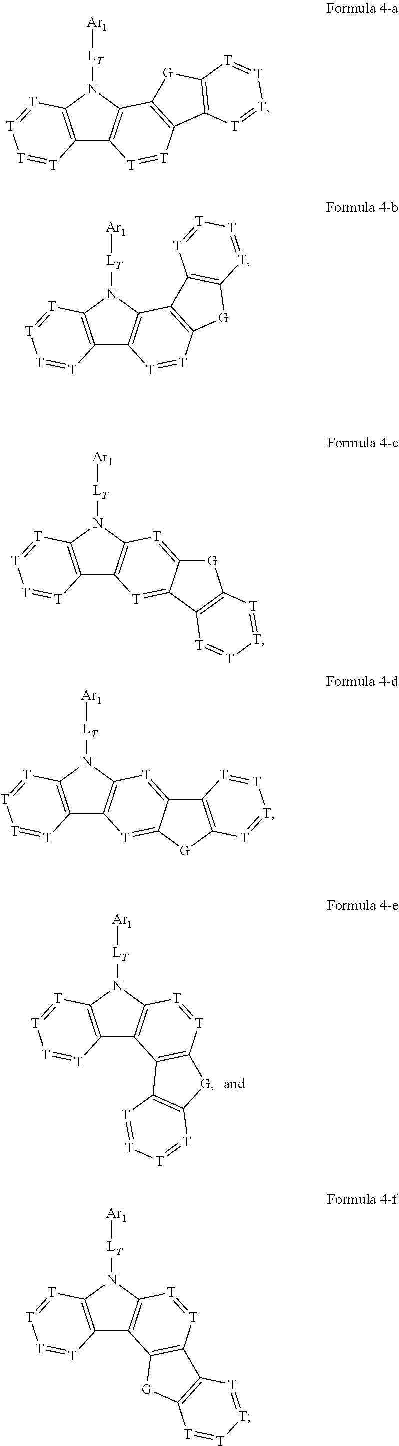
wherein in Formulas 3-a to 3-j, T, L_{T} and Ar₁ each have the same meaning as in Formula 3;
in Formulas 4-a to 4-f, T, G, L_{T} and Ar₁ each have the same meaning as in Formula 4.

15. A compound composition, comprising the compound according to claim 1.
